(19) United States
(12) Patent Application Publication
(10) Pub. No.: US 2026/0365708 A1
(43) Pub. Date: Nov. 19, 2026

(54) FUSED IL10 POLYPEPTIDES

(71) Applicant: Synthekine, Inc., Menlo Park, CA (US)

(72) Inventors: Andrew Morin, Menlo Park, CA (US); Mahalakshmi Ramadass, Fremont, CA (US); Tatjana Petojevic, Menlo Park, CA (US); Bhargavi Jayaraman, Menlo Park, CA (US); Sandro Vivona, Menlo Park, CA (US); Patrick Lupardus, Menlo Park, CA (US)

(21) Appl. No.: 19/487,781

(22) PCT Filed: May 31, 2024
(86) PCT No.: PCT/US2024/032054

§ 371 Date: Nov. 25, 2025

Related U.S. Application Data
(60) Provisional application No. 63/506,000, filed on Jun. 2, 2023.

(51) Int. Cl. C07K 14/54; A61K 38/00; A61P 37/06; C12N 15/86

(52) U.S. Cl. CPC C07K 14/5428; A61P 37/06; C12N 15/86; A61K 38/00; C07K 2319/30; C07K 2319/915

(57) ABSTRACT

The present disclosure relates generally to compositions and methods for modulating signal transduction mediated by interleukin-10 (IL10). In particular, the disclosure provides fused IL10 polypeptides comprising two IL10 monomers. Also provided are compositions and methods useful for producing such fused IL10 polypeptides, as well as methods for modulating IL10-mediated signaling, and/or for the treatment of conditions associated with the perturbation of signal transduction mediated by IL10.

### CROSS-REFERENCE TO RELATED APPLICATION

This application is a U.S. National Phase Application of PCT/US2024/032054, filed May 31, 2024, which claims priority to U.S. Provisional Patent Application No. 63/506,000, filed on Jun. 2, 2023, the disclosure of which is incorporated herein by reference in its entirety for all purposes.

### SEQUENCE LISTING

The contents of the electronic sequence listing (106249-1445592 002901PC_SL.xml; size 391,875 bytes; and date of creation Jun. 14, 2024) is herein incorporated by reference.

### BACKGROUND OF THE DISCLOSURE

Cytokine and growth-factor ligands typically signal through multimerization of cell surface receptor subunits. In some instances, cytokines act as multi-specific (e.g., bispecific or tri-specific) ligands which facilitate the association of the extracellular domains of receptor subunits and resultingly bring the intracellular domains of the receptor subunits into proximity facilitating intracellular signaling. The nature of the cytokine ligand and its interaction with the extracellular domains of receptor subunits determines which and how receptor subunits associate to form a ligand receptor complex and the intracellular signaling characteristic of such complex.

The intracellular domains of some cytokine receptor subunits possess Janus kinase (“JAK”) binding domains. JAK binding domains are typically located in the box1/box region of the intracellular domain of the cytokine receptor subunit near the interior surface of the cell membrane. Intracellular Janus kinases associate with and phosphorylate the JAK binding domains. Four Janus kinases have been identified in mammalian cells: JAK1, JAK2, JAK3 and TYK2. Ihle, et al. (1995) Nature 377(6550):591-4, 1995; O'Shea and Plenge (2012) Immunity 36(4):542-50. When the intracellular domains of cytokine receptor subunits containing JAK binding domains are brought into proximity, the Janus kinases facilitate transphosphorylation of the JAK binding domains. Phosphorylation of the JAK induces conformational changes in the JAK providing the ability of the JAK to further phosphorylate other intracellular proteins. The resulting phosphorylation cascade results in activation of multiple intracellular factors which transduce the intracellular signals associated with the receptor activation by the cytokine ligand. In some instances, the intracellular proteins which are phosphorylated by the JAKs are members of the signal transducer and activator of transcription (“STAT”) protein family. Seven members of the mammalian STAT family have been identified to date: STAT1, STAT2, STAT3, STAT4, STAT5a STAT5b, and STAT6. Delgoffe, et al., (2011) Curr Opin Immunol. 23(5):632-8; Levy and Darnell (2002) Nat Rev Mol Cell Biol. 3(9):651-62 and Murray, (2007) J Immunol. 178(5):2623-9. The selective interplay of activated JAK and STAT proteins, collectively referred to as the JAK/STAT pathway, are involved in the variety of intracellular responses observed in response to cytokine binding. Such intracellular responses initiated by the binding of a cytokine to its receptor are frequently referred to as downstream signaling.

The cytokine human interleukin-10 (hIL10), also known as human cytokine synthesis inhibitory factor (CSIF), is classified as a type (class)-2 cytokine, a group of cytokines that includes IL19, IL20, IL22, IL24 (Mda-7), and IL26, interferons (IFN-α, -β, -γ, -δ, -ε, -κ, -Ω, and -τ) and interferon-like molecules (such as limitin, IL28A, IL28B, and IL29). hIL10 is a non-covalent homodimer comprised of two hIL10 monomer polypeptides. Each hIL10 monomer polypeptide is a 160 amino acid polypeptide with two intramolecular disulfide bonds. Each hIL10 monomer is expressed as a proprotein comprised of 178 amino acids, the first 18 amino acids of which comprise a signal peptide. Although hIL10 is predominantly expressed by macrophages, expression has also been detected in activated T cells, B cells, mast cells, NK cells, dendritic cells, eosinophils, neutrophils and monocytes.

Human IL10 exerts its effect on cells through its interaction with the hIL10 receptor (hIL10R). hIL10R is a type II cytokine receptor comprising the hIL10Rα and hIL10Rβ subunits, which are also referred to as hIL10R1 and hIL10R2, respectively. The hIL10Rα receptor subunit is a “private” or “proprietary” subunit exclusive to the hIL10 receptor. In contrast, the hIL10Rβ subunit is shared with other cytokine receptors including IL22, IL26, IL28, and the interferon lambda L1 (IFNλ1) receptor complexes. Activation of the hIL10R is characterized by the binding of each hIL10 monomer to one hIL10Rα and one hIL10Rβ subunit of hIL10R. Each monomer of the dimeric hIL10 cytokine associates with one hIL10Rα and one hIL10Rβ subunit resulting in a hexameric ligand/receptor hIL10R complex comprised of two hIL10 monomers, two hIL10Rα subunits and two hIL10Rβ subunits.

The hIL10Rα receptor subunit is a transmembrane protein expressed as a 578 amino acid proprotein comprising a N-terminal 21 amino acid signal sequence. The amino acid sequence of the mature canonical hIL10Rα receptor subunit is a 557 amino acid polypeptide of the sequence:

(SEQ ID NO: 1)
HGTELPSPPSVWFEAEFFHHILHWTPIPNQSESTCYEVALLRYGIESWNS
ISNCSQTLSYDLTAVTLDLYHSNGYRARVRAVDGSRHSNWTVTNTRFSVD
EVTLTVGSVNLEIHNGFILGKIQLPRPKMAPANDTYESIFSHFREYEIAI
RKVPGNFTFTHKKVKHENFSLLTSGEVGEFCVQVKPSVASRSNKGMWSKE
ECISLTRQYFTVTNVIIFFAFVLLLSGALAYCLALQLYVRRRKKLPSVLL
FKKPSPFIFISQRPSPETQDTIHPLDEEAFLKVSPELKNLDLHGSTDSGF
GSTKPSLQTEEPQFLLPDPHPQADRTLGNREPPVLGDSCSSGSSNSTDSG
ICLQEPSLSPSTGPTWEQQVGSNSRGQDDSGIDLVQNSEGRAGDTQGGSA
LGHHSPPEPEVPGEEDPAAVAFQGYLRQTRCAEEKATKTGCLEEESPLTD
GLGPKFGRCLVDEAGLHPPALAKGYLKQDPLEMTLASSGAPTGQWNQPTE
EWSLLALSSCSDLGISDWSFAHDLAPLGCVAAPGGLLGSFNSDLVTLPLI
SSLQSSE

(UniProt Reference No. Q13651). Residues 22-235 of SEQ ID NO:1 (amino acids 1-214 of the mature hIL10Rβ protein) correspond to the extracellular domain (ECD), residues 236-256 of SEQ ID NO:1 (amino acids 215-235 of the mature hIL10Rβ protein) correspond to the transmembrane domain (TM) and residues 257-578 of SEQ ID NO:1 (amino acids 236-557) of the mature hIL10Rβ protein) correspond to the intracellular domain (ICD).

The human IL10Rβ (hIL10Rβ) receptor subunit is a transmembrane protein expressed as a 325 amino acid pro-protein comprising a 19 amino acid N-terminal signal. The amino acid sequence of the mature canonical hIL10Rb receptor subunit is a 306 amino acid polypeptide of the sequence:

(SEQ ID NO: 2)
MVPPPENVRMNSVNFKNILQWESPAFAKGNLTFTAQYLSYRIFQDKCMNT
TLTECDFSSLSKYGDHTLRVRAEFADEHSDWVNITFCPVDDTIIGPPGMQ
VEVLADSLHMRFLAPKIENEYETWTMKNVYNSWTYNVQYWKNGTDEKFQI
TPQYDFEVLRNLEPWTTYCVQVRGFLPDRNKAGEWSEPVCEQTTHDETVP
SWMVAVILMASVFMVCLALLGCFALLWCVYKKTKYAFSPRNSLPQHLKEF
LGHPHHNTLLFFSFPLSDENDVFDKLSVIAEDSESGKQNPGDSCSLGTPP
GQGPQS

(UniProt Reference No. Q08334). Amino acids 20-220 (amino acids 1-201 of the mature hIL10Rβ protein) correspond to the extracellular domain, amino acids 221-242 (amino acids 202-223 of the mature hIL10Rβ protein) correspond to the 22 amino acid transmembrane domain, and amino acids 243-325 (amino acids 224-306 of the mature hIL10Rβ protein) correspond to the intracellular domain.

The murine IL10Rβ (mIL10Rβ) receptor subunit is expressed as a 349 amino acid pro-protein comprising a 19 amino acid N-terminal signal sequence. The amino acid sequence of the mature canonical hIL10Rb receptor subunit is a 330 amino acid polypeptide of the sequence:

(SEQ ID NO: 3)
MIPPPEKVRMNSVNFKNILQWEVPAFPKTNLTFTAQYESYRSFQDHCKRT
ASTQCDFSHLSKYGDYTVRVRAELADEHSEWVNVTFCPVEDTIIGPPEMQ
IESLAESLHLRFSAPQIENEPETWTLKNIYDSWAYRVQYWKNGTNEKFQV
VSPYDSEVLRNLEPWTTYCIQVQGFLLDQNRTGEWSEPICERTGNDEITP
SWIVAIILIVSVLVVFLFLLGCFVVLWLIYKKTKHTFRSGTSLPQHLKEF
LGHPHHSTFLLFSFPPPEEAEVFDKLSIISEESEGSKQSPEDNCASEPPS
DPGPRELESKDEAPSPPHDDPKLLTSTSEV.

mIL10Rβ is referenced at UniProtKB database as entry Q61190. Amino acids 20-220 (amino acids 1-201 of the mature protein) correspond to the extracellular domain, amino acids 221-241 (amino acids 202-222 of the mature protein) correspond to the 21 amino acid transmembrane domain, and amino acids 242-349 (amino acids 223-330 of the mature protein) correspond to the intracellular domain.

The interaction of IL10 with its receptor and receptor subunits have been studied and described in the scientific literature. Pletnev, et al. provide information relating to the structures of the soluble receptor chain of IL10R2 and the ternary complex of IL10/sIL10R1/sIL10R2 and residues involved in ligand-receptor and receptor-receptor interactions. Pletnev, et al. (2005) BMC Structural Biology 5:10. Although the interaction between hIL10 and the hIL10Rα receptor subunit is a specific high-affinity interaction, the association of hIL10 with hIL10Rβ is a comparatively low affinity interaction. Reports suggest that the interaction of hIL10 with hIL10Rα induces a conformational change which in hIL10 and/or hIL10Rα which facilitates the binding of the [hIL10:hIL10Rα] complex to hIL10Rβ. The formation of the ternary [hIL10:hIL10Rα: hIL10Rβ] complex is suggested as the rate limiting factor in initiating hIL10 signaling.

The interaction of IL-10 with the IL10R effects the activation of JAK1 (associated with hIL10Rα) and Tyk2 (associated with hIL10Rβ) and induces the activation of STAT1, STAT3, and, in some cells, STAT5. STAT3 is recruited directly to the hIL-10/hIL-10R complex via either of two tyrosine residues in the hIL10Rα cytoplasmic domain that become phosphorylated in response to hIL-10 and are required for hIL-10 signaling.

Homodimerization of STAT3 results in its release from the receptor and translocation of the phosphorylated STAT homodimer into the nucleus, where it binds to STAT3-binding elements in the promoters of numerous genes, including the promoter of IL10, which is positively regulated by STAT3. The hIL10 receptor intracellular domain possesses sequences that are linked to its anti-inflammatory activity that are not shared by other STAT3 activating cytokine receptors. Riley, et al. (1999) Journal of Biological Chemistry 274(23):15967-16664.

The expression of the hIL10Rα and IL10Rβ receptor subunits vary with respect to cell type and the activation state of the cell. The activation state of cells that express hIL10Rα may result in substantial variability in the levels of expression. The expression hIL10Rα in hematopoietic cells, which constitutively express low levels of hIL10Rα, is frequently substantially upregulated by various stimuli. In contrast to hIL10Rα, which is expressed primarily on hematopoietic cells, the IL10Rβ receptor subunit is expressed ubiquitously. While certain cell types express hIL10Rβ at different levels, the level of hIL10Rβ expression in a given cell type is typically less affected by the activation state of the cell than hIL10Rα.

hIL10 is associated with a wide variety of functions and exhibits both immunosuppressive and immunostimulatory activities through its interaction with T cells, B cells, macrophages, and antigen presenting cells (APCs). The immunosuppressive activity of hIL10 is well documented. hIL10 is associated with suppressing the expression of IL1α, IL1β, IL6, IL8, TNFα, GM-CSF and G-CSF in activated monocytes and activated macrophages, as well as suppression of proinflammatory cytokine interferon-gamma (INFγ) production by NK cells. However, hIL10 also demonstrates an immunostimulatory effect by stimulation of the production of the proinflammatory cytokine IFN-γ by CD8+ T cells. The immunostimulatory and immunosuppressive properties of hIL10 have proved to be a challenge in the clinical application of hIL10 in the treatment of human disease.

Saxton, et al. describe the interaction of IL10 with the IL10Rβ subunit and amino acid residues that are involved in the binding of IL10 to IL10Rβ and that modification of such residues can potentially provide IL10 variants that retain the immunosuppressive function of IL10 on myeloid cells but have diminished pro-inflammatory activity on CD8+ T cells. Saxton, et al. (2021) Science 371(6535), eabc8433.

### SUMMARY OF THE DISCLOSURE

The present disclosure provides compositions that are useful for modulating signal transduction mediated by interleukin-10 (IL10). In particular, the disclosure provides fused IL10 polypeptides comprising two IL10 monomers, hIL10A and hIL10B, that can be connected with or without a linker and are each independently selected from the group consisting of a wild-type hIL10 (SEQ ID NO:4) and hIL10 muteins. Also provided are compositions and methods useful for producing such fused IL10 polypeptides, as well as methods for modulating IL10-mediated signaling, and for the treatment of conditions associated with the perturbation of signal transduction mediated by IL10.

In one aspect, present disclosure provides a fused human IL10 (hIL10) polypeptide comprising a polypeptide of the formula:

(hIL10A)-Lₙ-(hIL10B),
wherein:
(a) hIL10A and hIL10B are each human IL10 (hIL10) sequences independently selected from the group consisting of a wild-type hIL10 (SEQ ID NO:4) and hIL10 muteins, wherein the hIL10 muteins each independently comprises one or more amino acid substitutions at positions corresponding to residues T100, H14, N18, N21, M22, R24, D25, D28, R32, E74, H90, N92, S93, E96, and R104 of SEQ ID NO: 4, optionally the hIL10A has an amino-terminal deletion of 1, 2, 3, 4, 5, 6, 7, 8, 9, or 10 amino acid residues relative to SEQ ID NO:4, and/or the hIL10B has an amino-terminal deletion of 1, 2, 3, 4, 5, 6, 7, 8, 9, or 10 amino acid residues relative to SEQ ID NO:4;
(b) L is an amino acid linker of from 1 to 30 amino acids in length; and
(c) n=0 (absent) or 1 (present).

In some embodiments of the fused hIL10 polypeptide,
(a) the amino acid substitution at position T100 is selected from the group consisting of T100L, T100D, T100V, T100E, T100A, T100R, T100N, T100Q, T100E, T100I, T100K, T100M, and T100S;
(b) the amino acid substitution at position H14 is selected from the group consisting of H14C, H14F, H14P, H14W, H14G, H14A, H14D, H14E, H14I, H14K, H14L, H14M, H14N, H14Q, H14R, H14S, H14T, H14Y, and H14V;
(c) the amino acid substitution at position N18 is selected from the group consisting of N18Y, N18F, N18A, N18D, N18E, N18L, N18V, N18S, N18T, N18I, N18V, N18M, N18R, N18K, and N18H;
(d) the amino acid substitution at position N21 is selected from the group consisting of N21A, N21R, N21Q, N21H, N21K, N21S, N21V, N21I, N21L, N21M, N21T N21C, N21D, and N21E;
(e) the amino acid substitution at position M22 is selected from the group consisting of M22A, M22V, M22I, M22L, M22N, M22D, M22S, M22T, M22W, and M22Q;
(f) the amino acid substitution at position R24 is selected from the group consisting of R24E, R24D, R24N, R24Q, R24A, R24S, and R24T;
(g) the amino acid substitution at position D25 is selected from the group consisting of D25A, D25N, D25H, D25I, D25K, D25L, D25P, D25Q, and D25V;
(h) the amino acid substitution at position D28 is selected from the group consisting of D28A, D28E, D28L, D28V, D28S, D28T, D28I, D28V, D28M, D28H, D28K, and D28R;
(i) the amino acid substitution at position R32 is selected from the group consisting of R32A, R32D, R32E, R32L, R32V, R32S, R32T, R32I, R32V, R32M, R32N, R32Q, R32G, R32C, R32P, R32F, R32Y, and R32H;
(j) the amino acid substitution at position E74 is selected from the group consisting of E74A, E74D, E74L, E74V, E74S, E74T, E741, E74V, E74M, E74H, E74K, and E74R;
(k) the amino acid substitution at position H90 is selected from the group consisting of H90A, H90D, H90E, H90I, H90K, H90L, H90M, H90N, H90Q, H90R, H90S, H90T, H90Y, and H90V;
(l) the amino acid substitution at position N92 is selected from the group consisting of N92D, N92Q, N92E, N92H, N92K, N92S, N92V, N92I, N92L, N92M, N92T, and N92A;
(m) the amino acid substitution at position S93 is selected from the group consisting of S93E, S93A, S93R, S93N, S93D, S93Q, S93E, S93I, S93L, S93K, S93M, S93G, and S93V;
(n) the amino acid substitution at position E96 is selected from the group consisting of E96C, E96F, E96Y, E96W, E96A, E96N, E96D, E96Q, E96H, E96K, and E96S; and
(o) the amino acid substitution at position R104 is selected from the group consisting of R104A, R104W, R104Y, R104F, R104H, R104D, R104E, R104N, R104Q, R104S, R104T, R104I, R104L, R104V, and R104M.

In some embodiments, at least one of the hIL10A and the hIL10B is an hIL10 mutein. In particular embodiments, hIL10A and the hIL10B are both hIL10 muteins. In particular embodiments, hIL10A and the hIL10B are the same. In particular embodiments, hIL10A and the hIL10B are different.

In some embodiments, n=0 (absent). In some embodiments, n=1 (present) and L is a polypeptide having 1, 2, 3, 4, 5, 6, 7, 8, 9, 10, 11, 12, 13, 14 15, 16, 17, 18, 19, 20, 21, 22, 23, 24, 25, 26, 27, 28, 29, or 30 amino acids. In some embodiments, L is a GS linker. In particular embodiments, the GS linker comprises a sequence of GGGSGSGSGSG (SEQ ID NO:19).

In some embodiments, the hIL10A and the hIL10B are each independently selected from a wild-type hIL10 (SEQ ID NO:4) or an IL10 mutein comprising an amino acid substitution selected from the group consisting of N21D, N21E, N21K, M22A, M22S, M22T, M22D, M22W, R24E, D25K, E96K, E96Q, T100E, T100C and T100L. In particular embodiments, the hIL10A and/or the hIL10B comprises the amino acid substitution T100L. In some embodiments, the hIL10A and/or hIL10B comprises the amino acid substitution M22A or M22S. In some embodiments, the hIL10A and/or hIL10B comprises the amino acid substitution E96Q. In some embodiments, the hIL10A and/or hIL10B comprises the amino acid substitution R24E. In some embodiments, the hIL10A and/or hIL10B comprises the amino acid substitution D25K. In some embodiments, the hIL10A and/or hIL10B comprises the amino acid substitution N21K.

In some embodiments of the furst hIL10 polypeptides, hIL10A and/or hIL10B is each a polypeptide having at least 90% (e.g., 90%, 91%, 92%, 93%, 94%, 95%, 96%, 97%, 98%, 99%, or 100%) sequence identity to a polypeptide selected from the group consisting of SEQ ID NOS: 6, 7, 8, 9, 10, 11, 12, 13, 14, 15, 16, 17, 18, 148, 149, 150, 151, 152, 153, 154, 155, 156, 191, 192, 193, 194, 195, 196, 197, 198, 199, 200, 201, 202, 203, 204, and 205, optionally wherein one or both of hIL10A and hIL10B has an N-terminal deletion of 1, 2, 3, 4, 5, 6, 7, 8, 9, or 10 amino acid residues relative to the sequence of SEQ ID NO:4. In particular embodiments, the hIL10A and hIL10B are the same. In particular embodiments, the hIL10A and hIL10B are different. In particular embodiments, the hIL10A and the hIL10B each comprises an amino acid substitution selected from the group consisting of N21D, N21E, N21K, M22A, M22S, M22T, M22D, M22W, R24E, D25K, E96K, E96Q, T100E, T100C and T100L.

In particular embodiments, the hIL10A is a polypeptide is selected from the group consisting of SEQ ID NOS:191, 192, 193, 194, 195, 196, 197, 198, 199, 200, 201, 202, 203, 204, and 205 and hIL10B is a polypeptide is selected from the group consisting of SEQ ID NOS: 6, 7, 8, 9, 10, 11, 12, 13, 14, 15, 16, 17, 18, 148, 149, 150, 151, 152, 153, 154, 155, and 156.

In some embodiments, the hIL10A and the hIL10B are both hIL10 muteins comprising the amino acid substitution T100L. In particular embodiments, the hIL10A and the hIL10B are both hIL10 muteins comprising the amino acid substitution T100L, hIL10A is a polypeptide having 100% sequence identity to SEQ ID NO: 204 and hIL10 B is a polypeptide having 100% sequence identity to SEQ ID NO: 15.

In some embodiments, the fused hIL10 polypeptide is a polypeptide comprising an amino acid sequence having having at least 95%, alternatively at least 96%, alternatively at least 97%, alternatively at least 98%, alternatively at least 99%, alternatively 100% sequence identity to a polypeptide selected from the group consisting of SEQ ID NOS:24, 25, 26, 27, 28, 29, 30, 31, 32, 33, 34, 35, 36, 37, 38, 39, 40, 41, 42, 43, 44, 45, 46, 47, 48, 49, 50, 119, 157, 158, 159, 160, 161, 162, 163, 164, 165, 166, 167, 168, 169, 170, 171, 172, 173, 174, 175, 176, 177, 178, 179, 180, 181, 182, 183, 184, 185, and 186.

In some embodiments, the fused hIL10 polypeptide comprises an amino acid sequence having 100% sequence identity to a polypeptide selected from the group consisting of SEQ ID NOS: 44, 123, 124, 157, 158, 159, 160, 161, 162, 163, 164, 165, 166, 167, 168, 169, 170, 171, 172, 173, 174, 175, 176, 177, 178, 179, 180, 181, 182, 183, 184, 185, and 186.

In some embodiments, the fused hIL10 polypeptide comprises an amino acid sequence having at least 90% (e.g., 90%, 91%, 92%, 93%, 94%, 95%, 96%, 97%, 98%, 99%, or 100%) sequence identity to a polypeptide selected from the group consisting of SEQ ID NO: 44, SEQ ID NO: 123, SEQ ID NO: 124, SEQ ID NO: 170 and SEQ ID NO: 185. In particular embodiments, the fused hIL10 polypeptide comprises a polypeptide having 100% identity to the amino acid sequence of SEQ ID NO: 44. In particular embodiments, the fused hIL10 polypeptide comprises a polypeptide having 100% identity to the amino acid sequence of SEQ ID NO: 185.

In some embodiments, the fused hIL10 polypeptide modified to extend half-life in vivo. In particular embodiments, the modification to extend half-life in vivo is selected from the group consisting of PEGylation, acylation, albumination or conjugation to an Fc polypeptide. In particular embodiments, the modification to extend half-life in vivo is acylation. In particular embodiments, the modification to extend half-life in vivo is conjugation to an Fc polypeptide. In particular embodiments, the Fc polypeptide is an Fc domain from hIgG1, hIgG2, hIgG3, or hIgG4, or a variant thereof. In particular embodiments, the Fc polypeptide comprises a sequence that is modified from a wild-type Fc polypeptide sequence to reduce effector function. In some embodiments, the Fc polypeptide comprises one or more amino acid substitutions or deletions to promote heterodimerization.

In some embodiments, the fused hIL10 polypeptide is a heterodimeric Fc molecule comprising a first polypeptide of the formula #1:

IL10FP-L1ₐ-UH1-Fc1 [1]

and a second polypeptide of the formula #2:

UH2-Fc2 [2]
wherein:
   hIL10FP is a fused hIL10 polypeptide of the present disclosure (e.g., a fused hIL10 polypeptide of formula (hIL10A)-Lₙ-(hIL10B));
L1 is a linker and a is independently selected from 0 (absent) or 1 (present);
UH1 and UH2 are each an upper hinge domain of human immunoglobulin independently selected from the group consisting of the IgG1, IgG2, IgG3 and IgG4 upper hinge domains, optionally comprising the amino acid substitution C220S (EU numbering);
Fc1 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc2, and
Fc2 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc1,
and optionally wherein the polypeptide of formula [1] and the polypeptide of formula [2] are linked by at least one interchain disulfide bond.

In some embodiments, the modification to extend half-life in vivo is PEGylation. In particular embodiments, the PEG is a linear or branched polyethylene glycol molecule having a molecular weight of from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons. In particular embodiments, the PEG is linear. In particular embodiments, the PEG is branched. In some embodiments, the PEG is a 40 kD branched PEG molecule comprising two 20 kD arms. In particular embodiments, the PEG is covalently attached to the N-terminus of the polypeptide, optionally via a linker.

In particular embodiments of the fused hIL10 polypeptide, the PEG is a 40 kD branched PEG molecule comprising two 20 kD arms of the formula:

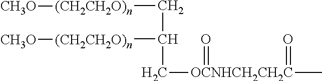

covalently bonded to the N-terminus of hIL10A, optionally via a linker. In particular embodiments, the PEG covalently bonded to the N-terminus of hIL10A via an aldehyde linker.

In some embodiments, the fused hIL10 polypeptide exhibits a greater fraction of an activity of wild-type hIL10 in activated human monocytes than the fraction of the activity of wild-type hIL10 in activated human CD8 T cells. In some embodiments, the activity of wild-type hIL10 is induction of intracellular STAT3 signaling. In some embodiments, the fused hIL10 polypeptide exhibits an Emax of at least 30%, optionally at least 40%, optionally at least 50% of level of activity of wild-type hIL10 in activated human monocytes wherein the activity of wild-type hIL10 in activated human monocytes is selected from the group consisting of inhibition of IL1β secretion and inhibition of TNFα secretion. In some embodiments, the fused hIL10 polypeptide exhibits an Emax less than 30%, optionally less than 20%, optionally less than 10% of level of activity of wild-type hIL10 in activated human CD8 T cells wherein the activity of wild-type hIL10 in activated human CD8 T cells is selected from the group consisting of IFNγ secretion, granzyme A secretion and granzyme B secretion.

In another aspect, the disclosure features a nucleic acid sequence encoding a fused hIL10 polypeptide described herein. In some embodiments, the nucleic acid sequence is an mRNA. In some embodiments, the nucleic acid sequence is a DNA.

In another aspect, the disclosure provides a vector comprising a nucleic acid sequence described herein operably linked with an expression control sequence. In some embodiments, the vector is a viral vector.

In another aspect, the disclosure provides a cell transformed with a vector described herein. In some embodiments, the cell is a mammalian cell.

In another aspect, the disclosure provides a pharmaceutical formulation comprising as an active ingredient: (a) a fused hIL10 polypeptide described herein; (b) a nucleic acid sequence encoding a fused hIL10 polypeptide described herein; (c) a vector comprising the nucleic acid sequence, or (d) a cell transformed with the vector comprising the nucleic acid sequence, and one or more pharmaceutically acceptable solvents, carriers, stabilizers, preservatives, or diluents.

In another aspect, the disclosure provides a method of preventing or treating a mammalian subject suffering from a disease, disorder, or condition, the method comprising administering to said subject: (a) a fused hIL10 polypeptide described herein; (b) a nucleic acid sequence encoding a fused hIL10 polypeptide described herein; (c) a vector comprising the nucleic acid sequence, or (d) a cell transformed with the vector comprising the nucleic acid sequence, or (e) a pharmaceutical formulation described herein.

In some embodiments, the disease, disorder, or condition is an autoimmune disease, disorder, or condition. In particular embodiments, the autoimmune disease, disorder, or condition is an inflammatory bowel disease (IBD). In particular embodiments, the IBD is Crohn's disease or ulcerative colitis.

In some embodiments, the disease, disorder, or condition is cancer. In particular embodiments, the cancer is a cancer arising from chronic inflammation.

In some embodiments, the disease, disorder, or condition is macrophage activation syndrome.

In some embodiments, the disease, disorder, or condition is interferon gamma induced anemia.

In another aspect, the disclosure provides a method of making a fused human IL10 (hIL10) polypeptide of the formula (hIL10A)-Lₙ-(hIL10B), the method comprising the steps of transfecting a host cell with a vector comprising a nucleic acid sequence encoding a fused IL10 polypeptide described herein, culturing said cell in a culture medium under conditions to permit expression of the nucleic acid sequence encoding the fused hIL10 polypeptide, and isolating the fused hIL10 polypeptide from the culture medium, optionally further comprising the step of purification of the fused hIL10 polypeptide.

In some embodiments, the host cell is a procaryotic cell. In some embodiments, the procaryotic cell is an E. coli cell.

In some embodiments, the hIL10A is a polypeptide selected from the group consisting of SEQ ID NOS:191, 192, 193, 194, 195, 196, 197, 198, 199, 200, 201, 202, 203, 204, and 205, and the hIL10B is a polypeptide selected from the group consisting of SEQ ID NOS: 6, 7, 8, 9, 10, 11, 12, 13, 14, 15, 16, 17, 18, 148, 149, 150, 151, 152, 153, 154, 155, and 156. In particular embodiments, the hIL10A is a polypeptide selected from the group consisting of SEQ ID NOS: 172, 173, 174, 175, 176, 177, 178, 179, 180, 181, 182, 183 184, 185, and 186.

### BRIEF DESCRIPTION OF THE DRAWINGS

FIG. 1A. shows the results of an assay to evaluate the levels of the production of STAT3 (y-axis) in activated monocytes of the indicated fused hIL10 polypeptides in response to varying concentrations (x-axis) of the fused hIL10 polypeptides as more fully described in the Examples.

FIG. 1B shows the results of an assay to evaluate the levels of the production of STAT3 (y-axis) in activated CD8 T cells of the indicated fused hIL10 polypeptides in response to varying concentrations (x-axis) of the fused hIL10 polypeptides as more fully described in the Examples.

FIG. 2A shows the results of an assay to evaluate the levels of the production of STAT3 (y-axis) in activated monocytes of the indicated fused hIL10 polypeptides in response to varying concentrations (x-axis) of the fused hIL10 polypeptides as more fully described in the Examples.

FIG. 2B shows the results of an assay to evaluate the levels of the production of STAT3 (y-axis) in activated CD8 T cells of the indicated fused hIL10 polypeptides in response to varying concentrations (x-axis) of the fused hIL10 polypeptides as more fully described in the Examples.

FIG. 3A shows the results of an assay to evaluate the levels of the production of STAT3 (y-axis) in activated monocytes of the indicated fused hIL10 polypeptides in response to varying concentrations (x-axis) of the fused hIL10 polypeptides as more fully described in the Examples.

FIG. 3B shows the results of an assay to evaluate the levels of the production of STAT3 (y-axis) in activated CD8 T cells of the indicated fused hIL10 polypeptides in response to varying concentrations (x-axis) of the fused hIL10 polypeptides as more fully described in the Examples.

FIG. 4A shows the results of an assay to evaluate the levels of the production of STAT3 (y-axis) in activated monocytes of the indicated fused hIL10 polypeptides in response to varying concentrations (x-axis) of the fused hIL10 polypeptides as more fully described in the Examples.

FIG. 4B shows the results of an assay to evaluate the levels of the production of STAT3 (y-axis) in activated CD8 T cells of the indicated fused hIL10 polypeptides in response to varying concentrations (x-axis) of the fused hIL10 polypeptides as more fully described in the Examples.

FIG. 5A shows the results of an assay to evaluate the levels of the production of STAT3 (y-axis) in activated monocytes of the indicated fused hIL10 polypeptides in response to varying concentrations (x-axis) of the fused hIL10 polypeptides as more fully described in the Examples.

FIG. 5B shows the results of an assay to evaluate the levels of the production of STAT3 (y-axis) in activated CD8 T cells of the indicated fused hIL10 polypeptides in response to varying concentrations (x-axis) of the fused hIL10 polypeptides as more fully described in the Examples.

FIG. 6A shows the results of an assay to evaluate the secretion of IL1β (y-axis) from activated monocytes in response to varying concentrations (x-axis) of the indicated fused hIL10 polypeptides as more fully described in the Examples.

FIG. 6B shows the results of an assay to evaluate the secretion of TNFα (y-axis) from activated monocytes in response to varying concentrations (x-axis) of the indicated fused hIL10 polypeptides as more fully described in the Examples.

FIG. 7A shows the results of an assay to evaluate the production of IFNγ (y-axis) in activated CD8+ T cells in response to varying concentrations (x-axis) of the indicated fused hIL10 polypeptides as more fully described in the Examples.

FIG. 7B shows the results of an assay to evaluate the production of granzyme A (y-axis) in activated CD8+ T cells in response to varying concentrations (x-axis) of the indicated fused hIL10 polypeptides as more fully described in the Examples.

FIG. 7C shows the results of an assay to evaluate the production of granzyme B (y-axis) in activated CD8+ T cells in response to varying concentrations (x-axis) of the indicated fused hIL10 polypeptides as more fully described in the Examples. As illustrated in FIGS. 7A-7C, the fused hIL10 polypeptide comprising a T100L mutation exhibited reduced induction of IFNγ, Granzyme A and Granzyme B in CD8 T cell blasts compared to the fused hIL10 polypeptide and exhibited a reduced Emax relative to the fused WT hIL10 polypeptide when evaluated up to a 100 nM concentration.

FIG. 8A shows the results of an assay to evaluate the levels of the production of STAT3 (y-axis) in activated monocytes of the indicated fused hIL10 polypeptides in response to varying concentrations (x-axis) of the fused hIL10 polypeptides as more fully described in the Examples.

FIG. 8B shows the results of an assay to evaluate the levels of the production of STAT3 (y-axis) in activated CD8 T cells of the indicated fused hIL10 polypeptides in response to varying concentrations (x-axis) of the fused hIL10 polypeptides as more fully described in the Examples.

FIG. 9A shows the results of an assay to evaluate the secretion of IL10 (y-axis) from LPS activated monocytes in response to varying concentrations (x-axis) of the indicated fused hIL10 polypeptides as more fully described in the Examples.

FIG. 9B shows the results of an assay to evaluate the secretion of TNFα (y-axis) from LPS activated monocytes in response to varying concentrations (x-axis) of the indicated fused hIL10 polypeptides as more fully described in the Examples.

FIG. 10A shows the results of an assay to evaluate the production of IFNγ (y-axis) in activated CD8+ T cells in response to varying concentrations (x-axis) of the indicated fused hIL10 polypeptides as more fully described in the Examples.

FIG. 10B shows the results of an assay to evaluate the production of granzyme A (y-axis) in activated CD8+ T cells in response to varying concentrations (x-axis) of the indicated fused hIL10 polypeptides as more fully described in the Examples.

FIG. 10C shows the results of an assay to evaluate the production of granzyme B (y-axis) in activated CD8+ T cells in response to varying concentrations (x-axis) of the indicated fused hIL10 polypeptides as more fully described in the Examples.

FIG. 11A shows the results of an assay to evaluate the levels of the production of STAT3 (y-axis) in activated mouse myeloid cells of the indicated fused mIL10 polypeptides in response to varying concentrations (x-axis) of the fused mIL10 polypeptides as more fully described in the Examples.

FIG. 11B shows the results of an assay to evaluate the levels of the production of STAT3 (y-axis) in activated mouse CD8 T cells of the indicated fused mIL10 polypeptides in response to varying concentrations (x-axis) of the fused mIL10 polypeptides as more fully described in the Examples.

FIG. 12A shows the results of an assay to evaluate the production of IFNγ (y-axis) in LPS activated mouse splenocytes in response to varying concentrations (x-axis) of the indicated fused mIL10 polypeptides as more fully described in the Examples.

FIG. 12B shows the results of an assay to evaluate the production of IL6 (y-axis) in LPS activated mouse splenocytes in response to varying concentrations (x-axis) of the indicated fused hIL10 polypeptides as more fully described in the Examples.

FIG. 13 shows the results of an assay to evaluate the levels of the secretion of granzyme B (y-axis) in activated mouse CD8 T cells of the indicated fused mIL10 polypeptides in response to varying concentrations (x-axis) of the fused mIL10 polypeptides as more fully described in the Examples.

FIG. 14 shows the results of an assay to evaluate the percent survival activated mouse CD8 T cells (y-axis) in response to varying concentrations of the indicated the fused mIL10 polypeptides (x-axis) as more fully described in the Examples. As illustrated, the mouse fused polypeptide with T100L mutation was weaker at inducing survival of CD8 T cell blasts and did not achieve Emax levels equivalent to the fused mIL10 WT polypeptide up to 100 nM concentration.

FIG. 15 shows the results of an assay to evaluate the inducing cell surface CD64 in mouse monocytes (y-axis) in response to varying concentrations of the indicated the fused mIL10 polypeptides (x-axis) as more fully described in the Examples. The data provided indicate that the fused polypeptide with T100L mutation (TP0015) was exhibited reduced induction of cell surface expression of CD64 on monocytes compared to the native mIL10 molecule.

FIG. 16 provides the results of a pharmacokinetic study to evaluate the effect of serum concentration in mice (y-axis) overtime (y-axis) in response to the single administration of varying indicated doses of the indicated pegylated murine fused mIL10 polypeptides comprising the T100L mutation as more fully described in the Example. The data provided indicate that the PEGylated versions of the fused mIL10 polyeptides exhibit a longer in vivo half life compared to the non-PEGyated fused mIL10 polypeptide post a single administration of the polypeptide.

FIG. 17 provides the results of analytical SEC monitoring pH-dependent stability of native IL10 (hIL10 WT) or fused IL10 WT (h_SS0052_AA). The data provided indicates that the native IL10 molecule dissociated into monomers at pH<5.0 while the fused hIL10 molecule remained stable under similar conditions.

FIGS. 18A-18D provide schematic representations of various configurations of fused IL10 polypeptides conjufated to Fc polypeptides. FIGS. 18A-C indicate knob-into-hole (KiH) Fc configurations where the fused IL10 polypeptide is conjugated to the “hole” subunit of the KiH dimeric Fc (18A), where the fused IL10 polypeptide is conjugated to the “knob” subunit of the KiH dimeric Fc (18B), where a first (white) fused IL10 polypeptide is conjugated to the “hole” Fc subunit and a second (black) fused IL10 polypeptide is conjugated to the “knob” Fc subunit of the KiH dimeric Fc (18C) wherein the first and second fused IL10 polypeptides are different. FIG. 18D illustrates a configuration wherein the Fc is not modified to promote heterodimerization and each of the Fe subunits of the Fe dimer are conjugated to a fused IL10 polypeptide.

FIGS. 19A and 19B provide the results of an evaluation of species cross reactivity of human molecules in mouse splenocytes showing IL6 (A, vertical axis) and TNFα (B, vertical axis) levels in response to varying concentrations of the test articles (horizontal axis) as more fully described in Example 11 herein.

FIG. 20 provides the results of an evaluation of Granzyme B secretion (vertical axis) as a percentage of wild-type IL10 activity in response to varying concentrations of the test articles (horizontal axis) as more fully described in Example 12 herein.

FIG. 21 provides the results of an evaluation of the activity of the test articles assessed by their ability to inhibit LPS-induced secretion of IL-6 (vertical axis) in cynomologous monkey whole blood n response to varying concentrations of the test articles (horizontal axis) as more fully described in Example 13 herein.

FIG. 22 provides the results of an evaluation of bodyweight (vertical axis) of mice over time (horizontal axis) treated with the various test articles at varying doses in the DSS model of ulcerative colitis as more fully described in Example 14 herein.

FIG. 23 provides the results of an evaluation of percentage of initial bodyweight (vertical axis) of mice over time (horizontal axis) treated with the various test articles at varying doses in the DSS model of ulcerative colitis as more fully described in Example 14 herein.

FIG. 24 provides the results of an evaluation of bodyweight (vertical axis) in response to the various test articles at varying doses (horizontal axis) upon conclusion of the DSS model of ulcerative colitis as more fully described in Example 14 herein.

FIG. 25 provides the results of an evaluation of colon length (vertical axis) in response to the various test articles at varying doses (horizontal axis) upon conclusion of the DSS model of ulcerative colitis as more fully described in Example 14 herein.

FIG. 26 provides hematocrit results (vertical axis) from mice on Day 4 of the DSS model of ulcerative colitis in response to the various test articles at varying doses (horizontal axis) as more fully described in Example 14 herein.

FIG. 27 provides the results of evaluations of the levels of the IFNγ, IL10, IL-1B, IL6, TNF-a and IL-4 serum cytokines (vertical axes) in response to the administration of the various test article treatments over time (Study Days, horizontal axis) as more fully described in Example 14 herein.

FIG. 28 provides the results of evaluations of the levels of IL-5, IP10, IL17A/F and MIP1a serum cytokines (vertical axes) in response to the administration of the various test article treatments over time (Study Days, horizontal axis) as more fully described in Example 14 herein.

FIG. 29 provides the results of an evaluation of CD163 expression (vertical axis) by peritoneal macrophages with respect to the administration of the various test articles (horizontal axis) as more fully described in Example 14.

FIG. 30 provides the results of an evaluation of CD64 expression (vertical axis) by peritoneal macrophages with respect to the administration of the various test articles (horizontal axis) as more fully described in Example 14.

FIG. 31 provides the results of an evaluation of percent epithelial damage (vertical axis) in mouse colon in response to the administration of the various test articles (horizontal axis) as more fully described in Example 14.

FIG. 32 provides the results of an evaluation of CD11B+ and TH17 cells (vertical axes) in mouse colon mucosa in response to the administration of the various test articles (horizontal axis) as more fully described in Example 14.

FIG. 33 provides the results of an evaluation of the levels of pSTAT3 induction in peripheral blood immune cells in response to the administration of the various test articles (horizontal axis) as more fully described in Example 15.

FIG. 34 provides the results of a pharmacokinetic study to evaluate the serum concentrations of the various test articles over time as more fully described in Example 16

FIG. 35 provides the results of an evaluation of IL6, KC/GRO, IL10 and TNFα serum cytokine levels (vertical axes) at 1.5 hours following LPS administration in the LPS shock model in response to the administration of the various test articles (horizontal axis) as more fully described in Example 17.

FIG. 36 provides the results of an evaluation of IL6, KC/GRO, IL10, TNFα, IFNγ, IL1b, and IL5 serum cytokine levels (vertical axes) at six (6) hours following LPS administration in the LPS shock model in response to the administration of the various test articles (horizontal axis) as more fully described in Example 17.

FIG. 37 provides the results of a pharmacokinetic study to evaluate the serum concentrations of the various test articles over time as more fully described in Example 18.

FIG. 38 provides the results of an evaluation of the test articles ability to induce pSTAT3 (vertical axis) in peripheral blood myeloid cells over time (horizontal axis) as more fully described in Example 19.

FIG. 39 provides the results of an evaluation of peritoneal exudate immune cell scavenger CD163 and CD64 receptor expression (vertical axis) over time in response to the administration of the various test articles (horizontal axis) as more fully described in Example 21.

FIG. 40 provides the results of an evaluation of peritoneal exudate immune cell scavenger CD16 expression (vertical axis) over time in response to the administration of the various test articles (horizontal axis) as more fully described in Example 22.

FIG. 41 provides the results of an evaluation of peritoneal exudate immune cell scavenger CD38 expression (vertical axis) over time in response to the administration of the various test articles (horizontal axis) as more fully described in Example 23.

FIG. 42 provides the results of an evaluation of CD38 and CD16 levels on peripheral blood immune cells (vertical axis) over time in response to the administration of the various test articles (horizontal axis) as more fully described in Example 24.

FIG. 43 provides the results of an evaluation of CD150 levels on peripheral blood B cells over time in response to the administration of the various test articles as more fully described in Example 25.

FIG. 44 provides hematocrit results (vertical axis) from mice in a IFNγ induced anemia model on Study Day 8 in response to the administration of the various test articles as more fully described in Example 26.

FIG. 45 provides data relating to secretion of IL1βb (vertical axis) from human monocytes stimulated with LPS in response to increasing concentrations (horizontal axis) of the various test articles as more fully described in Example 27.

FIG. 46 provides data relating to secretion of IL6 (vertical axis) from human monocytes stimulated with LPS in response to increasing concentrations (horizontal axis) of the various test articles as more fully described in Example 27.

FIG. 47 provides data relating to secretion of TNFα (vertical axis) from human monocytes stimulated with LPS in response to increasing concentrations (horizontal axis) of the various test articles as more fully described in Example 27.

FIG. 48 provides data relating to secretion of IL1βb (vertical axis) from human monocytes stimulated with LPS in response to increasing concentrations (horizontal axis) of the administration of the various test articles as more fully described in Example 28.

FIG. 49 provides data relating to secretion of TNFα (vertical axis) from human monocytes stimulated with LPS in response to increasing concentrations (horizontal axis) of the administration of the various test articles as more fully described in Example 28.

FIG. 50 provides data relating to secretion of IL1βb (vertical axis) from human monocytes stimulated with LPS in response to increasing concentrations (horizontal axis) of the administration of the various test articles as more fully described in Example 29.

FIG. 51 provides data relating to secretion of TNFα (vertical axis) from human monocytes stimulated with LPS in response to increasing concentrations (horizontal axis) of the administration of the various test articles as more fully described in Example 29.

FIG. 52 provides data relating to secretion of IL1βb (vertical axis) from human monocytes stimulated with LPS in response to increasing concentrations (horizontal axis) of the various test articles as more fully described in Example 30.

FIG. 53 provides data relating to secretion of IL6 (vertical axis) from human monocytes stimulated with LPS in response to increasing concentrations (horizontal axis) of the various test articles as more fully described in Example 30.

FIG. 54 provides data relating to secretion of TNFα (vertical axis) from human monocytes stimulated with LPS in response to increasing concentrations (horizontal axis) of the various test articles as more fully described in Example 30.

FIG. 55 provides data relating to secretion of IL1βb (vertical axis) from human monocytes stimulated with LPS in response to increasing concentrations (horizontal axis) of the various test articles as more fully described in Example 31.

FIG. 56 provides data relating to secretion of IL6 (vertical axis) from human monocytes stimulated with LPS in response to increasing concentrations (horizontal axis) of the various test articles as more fully described in Example 31.

FIG. 57 provides data relating to secretion of TNFα (vertical axis) from human monocytes stimulated with LPS in response to increasing concentrations (horizontal axis) of the various test articles as more fully described in Example 31.

FIG. 58 provides data relating to secretion of IL1βb (vertical axis) from human monocytes stimulated with LPS in response to increasing concentrations (horizontal axis) of the various test articles as more fully described in Example 32.

FIG. 59 provides data relating to secretion of IL6 (vertical axis) from human monocytes stimulated with LPS in response to increasing concentrations (horizontal axis) of the various test articles as more fully described in Example 32.

FIG. 60 provides data relating to secretion of TNFα (vertical axis) from human monocytes stimulated with LPS in response to increasing concentrations (horizontal axis) of the various test articles as more fully described in Example 32.

FIG. 61 provides data relating to secretion of IL1βb (vertical axis) from human monocytes stimulated with LPS in response to increasing concentrations (horizontal axis) of the various test articles as more fully described in Example 33.

FIG. 62 provides data relating to secretion of IL6 (vertical axis) from human monocytes stimulated with LPS in response to increasing concentrations (horizontal axis) of the various test articles as more fully described in Example 33.

FIG. 63 provides data relating to secretion of TNFα (vertical axis) from human monocytes stimulated with LPS in response to increasing concentrations (horizontal axis) of the various test articles as more fully described in Example 33.

FIG. 64 provides data relating to secretion of IL1βb (vertical axis) from human monocytes stimulated with LPS in response to increasing concentrations (horizontal axis) of the various test articles as more fully described in Example 34.

FIG. 65 provides data relating to secretion of IL6 (vertical axis) from human monocytes stimulated with LPS in response to increasing concentrations (horizontal axis) of the various test articles as more fully described in Example 34.

FIG. 66 provides data relating to secretion of TNFα (vertical axis) from human monocytes stimulated with LPS in response to increasing concentrations (horizontal axis) of the various test articles as more fully described in Example 34.

FIG. 67 provides data relating to secretion of IL1βb (vertical axis) from human monocytes stimulated with LPS in response to increasing concentrations (horizontal axis) of the various test articles as more fully described in Example 35.

FIG. 68 provides data relating to secretion of IL6 (vertical axis) from human monocytes stimulated with LPS in response to increasing concentrations (horizontal axis) of the various test articles as more fully described in Example 35.

FIG. 69 provides data relating to secretion of TNFα (vertical axis) from human monocytes stimulated with LPS in response to increasing concentrations (horizontal axis) of the various test articles as more fully described in Example 35.

FIG. 70 provides data relating to secretion of IL1βb (vertical axis) from human monocytes stimulated with LPS in response to increasing concentrations (horizontal axis) of the various test articles as more fully described in Example 36.

FIG. 71 provides data relating to secretion of IL6 (vertical axis) from human monocytes stimulated with LPS in response to increasing concentrations (horizontal axis) of the various test articles as more fully described in Example 36.

FIG. 72 provides data relating to secretion of TNFα (vertical axis) from human monocytes stimulated with LPS in response to increasing concentrations (horizontal axis) of the various test articles as more fully described in Example 36.

FIG. 73 provides data relating to secretion of IL1βb (vertical axis) from human monocytes stimulated with LPS in response to increasing concentrations (horizontal axis) of the various test articles as more fully described in Example 37.

FIG. 74 provides data relating to secretion of IL6 (vertical axis) from human monocytes stimulated with LPS in response to increasing concentrations (horizontal axis) of the various test articles as more fully described in Example 37.

FIG. 75 provides data relating to secretion of TNFα (vertical axis) from human monocytes stimulated with LPS in response to increasing concentrations (horizontal axis) of the various test articles as more fully described in Example 37.

FIG. 76 provides data relating to secretion of IL1βb (vertical axis) from human monocytes stimulated with LPS in response to increasing concentrations (horizontal axis) of the various test articles as more fully described in Example 38.

FIG. 77 provides data relating to secretion of IL6 (vertical axis) from human monocytes stimulated with LPS in response to increasing concentrations (horizontal axis) of the various test articles as more fully described in Example 38.

FIG. 78 provides data relating to secretion of TNFα (vertical axis) from human monocytes stimulated with LPS in response to increasing concentrations (horizontal axis) of the various test articles as more fully described in Example 38.

FIG. 79 provides data relating to secretion of IL1βb (vertical axis) from human monocytes stimulated with LPS in response to increasing concentrations (horizontal axis) of the various test articles as more fully described in Example 39.

FIG. 80 provides data relating to secretion of IL6 (vertical axis) from human monocytes stimulated with LPS in response to increasing concentrations (horizontal axis) of the various test articles as more fully described in Example 39.

FIG. 81 provides data relating to secretion of TNFα (vertical axis) from human monocytes stimulated with LPS in response to increasing concentrations (horizontal axis) of the various test articles as more fully described in Example 39.

FIG. 82 provides data relating to secretion of IL1βb (vertical axis) from human monocytes stimulated with LPS in response to increasing concentrations (horizontal axis) of the various test articles as more fully described in Example 40.

FIG. 83 provides data relating to secretion of IL6 (vertical axis) from human monocytes stimulated with LPS in response to increasing concentrations (horizontal axis) of the various test articles as more fully described in Example 40.

FIG. 84 provides data relating to secretion of TNFα (vertical axis) from human monocytes stimulated with LPS in response to increasing concentrations (horizontal axis) of the various test articles as more fully described in Example 40.

FIG. 85 provides data relating to secretion of IL1βb (vertical axis) from human monocytes stimulated with LPS in response to increasing concentrations (horizontal axis) of the various test articles as more fully described in Example 41.

FIG. 86 provides data relating to secretion of IL6 (vertical axis) from human monocytes stimulated with LPS in response to increasing concentrations (horizontal axis) of the various test articles as more fully described in Example 41.

FIG. 87 provides data relating to secretion of TNFα (vertical axis) from human monocytes stimulated with LPS in response to increasing concentrations (horizontal axis) of the various test articles as more fully described in Example 41.

FIG. 88 provides data relating to secretion of IL1βb (vertical axis) from human monocytes stimulated with LPS in response to increasing concentrations (horizontal axis) of the various test articles as more fully described in Example 42.

FIG. 89 provides data relating to secretion of IL6 (vertical axis) from human monocytes stimulated with LPS in response to increasing concentrations (horizontal axis) of the various test articles as more fully described in Example 42.

FIG. 90 provides data relating to secretion of TNFα (vertical axis) from human monocytes stimulated with LPS in response to increasing concentrations (horizontal axis) of the various test articles as more fully described in Example 42.

FIG. 91 provides data relating to secretion of IL1βb (vertical axis) from human monocytes stimulated with LPS in response to increasing concentrations (horizontal axis) of the various test articles as more fully described in Example 43.

FIG. 92 provides data relating to secretion of IL6 (vertical axis) from human monocytes stimulated with LPS in response to increasing concentrations (horizontal axis) of the various test articles as more fully described in Example 43.

FIG. 93 provides data relating to secretion of TNFα (vertical axis) from human monocytes stimulated with LPS in response to increasing concentrations (horizontal axis) of the various test articles as more fully described in Example 43.

FIG. 94 provides data relating to secretion of IL1βb (vertical axis) from human monocytes stimulated with LPS in response to increasing concentrations (horizontal axis) of the various test articles as more fully described in Example 44.

FIG. 95 provides data relating to secretion of IL6 (vertical axis) from human monocytes stimulated with LPS in response to increasing concentrations (horizontal axis) of the various test articles as more fully described in Example 44.

FIG. 96 provides data relating to secretion of TNFα (vertical axis) from human monocytes stimulated with LPS in response to increasing concentrations (horizontal axis) of the various test articles as more fully described in Example 44.

FIG. 97 provides data relating to IC50 values of the various test molecules in inhibiting IL1βb (vertical axis) from human monocytes stimulated with LPS as more fully described in Example 45.

FIG. 98 provides data relating to IC50 values of the various test molecules in inhibiting IL6 (vertical axis) from human monocytes stimulated with LPS as more fully described in Example 45.

FIG. 99 provides data relating to IC50 values of the various test molecules in inhibiting TNFα (vertical axis) from human monocytes stimulated with LPS as more fully described in Example 45.

FIG. 100 provides data relating to the secretion of Granzyme A of the various test articles normalized to the percentage maximum secretion induced by fused_WT IL-10 (h_DR2339_AA) as more fully described in Example 46.

FIG. 101 provides data relating to the secretion of Granzyme B of the various test articles normalized to the percentage maximum secretion induced by fused_WT IL-10 (h_DR2339_AA) as more fully described in Example 46.

FIG. 102 provides data relating to the secretion of IFNg of the various test articles normalized to the percentage maximum secretion induced by fused_WT IL-10 (h_DR2339_AA) as more fully described in Example 46.

FIG. 103 provides data relating to the secretion of IFNg in human CD8 T cell blasts normalized to the percentage maximum secretion induced by fused h_WT IL-10 (h_DR2339_AA) (vertical axis) in response to varying concentrations of the test articles (horizontal axis) from two human donors as more fully described in Example 47. Each panel of FIG. 103 represents data from cells obtained from a different human donor, upper panel donor RG3708 and lower panel donor RG2889.

FIG. 104 provides data relating to the secretion of Granzyme A in human CD8 T cell blasts normalized to the percentage maximum secretion induced by fused h_WT IL-10 (h_DR2339_AA) (vertical axis) in response to varying concentrations of the test articles (horizontal axis) of as more fully described in Example 48.

FIG. 105 provides data relating to the secretion of Granzyme B in human CD8 T cell blasts normalized to the percentage maximum secretion induced by fused h_WT IL-10 (h_DR2339_AA) (vertical axis) in response to varying concentrations of the test articles (horizontal axis) of as more fully described in Example 48.

FIG. 106 provides data relating to the secretion of IFNg in human CD8 T cell blasts normalized to the percentage maximum secretion induced by fused h_WT IL-10 (h_DR2339_AA) (vertical axis) in response to varying concentrations of the test articles (horizontal axis) of as more fully described in Example 48.

### DETAILED DESCRIPTION OF THE DISCLOSURE

To facilitate the understanding of present disclosure, certain terms and phrases are defined below as well as throughout the specification. The definitions provided herein are non-limiting and should be read in view of the knowledge of one of skill in the art.

Before the present methods and compositions are described, it is to be understood that this invention is not limited to a particular method or composition described. It is also to be understood that the terminology used herein is for the purpose of describing embodiments only and is not intended to be limiting.

Where a range of values is provided, it is understood that each intervening value, to the tenth of a unit, between the upper and lower limits of the stated range is also specifically disclosed unless the context clearly dictates otherwise. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, technical and scientific terms used herein are construed as commonly understood by one of ordinary skill in the art to which this invention belongs. Although any methods and materials similar or equivalent to those described herein may be used in the practice or testing of the present invention, some potential and preferred methods and materials are now described. All publications, patents, published patent applications, GenBank accession numbers and UniProt reference numbers mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless indicated otherwise, parts are parts by weight, molecular weight is weight average molecular weight, temperature is in degrees Celsius (°C), and pressure is at or near atmospheric. Standard abbreviations are used, including the following: bp=base pair(s); kb=kilobase(s); pl=picoliter(s); s or sec=second(s); min=minute(s); h or hr=hour(s); AA or aa=amino acid(s); kb=kilobase(s); nt=nucleotide(s); pg=picogram; ng=nanogram; g=microgram; mg=milligram; g=gram; kg=kilogram; dl or dL=deciliter; μl or μL=microliter; ml or mL=milliliter; 1 or L=liter; M=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=once weekly; QM=once monthly; HPLC=high performance liquid chromatography; BW=body weight; U=unit; ns=not statistically significant; PBS=phosphate-buffered saline; PCR=polymerase chain reaction; HSA=human serum albumin; MSA=mouse serum albumin; DMEM=Dulbeco's Modification of Eagle's Medium; EDTA=ethylenediaminetetraacetic acid.

It will be appreciated that throughout this disclosure reference is made to amino acids according to the single letter or three letter codes.

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. I-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

### Nomenclature of Amino Acid Substitutions and Deletions

The present disclosure provides variant polypeptides comprising amino acid substitutions relative to the wild-type or parent polypeptide. The following nomenclature is used herein to refer to substitutions, deletions or insertions. Residues may be designated herein by the one-letter or three-letter amino acid code of the naturally occurring amino acid found in the wild-type molecule. In the present disclosure, the numbering of amino acid residues of human IL10 polypeptide monomers is made in reference to the number of the residue of the “mature” form of the hIL10 polypeptide monomer as provided in SEQ ID NO:4. In reference to the human IL10 polypeptide monomers, substitutions are designated herein by the one letter amino acid code followed by the wild-type hIL10 (SEQ ID NO:4) amino acid position followed by the one letter amino acid code which is substituted. For example, a human IL10 polypeptide monomer having the modification “D25K” refers to a substitution of the aspartic acid (D) residue at position 25 of the (SEQ ID NO:4) with a lysine (K) residue at this position. A deletion of an amino acid residue is referred to as “des” or the symbol “A” followed by the amino acid residue and its position.

Immunoglobulin, Upper Hinge and Fc Residue Numbering: There are a variety of numbering conventions that are employed with respect to the numbering of amino acid residues of immunoglobulins including Kabat numbering, Chothia numbering, EU numbering and IMGT numbering conventions. In the context of the present disclosure, the numbering of amino acid residues of immunoglobulin molecules including domains thereof including the upper hinge and Fc domain (comprising the lower hinge, CH2 and CH3 domains) is made in accordance with EU Numbering conventions. Translation of EU numbering conventions used herein to Kabat numbering, Chothia numbering, or IMGT numbering conventions is readily understood by those of skill in the art. Dondelinger, et al. (2018) Understanding the Significance and Implications of Antibody Numbering and Antigen-Binding Surface/Residue Definition Frontiers in Immunology Volume 9 Article #:2278.

### DEFINITIONS

Unless otherwise indicated, the following terms are intended to have the meaning set forth below. Other terms are defined elsewhere throughout the specification.

About: The term “about” refers to a value that is plus or minus 10% of a numerical value described herein, such as plus or minus 0%, 1%, 2%, 3%, 4%, 5%, 6%, 7%, 8%, 9%, or 10% of numerical value described herein. The term “about” also applies to all numerical ranges described herein. All values described herein are understood to be modified by the term “about” whether or not the term “about” is explicitly recited in reference to a given value.

Activate: As used herein the term “activate” is used in reference to a receptor or receptor complex to reflect a biological effect, directly and/or by participation in a multicomponent signaling cascade, arising from the binding of an agonist ligand to a receptor responsive to the binding of the ligand. The term activate or activated may also be used in reference to a cell state in response to the exposure of the cell to an activating agent.

Activity: As used herein, the term “activity” is used with respect to a molecule to describe a property of the molecule with respect to a test system (e.g., an assay) or biological or chemical property (e.g., the degree of binding of the molecule to another molecule), the effect of an agent on a cell (e.g., activation of a cell) or of a physical property of a material or cell (e.g., modification of cell membrane potential). Examples of such biological functions include but are not limited to catalytic activity of a biological agent, the ability to stimulate intracellular signaling, gene expression, cell proliferation, the ability to modulate immunological activity such as inflammatory response. “Activity” is typically expressed as a level of a biological activity per unit of agent tested such as [catalytic activity]/[mg protein], [immunological activity]/[mg protein], international units (IU) of activity, [STAT3 phosphorylation]/[mg protein], [proliferation]/[mg protein], plaque forming units (pfu), etc. As used herein, the term proliferative activity refers to an activity that promotes cell proliferation and replication, including dysregulated cell division such as that observed in neoplastic diseases, inflammatory diseases, fibrosis, dysplasia, cell transformation, metastasis, and angiogenesis.

Administer/Administration: The terms “administration” and “administer” are used interchangeably herein to refer the act of contacting a subject, including contacting a cell, tissue, organ, or biological fluid of the subject in vitro, in vivo or ex vivo with an agent (e.g., a hIL10 mutein or an engineered cell expressing a hIL10 mutein, a chemotherapeutic agent, an antibody, or a pharmaceutical formulation comprising one or more of the foregoing). Administration of an agent may be achieved through any of a variety of art recognized methods including but not limited to the topical administration, intravascular injection (including intravenous or intraarterial infusion), intradermal injection, subcutaneous injection, intramuscular injection, intraperitoneal injection, intracranial injection, intratumoral injection, transdermal, transmucosal, iontophoretic delivery, intralymphatic injection, intragastric infusion, intraprostatic injection, intravesical infusion (e.g., bladder), inhalation (e.g respiratory inhalers including dry-powder inhalers), intraocular injection, intraabdominal injection, intralesional injection, intraovarian injection, intracerebral infusion or injection, intracerebroventricular injection (ICVJ), and the like. The term “administration” includes contact of an agent to the cell, tissue or organ as well as the contact of an agent to a fluid, where the fluid is in contact with the cell, tissue or organ.

Affinity: As used herein the term “affinity” refers to the degree of specific binding of a first molecule (e.g., a ligand) to a second molecule (e.g., a receptor) and is measured by the equilibrium dissociation constant (K_{D}), a ratio of the dissociation rate constant between the molecule and its target (K_{off}) and the association rate constant between the molecule and its target (Kₒₙ).

Agonist: As used herein, the term “agonist” refers a first agent that specifically binds a second agent (“target”) and interacts with the target to cause or promote an increase in the activation of the target. In some instances, agonists are activators of receptor proteins that modulate cell activity, enhance activation, sensitize cells to activation by a second agent, or up-regulate the expression of one or more genes, proteins, ligands, receptors, biological pathways, that may result in modulation of cellular activity including but not limited to cell activation and/or proliferation or the cell cycle. In some embodiments, an agonist is an agent that binds to a receptor and alters the receptor state resulting in a biological response that mimics the effect of the endogenous ligand of the receptor. In some embodiments, an agonist is a modified form of a cognate ligand that binds to its cognate receptor and alters the state of the cognate receptor in a biological response that mimics the biological effect of the interaction of the naturally occurring cognate ligand with its cognate receptor. The term “agonist” includes partial agonists, full agonists and superagonists. An agonist may be described as a “full agonist” when such agonist which leads to a substantially full biological response (i.e., the response associated with the naturally occurring ligand/receptor binding interaction). A “partial agonist” is a type of agonist that can produce a maximal response less than the endogenous agonist for the target receptor, and thus has a maximal activity of less than 100% of the native ligand for the target. In some embodiments, a superagonist exhibits less than 100% but greater than 10%, alternatively greater than 20%, alternatively greater than 30%, alternatively greater than 40%, alternatively greater than 50%, alternatively greater than 60%, alternatively greater than 70%, alternatively greater than 80% or alternatively greater than 90% of the response in an evaluable quantitative or qualitative parameter of the endogenous agonist for the target receptor when evaluated at similar concentrations in a comparable assay. A “superagonist” is a type of agonist that can produce a maximal response greater than the endogenous agonist for the target receptor, and thus has a maximal activity of more than 100% of the native ligand for the target. In some embodiments, a superagonist exhibits greater than 110%, alternatively greater than 120%, alternatively greater than 130%, alternatively greater than 140%, alternatively greater than 150%, alternatively greater than 160%, or alternatively greater than 170% of the response in an evaluable quantitative or qualitative parameter of the endogenous agonist for the target receptor when evaluated at similar concentrations in a comparable assay. It should be noted that the biological effects associated with the partial agonist, full agonist or superagonist may differ not only in degree but also may differ in kind from those biological effects of endogenous agonist for the target receptor.

Antagonist: As used herein, the term “antagonist” or “inhibitor” refers a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist. Inhibitors are molecules that decrease, block, prevent, delay activation, inactivate, desensitize, or down-regulate, e.g., a gene, protein, ligand, receptor, biological pathway including an immune checkpoint pathway, or cell.

Biological Sample: As used herein, the term “biological sample” or “sample” refers to a sample obtained (or derived) from a subject. By way of example, a biological sample comprises a material selected from the group consisting of body fluids, blood, whole blood, plasma, serum, mucus secretions, saliva, cerebrospinal fluid (CSF), bronchoalveolar lavage fluid (BALF), fluids of the eye (e.g., vitreous fluid, aqueous humor), lymph fluid, lymph node tissue, spleen tissue, bone marrow, tumor tissue, including immunoglobulin enriched or cell-type specific enriched fractions derived from one or more of such tissues.

Comparable: As used herein, the term “comparable” is used to describe the degree of difference in two measurements of an evaluable quantitative or qualitative parameter. For example, where a first measurement of an evaluable quantitative parameter and a second measurement of the evaluable parameter do not deviate beyond a range that the skilled artisan would recognize as not producing a statistically significant difference in effect between the two results in the circumstances, the two measurements would be considered “comparable.” In some instances, measurements may be considered “comparable” if one measurement deviates from another by less than 35%, alternatively by less than 30%, alternatively by less than 25%, alternatively by less than 20%, alternatively by less than 15%, alternatively by less than 10%, alternatively by less than 7%, alternatively by less than 5%, alternatively by less than 4%, alternatively by less than 3%, alternatively by less than 2%, or by less than 1%. In particular embodiments, one measurement is comparable to a reference standard if it deviates by less than 15%, alternatively by less than 10%, or alternatively by less than 5% from the reference standard.

Conservative Amino Acid Substitution: As used herein, the term “conservative amino acid substitution” refers to an amino acid replacement that changes a given amino acid to a different amino acid with similar biochemical properties (e.g., charge, hydrophobicity, and size). For example, the amino acids in each of the following groups can be considered as conservative amino acids of each other: (1) hydrophobic amino acids: alanine, isoleucine, leucine, tryptophan, phenylalanine, valine, proline, and glycine; (2) polar amino acids: glutamine, asparagine, histidine, serine, threonine, tyrosine, methionine, and cysteine; (3) basic amino acids: lysine and arginine; and (4) acidic amino acids: aspartic acid and glutamic acid.

Corresponding To: As used herein, the terms “correspondence” or “corresponding to” in the context of an amino acid or nucleotide in a polypeptide or polynucleotide, respectively, refers to the equivalent position (e.g., amino acid or nuclelotide) of a reference polypeptide or polynucleotide sequence when the reference sequence is aligned with a second sequence to maximize the percentage of sequence identity. For example, an “amino acid position corresponding to amino acid position [X]” of a specified IL10 polypeptide refers to equivalent positions, based on alignment, in other IL10 polypeptides, including structural homologues and variants or IL10 derived from different species (e.g., human IL10 and mouse IL10). The corresponding position can be based on a reference sequence, wild-type sequence or parental sequence, for example, with respect to hIL10 muteins a reference sequence may be the amino acid sequence of wild-type hIL10 (SEQ ID NO:4).

Derived From: As used herein, the term “derived from” in the context of polypeptide or polynucleotide variants or muteins is meant to indicate that the polypeptide or polynucleotide variant or mutein has a sequence that is based on that of a reference polypeptide (e.g., the amino acid sequence of wild-type hIL10 (SEQ ID NO:4) or polynucleotide sequence (e.g. the cDNA encoding wild-type hIL10). The term “derived from” is not to be understood as limiting with respect to the source or method in which the polypeptide or polynucleotide variants or mutein is made.

Effective Concentration (EC): As used herein, the terms “effective concentration” or its abbreviation “EC” are used interchangeably to refer to the concentration of an agent in an amount sufficient to effect a change in a given parameter in a test system. The abbreviation “E” refers to the magnitude of a given biological effect observed in a test system when that test system is exposed to a test agent. When the magnitude of the response is expressed as a factor of the concentration (“C”) of the test agent, the abbreviation “EC” is used. In the context of biological systems, the term Emax refers to the maximal magnitude of a given biological effect observed in response to a saturating concentration of an activating test agent. When the abbreviation EC is provided with a subscript (e.g., EC₄₀, EC₅₀, etc.) the subscript refers to the percentage of the Emax of the biological response observed at that concentration. For example, the concentration of a test agent sufficient to result in the induction of a measurable biological parameter in a test system that is 30% of the maximal level of such measurable biological parameter in response to such test agent, this is referred to as the “EC₃₀” of the test agent with respect to such biological parameter. Similarly, the term “EC₁₀₀” is used to denote the effective concentration of an agent that results in the maximal (100%) response of a measurable parameter in response to such agent. Similarly, the term EC₅₀ (which is commonly used in the field of pharmacodynamics) refers to the concentration of an agent sufficient to result in the half-maximal (about 50%) change in the measurable parameter. The term “saturating concentration” refers to the maximum possible quantity of a test agent that can dissolve in a standard volume of a specific solvent (e.g., water) under standard conditions of temperature and pressure. In pharmacodynamics, a saturating concentration of a drug is typically used to denote the concentration sufficient of the drug such that all available receptors are occupied by the drug, and EC₅₀ is the drug concentration to give the half-maximal effect.

Enriched: As used herein in the term “enriched” refers to a sample that is non-naturally manipulated so that a species (e.g., a molecule or cell) of interest is present in: (a) a greater concentration (e.g., at least 3-fold greater, alternatively at least 5-fold greater, alternatively at least 10-fold greater, alternatively at least 50-fold greater, alternatively at least 100-fold greater, or alternatively at least 1000-fold greater) than the concentration of the species in the starting sample, such as a biological sample (e.g., a sample in which the molecule naturally occurs or in which it is present after administration); or (b) a concentration greater than the environment in which the molecule was made (e.g., a recombinantly modified bacterial or mammalian cell).

Extracellular Domain: As used herein the term “extracellular domain” or its abbreviation “ECD” refers to the portion of a cell surface protein (e.g., a cell surface receptor) which is external to of the plasma membrane of a cell. The cell surface protein may be transmembrane protein, a cell surface or membrane associated protein. The cell surface protein may be a multi-pass transmembrane protein having multiple non-contiguous extracellular domains.

Fusion Protein: The term “fusion protein” refers to a polypeptide that comprises amino acid sequences derived from different proteins or synthetic sequences providing differing functions. Examples of fusion proteins include but are not limited to polypeptides comprising a protein and at least one additional functional domain including but not limited to immunogenic domains (e.g. diphtheria or tetanus toxin), polypeptide domains to facilitate expression (e.g. signal peptides) sequences to facilitate isolation and/or purification (e.g. chelating peptides), polypeptide targeting domains (e.g. single domain antibodies), a protein having a distinct additional (e.g. complementary, especially complementary therapeutic) function, polypeptide domains that promote extended duration of action in vivo (e.g. albumin). The domains of a fusion protein may further be joined by polypeptide linkers. The functional domains of the fusion protein may be provided in any N-terminal to C-terminal order. Fusion proteins are typically produced by translation of a single recombinant DNA sequence such that the protein and the additional functional domain of the fusion protein are covalently joined by peptide bonds. In some embodiments, a fusion protein also includes IL10 fusion protein homodimers of the present disclosure.

Identity: The term “identity,” as used herein in reference to polypeptide or DNA sequences, refers to the subunit sequence identity between two molecules. When a subunit position in both of the molecules is occupied by the same monomeric subunit (i.e., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as BLAST 2.0 algorithms, which are described in Altschul et al. (1990) J. Mol. Biol. 215: 403-410 and Altschul, et al. (1977) Nucleic Acids Res. 25: 3389-3402. Software for performing BLAST analyses is publicly available through the National Center for Biotechnology Information (NCBI) web site. The algorithm involves first identifying high scoring sequence pairs (HSPs) by identifying short words of length W of the query sequence, which either match or satisfy some positive-valued threshold score “T” when aligned with a word of the same length in a database sequence. T is referred to as the neighborhood word score threshold (Altschul, et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (the reward score for a pair of matching residues; always >0) and “N” (the penalty score for mismatching residues; always <0). For amino acid sequences, a scoring matrix is used to calculate the cumulative score. Extension of the word hits in each direction are halted when: (a) the cumulative alignment score falls off by the quantity X from its maximum achieved value; the cumulative score goes to zero or below, due to the accumulation of one or more negative-scoring residue alignments; or (b) the end of either sequence is reached. The BLAST algorithm parameters “W”, “T”, and “X” determine the sensitivity and speed of the alignment. The BLASTN program (for nucleotide sequences) functions similarly but uses as defaults a word size (“W”) of 28, an expectation (“E”) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, (1989) PNAS(USA) 89:10915-10919).

In An Amount Sufficient to Effect a Response: As used herein the phrase “in an amount sufficient to cause a response” is used in reference to the amount of a test agent sufficient to provide a detectable change in the level of an indicator measured before (e.g., a baseline level) and after the application of a test agent to a test system. In some embodiments, the test system is a cell, tissue or organism. In some embodiments, the test system is an in vitro test system such as a fluorescent assay. In some embodiments, the test system is an in vivo system which involves the measurement of a change in the level a parameter of a cell, tissue, or organism reflective of a biological function before and after the application of the test agent to the cell, tissue, or organism. In some embodiments, the indicator is reflective of biological function or state of development of a cell evaluated in an assay in response to the administration of a quantity of the test agent. In some embodiments, the test system involves the measurement of a change in the level an indicator of a cell, tissue, or organism reflective of a biological condition before and after the application of one or more test agents to the cell, tissue, or organism. The term “in an amount sufficient to effect a response” may be sufficient to be a therapeutically effective amount but may also be more or less than a therapeutically effective amount.

In Need of Treatment: The term “in need of treatment” as used herein refers to a judgment made by a physician or other caregiver with respect to a subject that the subject requires or will potentially benefit from treatment. This judgment is made based on a variety of factors that are in the realm of the physician's or caregiver's expertise.

In Need of Prevention: As used herein the term “in need of prevention” refers to a judgment made by a physician or other caregiver with respect to a subject that the subject requires or will potentially benefit from preventative care. This judgment is made based upon a variety of factors that are in the realm of a physician's or caregiver's expertise.

Inhibitor: As used herein the term “inhibitor” refers to a molecule that decreases, blocks, prevents, delays activation of, inactivates, desensitizes, antagonizes or down-regulates, e.g., a gene, protein, ligand, receptor, or cell. An inhibitor can also be defined as a molecule that reduces, blocks, or inactivates a constitutive activity of a cell or organism.

Intracellular Domain: As used herein the term “intracellular domain” or its abbreviation “ICD” refers to the portion(s) of a cell surface protein (e.g., a cell surface receptor) which is (are) inside of the plasma membrane of a cell. The ICD may include the entire cytoplasmic portion of a transmembrane protein or membrane associated protein, or intracellular protein. The cell surface protein may be a multi-pass transmembrane protein having multiple non-contiguous intracellular domains.

Isolated: As used herein the term “isolated” is used in reference to a molecule of interest that, if naturally occurring, is in an environment different from that in which it can naturally occurs. “Isolated” is meant to include molecule that are within samples that are substantially enriched for the molecule of interest and/or in which the molecule of interest is partially or substantially purified. Where the molecule is not naturally occurring, “isolated” indicates that the molecule has been separated from an environment in which it was synthesized.

Ligand: As used herein, the term “ligand” refers to a molecule that specifically binds a receptor and causes a change in the receptor so as to effect a change in the activity of the receptor or cause a measurable response in cell that expresses the receptor. In one embodiment, the term “ligand” refers to a molecule or complex thereof that can act as an agonist or antagonist of a receptor. As used herein, the term “ligand” encompasses natural and synthetic ligands. “Ligand” also encompasses small molecules, peptide mimetics of cytokines and antibodies. The complex of a ligand and receptor is termed a “ligand-receptor complex.” A ligand may comprise one domain of a polyprotein or fusion protein (e.g., either domain of an antibody/ligand fusion protein).

Linker: As used herein the terms “linker” refers to a molecule that is used to join a first and second heterologous molecules. In some embodiments, a linker may be a chemical linker. In some embodiments the linker is “polypeptide linker” are to describe a polypeptide employed to join functional subunits of a polypeptide composed of multiple functional domains or subunits (e.g. a fusion protein).

Modified: As used herein, the term “modified” refers to a molecule, such as a polypeptide, whose structure has been changed relative to an unmodified parental molecule. A modified polypeptide typically retains one or more activities or functions of the unmodified parental molecule. For example, a hIL10 mutein monomer can activate IL10 signaling in a cell expressing the IL10 receptor as part of a homodimer, but can have improved properties relative to the unmodified polypeptide. The term modified includes amino acid substitutions that are not present in a parental or wild-type IL10, and includes variants and muteins of an IL10 polypeptide.

Modulate: As used herein, the terms “modulate”, “modulation” and the like refer to the ability of a test agent to cause a response, either positive or negative or directly or indirectly, in a system, including a biological system, or biochemical pathway. The term modulator includes both agonists (including partial agonists, full agonists and superagonists) and antagonists.

Mutein: As used herein, the term “mutein” is used to refer to a variant of a wild-type polypeptide comprising modifications to the primary structure (i.e. amino acid sequence) of such polypeptide. A mutein may have have at at least 99% sequence identity, alternatively at least 98% sequence identity, alternatively at least about 97% sequence identity, alternatively at least 96% sequence identity, alternatively at least 95% sequence identity, alternatively at least about 94% identical, alternatively at least 93% sequence identity, alternatively at least 92% identical, alternatively at least 91% sequence identity, or alternatively at least 90% sequence identity, to the parent polypeptide from which the mutein was derived.

Nucleic Acid: As used herein, the terms “nucleic acid”, “nucleic acid molecule”, “polynucleotide” and the like are used interchangeably herein to refer to a polymeric form of nucleotides (including deoxyribonucleotides or ribonucleotides) of any length. Non-limiting examples of polynucleotides include linear and circular nucleic acids, messenger RNA (mRNA), small nuclear RNA (snRNA), short interfering RNA (siRNA), guide RNA (gRNA) complementary DNA (cDNA), recombinant polynucleotides, recombinant viral or non-viral vectors, recombinant viral or non-viral expression vectors, hybridization probes, PCR primers, and the like.

One or More Amino Acid Substitutions: As used herein, the term “one or more amino acid substitutions” refers to a single amino acid substitution, or one, two, three, four, five or more amino acid substitutions relative to a reference sequence. In some embodiments, the reference sequence is wild-type hIL10 monomer (SEQ ID NO:4).

Operably Linked: The term “operably linked” is used herein to refer to the relationship between a first and second component molecules, typically polypeptides or nucleic acids, which are arranged in a construct such that at least one of the functions of the component molecules is retained in the construct although the operable linkage may result in a construct exhibiting modulated activity, either positively or negatively, of the individual components of the construct. For example, the operable linkage of a polyethylene glycol (PEG) molecule to a wild-type protein may result in a construct where the biological activity of the protein is diminished relative to the to the wild-type molecule, however the two are nevertheless considered operably linked. When the term “operably linked” is applied to the relationship of multiple nucleic acid sequences encoding differing functions, the multiple nucleic acid sequences when combined into a single nucleic acid molecule that, for example, when introduced into a cell using recombinant technology, provides a nucleic acid which is capable of effecting the transcription and/or translation of one or more of the nucleic acid sequences, and expression of one or more proteins in the cell. For example, the nucleic acid sequence encoding a signal sequence may be considered operably linked to DNA encoding a polypeptide if it results in the expression of a preprotein whereby the signal sequence facilitates the secretion of the polypeptide; a promoter or enhancer is considered operably linked to a coding sequence if it affects the transcription of the sequence; or a ribosome binding site is considered operably linked to a coding sequence if it is positioned so as to facilitate translation. Generally, in the context of nucleic acid molecules, the term “operably linked” means that the nucleic acid sequences being linked are contiguous, and, in the case of a secretory leader or associated subdomains of a molecule, contiguous and in reading phase. However, certain genetic elements such as enhancers may function at a distance and need not be contiguous with respect to the sequence to which they provide their effect but nevertheless may be considered operably linked.

Parent Polypeptide: As used herein, the terms “parent polypeptide” or “parent protein” are used interchangeably to designate the source of a second polypeptide (e.g., a derivative, mutein or variant) which is modified with respect to a first “parent” polypeptide. In some instances, the parent polypeptide is a wild-type or naturally occurring form of a protein. In some instance, the parent polypeptide may be a modified form a naturally occurring protein that is further modified. The term parent polypeptide can also be used interchangeably with “reference polypeptide.”

Partial Agonist: As used herein, the term “partial agonist” refers to a molecule that specifically binds that bind to and activate a given receptor but possess only partial activation the receptor relative to a full agonist. Partial agonists may display both agonistic and antagonistic effects. For example, when both a full agonist and partial agonist are present, the partial agonist may act as a competitive inhibitor of the full agonist by competing with the full agonist for the receptor binding resulting in net decrease in receptor activation relative to the contact of the receptor with the full agonist in the absence of the partial agonist. Partial agonists can be used to activate receptors to give a desired submaximal response in a subject in the absence or depleted levels of the endogenous ligand are present in the subject. Partial agonists can be used to reduce the overstimulation of receptors when excess amounts of the endogenous ligand are present. The maximum response (Emax) produced by a partial agonist is called its intrinsic activity and may be expressed on a percentage scale where a full agonist produces a 100% response. An partial agonist may have greater than 10% but less than 100%, alternatively greater than 20% but less than 100%, alternatively greater than 30% but less than 100%, alternatively greater than 40% but less than 100%, alternatively greater than 50% but less than 100%, alternatively greater than 60% but less than 100%, alternatively greater than 70% but less than 100%, alternatively greater than 80% but less than 100%, or alternatively greater than 90% but less than 100%, of the activity of the reference full agonist polypeptide ligand when evaluated at similar concentrations in a given assay system.

Percent Identity: As used herein, the term “percent (%) sequence identity” used in the context of nucleic acids or polypeptides, refers to a sequence that has at least 50% sequence identity with a reference sequence. Alternatively, percent sequence identity can be any integer from 50% to 100%. In some embodiments, a sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined with BLAST using standard parameters, as described below.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window includes reference to a segment of any one of a number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁻⁵, and most preferably less than about 10⁻²⁰.

Polypeptide: As used herein the terms “polypeptide,” “peptide,” and “protein”, used interchangeably herein, refer to a polymeric form of amino acids of any length. A polypeptide may include genetically coded and non-genetically coded amino acids, chemically or biochemically modified or derivatized amino acids, and polypeptides having modified polypeptide backbones. The term polypeptide includes fusion proteins. The term polypeptide also includes fusion proteins of a first polypeptide and a second heterologous polypeptide carrier protein (e.g., human serum albumin (HSA)).

Prevent: As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof. A course of action to prevent a disease, disorder or condition in a subject is typically applied in the context of a subject who is predisposed to developing a disease, disorder or condition due to genetic, experiential or environmental factors of developing a particular disease, disorder or condition.

Receptor: As used herein, the term “receptor” refers to a polypeptide having a domain that specifically binds a ligand that binding of the ligand results in a change to at least one biological property of the polypeptide. In some embodiments, the receptor is a cell membrane associated protein that comprises and extracellular domain (ECD) and a membrane associated domain which serves to anchor the ECD to the cell surface. In some embodiments of cell surface receptors, the receptor is a membrane spanning polypeptide comprising an intracellular domain (ICD) and extracellular domain (ECD) linked by a membrane spanning domain typically referred to as a transmembrane domain (TM). The binding of a cognate ligand to the receptor results in a change in the receptor resulting in a measurable biological effect. In some instances, where the receptor is a membrane spanning polypeptide comprising an ECD, TM and ICD, the binding of the ligand to the ECD results in a measurable intracellular biological effect mediated by one or more domains of the ICD in response to the binding of the ligand to the ECD. In some embodiments, a receptor is a component of a multi-component complex that, when assembled, results in intracellular signaling. For example, the ligand may bind a cell surface receptor that is not associated with any intracellular signaling alone but upon ligand binding facilitates the formation of a heteromultimeric (including heterodimeric, heterotrimeric, etc.) or homomultimeric (including homodimeric, homotrimeric, homotetrameric, etc.) complex that results in a measurable biological effect in the cell such as activation of an intracellular signaling cascade (e.g., the Jak/STAT pathway). In some embodiments, a receptor is a membrane spanning single chain polypeptide comprising ECD, TM and ICD domains wherein the ECD, TM and ICD domains are derived from the same or differing naturally occurring receptor variants or synthetic functional equivalents thereof.

Recombinant: As used herein, the term “recombinant” is used as an adjective to refer to the method by which a polypeptide, nucleic acid, or cell was modified using recombinant DNA technology. A “recombinant protein” is a protein produced using recombinant DNA technology and is frequently abbreviated with a lower case “r” preceding the protein name to denote the method by which the protein was produced (e.g., recombinantly produced human growth hormone is commonly abbreviated “rhGH”). Similarly, a cell is referred to as a “recombinant cell” if the cell has been modified by the incorporation (e.g., transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. The techniques and protocols for recombinant DNA technology are well known in the art such as those can be found in Sambrook, et al. (1989) Molecular Cloning: A Laboratory Manual (2d ed., Cold Spring Harbor Laboratory Press, Plainview, N.Y.) and other standard molecular biology laboratory manuals.

Response: The term “response,” for example, of a cell, tissue, organ, or organism, encompasses a quantitative or qualitative change in a evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation) where the change is correlated with the activation, stimulation, or treatment, with or contact with exogenous agents or internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects. A “response” may be evaluated in vitro such as through the use of assay systems, flow cytometric assays, surface plasmon resonance, enzymatic activity, mass spectroscopy, amino acid or protein sequencing technologies. A “response” may be evaluated in vivo quantitatively by evaluation of objective physiological parameters such as body temperature, bodyweight, tumor volume, blood pressure, results of X-ray or other imaging technology or qualitatively through changes in reported subjective feelings of well-being, depression, agitation, or pain. In some embodiments, the level of activation of T cells in response to the administration of a test agent may be determined by flow cytometric methods as described as determined by the level of one or more STATs (e.g., STAT1, STAT3, or STAT5) in accordance with methods well known in the art.

Significantly Reduced Binding: As used herein, the term “exhibits significantly reduced binding” is used with respect a variant of a first molecule (e.g., a ligand or antibody) which exhibits a significant reduction in the affinity for a second molecule (e.g., receptor or antigen) relative the parent form of the first molecule. With respect to antibody variants, an antibody variant “exhibits significantly reduced binding” if the affinity of the variant antibody for an antigen if the variant binds to the native form of the receptor with and affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent antibody from which the variant was derived. With respect to variant ligands or ligand muteins, a variant ligand or mutein “exhibits significantly reduced binding” if the affinity of the variant ligand or mutein binds to a receptor with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent ligand from which the variant ligand was derived. Similarly, with respect to variant receptors, a variant receptor “exhibits significantly reduced binding” if the affinity of the variant receptors to a ligand binds with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent receptor from which the variant receptor was derived.

Specifically Binds: As used herein the term “specifically binds” refers to the degree of affinity for which a first molecule exhibits with respect to a second molecule. In the context of binding pairs (e.g., ligand/receptor, antibody/antigen) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample. Specific binding may be assessed using techniques known in the art including but not limited to competition ELISA assays, radioactive ligand binding assays (e.g., saturation binding, Scatchard plot, nonlinear curve fitting programs and competition binding assays); non-radioactive ligand binding assays (e.g., fluorescence polarization (FP), fluorescence resonance energy transfer (FRET); liquid phase ligand binding assays (e.g., real-time polymerase chain reaction (RT-qPCR), and immunoprecipitation); and solid phase ligand binding assays (e.g., multiwell plate assays, on-bead ligand binding assays, on-column ligand binding assays, and filter assays)) and surface plasmon resonance assays (see, e.g., Drescher et al., (2009) Methods Mol Biol 493:323-343 with commercially available instrumentation such as the Biacore 8K, Biacore 8K+, Biacore 5200, Biacore T200 (Cytiva, 100 Results Way, Marlborough MA 01752).

Subject: The terms “recipient”, “individual”, “subject”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

Substantially Pure: As used herein, the term “substantially pure” indicates that a component of a composition makes up greater than about 50%, alternatively greater than about 60%, alternatively greater than about 70%, alternatively greater than about 80%, alternatively greater than about 90%, alternatively greater than about 95% of the total content of the composition. A protein that is “substantially pure” comprises greater than about 50%, alternatively greater than about 60%, alternatively greater than about 70%, alternatively greater than about 80%, alternatively greater than about 90%, alternatively greater than about 95% of the total content of the composition.

Suffering From: As used herein, the term “suffering from” refers to a determination made by a physician with respect to a subject based on the available objective or subjective information accepted in the field for the identification of the presence of a disease, disorder or condition in a subject including but not limited to X-ray, CT-scans, conventional laboratory diagnostic tests (e.g., blood count, etc.), genomic data, protein expression data, immunohistochemistry, that the subject requires or will benefit from treatment.

T-cell: As used herein the term “T-cell” or “T cell” is used in its conventional sense to refer to lymphocytes that differentiate in the thymus. In some embodiments the term T cell includes, without limitation, naïve CD8⁺ T cells, cytotoxic CD8⁺ T cells, naïve CD4⁺ T cells, helper T cells, e.g., T_{H}1, T_{H}2, T_{H}9, T_{H}11, T_{H}22, T_{FH}; regulatory T cells, e.g., T_{R}1, Tregs, inducible Tregs; memory T cells, e.g., central memory T cells, effector memory T cells, NKT cells, tumor infiltrating lymphocytes (TILs) and engineered variants of such T-cells including but not limited to CAR-T cells, recombinantly modified TILs and TCR-engineered cells.

Terminus/Terminal: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” refers to the position of a first amino acid residue relative to a second amino acid residue in a contiguous polypeptide sequence, the first amino acid being closer to the N-terminus of the polypeptide. “Immediately C-terminal” refers to the position of a first amino acid residue relative to a second amino acid residue in a contiguous polypeptide sequence, the first amino acid being closer to the C-terminus of the polypeptide.

Therapeutically Effective Amount: As used herein to the phrase “therapeutically effective amount” refers to the quantity of an agent when administered to a subject, either alone or as part of a pharmaceutical composition or treatment regimen, in a single dose or as part of a series of doses, provides a positive effect on any quantitative or qualitative symptom, aspect, or characteristic of a disease, disorder or condition. A therapeutically effective amount can be ascertained by measuring relevant physiological effects, and it may be adjusted in connection with a dosing regimen and in response to diagnostic analysis of the subject's condition. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, biomarkers (such as inflammatory cytokines, IFN-γ, granzyme, and the like), reduction in serum tumor markers, improvement in, increase in duration of survival, extended duration of progression free survival, extension of the time to progression, increased time to treatment failure, extended duration of event free survival, extension of time to next treatment, improvement objective response rate, improvement in the duration of response, reduction of tumor burden, complete response, partial response, stable disease, and the like that that are relied upon by clinicians in the field for the assessment of an improvement in the condition of the subject in response to administration of an agent. In one embodiment, a therapeutically effective amount is an amount of an agent when used alone or in combination with another agent provides a improvement in any quantitative or qualitative symptom, aspect, or characteristic of a disease, disorder or condition and does not result in non-reversible serious adverse events in the course of administration of the agent to the mammalian subject.

Treat: The terms “treat”, “treating”, treatment” and the like refer to a course of action (such as contacting the subject with pharmaceutical composition comprising a hIL10 variant polypeptide monomer alone or in combination with a supplementary agent) that is initiated with respect to a subject in response to a diagnosis that the subject is suffering from a disease, disorder or condition, or a symptom thereof, the course of action being initiated so as to eliminate, reduce, suppress, mitigate, or ameliorate, either temporarily or permanently, at least one of: (a) the underlying causes of such disease, disorder, or condition afflicting a subject; and/or (b) at least one of the symptoms associated with such disease, disorder, or condition. In some embodiments, treating includes a course of action taken with respect to a subject suffering from a disease where the course of action results in the inhibition (e.g., arrests the development of the disease, disorder or condition or ameliorates one or more symptoms associated therewith) of the disease in the subject. In some embodiments, the term “treat” is used to refer to a course of action that slows the the progression of a disease, disorder or condition from an existing state to a more deleterious state.

Variant: The terms “variant”, “protein variant” or “variant protein” or “variant polypeptide” are used interchangeably herein to refer to a polypeptide that differs from a parent polypeptide by virtue of at least one amino acid modification, substitution, or deletion. The parent polypeptide may be a naturally occurring or wild-type (WT) polypeptide or may be a modified version of a WT polypeptide. The term variant polypeptide may refer to the polypeptide itself, a composition comprising the polypeptide, or the nucleic acid sequence that encodes it. In some embodiments, the variant polypeptide comprises from about one to about ten, alternatively about one to about eight, alternatively about one to about seven, alternatively about one to about five, alternatively about one to about four, alternatively from about one to about three alternatively from one to two amino acid modifications, substitutions, or deletions, or alternatively a single amino acid amino acid modification, substitution, or deletion compared to the parent polypeptide. A variant may be at least about 99% identical, alternatively at least about 98% identical, alternatively at least about 97% identical, alternatively at least about 95% identical, or alternatively at least about 90% identical to the parent polypeptide from which the variant is derived. The term “variant” also includes a nucleic acid molecule that encodes a protein or polypeptide having an altered or modified amino acid sequence compared to a parent polypeptide.

Wild-type: By “wild-type” or “WT” or “native” herein is meant an amino acid sequence or a nucleotide sequence that is found in nature, including allelic variations. A wild-type protein, polypeptide, antibody, immunoglobulin, IgG, etc. has an amino acid sequence or a nucleotide sequence that has not been modified by the hand of man.

It will be understood that individual embodiments, which are separately described herein for clarity and brevity, can be combined without limitation. Thus, the present disclosure includes one or more, or all, combinations of the embodiments described herein as if each and every combination was individually and explicitly disclosed. This also applies to any and all sub-combinations of the embodiments disclosed herein, such that the present disclosure includes one or more, or all, sub-combinations of the embodiments described herein as if each and every sub-combination was individually and explicitly disclosed.

### Human IL10

Human IL10 (hIL10) is non-covalently linked homodimeric protein comprising two identical subunits. Each hIL10 monomer is expressed as a 178 amino acid pre-protein comprising an 18 amino acid signal sequence (SEQ ID NO:5), which is post-translationally removed to render a 160 amino acid mature protein. The canonical amino acid sequence of the mature (“wild-type”) IL10 protein (UniProt Reference No. P22301) without the signal sequence (corresponding to amino acids 19-178 of the pre-protein) has the amino acid sequence:

(SEQ ID NO: 4)
SPGQGTQSENSCTHFPGNLPNMLRDLRDAFSRVKTFFQMKDQLDNLLLKE
SLLEDFKGYLGCQALSEMIQFYLEEVMPQAENQDPDIKAHVNSLGENLKT
LRLRLRRCHRFLPCENKSKAVEQVKNAFNKLQEKGIYKAMSEFDIFINYI
EAYMTMKIRN

### Fused Human IL10 Polypeptides

The present disclosure provides a fused human IL10 (hIL10) polypeptide of the formula:

(hIL10A)-Lₙ-(hIL10B),

wherein hIL10A and hIL10B are independently selected from the group consisting of a wild-type hIL10 (SEQ ID NO:4) and an hIL10 mutein, wherein at least one of hIL10A or hIL10B is an hIL10 mutein; L is a linker and n=0 (absent) or 1 (present).
hIL10 Muteins

In some embodiments, at least one of hIL10A and hIL10B in a fused hIL10 polypeptide of the formula (hIL10A)-Lₙ-(hIL10B) is a hIL10 mutein. In some cases, a hIL10 mutein can comprise one or more amino acid substitutions at a position corresponding to residues T100, H14, N18, N21, M22, R24, D25, D28, R32, E74, H90, N92, S93, E96, and R104 of SEQ ID NO: 4. In some embodiments, the hIL10 mutein comprises a sequence having at least 90% (e.g., 90%, 91%, 92%, 93%, 94%, 95%, 96%, 97%, 98%, or 99%) identity to SEQ ID NO:4 and comprises one or more amino acid substitutions at positions corresponding to residues T100, H14, N18, N21, M22, R24, D25, D28, R32, E74, H90, N92, S93, E96, and R104 of SEQ ID NO: 4 of SEQ ID NO: 4. In some cases, a hIL10 mutein can comprise one or more amino acid substitutions at a position corresponding to residues T100, H14, N18, N21, M22, R24, D25, D28, R32, E74, H90, N92, S93, E96, and R104 of SEQ ID NO: 4. In some embodiments, the hIL10 mutein comprises a sequence having at least 90% (e.g., 90%, 91%, 92%, 93%, 94%, 95%, 96%, 97%, 98%, or 99%) identity to SEQ ID NO:4 and comprises one or more amino acid substitutions at positions corresponding to residues T100, H14, N18, N21, M22, R24, D25, D28, R32, E74, H90, N92, S93, E96, and R104 of SEQ ID NO: 4.

In some embodiments, the hIL10 mutein comprises an amino acid substitution at position T100, for example, T100D, T100V, T100E, T100A, T100R, T100N, T100Q, T100E, T100I, T100L, T100K, T100M, or T100S.

In some embodiments, the hIL10 mutein comprises an amino acid substitution at position H14, for example, H14C, H14F, H14P, H14W, H14G, H14A, H14D, H14E, H14I, H14K, H14L, H14M, H14N, H14Q, H14R, H14S, H14T, H14Y, or H14V.

In some embodiments, the hIL10 mutein comprises an amino acid substitution at position N18, for example, N18Y, N18F, N18A, N18D, N18E, N18L, N18V, N18S, N18T, N18I, N18V, N18M, N18R and N18K, or N18H.

In some embodiments, the hIL10 mutein comprises an amino acid substitution at position N21, for example, N21A, N21R, N21Q, N21H, N21K, N21S, N21V, N21I, N21L, N21M, N21T N21C, N21D, or N21E.

In some embodiments, the hIL10 mutein comprises an amino acid substitution at position M22, for example, M22A, M22V, M22I, M22L, M22N, M22D, M22S, M22T, M22W, or M22Q.

In some embodiments, the hIL10 mutein comprises an amino acid substitution at position R24, for example, R24E, R24D, R24N, R24Q, R24A, R24S, or R24T.

In some embodiments, the hIL10 mutein comprises an amino acid substitution at position D25, for example, D25A, D25N, D25H, D25I, D25K, D25L, D25P, D25Q, or D25V.

In some embodiments, the hIL10 mutein comprises an amino acid substitution at position D28, for example, D28A, D28E, D28L, D28V, D28S, D28T, D28I, D28V, D28M, D28H, D28K, or D28R.

In some embodiments, the hIL10 mutein comprises an amino acid substitution at position R32, for example, R32A, R32D, R32E, R32L, R32V, R32S, R32T, R32I, R32V, R32M, R32N, R32Q, R32G, R32C, R32P, R32F, R32Y, or R32H.

In some embodiments, the hIL10 mutein comprises an amino acid substitution at position E74, for example, E74A, E74D, E74L, E74V, E74S, E74T, E741, E74V, E74M, E74H, E74K, or E74R.

In some embodiments, the hIL10 mutein comprises an amino acid substitution at position H90, for example, H90A, H90D, H90E, H90I, H90K, H90L, H90M, H90N, H90Q, H90R, H90S, H90T, H90Y, or H90V.

In some embodiments, the hIL10 mutein comprises an amino acid substitution at position N92, for example, N92D, N92Q, N92E, N92H, N92K, N92S, N92V, N92I, N92L, N92M, N92T, or N92A.

In some embodiments, the hIL10 mutein comprises an amino acid substitution at position S93, for example, S93E, S93A, S93R, S93N, S93D, S93Q, S93E, S93I, S93L, S93K, S93M, S93G, or S93V.

In some embodiments, the hlL10 mutein comprises an amino acid substitution at position E96, for example, E96C, E96F, E96Y, E96W, E96A, E96N, E96D3, E96Q, E96H, E96K, or E96S.

In some embodiments, the hlL10 mutein comprises an amino acid substitution at position R104, for example, R104A, R104W, R104Y, R104F, R104H, R104I D, R104E, R1004N, R1004Q, R104S, R1004T, R104I, R104L, R104V, or R104M.

Examples of hIL10 muteins that can be used in a fused hIL10 polypeptide of the formula (hIL10A)-Lₙ-(hIL10B) are provided in the Table 1A below.

TABLE 1A

| SEQ ID NO | Sequence | Description |
|---|---|---|
| 6 | SPGQGTQSENSCTHFPGNLPNMLRKLRDAFSRVKT | hIL10 1-160 |
| | FFQMKDQLDNLLLKESLLEDFKGYLGCQALSEMIQ | D25K |
| | FYLEEVMPQAENQDPDIKAHVNSLGENLKTLRLRL | |
| | RRCHRFLPCENKSKAVEQVKNAFNKLQEKGIYKA | |
| | MSEFDIFINYIEAYMTMKIRN (SEQ ID NO: 6) | |
| 7 | SCTHFPGNLPNMLRKLRDAFSRVKTFFQMKDQLDN | hIL10 11-160 |
| | LLLKESLLEDFKGYLGCQALSEMIQFYLEEVMPQA | D25K |
| | ENQDPDIKAHVNSLGENLKTLRLRLRRCHRFLPCEN | |
| | KSKAVEQVKNAFNKLQEKGIYKAMSEFDIFINYIEA | |
| | YMTMKIRN (SEQ ID NO: 7) | |
| 8 | SPGQGTQSENSCTHFPGNLPKMLRDLRDAFSRVKT | hIL10 1-160 |
| | FFQMKDQLDNLLLKESLLEDFKGYLGCQALSEMIQ | N21K |
| | FYLEEVMPQAENQDPDIKAHVNSLGENLKTLRLRL | |
| | RRCHRFLPCENKSKAVEQVKNAFNKLQEKGIYKA | |
| | MSEFDIFINYIEAYMTMKIRN (SEQ ID NO: 8) | |
| 9 | SCTHFPGNLPKMLRDLRDAFSRVKTFFQMKDQLDN | hIL10 11-160 |
| | LLLKESLLEDFKGYLGCQALSEMIQFYLEEVMPQA | N21K |
| | ENQDPDIKAHVNSLGENLKTLRLRLRRCHRFLPCEN | |
| | KSKAVEQVKNAFNKLQEKGIYKAMSEFDIFINYIEA | |
| | YMTMKIRN (SEQ ID NO: 9) | |
| 10 | SPGQGTQSENSCTHFPGNLPNALRDLRDAFSRVKTF | hIL10 1-160 |
| | FQMKDQLDNLLLKESLLEDFKGYLGCQALSEMIQF | M22A |
| | YLEEVMPQAENQDPDIKAHVNSLGENLKTLRLRLR | |
| | RCHRFLPCENKSKAVEQVKNAFNKLQEKGIYKAMS | |
| | EFDIFINYIEAYMTMKIRN (SEQ ID NO: 10) | |
| 11 | SCTHFPGNLPNALRDLRDAFSRVKTFFQMKDQLDN | hIL10 11-160 |
| | LLLKESLLEDFKGYLGCQALSEMIQFYLEEVMPQA | M22A |
| | ENQDPDIKAHVNSLGENLKTLRLRLRRCHRFLPCEN | |
| | KSKAVEQVKNAFNKLQEKGIYKAMSEFDIFINYIEA | |
| | YMTMKIRN (SEQ ID NO: 11) | |
| 12 | SPGQGTQSENSCTHFPGNLPNSLRDLRDAFSRVKTF | hIL10 1-160 |
| | FQMKDQLDNLLLKESLLEDFKGYLGCQALSEMIQF | M22S |
| | YLEEVMPQAENQDPDIKAHVNSLGENLKTLRLRLR | |
| | RCHRFLPCENKSKAVEQVKNAFNKLQEKGIYKAMS | |
| | EFDIFINYIEAYMTMKIRN (SEQ ID NO: 12) | |
| 13 | SCTHFPGNLPNSLRDLRDAFSRVKTFFQMKDQLDN | hIL10 11-160 |
| | LLLKESLLEDFKGYLGCQALSEMIQFYLEEVMPQA | M22S |
| | ENQDPDIKAHVNSLGENLKTLRLRLRRCHRFLPCEN | |
| | KSKAVEQVKNAFNKLQEKGIYKAMSEFDIFINYIEA | |
| | YMTMKIRN (SEQ ID NO: 13) | |
| 14 | SPGQGTQSENSCTHFPGNLPNMLRDLRDAFSRVKT | hIL10 1-160 |
| | FFQMKDQLDNLLLKESLLEDFKGYLGCQALSEMIQ | T100L |
| | FYLEEVMPQAENQDPDIKAHVNSLGENLKLLRLRL | |
| | RRCHRFLPCENKSKAVEQVKNAFNKLQEKGIYKA | |
| | MSEFDIFINYIEAYMTMKIRN (SEQ ID NO: 14) | |
| 15 | SCTHFPGNLPNMLRDLRDAFSRVKTFFQMKDQLDN | hIL10 11-160 |
| | LLLKESLLEDFKGYLGCQALSEMIQFYLEEVMPQA | T100L |
| | ENQDPDIKAHVNSLGENLKLLRLRLRRCHRFLPCEN | |
| | KSKAVEQVKNAFNKLQEKGIYKAMSEFDIFINYIEA | |
| | YMTMKIRN (SEQ ID NO: 15) | |
| 16 | SPGQGTQSENSCTHFPGNLPNMLRDLRDAFSRVKT | hIL10 1-160 |
| | FFQMKDQLDNLLLKESLLEDFKGYLGCQALSEMIQ | E96Q |
| | FYLEEVMPQAENQDPDIKAHVNSLGQNLKTLRLRL | |
| | RRCHRFLPCENKSKAVEQVKNAFNKLQEKGIYKA | |
| | MSEFDIFINYIEAYMTMKIRN (SEQ ID NO: 16) | |
| 17 | SCTHFPGNLPNMLRDLRDAFSRVKTFFQMKDQLDN | hIL10 11-160 |
| | LLLKESLLEDFKGYLGCQALSEMIQFYLEEVMPQA | E96Q |
| | ENQDPDIKAHVNSLGQNLKTLRLRLRRCHRFLPCE | |
| | NKSKAVEQVKNAFNKLQEKGIYKAMSEFDIFINYIE | |
| | AYMTMKIRN (SEQ ID NO: 17) | |
| 18 | SENSCTHFPGNLPNMLRDLRDAFSRVKTFFQMKDQ | hIL10 WT N- |
| | LDNLLLKESLLEDFKGYLGCQALSEMIQFYLEEVM | term del 8-160 |
| | PQAENQDPDIKAHVNSLGENLKTLRLRLRRCHRFLP | |
| | CENKSKAVEQVKNAFNKLQEKGIYKAMSEFDIFIN | |
| | YIEAYMTMKIRN (SEQ ID NO: 18) | |
| 148 | SPGQGTQSENSCTHFPGNLPDMLRDLRDAFSRVKT | hIL10 1-160 |
| | FFQMKDQLDNLLLKESLLEDFKGYLGCQALSEMIQ | N21D |
| | FYLEEVMPQAENQDPDIKAHVNSLGENLKTLRLRL | |
| | RRCHRFLPCENKSKAVEQVKNAFNKLQEKGIYKA | |
| | MSEFDIFINYIEAYMTMKIRN (SEQ ID NO: 148) | |
| 149 | SPGQGTQSENSCTHFPGNLPEMLRDLRDAFSRVKTF | hIL10 1-160 |
| | FQMKDQLDNLLLKESLLEDFKGYLGCQALSEMIQF | N21E |
| | YLEEVMPQAENQDPDIKAHVNSLGENLKTLRLRLR | |
| | RCHRFLPCENKSKAVEQVKNAFNKLQEKGIYKAMS | |
| | EFDIFINYIEAYMTMKIRN (SEQ ID NO: 149) | |
| 150 | SPGQGTQSENSCTHFPGNLPDTLRDLRDAFSRVKTF | hIL10 1-160 |
| | FQMKDQLDNLLLKESLLEDFKGYLGCQALSEMIQF | M22T |
| | YLEEVMPQAENQDPDIKAHVNSLGENLKTLRLRLR | |
| | RCHRFLPCENKSKAVEQVKNAFNKLQEKGIYKAMS | |
| | EFDIFINYIEAYMTMKIRN (SEQ ID NO: 150) | |
| 151 | SPGQGTQSENSCTHFPGNLPDDLRDLRDAFSRVKTF | hIL10 1-160 |
| | FQMKDQLDNLLLKESLLEDFKGYLGCQALSEMIQF | M22D |
| | YLEEVMPQAENQDPDIKAHVNSLGENLKTLRLRLR | |
| | RCHRFLPCENKSKAVEQVKNAFNKLQEKGIYKAMS | |
| | EFDIFINYIEAYMTMKIRN (SEQ ID NO: 151) | |
| 152 | SPGQGTQSENSCTHFPGNLPDWLRDLRDAFSRVKT | hIL10 1-160 |
| | FFQMKDQLDNLLLKESLLEDFKGYLGCQALSEMIQ | M22W |
| | FYLEEVMPQAENQDPDIKAHVNSLGENLKTLRLRL | |
| | RRCHRFLPCENKSKAVEQVKNAFNKLQEKGIYKA | |
| | MSEFDIFINYIEAYMTMKIRN (SEQ ID NO: 152) | |
| 153 | SPGQGTQSENSCTHFPGNLPNMLEDLRDAFSRVKT | hIL10 1-160 |
| | FFQMKDQLDNLLLKESLLEDFKGYLGCQALSEMIQ | R24E |
| | FYLEEVMPQAENQDPDIKAHVNSLGENLKTLRLRL | |
| | RRCHRFLPCENKSKAVEQVKNAFNKLQEKGIYKA | |
| | MSEFDIFINYIEAYMTMKIRN (SEQ ID NO: 153) | |
| 154 | SPGQGTQSENSCTHFPGNLPNMLRDLRDAFSRVKT | hIL10 1-160 |
| | FFQMKDQLDNLLLKESLLEDFKGYLGCQALSEMIQ | E96K |
| | FYLEEVMPQAENQDPDIKAHVNSLGKNLKTLRLRL | |
| | RRCHRFLPCENKSKAVEQVKNAFNKLQEKGIYKA | |
| | MSEFDIFINYIEAYMTMKIRN (SEQ ID NO: 154) | |
| 155 | SPGQGTQSENSCTHFPGNLPNMLRDLRDAFSRVKT | hIL10 1-160 |
| | FFQMKDQLDNLLLKESLLEDFKGYLGCQALSEMIQ | T100E |
| | FYLEEVMPQAENQDPDIKAHVNSLGENLKELRLRL | |
| | RRCHRFLPCENKSKAVEQVKNAFNKLQEKGIYKA | |
| | MSEFDIFINYIEAYMTMKIRN (SEQ ID NO: 155) | |
| 156 | SPGQGTQSENSCTHFPGNLPNMLRDLRDAFSRVKT | hIL10 1-160 |
| | FFQMKDQLDNLLLKESLLEDFKGYLGCQALSEMIQ | T100C |
| | FYLEEVMPQAENQDPDIKAHVNSLGENLKCLRLRL | |
| | RRCHRFLPCENKSKAVEQVKNAFNKLQEKGIYKA | |
| | MSEFDIFINYIEAYMTMKIRN (SEQ ID NO: 156) | |

Examples of hIL10 muteins that can be used in a fused hIL10 polypeptide of the formula (hIL10A)-Lₙ-(hIL10B) are provided in the Table 1B below.

TABLE 1B

| SEQ ID NO | Sequence | Description |
|---|---|---|
| 191 | PGQGTQSENSCTHFPGNLPNMLRKLRDAFSRVKTF | hIL10 2-160 |
| | FQMKDQLDNLLLKESLLEDFKGYLGCQALSEMIQF | D25K |
| | YLEEVMPQAENQDPDIKAHVNSLGENLKTLRLRLR | |
| | RCHRFLPCENKSKAVEQVKNAFNKLQEKGIYKAMS | |
| | EFDIFINYIEAYMTMKIRN (SEQ ID NO: 191) | |
| 192 | PGQGTQSENSCTHFPGNLPKMLRDLRDAFSRVKTF | hIL10 2-160 |
| | FQMKDQLDNLLLKESLLEDFKGYLGCQALSEMIQF | N21K |
| | YLEEVMPQAENQDPDIKAHVNSLGENLKTLRLRLR | |
| | RCHRFLPCENKSKAVEQVKNAFNKLQEKGIYKAMS | |
| | EFDIFINYIEAYMTMKIRN (SEQ ID NO: 192) | |
| 193 | PGQGTQSENSCTHFPGNLPNALRDLRDAFSRVKTFF | hIL10 2-160 |
| | QMKDQLDNLLLKESLLEDFKGYLGCQALSEMIQFY | M22A |
| | LEEVMPQAENQDPDIKAHVNSLGENLKTLRLRLRR | |
| | CHRFLPCENKSKAVEQVKNAFNKLQEKGIYKAMSE | |
| | FDIFINYIEAYMTMKIRN (SEQ ID NO: 193) | |
| 194 | PGQGTQSENSCTHFPGNLPNSLRDLRDAFSRVKTFF | hIL10 2-160 |
| | QMKDQLDNLLLKESLLEDFKGYLGCQALSEMIQFY | M22S |
| | LEEVMPQAENQDPDIKAHVNSLGENLKTLRLRLRR | |
| | CHRFLPCENKSKAVEQVKNAFNKLQEKGIYKAMSE | |
| | FDIFINYIEAYMTMKIRN (SEQ ID NO: 194) | |
| 195 | PGQGTQSENSCTHFPGNLPNMLRDLRDAFSRVKTF | hIL10 2-160 |
| | FQMKDQLDNLLLKESLLEDFKGYLGCQALSEMIQF | T100L |
| | YLEEVMPQAENQDPDIKAHVNSLGENLKLLRLRLR | |
| | RCHRFLPCENKSKAVEQVKNAFNKLQEKGIYKAMS | |
| | EFDIFINYIEAYMTMKIRN (SEQ ID NO: 195) | |
| 196 | PGQGTQSENSCTHFPGNLPNMLRDLRDAFSRVKTF | hIL10 2-160 |
| | FQMKDQLDNLLLKESLLEDFKGYLGCQALSEMIQF | E96Q |
| | YLEEVMPQAENQDPDIKAHVNSLGQNLKTLRLRLR | |
| | RCHRFLPCENKSKAVEQVKNAFNKLQEKGIYKAMS | |
| | EFDIFINYIEAYMTMKIRN (SEQ ID NO: 196) | |
| 197 | PGQGTQSENSCTHFPGNLPDMLRDLRDAFSRVKTF | hIL10 2-160 |
| | FQMKDQLDNLLLKESLLEDFKGYLGCQALSEMIQF | N21D |
| | YLEEVMPQAENQDPDIKAHVNSLGENLKTLRLRLR | |
| | RCHRFLPCENKSKAVEQVKNAFNKLQEKGIYKAMS | |
| | EFDIFINYIEAYMTMKIRN (SEQ ID NO: 197) | |
| 198 | PGQGTQSENSCTHFPGNLPEMLRDLRDAFSRVKTFF | hIL10 2-160 |
| | QMKDQLDNLLLKESLLEDFKGYLGCQALSEMIQFY | N21E |
| | LEEVMPQAENQDPDIKAHVNSLGENLKTLRLRLRR | |
| | CHRFLPCENKSKAVEQVKNAFNKLQEKGIYKAMSE | |
| | FDIFINYIEAYMTMKIRN (SEQ ID NO: 198) | |
| 199 | PGQGTQSENSCTHFPGNLPDTLRDLRDAFSRVKTFF | hIL10 2-160 |
| | QMKDQLDNLLLKESLLEDFKGYLGCQALSEMIQFY | M22T |
| | LEEVMPQAENQDPDIKAHVNSLGENLKTLRLRLRR | |
| | CHRFLPCENKSKAVEQVKNAFNKLQEKGIYKAMSE | |
| | FDIFINYIEAYMTMKIRN (SEQ ID NO: 199) | |
| 200 | PGQGTQSENSCTHFPGNLPDDLRDLRDAFSRVKTFF | hIL10 2-160 |
| | QMKDQLDNLLLKESLLEDFKGYLGCQALSEMIQFY | M22D |
| | LEEVMPQAENQDPDIKAHVNSLGENLKTLRLRLRR | |
| | CHRFLPCENKSKAVEQVKNAFNKLQEKGIYKAMSE | |
| | FDIFINYIEAYMTMKIRN (SEQ ID NO: 200) | |
| 201 | PGQGTQSENSCTHFPGNLPDWLRDLRDAFSRVKTF | hIL10 2-160 |
| | FQMKDQLDNLLLKESLLEDFKGYLGCQALSEMIQF | M22W |
| | YLEEVMPQAENQDPDIKAHVNSLGENLKTLRLRLR | |
| | RCHRFLPCENKSKAVEQVKNAFNKLQEKGIYKAMS | |
| | EFDIFINYIEAYMTMKIRN (SEQ ID NO: 201) | |
| 202 | PGQGTQSENSCTHFPGNLPNMLEDLRDAFSRVKTF | hIL10 2-160 |
| | FQMKDQLDNLLLKESLLEDFKGYLGCQALSEMIQF | R24E |
| | YLEEVMPQAENQDPDIKAHVNSLGENLKTLRLRLR | |
| | RCHRFLPCENKSKAVEQVKNAFNKLQEKGIYKAMS | |
| | EFDIFINYIEAYMTMKIRN (SEQ ID NO: 202) | |
| 203 | PGQGTQSENSCTHFPGNLPNMLRDLRDAFSRVKTF | hIL10 2-160 |
| | FQMKDQLDNLLLKESLLEDFKGYLGCQALSEMIQF | E96K |
| | YLEEVMPQAENQDPDIKAHVNSLGKNLKTLRLRLR | |
| | RCHRFLPCENKSKAVEQVKNAFNKLQEKGIYKAMS | |
| | EFDIFINYIEAYMTMKIRN (SEQ ID NO: 203) | |
| 204 | PGQGTQSENSCTHFPGNLPNMLRDLRDAFSRVKTF | hIL10 2-160 |
| | FQMKDQLDNLLLKESLLEDFKGYLGCQALSEMIQF | T100E |
| | YLEEVMPQAENQDPDIKAHVNSLGENLKELRLRLR | |
| | RCHRFLPCENKSKAVEQVKNAFNKLQEKGIYKAMS | |
| | EFDIFINYIEAYMTMKIRN (SEQ ID NO: 204) | |
| 205 | PGQGTQSENSCTHFPGNLPNMLRDLRDAFSRVKTF | hIL10 2-160 |
| | FQMKDQLDNLLLKESLLEDFKGYLGCQALSEMIQF | T100C |
| | YLEEVMPQAENQDPDIKAHVNSLGENLKCLRLRLR | |
| | RCHRFLPCENKSKAVEQVKNAFNKLQEKGIYKAMS | |
| | EFDIFINYIEAYMTMKIRN (SEQ ID NO: 205) | |

Optionally in some embodiments, in addition to the one or more amino acid substitutions relative to SEQ ID NO: 4, the hlL10 mutein can further comprise a deletion of 1, 2, 3, 4, 5, 6, 7, 8, 9, or 10 amino acid residues at the N-terminus of the mutein. In certain embodiments, the hIL10A in the fused hIL10 polypeptide has an amino-terminal deletion of 1, 2, 3, 4, 5, 6, 7, 8, 9, or 10 amino acid residues relative to SEQ ID NO: 4. In other embodiments, the hIL10B in the fused hIL10 polypeptide has an amino-terminal deletion of 1, 2, 3, 4, 5, 6, 7, 8, 9, or 10 amino acid residues relative to SEQ ID NO: 4. In certain embodiments, the hIL10A in the fused hIL10 polypeptide has an amino-terminal deletion of 1, 2, 3, 4, 5, 6, 7, 8, 9, or 10 amino acid residues relative to an amino acid sequence of Table 1A. In other embodiments, the hIL10B in the fused hIL10 polypeptide has an amino-terminal deletion of 1, 2, 3, 4, 5, 6, 7, 8, 9, or 10 amino acid residues relative to an amino acid sequence of Table 1A. In some embodiments, hIL10A is selected from an amino acid sequence of Table 1B and hIL10B is selected from an amino acid sequence of Table 1A. In some embodiments, the present disclosure provides a fused hIL10 polypeptide of the formula of (hIL10A)-Lₙ-(hIL10B) wherein hIL10A is a polypeptide is selected from the group consisting of SEQ ID NOS:191, 192, 193, 194, 195, 196, 197, 198, 199, 200, 201, 202, 203, 204, and 205 and hIL10B is a polypeptide is selected from the group consisting of SEQ ID NOS: 6, 7, 8, 9, 10, 11, 12, 13, 14, 15, 16, 17, 18, 148, 149, 150, 151, 152, 153, 154, 155, and 156.

### Linkers

In some embodiments, the hIL10A and hIL10B in the formula of (hIL10A)-Lₙ-(hIL10B) are to be linked or fused to each other by way of a linker, L, i.e., n is 1 in the formula.

In some embodiments, the linker is a polypeptide of from 1 amino acid (e.g., Thr) to 50 amino acids (e.g., 1, 2, 3, 4, 5, 6, 7, 8, 9, 10, 10-20, 20-30, 30-40, 40-50, 10-30, 20-40, or 30-50 amino acids) or more than 50 amino acids.

Examples of linkers can include, but are not limited to, (G)ₓ, (A)ₓ, (S)ₓ, (GA)ₓ, (GS)ₓ, (AS)ₓ, (AG)ₓ, (SG)ₓ, (SA)ₓ, in which x can be an integer, e.g., an integer between 1 and 50. Examples of linkers containing glycine and serine can include, but are not limited to, (GmSo)_{z} (SEQ ID NO:129), (GSGGS)_{z} (SEQ ID NO:130), (GmSoGm)_{z} (SEQ ID NO:131), (GmSoGmSoGm)_{z} (SEQ ID NO:132), (GSGGSm)_{z} (SEQ ID NO:133), (GSGSmG)_{z} (SEQ ID NO:134) and (GGGSm)_{z} (SEQ ID NO:135), and combinations thereof, where m, z, and o are each independently selected from an integer of at least 1 to 20 (e.g., 1-18, 2-16, 3-14, 4-12, 5-10, 1, 2, 3, 4, 5, 6, 7, 8, 9, 10, 11, 12, 13, 14, 15, 16, 17, 18, 19, or 20), and other flexible linkers. In some embodiments, the polypeptide linker is a glycine-serine polymer of the structure (GGGGSₘ)ₙ (SEQ ID NO: 136), (GGGSₘ)ₙ (SEQ ID NO: 137), (GGGAₘ)ₙ (SEQ ID NO:138) and (GGGGAₘ)ₙ (SEQ ID NO:139), and combinations thereof, where m, n, and o are each independently selected from 1, 2, 3 or 4. Exemplary glycine-serine linkers include but are not limited to the monomers: GGGGS (referred to as “G4S”; SEQ ID NO:104), GGGGA (referred to as “G4A”; SEQ ID NO:112), GGGS (referred to as “G3S”; SEQ ID NO:103)) and GGGA (referred to as “G3A” (SEQ ID NO:140)), or homopolymers (e.g. “GGGGSGGGGS” also referred to as (G4S)₂; SEQ ID NO:111) or heteropolymers thereof (for example: GGGGSGGGS also referred to as G4S-G3S; SEQ ID NO: 108). Particular linker sequences useful in the practice of the present disclosure include, but are not limited to: GSGG (SEQ ID NO:101), GGSG (SEQ ID NO:102), GGGS (SEQ ID NO:103), GGGGS (SEQ ID NO:104), GGGSGG (SEQ ID NO:105), GGGGSG (SEQ ID NO:106), GGGSGGGS (SEQ ID NO:107), GGGGSGGGS (SEQ ID NO:108), GGGGGSGGGS (SEQ ID NO:109), GGGSGGGGS (SEQ ID NO:110), GGGGSGGGGS (SEQ ID NO:111), GGGGA (SEQ ID NO:112), GGGGAGGGGS (SEQ ID NO:113), GGGGAGGGGSGGGGA (SEQ ID NO:114), GGGAGGGS (SEQ ID NO:115), GGGAGGGA (SEQ ID NO:116), GGGSGGGA (SEQ ID NO:117), and GGGGAGGGS (SEQ ID NO:118). In the construction of such polymers, it may be desirable to avoid repeated “GSG” sequences which potentially may provide the introduction of a non-naturally occurring glycosylation site.

In some embodiments, a polypeptide linker of a fused hIL10 polypeptide of formula (hIL10A)-Lₙ-(hIL10B) has a sequence selected from GGGSGSGSGSG (SEQ ID NO:19) or NQMFDQKYDDP (SEQ ID NO:20). In some embodiments, the linker in a fused hIL10 polypeptide of formula (hIL10A)-Lₙ-(hIL10B) is a Thr.

In some embodiments, a linker in a fused hIL10 polypeptide of formula (hIL10A)-Lₙ-(hIL10B) can be a single amino acid. For example, in some embodiments, the single amino acid is T, S, A, or G (e.g., T).

Fused hIL10 Polypeptides

In some embodiments, a fused hIL10 polypeptide of formula (hIL10A)-Lₙ-(hIL10B) can include two IL10 monomers that are the same, i.e., hIL10A and hIL10B in the formula are both wild-type IL10 molecules (e.g., SEQ ID NO:4) or are both the same hIL10 mutein. In some embodiments, a fused hIL10 polypeptide of formula (hIL10A)-Lₙ-(hIL10B) can include two different IL10 monomers, i.e., one of hIL10A and hIL10B is a wild-type IL10 molecule (e.g., SEQ ID NO:4) and the other is a hIL10 mutein, or both are different hIL10 muteins.

In some embodiments, hIL10A and hIL10B are directly fused to each other, i.e., n is 0 (no linker) in the formula of (hIL10A)-Lₙ-(hIL10B). The hIL10A and hIL10B can be fused to each other via their termini, i.e., the N-terminus of hIL10B is fused to the C-terminus of hIL10A. In some embodiments, hIL10A and hIL10B are fused to each other via a linker, i.e., n is 1. Examples of linkers are described herein.

In some embodiments, a fused hIL10 polypeptide with the formula of (hIL10A)-Lₙ-(hIL10B) can comprise a hIL10A comprising a sequence having at least one amino acid substitution and at least 90% (e.g., 90%, 91%, 92%, 93%, 94%, 95%, 96%, 97%, or 99%) identity relative to the sequence of SEQ ID NO:4, and a hIL10B comprising a sequence having at least one amino acid substitution and at least 90% (e.g., 90%, 91%, 92%, 93%, 94%, 95%, 96%, 97%, or 99%) identity relative to the sequence of SEQ ID NO:4. In some embodiments, a fused hIL10 polypeptide with the formula of (hIL10A)-Lₙ-(hIL10B) can comprise a hIL10A comprising a sequence having one, two, three, four, five, six, seven, eight, nine, or ten amino acid substitutions or deletions relative to the sequence of SEQ ID NO:4, and a hIL10B comprising a sequence having one, two, three, four, five, six, seven, eight, nine, or ten amino acid substitutions or deletions relative to the sequence of SEQ ID NO:4.

In some embodiments, a fused hIL10 polypeptide with the formula of (hIL10A)-Lₙ-(hIL10B) can comprise a hIL10A comprising a sequence having one, two, three, four, five, six, seven, eight, nine, or ten amino acid substitutions at one or more positions corresponding to residues T100, H14, N18, N21, M22, R24, D25, D28, R32, E74, H90, N92, S93, E96, and R104 of SEQ ID NO: 4 and at least 90% (e.g., 90%, 91%, 92%, 93%, 94%, 95%, 96%, 97%, or 99%) identity relative to the sequence of SEQ ID NO:4, and a hIL10B comprising a sequence having one, two, three, four, five, six, seven, eight, nine, or ten amino acid substitutions at one or more positions corresponding to residues T100, H14, N18, N21, M22, R24, D25, D28, R32, E74, H90, N92, S93, E96, and R104 of SEQ ID NO: 4 and at least 90% (e.g., 90%, 91%, 92%, 93%, 94%, 95%, 96%, 97%, or 99%) identity relative to the sequence of SEQ ID NO:4. In some embodiments, a fused hIL10 polypeptide with the formula of (hIL10A)-Lₙ-(hIL10B) can comprise a hIL10A comprising a sequence having one, two, three, four, five, six, seven, eight, nine, or ten amino acid substitutions at one or more positions corresponding to residues T100, H14, N18, N21, M22, D25, R32, S93, and E96 of SEQ ID NO:4 and at least 90% (e.g., 90%, 91%, 92%, 93%, 94%, 95%, 96%, 97%, or 99%) identity relative to the sequence of SEQ ID NO:4, and a hIL10B comprising a sequence having one, two, three, four, five, six, seven, eight, nine, or ten amino acid substitutions at one or more positions corresponding to residues T100, H14, N18, N21, M22, D25, R32, S93, and E96 of SEQ ID NO:4 and at least 90% (e.g., 90%, 91%, 92%, 93%, 94%, 95%, 96%, 97%, or 99%) identity relative to the sequence of SEQ ID NO:4.

In some embodiments, a fused hIL10 polypeptide with the formula of (hIL10A)-Lₙ-(hIL10B) can comprise a hIL10A comprising a sequence of a wild-type IL10 molecule (e.g., SEQ ID NO:4), and a hIL10B comprising a sequence having one, two, three, four, five, six, seven, eight, nine, or ten amino acid substitutions and at least 90% (e.g., 90%, 91%, 92%, 93%, 94%, 95%, 96%, 97%, or 99%) identity relative to the sequence of SEQ ID NO:4.

In some embodiments, a fused hIL10 polypeptide with the formula of (hIL10A)-Lₙ-(hIL10B) can comprise a hIL10A comprising a sequence having one, two, three, four, five, six, seven, eight, nine, or ten amino acid substitutions and at least 90% (e.g., 90%, 91%, 92%, 93%, 94%, 95%, 96%, 97%, or 99%) identity relative to the sequence of SEQ ID NO:4, and a hIL10B comprising a sequence of a wild-type IL10 molecule (e.g., SEQ ID NO: 4).

In some embodiments, a fused hIL10 polypeptide with the formula of (hIL10A)-Lₙ-(hIL10B) can comprise a hIL10A comprising a sequence having an amino acid substitution at a position corresponding to residues T100, H14, N18, N21, M22, R24, D25, D28, R32, E74, H90, N92, S93, E96, and R104 of SEQ ID NO:4 and a hIL10B comprising a sequence having an amino acid substitution at a position corresponding to residues T100, H14, N18, N21, M22, R24, D25, D28, R32, E74, H90, N92, S93, E96, and R104 of SEQ ID NO:4. In some embodiments, a fused hIL10 polypeptide with the formula of (hIL10A)-Lₙ-(hIL10B) can comprise a hIL10A comprising a sequence having an amino acid substitution at a position corresponding to residues T100, H14, N18, N21, M22, D25, R32, S93, or E96 of SEQ ID NO:4 and a hIL10B comprising a sequence having an amino acid substitution at a position corresponding to residues T100, H14, N18, N21, M22, D25, R32, S93, and E96 of SEQ ID NO:4.

In some embodiments, one or both of hIL10A and hIL10B in the fused hIL10 polypeptide with the formula of (hIL10A)-Lₙ-(hIL10B) can have an N-terminal deletion of 1, 2, 3, 4, 5, 6, 7, 8, 9, or 10 amino acid residues relative to the sequence of SEQ ID NO:4. In certain embodiments, hIL10A can have an N-terminal deletion of 1, 2, 3, 4, 5, 6, 7, 8, 9, or 10 amino acid residues relative to the sequence of SEQ ID NO:4. In certain embodiments, hIL10B can have an N-terminal deletion of 1, 2, 3, 4, 5, 6, 7, 8, 9, or 10 amino acid residues relative to the sequence of SEQ ID NO:4.

In some embodiments, a fused hIL10 polypeptide with the formula of (hIL10A)-Lₙ-(hIL10B) can comprise a hIL10A comprising a sequence having at least one (e.g., one, two, three, four, five, six, seven, eight, nine, or ten) amino acid substitution and at least 90% (e.g., 90%, 91%, 92%, 93%, 94%, 95%, 96%, 97%, or 99%) identity relative to the sequence of SEQ ID NO:4, and a hIL10B comprising a sequence having at least one (e.g., one, two, three, four, five, six, seven, eight, nine, or ten) amino acid substitution and at least 90% (e.g., 90%, 91%, 92%, 93%, 94%, 95%, 96%, 97%, or 99%) identity relative to the sequence of SEQ ID NO:4, wherein one or both of hIL10A and hIL10B has an N-terminal deletion of 1, 2, 3, 4, 5, 6, 7, 8, 9, or 10 amino acid residues relative to the sequence of SEQ ID NO:4.

In some embodiments, a fused hIL10 polypeptide with the formula of (hIL10A)-Lₙ-(hIL10B) can comprise a hIL10A comprising a sequence having an amino acid substitution at a position corresponding to residues T100, H14, N18, N21, M22, R24, D25, D28, R32, E74, H90, N92, S93, E96, and R104 of SEQ ID NO:4 and a hIL10B comprising a sequence having an amino acid substitution at a position corresponding to residues T100, H14, N18, N21, M22, R24, D25, D28, R32, E74, H90, N92, S93, E96, and R104 of SEQ ID NO:4, wherein one or both of hIL10A and hIL10B has an N-terminal deletion of 1, 2, 3, 4, 5, 6, 7, 8, 9, or 10 amino acid residues relative to the sequence of SEQ ID NO:4. In some embodiments, a fused hIL10 polypeptide with the formula of (hIL10A)-Lₙ-(hIL10B) can comprise a hIL10A comprising a sequence having an amino acid substitution at a position corresponding to residues T100, H14, N18, N21, M22, D25, R32, S93, or E96 of SEQ ID NO:4 and a hIL10B comprising a sequence having an amino acid substitution at a position corresponding to residues T100, H14, N18, N21, M22, D25, R32, S93, or E96 of SEQ ID NO:4, wherein one or both of hIL10A and hIL10B has an N-terminal deletion of 1, 2, 3, 4, 5, 6, 7, 8, 9, or 10 amino acid residues relative to the sequence of SEQ ID NO:4.

In some embodiments, the present disclosure provides a fused hIL10 polypeptide of formula (hIL10A)-Lₙ-(hIL10B) wherein at least one of hIL10A or hIL10B is an hIL10 mutein selected from the group consisting of SEQ ID NOS:6, 7, 8, 9, 10, 11, 12, 13, 14, 15, 16, 17, 18, 148, 149, 150, 151, 152, 153, 154, 155, 156, 191, 192, 193, 194, 195, 196, 197, 198, 199, 200, 201, 202, 203, 204, and 205, optionally wherein one or both of hIL10A and hIL10B has an N-terminal deletion of 1, 2, 3, 4, 5, 6, 7, 8, 9, or 10 amino acid residues relative to the sequence of SEQ ID NO:4.

In some embodiments, the present disclosure provides a fused hIL10 polypeptide of formula (hIL10A)-Lₙ-(hIL10B) wherein hIL10A and hIL10B are hIL10 muteins independently selected from the group consisting of SEQ ID NOS: 6, 7, 8, 9, 10, 11, 12, 13, 14, 15, 16, 17, 18, 148, 149, 150, 151, 152, 153, 154, 155, 156, 191, 192, 193, 194, 195, 196, 197, 198, 199, 200, 201, 202, 203, 204, and 205, optionally wherein one or both of hIL10A and hIL10B has an N-terminal deletion of 1, 2, 3, 4, 5, 6, 7, 8, 9, or 10 amino acid residues relative to the sequence of SEQ ID NO:4.

In some embodiments, the present disclosure provides a fused hIL10 polypeptide of formula (hIL10A)-Lₙ-(hIL10B) wherein both of hIL10A and hIL10B is an hIL10 mutein selected from the group consisting of SEQ ID NOS:6, 7, 8, 9, 10, 11, 12, 13, 14, 15, 16, 17, 18, 148, 149, 150, 151, 152, 153 154, 155, 156, 191, 192, 193, 194, 195, 196, 197, 198, 199, 200, 201, 202, 203, 204, and 205 optionally wherein one or both of hIL10A and hIL10B has an N-terminal deletion of 1, 2, 3, 4, 5, 6, 7, 8, 9, or 10 amino acid residues relative to the sequence of SEQ ID NO:4.

In some embodiments, the present disclosure provides a fused hIL10 polypeptide of formula (hIL10A)-Lₙ-(hIL10B) wherein hIL10A comprises an N-terminal deletion of 1, 2, 3, 4, 5, 6, 7, 8, 9, or 10 amino acid residues. In some embodiments, deletion of N-terminal amino acids (e.g., the first three or six amino acids of the N-terminus of the fused IL10 molecules are deleted relative to the wild-type sequence), the resulting N-terminal amino acid is glutamine (Q). N-terminal glutamine residues have been observed to spontaneously cyclize to form pyroglutamate (pE) at or near physiological conditions. (See e.g., Liu, et al (2011) J. Biol. Chem. 286(13): 11211-11217). In some embodiments, the formation of pyroglutamate complicates N-terminal PEG conjugation particularly when aldehyde chemistry is used for N-terminal PEGylation. Consequently, when PEGylating fused IL10 molecules comprising an N-terminal glutamine, for example by deletion of the N-terminal 3 or 6 residues from the wild-type sequence, the N-terminal glutamine is substituted with an alternative amino acid. In some embodiments, the N-terminal glutamine residue is selected from the group consisting of E and D.

In some embodiments, the present disclosure provides a fused hIL10 polypeptide of formula (hIL10A)-Lₙ-(hIL10B) wherein hIL10A comprises an N-terminal deletion of 3 amino acids and an amino acid substitution selected from the group consisting of Q4E and Q4D. In some embodiments, the present disclosure provides a fused hIL10 polypeptide of formula (hIL10A)-Lₙ-(hIL10B) wherein hIL10A comprises an N-terminal deletion of 6 amino acids and an amino acid substitution selected from the group consisting of Q7E and Q7D.

In particular embodiments, a fused hIL10 polypeptide with the formula of (hIL10A)-Lₙ-(hIL10B) can comprise a hIL10A comprising a sequence having the amino acid substitution D25K and at least 90% (e.g., 90%, 91%, 92%, 93%, 94%, 95%, 96%, 97%, or 99%) identity relative to the sequence of SEQ ID NO:4, and a hIL10B comprising a sequence having the amino acid substitution D25K and at least 90% (e.g., 90%, 91%, 92%, 93%, 94%, 95%, 96%, 97%, or 99%) identity relative to the sequence of SEQ ID NO:4, wherein one or both of hIL10A and hIL10B has an N-terminal deletion of SPGQGTQSEN (SEQ ID NO:21) relative to the sequence of SEQ ID NO:4 (e.g., hIL10B has an N-terminal deletion of SPGQGTQSEN (SEQ ID NO:21) relative to the sequence of SEQ ID NO:4).

In particular embodiments, a fused hIL10 polypeptide with the formula of (hIL10A)-Lₙ-(hIL10B) can comprise a hIL10A comprising a sequence having the amino acid substitution N21K and at least 90% (e.g., 90%, 91%, 92%, 93%, 94%, 95%, 96%, 97%, or 99%) identity relative to the sequence of SEQ ID NO:4, and a hIL10B comprising a sequence having the amino acid substitution N21K and at least 90% (e.g., 90%, 91%, 92%, 93%, 94%, 95%, 96%, 97%, or 99%) identity relative to the sequence of SEQ ID NO:4, wherein one or both of hIL10A and hIL10B has an N-terminal deletion of SPGQGTQSEN (SEQ ID NO:21) relative to the sequence of SEQ ID NO:4 (e.g., hIL10B has an N-terminal deletion of SPGQGTQSEN (SEQ ID NO:21) relative to the sequence of SEQ ID NO:4).

In particular embodiments, a fused hIL10 polypeptide with the formula of (hIL10A)-Lₙ-(hIL10B) can comprise a hIL10A comprising a sequence having the amino acid substitution M22A and at least 90% (e.g., 90%, 91%, 92%, 93%, 94%, 95%, 96%, 97%, or 99%) identity relative to the sequence of SEQ ID NO:4, and a hIL10B comprising a sequence having the amino acid substitution M22A and at least 90% (e.g., 90%, 91%, 92%, 93%, 94%, 95%, 96%, 97%, or 99%) identity relative to the sequence of SEQ ID NO:4, wherein one or both of hIL10A and hIL10B has an N-terminal deletion of SPGQGTQSEN (SEQ ID NO:21) relative to the sequence of SEQ ID NO:4 (e.g., hIL10B has an N-terminal deletion of SPGQGTQSEN (SEQ ID NO:21) relative to the sequence of SEQ ID NO:4).

In particular embodiments, a fused hIL10 polypeptide with the formula of (hIL10A)-Lₙ-(hIL10B) can comprise a hIL10A comprising a sequence having the amino acid substitution M22S and at least 90% (e.g., 90%, 91%, 92%, 93%, 94%, 95%, 96%, 97%, or 99%) identity relative to the sequence of SEQ ID NO:4, and a hIL10B comprising a sequence having the amino acid substitution M22S and at least 90% (e.g., 90%, 91%, 92%, 93%, 94%, 95%, 96%, 97%, or 99%) identity relative to the sequence of SEQ ID NO:4, wherein one or both of hIL10A and hIL10B has an N-terminal deletion of SPGQGTQSEN (SEQ ID NO:21) relative to the sequence of SEQ ID NO:4 (e.g., hIL10B has an N-terminal deletion of SPGQGTQSEN (SEQ ID NO:21) relative to the sequence of SEQ ID NO:4).

In particular embodiments, a fused hIL10 polypeptide with the formula of (hIL10A)-Lₙ-(hIL10B) can comprise a hIL10A comprising a sequence having the amino acid substitution T100L and at least 90% (e.g., 90%, 91%, 92%, 93%, 94%, 95%, 96%, 97%, or 99%) identity relative to the sequence of SEQ ID NO:4, and a hIL10B comprising a sequence having the amino acid substitution T100L and at least 90% (e.g., 90%, 91%, 92%, 93%, 94%, 95%, 96%, 97%, or 99%) identity relative to the sequence of SEQ ID NO:4, wherein one (e.g., only hIL10A or only hIL10B) or both of hIL10A and hIL10B has an N-terminal deletion of SPGQGTQSEN (SEQ ID NO:21) relative to the sequence of SEQ ID NO:4 (e.g., hIL10B has an N-terminal deletion of SPGQGTQSEN (SEQ ID NO:21) relative to the sequence of SEQ ID NO:4).

In particular embodiments, a fused hIL10 polypeptide with the formula of (hIL10A)-Lₙ-(hIL10B) can comprise a hIL10A comprising a sequence having the amino acid substitution T100L, and a hIL10B comprising a sequence having the amino acid substitution T100L, wherein hIL10B further has an N-terminal deletion of SPGQGTQSEN (SEQ ID NO:21) relative to the sequence of SEQ ID NO:4, n is 1, and L is GGGSGSGSGSG (SEQ ID NO:19). In certain embodiments, the fused hIL10 polypeptide has the sequence of SEQ ID NO:36.

In particular embodiments, a fused hIL10 polypeptide with the formula of (hIL10A)-Lₙ-(hIL10B) can comprise a hIL10A comprising a sequence having the amino acid substitution T100L, and a hIL10B comprising a sequence having the amino acid substitution T100L, wherein n is 1, and L is T. In certain embodiments, the fused hIL10 polypeptide has the sequence of SEQ ID NO:37.

In particular embodiments, a fused hIL10 polypeptide with the formula of (hIL10A)-Lₙ-(hIL10B) can comprise a hIL10A comprising a sequence having the amino acid substitution T100L, and a hIL10B comprising a sequence having the amino acid substitution T100L, wherein hIL10B also has an N-terminal deletion of SPGQGTQSEN (SEQ ID NO:21) relative to the sequence of SEQ ID NO:4, n is 1, and L is NQMFDQKYDDP (SEQ ID NO:20). In certain embodiments, the fused hIL10 polypeptide has the sequence of SEQ ID NO:38.

In particular embodiments, a fused hIL10 polypeptide with the formula of (hIL10A)-Lₙ-(hIL10B) can comprise a hIL10A comprising a sequence having the amino acid substitution T100L, and a hIL10B comprising a sequence having the amino acid substitution T100L, wherein n is 0. In certain embodiments, the fused hIL10 polypeptide has a sequence having at least 90% (e.g., 90%, 91%, 92%, 93%, 94%, 95%, 96%, 97%, 98%, 99%, or 100%) identity to the sequence of SEQ ID NO:44.

In particular embodiments, a fused hIL10 polypeptide with the formula of (hIL10A)-Lₙ-(hIL10B) can comprise a hIL10A comprising a sequence having the amino acid substitution E96Q and at least 90% (e.g., 90%, 91%, 92%, 93%, 94%, 95%, 96%, 97%, or 99%) identity relative to the sequence of SEQ ID NO:4, and a hIL10B comprising a sequence having the amino acid substitution E96Q and at least 90% (e.g., 90%, 91%, 92%, 93%, 94%, 95%, 96%, 97%, or 99%) identity relative to the sequence of SEQ ID NO:4, wherein one or both of hIL10A and hIL10B has an N-terminal deletion of SPGQGTQSEN (SEQ ID NO:21) relative to the sequence of SEQ ID NO:4 (e.g., hIL10B has an N-terminal deletion of SPGQGTQSEN (SEQ ID NO:21) relative to the sequence of SEQ ID NO:4).

In particular embodiments, a fused hIL10 polypeptide with the formula of (hIL10A)-Lₙ-(hIL10B) can comprise a hIL10A comprising a sequence having the amino acid substitution T100L and at least 90% (e.g., 90%, 91%, 92%, 93%, 94%, 95%, 96%, 97%, or 99%) identity relative to the sequence of SEQ ID NO:4, and a hIL10B comprising a sequence having at least 90% (e.g., 90%, 91%, 92%, 93%, 94%, 95%, 96%, 97%, 99%, or 100%) identity relative to the sequence of SEQ ID NO:4.

In particular embodiments, a fused hIL10 polypeptide with the formula of (hIL10A)-Lₙ-(hIL10B) can comprise a hIL10A comprising a sequence having at least 90% (e.g., 90%, 91%, 92%, 93%, 94%, 95%, 96%, 97%, 99%, or 100%) identity relative to the sequence of SEQ ID NO:4, and a hIL10B comprising a sequence having at least 90% (e.g., 90%, 91%, 92%, 93%, 94%, 95%, 96%, 97%, 99%, or 100%) identity relative to the sequence of SEQ ID NO:4, wherein one or both of hIL10A and hIL10B has an N-terminal deletion of SPGQGTQ (SEQ ID NO:22) or SPGQGTQSEN (SEQ ID NO:21) relative to the sequence of SEQ ID NO:4 (e.g., hIL10A has an N-terminal deletion of SPGQGTQSEN (SEQ ID NO:21) relative to the sequence of SEQ ID NO:4).

Examples of fused hIL10 polypeptides and the nucleic acid sequences encoding them are shown in the Table 2 below. The linker sequence (L) is highlighted in bold typeface. In some embodiments, the fused hIL10 polypeptide comprises one of the polypeptide sequences in Table 2, optionally with the linker depicted, or having a different linker, for example as described herein, or lacking a linker). In some embodiments, for each of the fused hIL10 polypeptide sequence listed below, a His-tag can be fused to the N-terminus of the sequence with or without a linker, i.e., GGSHHHHHHHH (SEQ ID NO:23) can be fused to the N-terminus of a fused hIL10 polypeptide listed below. The fused hIL10 polypeptides listed in Table 2 are made with mature hIL10 monomers that have the 18 amino acid signal sequence (SEQ ID NO:5) cleaved.

In some embodiments, the present disclosure provides a fused IL10 polypeptide the formula of (hIL10A)-Lₙ-(hIL10B) having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to a polypeptide selected from the group consisting of SEQ ID NOS:24, 25, 26, 27, 28, 29, 30, 31, 32, 33, 34, 35, 36, 37, 38, 39, 40, 41, 42, 43, 44, 45, 46, 47, 48, 49, 50, 119, 157, 158, 159, 160, 161, 162, 163, 164, 165, 166, 167, 168, 169, 170, 171, 172, 173, 174, 175, 176, 177, 178, 179, 180, 181, 182, 183 184, 185 and 186.

The disclosure provides a fused IL10 polypeptide that has at least 90% (e.g., 90%, 91%, 92%, 93%, 94%, 95%, 96%, 97%, 99%, or 100%) identity to any one of the polypeptide sequences of Table 2. In some embodiments, the disclosure provides a fused IL10 polypeptide that has at least 90% alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to any one of the polypeptide sequences of Table 2, optionally with the linker depicted, or having a different linker, for example as described herein, or lacking a linker.

In some embodiments the fused IL10 polypeptide the formula of (hIL10A)-Lₙ-(hIL10B) is selected from the group consisting of SEQ ID NOS: 24, 25, 26, 27, 28, 29, 30, 31, 32, 33, 34, 35, 36, 37, 38, 39, 40, 41, 42, 43, 44, 45, 46, 47, 48, 49, 50, and 119.

TABLE 2
Exemplary Fused IL10 Polypeptides and Nucleic Acids Encoding Same

| Name | Protein Sequence | DNA Sequence |
|---|---|---|
| TP0002 | SPGQGTQSENSCTHFPGNL | agccccggccaaggcacacagagcgagaactcatgtacccattttcccg |
| | PNMLRKLRDAFSRVKTFFQ | ggaacctgcccaacatgctgcggaagctgagagacgccttcagcagagt |
| | MKDQLDNLLLKESLLEDF | gaagactttctttcagatgaaggatcagctggacaaccttttactgaaggag |
| | KGYLGCQALSEMIQFYLEE | agtctgctagaagacttcaagggatacctgggctgtcaagccttgtcggag |
| | VMPQAENQDPDIKAHVNS | atgattcaattctacctggaagaagtgatgccccaagctgagaaccaaga |
| | LGENLKTLRLRLRRCHRFL | ccccgatatcaaggcccatgtgaacagcctgggcgagaatctcaaaacc |
| | PCENKSKAVEQVKNAFNK | ctgcgcttaaggctcagaagatgccacagatttctgccatgcgaaaacaa |
| | LQEKGIYKAMSEFDIFINYI | gagcaaggcagtggaacaagtgaaaaacgccttcaacaagctgcaaga |
| | EAYMTMKIRNGGGSGSGS | gaagggcatctacaaagccatgagcgagttcgacattttcatcaactatatt |
| | GSGSCTHFPGNLPNMLRKL | gaggcctacatgacgatgaagattcgaaatggcggagggtcgggctccg |
| | RDAFSRVKTFFQMKDQLD | gctctggcagcggctcctgcacccatttccccggcaacctgcccaatatg |
| | NLLLKESLLEDFKGYLGCQ | ctgagaaagctgcgtgacgccttctctagagtaaaaaccttctttcagatga |
| | ALSEMIQFYLEEVMPQAEN | aggatcagctggacaacttgttactgaaggagagcctgctcgaggacttc |
| | QDPDIKAHVNSLGENLKTL | aaaggctacctgggctgccaagccctgagcgagatgattcagttttacctt |
| | RLRLRRCHRFLPCENKSKA | gaagaggtgatgccccaagccgagaaccaagaccccgacatcaaagcc |
| | VEQVKNAFNKLQEKGIYK | cacgtcaactcgctgggcgaaaacctgaagactcttcggttgagacttcga |
| | AMSEFDIFINYIEAYMTMKI | agatgtcatcggttcttgccctgcgagaataagagcaaggcggtggagca |
| | RN (SEQ ID NO: 24) | agtgaagaacgcattcaataagctgcaagagaaaggtatctacaaggcta |
| | | tgagcgaatttgacatctttattaattacatcgaggcctatatgaccatgaaa |
| | | atcagaaat (SEQ ID NO: 51) |
| TP0003 | SPGQGTQSENSCTHFPGNL | agtcccggacaaggcacacagagcgagaacagctgcacgcatttcccc |
| | PNMLRKLRDAFSRVKTFFQ | ggcaacctgcccaacatgctgcgtaaactcagagatgctttcagcagagt |
| | MKDQLDNLLLKESLLEDF | gaaaacgttcttccaaatgaaagaccaactcgataacctgttgcttaagga |
| | KGYLGCQALSEMIQFYLEE | gtcactgctggaagatttcaaaggctatcttggctgtcaagccctgagcga |
| | VMPQAENQDPDIKAHVNS | aatgatacagttctacctggaagaggtgatgcctcaagccgagaaccaag |
| | LGENLKTLRLRLRRCHRFL | acccggatattaaggcccatgtaaattccctgggcgagaacctgaaaacc |
| | PCENKSKAVEQVKNAFNK | ctcagactaagactgcgacgatgtcatagattcctaccctgtgaaaacaaa |
| | LQEKGIYKAMSEFDIFINYI | tctaaggccgtggagcaagtgaagaatgcttttaacaaactgcaagagaa |
| | EAYMTMKIRNTSPGQGTQ | gggcatttacaaggccatgagcgaatttgacatcttcatcaattacatcgaa |
| | SENSCTHFPGNLPNMLRKL | gcctacatgaccatgaaaattagaaacacgtcacccggccaaggcacac |
| | RDAFSRVKTFFQMKDQLD | agtctgaaaacagctgcacccactttcccggcaaccttcccaacatgttgc |
| | NLLLKESLLEDFKGYLGCQ | gaaagctgagagacgcctttagccgcgtgaaaaccttcttccaaatgaaa |
| | ALSEMIQFYLEEVMPQAEN | gatcagctggacaatctgctgctcaaggaaagcctgctcgaggacttcaa |
| | QDPDIKAHVNSLGENLKTL | gggctacctcggctgccaagccctcagcgagatgattcagttttaccttga |
| | RLRLRRCHRFLPCENKSKA | agaagtcatgcctcaagccgaaaaccaagaccccgatataaaagcccat |
| | VEQVKNAFNKLQEKGIYK | gtgaacagcttgggcgagaacttaaagaccctgagattaagattacggcg |
| | AMSEFDIFINYIEAYMTMKI | gtgccaccgattcttgccgtgcgagaacaagtcaaaggcggtcgagcaa |
| | RN (SEQ ID NO: 25) | gtgaagaatgcattcaataaattacaagagaagggcatctataaggctatg |
| | | agtgagttcgatattttcatcaactatatcgaggcttacatgacaatgaaaat |
| | | cagaaac (SEQ ID NO: 52) |
| TP0004 | SPGQGTQSENSCTHFPGNL | tcacccggccaaggcacacagagcgagaactcctgcacccacttccccg |
| | PNMLRKLRDAFSRVKTFFQ | ggaacttgcccaacatgctgcgcaagctgagagatgccttcagcagagtg |
| | MKDQLDNLLLKESLLEDF | aagactttctttcagatgaaggatcagcttgacaaccttctcctgaaggaga |
| | KGYLGCQALSEMIQFYLEE | gtctgctagaagacttcaagggatacctgggctgtcaagccttgtcggaga |
| | VMPQAENQDPDIKAHVNS | tgattcagttttatctggaagaggtgatgccccaagctgagaatcaagacc |
| | LGENLKTLRLRLRRCHRFL | ccgacatcaaagcccatgtgaacagcctgggcgagaatctcaaaaccct |
| | ENQDPDIKAHVNSLGENLK | gcgccttcgattgagaagatgccacagatttctgccatgcgaaaacaaga |
| | PCENKSKAVEQVKNAFNK | gcaaggcagtggaacaagtgaaaaatgccttcaacaagctgcaagagaa |
| | LQEKGIYKAMSEFDIFINYI | gggcatctacaaagccatgagcgagttcgacattttcatcaactacataga |
| | EAYMTMKIRNNQMFDQK | ggcttacatgaccatgaagatcagaaacaaccaaatgttcgatcagaagta |
| | YDDPSCTHFPGNLPNMLR | cgacgaccctagctgcactcacttcccgggcaatctgccaaatatgttacg |
| | KLRDAFSRVKTFFQMKDQ | taaactgcgggacgccttcagccgagttaaaactttctttcagatgaaggat |
| | LDNLLLKESLLEDFKGYLG | cagctggacaacctgctgttaaaggagagcctgctggaggacttcaagg |
| | CQALSEMIQFYLEEVMPQA | gttatctgggctgccaagccctgagcgagatgatccaattttatctggaaga |
| | TLRLRLRRCHRFLPCENKS | ggtcatgccccaagccgaaaatcaagaccccgatattaaggcccatgtca |
| | KAVEQVKNAFNKLQEKGI | attctctcggggagaacctaaagaccttgcgccttcggttaagaagatgtc |
| | YKAMSEFDIFINYIEAYMT | accgctttctcccctgtgagaacaagtctaaggccgttgaacaagtgaaga |
| | MKIRN (SEQ ID NO: 26) | acgccttcaataagctccaagagaaaggcatttataaggccatgtcggagt |
| | | tcgatatattcatcaattacatcgaggcgtatatgacaatgaagattaggaa |
| | | c (SEQ ID NO: 53) |
| TP0005 | SPGQGTQSENSCTHFPGNL | agccccggccaaggcacacagagcgagaactcatgtacccattttcccg |
| | PKMLRDLRDAFSRVKTFFQ | ggaacctgcccaagatgctgcgggacctgagagacgccttcagcagagt |
| | MKDQLDNLLLKESLLEDF | gaagactttctttcagatgaaggatcagctggacaaccttttactgaaggag |
| | KGYLGCQALSEMIQFYLEE | agtctgctagaagacttcaagggatacctgggctgtcaagccttgtcggag |
| | VMPQAENQDPDIKAHVNS | atgattcaattctacctggaagaagtgatgccccaagctgagaaccaaga |
| | LGENLKTLRLRLRRCHRFL | ccccgatatcaaggcccatgtgaacagcctgggcgagaatctcaaaacc |
| | PCENKSKAVEQVKNAFNK | ctgcgcttaaggctcagaagatgccacagatttctgccatgcgaaaacaa |
| | LQEKGIYKAMSEFDIFINYI | gagcaaggcagtggaacaagtgaaaaacgccttcaacaagctgcaaga |
| | EAYMTMKIRNGGGSGSGS | gaagggcatctacaaagccatgagcgagttcgacattttcatcaactatatt |
| | GSGSCTHFPGNLPKMLRDL | gaggcctacatgacgatgaagattcgaaatggcggagggtcgggctccg |
| | RDAFSRVKTFFQMKDQLD | gctctggcagcggctcctgcacccatttccccggcaacctgcccaaaatg |
| | NLLLKESLLEDFKGYLGCQ | ctgagagacctgcgtgacgccttctctagagtaaaaaccttctttcagatga |
| | ALSEMIQFYLEEVMPQAEN | aggatcagctggacaacttgttactgaaggagagcctgctcgaggacttc |
| | QDPDIKAHVNSLGENLKTL | aaaggctacctgggctgccaagccctgagcgagatgattcagttttacctt |
| | RLRLRRCHRFLPCENKSKA | gaagaggtgatgccccaagccgagaaccaagaccccgacatcaaagcc |
| | VEQVKNAFNKLQEKGIYK | cacgtcaactcgctgggcgaaaacctgaagactcttcggttgagacttcga |
| | AMSEFDIFINYIEAYMTMKI | agatgtcatcggttcttgccctgcgagaataagagcaaggcggtggagca |
| | RN (SEQ ID NO: 27) | agtgaagaacgcattcaataagctgcaagagaaaggtatctacaaggcta |
| | | tgagcgaatttgacatctttattaattacatcgaggcctatatgaccatgaaa |
| | | atcagaaatg (SEQ ID NO: 54) |
| TP0006 | SPGQGTQSENSCTHFPGNL | agtcccggacaaggcacacagagcgagaacagctgcacgcatttcccc |
| | PKMLRDLRDAFSRVKTFFQ | ggcaacctgcccaagatgctgcgtgatctcagagatgctttcagcagagt |
| | MKDQLDNLLLKESLLEDF | gaaaacgttcttccaaatgaaagaccaactcgataacctgttgcttaagga |
| | KGYLGCQALSEMIQFYLEE | gtcactgctggaagatttcaaaggctatcttggctgtcaagccctgagcga |
| | VMPQAENQDPDIKAHVNS | aatgatacagttctacctggaagaggtgatgcctcaagccgagaaccaag |
| | LGENLKTLRLRLRRCHRFL | acccggatattaaggcccatgtaaattccctgggcgagaacctgaaaacc |
| | PCENKSKAVEQVKNAFNK | ctcagactaagactgcgacgatgtcatagattcctaccctgtgaaaacaaa |
| | LQEKGIYKAMSEFDIFINYI | tctaaggccgtggagcaagtgaagaatgcttttaacaaactgcaagagaa |
| | EAYMTMKIRNTSPGQGTQ | gggcatttacaaggccatgagcgaatttgacatcttcatcaattacatcgaa |
| | SENSCTHFPGNLPKMLRDL | gcctacatgaccatgaaaattagaaacacgtcacccggccaaggcacac |
| | RDAFSRVKTFFQMKDQLD | agtctgaaaacagctgcacccactttcccggcaaccttoccaagatgttgc |
| | NLLLKESLLEDFKGYLGCQ | gagacctgagagacgcctttagccgcgtgaaaaccttcttccaaatgaaa |
| | ALSEMIQFYLEEVMPQAEN | gatcagctggacaatctgctgctcaaggaaagcctgctcgaggacttcaa |
| | QDPDIKAHVNSLGENLKTL | gggctacctcggctgccaagccctcagcgagatgattcagttttaccttga |
| | RLRLRRCHRFLPCENKSKA | agaagtcatgcctcaagccgaaaaccaagaccccgatataaaagcccat |
| | VEQVKNAFNKLQEKGIYK | gtgaacagcttgggcgagaacttaaagaccctgagattaagattacggcg |
| | AMSEFDIFINYIEAYMTMKI | gtgccaccgattcttgccgtgcgagaacaagtcaaaggcggtcgagcaa |
| | RN (SEQ ID NO: 28) | gtgaagaatgcattcaataaattacaagagaagggcatctataaggctatg |
| | | agtgagttcgatattttcatcaactatatcgaggcttacatgacaatgaaaat |
| | | cagaaac (SEQ ID NO: 55) |
| TP0007 | SPGQGTQSENSCTHFPGNL | tcacccggccaaggcacacagagcgagaactcctgcacccacttccccg |
| | PKMLRDLRDAFSRVKTFFQ | ggaacttgcccaagatgctgcgcgacctgagagatgccttcagcagagt |
| | MKDQLDNLLLKESLLEDF | gaagactttctttcagatgaaggatcagcttgacaaccttctcctgaaggag |
| | KGYLGCQALSEMIQFYLEE | agtctgctagaagacttcaagggatacctgggctgtcaagccttgtcggag |
| | VMPQAENQDPDIKAHVNS | atgattcagttttatctggaagaggtgatgccccaagctgagaatcaagac |
| | LGENLKTLRLRLRRCHRFL | cccgacatcaaagcccatgtgaacagcctgggcgagaatctcaaaaccc |
| | PCENKSKAVEQVKNAFNK | tgcgccttcgattgagaagatgccacagatttctgccatgcgaaaacaaga |
| | LQEKGIYKAMSEFDIFINYI | gcaaggcagtggaacaagtgaaaaatgccttcaacaagctgcaagagaa |
| | EAYMTMKIRNNQMFDQK | gggcatctacaaagccatgagcgagttcgacattttcatcaactacataga |
| | YDDPSCTHFPGNLPKMLR | ggcttacatgaccatgaagatcagaaacaaccaaatgttcgatcagaagta |
| | DLRDAFSRVKTFFQMKDQ | cgacgaccctagctgcactcacttcccgggcaatctgccaaaaatgttacg |
| | LDNLLLKESLLEDFKGYLG | tgatctgcgggacgccttcagccgagttaaaactttctttcagatgaaggat |
| | CQALSEMIQFYLEEVMPQA | cagctggacaacctgctgttaaaggagagcctgctggaggacttcaagg |
| | ENQDPDIKAHVNSLGENLK | gttatctgggctgccaagccctgagcgagatgatccaattttatctggaaga |
| | TLRLRLRRCHRFLPCENKS | ggtcatgccccaagccgaaaatcaagaccccgatattaaggcccatgtca |
| | KAVEQVKNAFNKLQEKGI | attctctcggggagaacctaaagaccttgcgccttcggttaagaagatgtc |
| | YKAMSEFDIFINYIEAYMT | accgctttctcccctgtgagaacaagtctaaggccgttgaacaagtgaaga |
| | MKIRN (SEQ ID NO: 29) | acgccttcaataagctccaagagaaaggcatttataaggccatgtcggagt |
| | | tcgatatattcatcaattacatcgaggcgtatatgacaatgaagattaggaa |
| | | c (SEQ ID NO: 56) |
| TP0008 | SPGQGTQSENSCTHFPGNL | agccccggccaaggcacacagagcgagaactcctgcacccacttcccc |
| | PNALRDLRDAFSRVKTFFQ | gggaacctgcccaacgccctgcgggacctgagagacgccttcagcaga |
| | MKDQLDNLLLKESLLEDF | gtgaagactttctttcagatgaaggatcagctggacaaccttcttctgaagg |
| | KGYLGCQALSEMIQFYLEE | agagtctgctagaagacttcaaaggttacctgggctgtcaagccttgtcgg |
| | VMPQAENQDPDIKAHVNS | agatgattcagttttatctggaagaggtcatgccccaagccgagaaccaag |
| | LGENLKTLRLRLRRCHRFL | accccgatatcaaagcccacgtaaatagcctgggagagaatctcaaaacc |
| | PCENKSKAVEQVKNAFNK | ttacggcttcgactgagaagatgccacagatttctgccatgtgaaaacaaa |
| | LQEKGIYKAMSEFDIFINYI | agcaaagcagtggaacaagtgaaaaacgccttcaacaagctgcaagag |
| | EAYMTMKIRNGGGSGSGS | aagggcatctataaagccatgagcgagttcgacattttcatcaattacatcg |
| | GSGSCTHFPGNLPNALRDL | aggcctacatgaccatgaaaatcagaaacgggggcggctccggctctgg |
| | RDAFSRVKTFFQMKDQLD | cagcggcagcggtagttgcacccatttccccggcaacctgcccaatgcg |
| | NLLLKESLLEDFKGYLGCQ | ctgagagacctaagagacgcctttagccgagttaagaccttctttcagatg |
| | ALSEMIQFYLEEVMPQAEN | aaggaccaactggacaacttactgctgaaggagagcctcctggaggactt |
| | QDPDIKAHVNSLGENLKTL | taagggttatctgggttgccaagcccttagcgagatgatccaattctatctg |
| | RLRLRRCHRFLPCENKSKA | gaggaagtgatgccccaagcagagaatcaagaccccgacataaaggcc |
| | VEQVKNAFNKLQEKGIYK | catgtcaacagcctgggggaaaacctgaaaacgctccgacttagattacg |
| | AMSEFDIFINYIEAYMTMKI | gcgctgtcatagatttctgccctgcgaaaacaagtccaaggcggtggagc |
| | RN (SEQ ID NO: 30) | aagtgaagaatgcctttaacaagttacaagagaaaggcatctacaaagcc |
| | | atgtcggagttcgacatattcattaactacattgaagcatatatgaccatgaa |
| | | gatcagaaac (SEQ ID NO: 57) |
| TP0009 | SPGQGTQSENSCTHFPGNL | agtcccggacaaggcacacagagcgagaacagctgcacgcatttcccc |
| | PNALRDLRDAFSRVKTFFQ | ggcaacctgcccaacgccctgcgtgatctcagagatgctttcagcagagt |
| | MKDQLDNLLLKESLLEDF | gaaaacgttcttccaaatgaaagaccaactcgataacctgttgcttaagga |
| | KGYLGCQALSEMIQFYLEE | gtcactgctggaagatttcaaaggctatcttggctgtcaagccctgagcga |
| | VMPQAENQDPDIKAHVNS | aatgatacagttctacctggaagaggtgatgcctcaagccgagaaccaag |
| | LGENLKTLRLRLRRCHRFL | acccggatattaaggcccatgtaaattccctgggcgagaacctgaaaacc |
| | PCENKSKAVEQVKNAFNK | ctcagactaagactgcgacgatgtcatagattcctaccctgtgaaaacaaa |
| | LQEKGIYKAMSEFDIFINYI | tctaaggccgtggagcaagtgaagaatgcttttaacaaactgcaagagaa |
| | EAYMTMKIRNTSPGQGTQ | gggcatttacaaggccatgagcgaatttgacatcttcatcaattacatcgaa |
| | SENSCTHFPGNLPNALRDL | gcctacatgaccatgaaaattagaaacacgtcacccggccaaggcacac |
| | RDAFSRVKTFFQMKDQLD | agtctgaaaacagctgcacccactttcccggcaaccttoccaacgccttgc |
| | NLLLKESLLEDFKGYLGCQ | gagacctgagagacgcctttagccgcgtgaaaaccttcttccaaatgaaa |
| | ALSEMIQFYLEEVMPQAEN | gatcagctggacaatctgctgctcaaggaaagcctgctcgaggacttcaa |
| | QDPDIKAHVNSLGENLKTL | gggctacctcggctgccaagccctcagcgagatgattcagttttaccttga |
| | RLRLRRCHRFLPCENKSKA | agaagtcatgcctcaagccgaaaaccaagaccccgatataaaagcccat |
| | VEQVKNAFNKLQEKGIYK | gtgaacagcttgggcgagaacttaaagaccctgagattaagattacggcg |
| | AMSEFDIFINYIEAYMTMKI | gtgccaccgattcttgccgtgcgagaacaagtcaaaggcggtcgagcaa |
| | RN (SEQ ID NO: 31) | gtgaagaatgcattcaataaattacaagagaagggcatctataaggctatg |
| | | agtgagttcgatattttcatcaactatatcgaggcttacatgacaatgaaaat |
| | | cagaaac (SEQ ID NO: 58) |
| TP0010 | SPGQGTQSENSCTHFPGNL | tcacccggccaaggcacacagagcgagaactcctgcacccacttccccg |
| | PNALRDLRDAFSRVKTFFQ | ggaacttgcccaacgccctgcgcgacctgagagatgccttcagcagagt |
| | MKDQLDNLLLKESLLEDF | gaagactttctttcagatgaaggatcagcttgacaaccttctcctgaaggag |
| | KGYLGCQALSEMIQFYLEE | agtctgctagaagacttcaagggatacctgggctgtcaagccttgtcggag |
| | VMPQAENQDPDIKAHVNS | atgattcagttttatctggaagaggtgatgccccaagctgagaatcaagac |
| | LGENLKTLRLRLRRCHRFL | cccgacatcaaagcccatgtgaacagcctgggcgagaatctcaaaaccc |
| | PCENKSKAVEQVKNAFNK | tgcgccttcgattgagaagatgccacagatttctgccatgcgaaaacaaga |
| | LQEKGIYKAMSEFDIFINYI | gcaaggcagtggaacaagtgaaaaatgccttcaacaagctgcaagagaa |
| | EAYMTMKIRNNQMFDQK | gggcatctacaaagccatgagcgagttcgacattttcatcaactacataga |
| | YDDPSCTHFPGNLPNALRD | ggcttacatgaccatgaagatcagaaacaaccaaatgttcgatcagaagta |
| | LRDAFSRVKTFFQMKDQL | cgacgaccctagctgcactcacttcccgggcaatctgccaaatgccttacg |
| | DNLLLKESLLEDFKGYLGC | tgatctgcgggacgccttcagccgagttaaaactttctttcagatgaaggat |
| | QALSEMIQFYLEEVMPQAE | cagctggacaacctgctgttaaaggagagcctgctggaggacttcaagg |
| | NQDPDIKAHVNSLGENLKT | gttatctgggctgccaagccctgagcgagatgatccaattttatctggaaga |
| | LRLRLRRCHRFLPCENKSK | ggtcatgccccaagccgaaaatcaagaccccgatattaaggcccatgtca |
| | AVEQVKNAFNKLQEKGIY | attctctcggggagaacctaaagaccttgcgccttcggttaagaagatgtc |
| | KAMSEFDIFINYIEAYMTM | accgctttctcccctgtgagaacaagtctaaggccgttgaacaagtgaaga |
| | KIRN (SEQ ID NO: 32) | acgccttcaataagctccaagagaaaggcatttataaggccatgtcggagt |
| | | tcgatatattcatcaattacatcgaggcgtatatgacaatgaagattaggaa |
| | | c (SEQ ID NO: 59) |
| TP0011 | SPGQGTQSENSCTHFPGNL | agccccggccaaggcacacagagcgagaactcctgcacccacttcccc |
| | PNSLRDLRDAFSRVKTFFQ | gggaacctgcccaacagcctgcgggacctgagagacgccttcagcaga |
| | MKDQLDNLLLKESLLEDF | gtgaagactttctttcagatgaaggatcagctggacaaccttcttctgaagg |
| | KGYLGCQALSEMIQFYLEE | agagtctgctagaagacttcaaaggctatctaggctgtcaagccttgtcgg |
| | VMPQAENQDPDIKAHVNS | agatgattcagttttatctggaagaggttatgccccaagccgagaaccaag |
| | LGENLKTLRLRLRRCHRFL | accccgatatcaaagcgcatgtcaatagcctgggcgagaatctcaaaacc |
| | PCENKSKAVEQVKNAFNK | ttacggcttcgactgagaagatgccacagatttctgccatgcgagaacaag |
| | LQEKGIYKAMSEFDIFINYI | agcaaggctgtggaacaagtgaaaaacgccttcaacaagctgcaagaga |
| | EAYMTMKIRNGGGSGSGS | agggaatctacaaagcaatgagcgagttcgacattttcatcaattacatcga |
| | GSGSCTHFPGNLPNSLRDL | ggcctacatgaccatgaaaatcagaaacgggggcggctccggctctggc |
| | RDAFSRVKTFFQMKDQLD | agcggcagcggttcctgcacccatttccccggcaacctgcccaattccct |
| | NLLLKESLLEDFKGYLGCQ | gagagacctaagagacgcctttagccgagttaagaccttctttcagatgaa |
| | ALSEMIQFYLEEVMPQAEN | ggatcagcttgacaacttactgctgaaggagagcctcctggaggactttaa |
| | QDPDIKAHVNSLGENLKTL | gggctacctaggttgtcaagcactgagcgagatgatccaattctatctgga |
| | RLRLRRCHRFLPCENKSKA | agaggtgatgccccaagctgaaaaccaagaccccgacataaaggcccat |
| | VEQVKNAFNKLQEKGIYK | gtgaactcactgggggagaacctgaaaacgctccgacttagattacggcg |
| | AMSEFDIFINYIEAYMTMKI | ctgtcatagatttctgccctgcgaaaacaagagcaaagccgtggagcaag |
| | RN (SEQ ID NO: 33) | tgaagaatgcctttaacaagttacaagagaaaggcatctacaaagccatgt |
| | | cggagttcgacatattcattaactacattgaagcatatatgaccatgaagatc |
| | | agaaac (SEQ ID NO: 60) |
| TP0012 | SPGQGTQSENSCTHFPGNL | agtcccggacaaggcacacagagcgagaacagctgcacgcatttcccc |
| | PNSLRDLRDAFSRVKTFFQ | ggcaacctgcccaacagcctgcgtgatctcagagatgctttcagcagagt |
| | MKDQLDNLLLKESLLEDF | gaaaacgttcttccaaatgaaagaccaactcgataacctgttgcttaagga |
| | KGYLGCQALSEMIQFYLEE | gtcactgctggaagatttcaaaggctatcttggctgtcaagccctgagcga |
| | VMPQAENQDPDIKAHVNS | aatgatacagttctacctggaagaggtgatgcctcaagccgagaaccaag |
| | LGENLKTLRLRLRRCHRFL | acccggatattaaggcccatgtaaattccctgggcgagaacctgaaaacc |
| | PCENKSKAVEQVKNAFNK | ctcagactaagactgcgacgatgtcatagattcctaccctgtgaaaacaaa |
| | LQEKGIYKAMSEFDIFINYI | tctaaggccgtggagcaagtgaagaatgcttttaacaaactgcaagagaa |
| | EAYMTMKIRNTSPGQGTQ | gggcatttacaaggccatgagcgaatttgacatcttcatcaattacatcgaa |
| | SENSCTHFPGNLPNSLRDL | gcctacatgaccatgaaaattagaaacacgtcacccggccaaggcacac |
| | RDAFSRVKTFFQMKDQLD | agtctgaaaacagctgcacccactttcccggcaaccttcccaacagcttgc |
| | NLLLKESLLEDFKGYLGCQ | gagacctgagagacgcctttagccgcgtgaaaaccttcttccaaatgaaa |
| | ALSEMIQFYLEEVMPQAEN | gatcagctggacaatctgctgctcaaggaaagcctgctcgaggacttcaa |
| | QDPDIKAHVNSLGENLKTL | gggctacctcggctgccaagccctcagcgagatgattcagttttaccttga |
| | RLRLRRCHRFLPCENKSKA | agaagtcatgcctcaagccgaaaaccaagaccccgatataaaagcccat |
| | VEQVKNAFNKLQEKGIYK | gtgaacagcttgggcgagaacttaaagaccctgagattaagattacggcg |
| | AMSEFDIFINYIEAYMTMKI | gtgccaccgattcttgccgtgcgagaacaagtcaaaggcggtcgagcaa |
| | RN (SEQ ID NO: 34) | gtgaagaatgcattcaataaattacaagagaagggcatctataaggctatg |
| | | agtgagttcgatattttcatcaactatatcgaggcttacatgacaatgaaaat |
| | | cagaaac (SEQ ID NO: 61) |
| TP0013 | SPGQGTQSENSCTHFPGNL | tcacccggccaaggcacacagagcgagaactcctgcacccacttccccg |
| | PNSLRDLRDAFSRVKTFFQ | ggaacttgcccaacagcctgcgcgacctgagagatgccttcagcagagt |
| | MKDQLDNLLLKESLLEDF | gaagactttctttcagatgaaggatcagcttgacaaccttctcctgaaggag |
| | KGYLGCQALSEMIQFYLEE | agtctgctagaagacttcaagggatacctgggctgtcaagccttgtcggag |
| | VMPQAENQDPDIKAHVNS | atgattcagttttatctggaagaggtgatgccccaagctgagaatcaagac |
| | LGENLKTLRLRLRRCHRFL | cccgacatcaaagcccatgtgaacagcctgggcgagaatctcaaaaccc |
| | PCENKSKAVEQVKNAFNK | tgcgccttcgattgagaagatgccacagatttctgccatgcgaaaacaaga |
| | LQEKGIYKAMSEFDIFINYI | gcaaggcagtggaacaagtgaaaaatgccttcaacaagctgcaagagaa |
| | EAYMTMKIRNNQMFDQK | gggcatctacaaagccatgagcgagttcgacattttcatcaactacataga |
| | YDDPSCTHFPGNLPNSLRD | ggcttacatgaccatgaagatcagaaacaaccaaatgttcgatcagaagta |
| | LRDAFSRVKTFFQMKDQL | cgacgaccctagctgcactcacttcccgggcaatctgccaaatagcttacg |
| | DNLLLKESLLEDFKGYLGC | tgatctgcgggacgccttcagccgagttaaaactttctttcagatgaaggat |
| | QALSEMIQFYLEEVMPQAE | cagctggacaacctgctgttaaaggagagcctgctggaggacttcaagg |
| | NQDPDIKAHVNSLGENLKT | gttatctgggctgccaagccctgagcgagatgatccaattttatctggaaga |
| | LRLRLRRCHRFLPCENKSK | ggtcatgccccaagccgaaaatcaagaccccgatattaaggcccatgtca |
| | AVEQVKNAFNKLQEKGIY | attctctcggggagaacctaaagaccttgcgccttcggttaagaagatgtc |
| | KAMSEFDIFINYIEAYMTM | accgctttctcccctgtgagaacaagtctaaggccgttgaacaagtgaaga |
| | KIRN (SEQ ID NO: 35) | acgccttcaataagctccaagagaaaggcatttataaggccatgtcggagt |
| | | tcgatatattcatcaattacatcgaggcgtatatgacaatgaagattaggaa |
| | | c (SEQ ID NO: 62) |
| TP0014 | SPGQGTQSENSCTHFPGNL | agccccggccaaggcacacagagcgagaactcatgtacccattttcccg |
| | PNMLRDLRDAFSRVKTFFQ | ggaacctgcccaacatgctgcgggacctgagagacgccttcagcagagt |
| | MKDQLDNLLLKESLLEDF | gaagactttctttcagatgaaggatcagctggacaaccttttactgaaggag |
| | KGYLGCQALSEMIQFYLEE | agtctgctagaagacttcaagggatacctgggctgtcaagccttgtcggag |
| | VMPQAENQDPDIKAHVNS | atgattcaattctacctggaagaagtgatgccccaagctgagaaccaaga |
| | LGENLKLLRLRLRRCHRFL | ccccgatatcaaggcccatgtgaacagcctgggcgagaatctcaaactgc |
| | PCENKSKAVEQVKNAFNK | tgcgcttaaggctcagaagatgccacagatttctgccatgcgaaaacaag |
| | LQEKGIYKAMSEFDIFINYI | agcaaggcagtggaacaagtgaaaaacgccttcaacaagctgcaagag |
| | EAYMTMKIRNGGGSGSGS | aagggcatctacaaagccatgagcgagttcgacattttcatcaactatattg |
| | GSGSCTHFPGNLPNMLRDL | aggcctacatgacgatgaagattcgaaatggcggagggtcgggctccgg |
| | RDAFSRVKTFFQMKDQLD | ctctggcagcggctcctgcacccatttccccggcaacctgcccaatatgct |
| | NLLLKESLLEDFKGYLGCQ | gagagacctgcgtgacgccttctctagagtaaaaaccttctttcagatgaa |
| | ALSEMIQFYLEEVMPQAEN | ggatcagctggacaacttgttactgaaggagagcctgctcgaggacttca |
| | QDPDIKAHVNSLGENLKLL | aaggctacctgggctgccaagccctgagcgagatgattcagttttaccttg |
| | RLRLRRCHRFLPCENKSKA | aagaggtgatgccccaagccgagaaccaagaccccgacatcaaagccc |
| | VEQVKNAFNKLQEKGIYK | acgtcaactcgctgggcgaaaacctgaagcttcttcggttgagacttcgaa |
| | AMSEFDIFINYIEAYMTMKI | gatgtcatcggttcttgccctgcgagaataagagcaaggcggtggagcaa |
| | RN (SEQ ID NO: 36) | gtgaagaacgcattcaataagctgcaagagaaaggtatctacaaggctat |
| | | gagcgaatttgacatctttattaattacatcgaggcctatatgaccatgaaaa |
| | | tcagaaat (SEQ ID NO: 63) |
| TP0015 | SPGQGTQSENSCTHFPGNL | agtcccggacaaggcacacagagcgagaacagctgcacgcatttcccc |
| | PNMLRDLRDAFSRVKTFFQ | ggcaacctgcccaacatgctgcgtgatctcagagatgctttcagcagagt |
| | MKDQLDNLLLKESLLEDF | gaaaacgttcttccaaatgaaagaccaactcgataacctgttgcttaagga |
| | KGYLGCQALSEMIQFYLEE | gtcactgctggaagatttcaaaggctatcttggctgtcaagccctgagcga |
| | VMPQAENQDPDIKAHVNS | aatgatacagttctacctggaagaggtgatgcctcaagccgagaaccaag |
| | LGENLKLLRLRLRRCHRFL | acccggatattaaggcccatgtaaattccctgggcgagaacctgaaactg |
| | PCENKSKAVEQVKNAFNK | ctcagactaagactgcgacgatgtcatagattcctaccctgtgaaaacaaa |
| | LQEKGIYKAMSEFDIFINYI | tctaaggccgtggagcaagtgaagaatgcttttaacaaactgcaagagaa |
| | EAYMTMKIRNTSPGQGTQ | gggcatttacaaggccatgagcgaatttgacatcttcatcaattacatcgaa |
| | SENSCTHFPGNLPNMLRDL | gcctacatgaccatgaaaattagaaacacgtcacccggccaaggcacac |
| | RDAFSRVKTFFQMKDQLD | agtctgaaaacagctgcacccactttcccggcaaccttoccaacatgttgc |
| | NLLLKESLLEDFKGYLGCQ | gagacctgagagacgcctttagccgcgtgaaaaccttcttccaaatgaaa |
| | ALSEMIQFYLEEVMPQAEN | gatcagctggacaatctgctgctcaaggaaagcctgctcgaggacttcaa |
| | QDPDIKAHVNSLGENLKLL | gggctacctcggctgccaagccctcagcgagatgattcagttttaccttga |
| | RLRLRRCHRFLPCENKSKA | agaagtcatgcctcaagccgaaaaccaagaccccgatataaaagcccat |
| | VEQVKNAFNKLQEKGIYK | gtgaacagcttgggcgagaacttaaagctgctgagattaagattacggcg |
| | AMSEFDIFINYIEAYMTMKI | gtgccaccgattcttgccgtgcgagaacaagtcaaaggcggtcgagcaa |
| | RN (SEQ ID NO: 37) | gtgaagaatgcattcaataaattacaagagaagggcatctataaggctatg |
| | | agtgagttcgatattttcatcaactatatcgaggcttacatgacaatgaaaat |
| | | cagaaac (SEQ ID NO: 64) |
| TP0016 | SPGQGTQSENSCTHFPGNL | tcacccggccaaggcacacagagcgagaactcctgcacccacttccccg |
| | PNMLRDLRDAFSRVKTFFQ | ggaacttgcccaacatgctgcgcgacctgagagatgccttcagcagagtg |
| | MKDQLDNLLLKESLLEDF | aagactttctttcagatgaaggatcagcttgacaaccttctcctgaaggaga |
| | KGYLGCQALSEMIQFYLEE | gtctgctagaagacttcaagggatacctgggctgtcaagccttgtcggaga |
| | VMPQAENQDPDIKAHVNS | tgattcagttttatctggaagaggtgatgccccaagctgagaatcaagacc |
| | LGENLKLLRLRLRRCHRFL | ccgacatcaaagcccatgtgaacagcctgggcgagaatctcaaactgctg |
| | PCENKSKAVEQVKNAFNK | cgccttcgattgagaagatgccacagatttctgccatgcgaaaacaagag |
| | LQEKGIYKAMSEFDIFINYI | caaggcagtggaacaagtgaaaaatgccttcaacaagctgcaagagaag |
| | EAYMTMKIRNNQMFDQK | ggcatctacaaagccatgagcgagttcgacattttcatcaactacatagag |
| | YDDPSCTHFPGNLPNMLR | gcttacatgaccatgaagatcagaaacaaccaaatgttcgatcagaagtac |
| | DLRDAFSRVKTFFQMKDQ | gacgaccctagctgcactcacttcccgggcaatctgccaaatatgttacgt |
| | LDNLLLKESLLEDFKGYLG | gatctgcgggacgccttcagccgagttaaaactttctttcagatgaaggatc |
| | CQALSEMIQFYLEEVMPQA | agctggacaacctgctgttaaaggagagcctgctggaggacttcaagggt |
| | ENQDPDIKAHVNSLGENLK | tatctgggctgccaagccctgagcgagatgatccaattttatctggaagag |
| | LLRLRLRRCHRFLPCENKS | gtcatgccccaagccgaaaatcaagaccccgatattaaggcccatgtcaa |
| | KAVEQVKNAFNKLQEKGI | ttctctcggggagaacctaaagctgttgcgccttcggttaagaagatgtcac |
| | YKAMSEFDIFINYIEAYMT | cgctttctcccctgtgagaacaagtctaaggccgttgaacaagtgaagaac |
| | MKIRN (SEQ ID NO: 38) | gccttcaataagctccaagagaaaggcatttataaggccatgtcggagttc |
| | | gatatattcatcaattacatcgaggcgtatatgacaatgaagattaggaac |
| | | (SEQ ID NO: 65) |
| TP0017 | SPGQGTQSENSCTHFPGNL | agccccggccaaggcacacagagcgagaactcatgtacccattttcccg |
| | PNMLRDLRDAFSRVKTFFQ | ggaacctgcccaacatgctgcgggacctgagagacgccttcagcagagt |
| | MKDQLDNLLLKESLLEDF | gaagactttctttcagatgaaggatcagctggacaaccttttactgaaggag |
| | KGYLGCQALSEMIQFYLEE | agtctgctagaagacttcaagggatacctgggctgtcaagccttgtcggag |
| | VMPQAENQDPDIKAHVNS | atgattcaattctacctggaagaagtgatgccccaagctgagaaccaaga |
| | LGQNLKTLRLRLRRCHRFL | ccccgatatcaaggcccatgtgaacagcctggggcagaatctcaaaacc |
| | PCENKSKAVEQVKNAFNK | ctgcgcttaaggctcagaagatgccacagatttctgccatgcgaaaacaa |
| | LQEKGIYKAMSEFDIFINYI | gagcaaggcagtggaacaagtgaaaaacgccttcaacaagctgcaaga |
| | EAYMTMKIRNGGGSGSGS | gaagggcatctacaaagccatgagcgagttcgacattttcatcaactatatt |
| | GSGSCTHFPGNLPNMLRDL | gaggcctacatgacgatgaagattcgaaatggcggagggtcgggctccg |
| | RDAFSRVKTFFQMKDQLD | gctctggcagcggctcctgcacccatttccccggcaacctgcccaatatg |
| | NLLLKESLLEDFKGYLGCQ | ctgagagacctgcgtgacgccttctctagagtaaaaaccttctttcagatga |
| | ALSEMIQFYLEEVMPQAEN | aggatcagctggacaacttgttactgaaggagagcctgctcgaggacttc |
| | QDPDIKAHVNSLGQNLKTL | aaaggctacctgggctgccaagccctgagcgagatgattcagttttacctt |
| | RLRLRRCHRFLPCENKSKA | gaagaggtgatgccccaagccgagaaccaagaccccgacatcaaagcc |
| | VEQVKNAFNKLQEKGIYK | cacgtcaactcgctgggccaaaacctgaagactcttcggttgagacttcga |
| | AMSEFDIFINYIEAYMTMKI | agatgtcatcggttcttgccctgcgagaataagagcaaggcggtggagca |
| | RN (SEQ ID NO: 39) | agtgaagaacgcattcaataagctgcaagagaaaggtatctacaaggcta |
| | | tgagcgaatttgacatctttattaattacatcgaggcctatatgaccatgaaa |
| | | atcagaaat (SEQ ID NO: 66) |
| TP0018 | SPGQGTQSENSCTHFPGNL | agtcccggacaaggcacacagagcgagaacagctgcacgcatttcccc |
| | PNMLRDLRDAFSRVKTFFQ | ggcaacctgcccaacatgctgcgtgatctcagagatgctttcagcagagt |
| | MKDQLDNLLLKESLLEDF | gaaaacgttcttccaaatgaaagaccaactcgataacctgttgcttaagga |
| | KGYLGCQALSEMIQFYLEE | gtcactgctggaagatttcaaaggctatcttggctgtcaagccctgagcga |
| | VMPQAENQDPDIKAHVNS | aatgatacagttctacctggaagaggtgatgcctcaagccgagaaccaag |
| | LGQNLKTLRLRLRRCHRFL | acccggatattaaggcccatgtaaattccctggggcagaacctgaaaacc |
| | PCENKSKAVEQVKNAFNK | ctcagactaagactgcgacgatgtcatagattcctaccctgtgaaaacaaa |
| | LQEKGIYKAMSEFDIFINYI | tctaaggccgtggagcaagtgaagaatgcttttaacaaactgcaagagaa |
| | EAYMTMKIRNTSPGQGTQ | gggcatttacaaggccatgagcgaatttgacatcttcatcaattacatcgaa |
| | SENSCTHFPGNLPNMLRDL | gcctacatgaccatgaaaattagaaacacgtcacccggccaaggcacac |
| | RDAFSRVKTFFQMKDQLD | agtctgaaaacagctgcacccactttcccggcaaccttcccaacatgttgc |
| | NLLLKESLLEDFKGYLGCQ | gagacctgagagacgcctttagccgcgtgaaaaccttcttccaaatgaaa |
| | ALSEMIQFYLEEVMPQAEN | gatcagctggacaatctgctgctcaaggaaagcctgctcgaggacttcaa |
| | QDPDIKAHVNSLGQNLKTL | gggctacctcggctgccaagccctcagcgagatgattcagttttaccttga |
| | RLRLRRCHRFLPCENKSKA | agaagtcatgcctcaagccgaaaaccaagaccccgatataaaagcccat |
| | VEQVKNAFNKLQEKGIYK | gtgaacagcttggggcagaacttaaagaccctgagattaagattacggcg |
| | AMSEFDIFINYIEAYMTMKI | gtgccaccgattcttgccgtgcgagaacaagtcaaaggcggtcgagcaa |
| | RN (SEQ ID NO: 40) | gtgaagaatgcattcaataaattacaagagaagggcatctataaggctatg |
| | | agtgagttcgatattttcatcaactatatcgaggcttacatgacaatgaaaat |
| | | cagaaac (SEQ ID NO: 67) |
| TP0019 | SPGQGTQSENSCTHFPGNL | tcacccggccaaggcacacagagcgagaactcctgcacccacttccccg |
| | PNMLRDLRDAFSRVKTFFQ | ggaacttgcccaacatgctgcgcgacctgagagatgccttcagcagagtg |
| | MKDQLDNLLLKESLLEDF | aagactttctttcagatgaaggatcagcttgacaaccttctcctgaaggaga |
| | KGYLGCQALSEMIQFYLEE | gtctgctagaagacttcaagggatacctgggctgtcaagccttgtcggaga |
| | VMPQAENQDPDIKAHVNS | tgattcagttttatctggaagaggtgatgccccaagctgagaatcaagacc |
| | LGQNLKTLRLRLRRCHRFL | ccgacatcaaagcccatgtgaacagcctggggcagaatctcaaaaccct |
| | PCENKSKAVEQVKNAFNK | gcgccttcgattgagaagatgccacagatttctgccatgcgaaaacaaga |
| | LQEKGIYKAMSEFDIFINYI | gcaaggcagtggaacaagtgaaaaatgccttcaacaagctgcaagagaa |
| | EAYMTMKIRNNQMFDQK | gggcatctacaaagccatgagcgagttcgacattttcatcaactacataga |
| | YDDPSCTHFPGNLPNMLR | ggcttacatgaccatgaagatcagaaacaaccaaatgttcgatcagaagta |
| | DLRDAFSRVKTFFQMKDQ | cgacgaccctagctgcactcacttcccgggcaatctgccaaatatgttacg |
| | LDNLLLKESLLEDFKGYLG | tgatctgcgggacgccttcagccgagttaaaactttctttcagatgaaggat |
| | CQALSEMIQFYLEEVMPQA | cagctggacaacctgctgttaaaggagagcctgctggaggacttcaagg |
| | ENQDPDIKAHVNSLGQNL | gttatctgggctgccaagccctgagcgagatgatccaattttatctggaaga |
| | KTLRLRLRRCHRFLPCENK | ggtcatgccccaagccgaaaatcaagaccccgatattaaggcccatgtca |
| | SKAVEQVKNAFNKLQEKG | attctctcgggcagaacctaaagaccttgcgccttcggttaagaagatgtc |
| | IYKAMSEFDIFINYIEAYMT | accgctttctcccctgtgagaacaagtctaaggccgttgaacaagtgaaga |
| | MKIRN (SEQ ID NO: 41) | acgccttcaataagctccaagagaaaggcatttataaggccatgtcggagt |
| | | tcgatatattcatcaattacatcgaggcgtatatgacaatgaagattaggaa |
| | | c (SEQ ID NO: 68) |
| TP0055 | SPGQGTQSENSCTHFPGNL | tctccgggccaaggcacacagagcgagaacagctgcacgcacttcccc |
| | PNMLRDLRDAFSRVKTFFQ | ggcaacctgcccaacatgctgcgggatctcagagatgctttcagcagagt |
| | MKDQLDNLLLKESLLEDF | gaaaacgttctttcagatgaaggaccaactcgacaacctgctgctgaagg |
| | KGYLGCQALSEMIQFYLEE | agtcactgctggaagatttcaaaggctatcttggctgtcaagccctgagcg |
| | VMPQAENQDPDIKAHVNS | aaatgatacagttctacctggaagaggtgatgccacaagccgagaaccaa |
| | LGENLKLLRLRLRRCHRFL | gacccggatattaaggcccatgtaaattccctgggcgagaacctgaaact |
| | PCENKSKAVEQVKNAFNK | gctcagactgcgacttagaagatgtcaccgcttcctgccgtgcgagaaca |
| | LQEKGIYKAMSEFDIFINYI | agagcaaggcagtggagcaagtgaaaaacgcctttaacaaactgcaaga |
| | EAYMTMKIRNTSPGQGTQ | gaaggggatctataaagccatgtccgagtttgacatttttattaactatatcg |
| | SENSCTHFPGNLPNMLRDL | aggcctatatgaccatgaaaatcagaaacacaagccccggccaaggcac |
| | RDAFSRVKTFFQMKDQLD | tcagtctgagaactcgtgcacccattttcccggcaatctgcccaacatgctg |
| | NLLLKESLLEDFKGYLGCQ | agggacctgagagatgccttctcgagagtgaaaaccttctttcaaatgaaa |
| | ALSEMIQFYLEEVMPQAEN | gatcagctggacaacttgctgctgaaagagagccttctggaggacttcaa |
| | QDPDIKAHVNSLGENLKTL | gggctatctcggctgccaagccctgagcgagatgattcagttttatctgga |
| | RLRLRRCHRFLPCENKSKA | agaggtgatgccccaagccgaaaaccaagaccccgacatcaaggccca |
| | VEQVKNAFNKLQEKGIYK | cgtcaacagcctgggtgagaacctgaagacccttcgactgagactgaga |
| | AMSEFDIFINYIEAYMTMKI | cgatgtcatcggttcctgccctgtgagaacaaatcgaaggccgtagagca |
| | RN (SEQ ID NO: 42) | agtgaagaatgcttttaataagctgcaagagaaaggaatatataaagctat |
| | | gtcagagttcgacatctttatcaactacatcgaggcctacatgaccatgaag |
| | | atccggaat (SEQ ID NO: 69) |
| SS0052 | SPGQGTQSENSCTHFPGNL | agccccggccagggtacacagtcagagaatagttgcacacactttcctgg |
| | PNMLRDLRDAFSRVKTFFQ | caatctgcctaatatgctacgcgatctcagggatgccttttctcgcgttaaaa |
| | MKDQLDNLLLKESLLEDF | cttttttccaaatgaaggaccagcttgataacctgctactcaaagaaagcctt |
| | KGYLGCQALSEMIQFYLEE | ttggaggattttaaggggtacctgggttgccaggccctgtcggaaatgatt |
| | VMPQAENQDPDIKAHVNS | cagttttatttggaggaggtgatgccccaggcagaaaaccaagatcctga |
| | LGENLKTLRLRLRRCHRFL | catcaaggcacatgtaaattcccttggagagaacctcaaaacactccgtctt |
| | PCENKSKAVEQVKNAFNK | aggctccgacgctgccatcgattcctgccttgcgagaacaagtccaaggc |
| | LQEKGIYKAMSEFDIFINYI | agtggagcaagtcaagaacgcctttaataaattacaagagaagggcatct |
| | EAYMTMKIRNSPGQGTQS | acaaagcgatgtccgagtttgacatcttcattaactacattgaagcttacatg |
| | ENSCTHFPGNLPNMLRDLR | accatgaaaataaggaacagcccgggacagggcacccagagtgaaaac |
| | DAFSRVKTFFQMKDQLDN | tcttgtacgcacttccccgggaatctgcccaacatgctgagagatctgcgg |
| | LLLKESLLEDFKGYLGCQA | gacgccttctcaagagtgaagaccttcttccaaatgaaagaccagttggac |
| | LSEMIQFYLEEVMPQAENQ | aatctgctgctgaaggaaagcttattagaggatttcaaaggctatctaggat |
| | DPDIKAHVNSLGENLKTLR | gtcaggctctgtcagaaatgatccagttctatctggaggaggtgatgccac |
| | LRLRRCHRFLPCENKSKAV | aggccgagaatcaggacccagacattaaagcgcacgtcaattctctggg |
| | EQVKNAFNKLQEKGIYKA | agaaaacctcaagactctccggttgcggctgagacggtgtcatagattctt |
| | MSEFDIFINYIEAYMTMKIR | gccatgtgaaaacaagtccaaagctgttgaacaggtgaaaaatgctttcaa |
| | N (SEQ ID NO: 43) | taagcttcaggaaaaggggatctacaaggccatgagtgagtttgacatattt |
| | | atcaactacattgaggcatatatgaccatgaagatcaggaac (SEQ ID |
| | | NO: 70) |
| SS0053 | SPGQGTQSENSCTHFPGNL | agtcccggacaaggcacacagagcgagaacagctgcacgcatttcccc |
| | PNMLRDLRDAFSRVKTFFQ | ggcaacctgcccaacatgctgcgtgatctcagagatgctttcagcagagt |
| | MKDQLDNLLLKESLLEDF | gaaaacgttcttccaaatgaaagaccaactcgataacctgttgcttaagga |
| | KGYLGCQALSEMIQFYLEE | gtcactgctggaagatttcaaaggctatcttggctgtcaagccctgagcga |
| | VMPQAENQDPDIKAHVNS | aatgatacagttctacctggaagaggtgatgcctcaagccgagaaccaag |
| | LGENLKLLRLRLRRCHRFL | acccggatattaaggcccatgtaaattccctgggcgagaacctgaaactg |
| | PCENKSKAVEQVKNAFNK | ctcagactaagactgcgacgatgtcatagattcctaccctgtgaaaacaaa |
| | LQEKGIYKAMSEFDIFINYI | tctaaggccgtggagcaagtgaagaatgcttttaacaaactgcaagagaa |
| | EAYMTMKIRNSPGQGTQS | gggcatttacaaggccatgagcgaatttgacatcttcatcaattacatcgaa |
| | ENSCTHFPGNLPNMLRDLR | gcctacatgaccatgaaaattagaaactcacccggccaaggcacacagtc |
| | DAFSRVKTFFQMKDQLDN | tgaaaacagctgcacccactttcccggcaaccttcccaacatgttgcgaga |
| | LLLKESLLEDFKGYLGCQA | cctgagagacgcctttagccgcgtgaaaaccttcttccaaatgaaagatca |
| | LSEMIQFYLEEVMPQAENQ | gctggacaatctgctgctcaaggaaagcctgctcgaggacttcaagggct |
| | DPDIKAHVNSLGENLKLLR | acctcggctgccaagccctcagcgagatgattcagttttaccttgaagaag |
| | LRLRRCHRFLPCENKSKAV | tcatgcctcaagccgaaaaccaagaccccgatataaaagcccatgtgaac |
| | EQVKNAFNKLQEKGIYKA | agcttgggcgagaacttaaagctgctgagattaagattacggcggtgcca |
| | MSEFDIFINYIEAYMTMKIR | ccgattcttgccgtgcgagaacaagtcaaaggcggtcgagcaagtgaag |
| | N (SEQ ID NO: 44) | aatgcattcaataaattacaagagaagggcatctataaggctatgagtgag |
| | | ttcgatattttcatcaactatatcgaggcttacatgacaatgaaaatcagaaa |
| | | c (SEQ ID NO: 71) |
| DR1063 | SPGQGTQSENSCTHFPGNL | agcccaggacagggaactcagagcgaaaactcttgtacacactttcctgg |
| | PNMLRDLRDAFSRVKTFFQ | aaatctgcccaacatgctgcgcgacctcagagatgccttcagccgcgtga |
| | MKDQLDNLLLKESLLEDF | aaaccttcttccagatgaaggaccaactcgataacttgctgttaaaggaaa |
| | KGYLGCQALSEMIQFYLEE | gcctgttggaagatttcaagggctacctaggttgtcaggcattgtccgaaat |
| | VMPQAENQDPDIKAHVNS | gatacagttttacttggaggaagtcatgccccaagcagagaaccaagacc |
| | LGENLKTLRLRLRRCHRFL | ctgatatcaaagcccacgtgaatagtctcggagagaatttaaaaactctgc |
| | PCENKSKAVEQVKNAFNK | ggctcagactccgacgatgtcataggtttctgccgtgcgagaacaagtca |
| | LQEKGIYKAMSEFDIFINYI | aaggcagtggagcaggtaaagaacgcgttcaataagcttcaggaaaaag |
| | EAYMTMKIRNGSGSGSGS | ggatctacaaagctatgtctgagtttgacatctttataaattatatcgaggctt |
| | GSGSCTHFPGNLPNMLRDL | atatgacaatgaaaatcaggaatgggtccggttcgggctccggtagtggg |
| | RDAFSRVKTFFQMKDQLD | tctggctcctgcacccatttccctggaaacctgccaaatatgctgcgggatc |
| | NLLLKESLLEDFKGYLGCQ | tgcgtgacgcctttagcagagtgaagaccttcttccagatgaaagaccagc |
| | ALSEMIQFYLEEVMPQAEN | tggacaacttgctactgaaggagtcacttctggaagactttaaagggtacct |
| | QDPDIKAHVNSLGENLKTL | gggctgccaagccctgtccgagatgattcagttctacttagaagaagtgat |
| | RLRLRRCHRFLPCENKSKA | gccccaggccgaaaatcaggacccagatattaaagctcatgttaacagtct |
| | VEQVKNAFNKLQEKGIYK | aggcgagaatcttaaaacactccggctccggcttaggcgctgccacagat |
| | AMSEFDIFINYIEAYMTMKI | ttcttccctgtgagaataagtctaaggcggttgagcaagtcaaaaacgcttt |
| | RN (SEQ ID NO: 45) | caataagctgcaggagaagggcatctataaggcaatgagcgagtttgata |
| | | ttttcattaactatatcgaagcctacatgacgatgaagattaggaac (SEQ |
| | | ID NO: 72) |
| DR1064 | SPGQGTQSENSCTHFPGNL | agccctggccaaggcacccagagcgaaaacagctgtacccatttcccgg |
| | PNMLRDLRDAFSRVKTFFQ | gcaatctgcccaatatgctccgcgatctaagagatgcattttcccgggtcaa |
| | MKDQLDNLLLKESLLEDF | gacattctttcagatgaaggaccaactggataatttgcttctgaaggaatcc |
| | KGYLGCQALSEMIQFYLEE | ctcctcgaagatttcaagggttacttggggtgccaggcactgtctgagatg |
| | VMPQAENQDPDIKAHVNS | atacagttctacctagaagaggttatgccacaagcggaaaatcaggaccc |
| | LGENLKTLRLRLRRCHRFL | agacattaaggcacacgtgaactctttaggtgagaacttgaagacactccg |
| | PCENKSKAVEQVKNAFNK | gcttcggctccgtcgatgccatcgcttccttccatgtgagaacaagtccaa |
| | LQEKGIYKAMSEFDIFINYI | ggctgttgagcaagtgaagaacgcttttaataaactacaggagaaaggca |
| | EAYMTMKIRNGSGSGSGSS | tctacaaggccatgtcagagtttgatattttcatcaattacatcgaagcctac |
| | CTHFPGNLPNMLRDLRDA | atgacgatgaaaatcaggaatggaagcggttcgggatctgggagctcatg |
| | FSRVKTFFQMKDQLDNLL | tactcatttccccggcaacctgcccaatatgctcagggacctcagagacgc |
| | LKESLLEDFKGYLGCQALS | ctttagccgcgtgaaaaccttctttcagatgaaagaccagcttgacaacttg |
| | EMIQFYLEEVMPQAENQD | ctgctgaaggaaagtttgttagaggatttcaaaggatacctggggtgtcag |
| | PDIKAHVNSLGENLKTLRL | gccctgtccgaaatgatccaattttatctggaggaggtaatgcctcaggcc |
| | RLRRCHRFLPCENKSKAVE | gagaatcaggaccccgatattaaagcacacgtgaatagtctgggagaaa |
| | QVKNAFNKLQEKGIYKAM | acttaaagactctgcgactgagactgaggaggtgccacagattcctgcctt |
| | SEFDIFINYIEAYMTMKIRN | gcgaaaataaaagtaaagctgtcgagcaggtgaagaacgccttcaacaa |
| | (SEQ ID NO: 46) | gcttcaggagaaggggatttataaagcgatgtctgagtttgacatatttatta |
| | | actatatcgaagcttatatgacaatgaaaatccggaac (SEQ ID |
| | | NO: 73) |
| DR1065 | SPGQGTQSENSCTHFPGNL | agccccggccagggtacacagtcagagaatagttgcacacactttcctgg |
| | PNMLRDLRDAFSRVKTFFQ | caatctgcctaatatgctacgcgatctcagggatgccttttctcgcgttaaaa |
| | MKDQLDNLLLKESLLEDF | cttttttccaaatgaaggaccagcttgataacctgctactcaaagaaagcctt |
| | KGYLGCQALSEMIQFYLEE | ttggaggattttaaggggtacctgggttgccaggccctgtcggaaatgatt |
| | VMPQAENQDPDIKAHVNS | cagttttatttggaggaggtgatgccccaggcagaaaaccaagatcctga |
| | LGENLKTLRLRLRRCHRFL | catcaaggcacatgtaaattcccttggagagaacctcaaaacactccgtctt |
| | PCENKSKAVEQVKNAFNK | aggctccgacgctgccatcgattcctgccttgcgagaacaagtccaaggc |
| | LQEKGIYKAMSEFDIFINYI | agtggagcaagtcaagaacgcctttaataaattacaagagaagggcatct |
| | EAYMTMKIRNTSPGQGTQ | acaaagcgatgtccgagtttgacatcttcattaactacattgaagcttacatg |
| | SENSCTHFPGNLPNMLRDL | accatgaaaataaggaacactagcccgggacagggcacccagagtgaa |
| | RDAFSRVKTFFQMKDQLD | aactcttgtacgcacttccccgggaatctgcccaacatgctgagagatctg |
| | NLLLKESLLEDFKGYLGCQ | cgggacgccttctcaagagtgaagaccttcttccaaatgaaagaccagttg |
| | ALSEMIQFYLEEVMPQAEN | gacaatctgctgctgaaggaaagcttattagaggatttcaaaggctatctag |
| | QDPDIKAHVNSLGENLKTL | gatgtcaggctctgtcagaaatgatccagttctatctggaggaggtgatgc |
| | RLRLRRCHRFLPCENKSKA | cacaggccgagaatcaggacccagacattaaagcgcacgtcaattctctg |
| | VEQVKNAFNKLQEKGIYK | ggagaaaacctcaagactctccggttgcggctgagacggtgtcatagatt |
| | AMSEFDIFINYIEAYMTMKI | cttgccatgtgaaaacaagtccaaagctgttgaacaggtgaaaaatgctttc |
| | RN (SEQ ID NO: 47) | aataagcttcaggaaaaggggatctacaaggccatgagtgagtttgacat |
| | | atttatcaactacattgaggcatatatgaccatgaagatcaggaac (SEQ |
| | | ID NO: 74) |
| DR1066 | SPGQGTQSENSCTHFPGNL | agccctgggcagggcacacaaagcgaaaactcttgtacccatttcccagg |
| | PNMLRDLRDAFSRVKTFFQ | gaatctccccaacatgctgcgagatctgagggatgctttcagtagggtgaa |
| | MKDQLDNLLLKESLLEDF | gacctttttccaaatgaaggaccagctcgacaatctgttattaaaagagtca |
| | KGYLGCQALSEMIQFYLEE | ctgctggaggacttcaaaggctacttgggctgccaggctctgagtgaaat |
| | VMPQAENQDPDIKAHVNS | gatccagttctatttggaggaggttatgccccaagcagagaaccaggacc |
| | LGENLKTLRLRLRRCHRFL | ccgatatcaaggcgcacgtcaattccctaggcgagaatctcaagacgctt |
| | PCENKSKAVEQVKNAFNK | cggcttcgcctgcgccggtgccacaggtttctgccatgcgagaataagtc |
| | LQEKGIYKAMSEFDIFINYI | aaaggcggttgaacaggtgaagaacgccttcaacaaactccaggagaaa |
| | EAYMTMKIRNGQGTQSEN | ggaatctacaaggctatgtccgaatttgatatattcattaactatatcgaagc |
| | SCTHFPGNLPNMLRDLRD | ctacatgactatgaaaattagaaatggacaaggaacacagagcgagaact |
| | AFSRVKTFFQMKDQLDNL | cttgtacccattttcctggtaatttacccaacatgctgagagacctgagggat |
| | LLKESLLEDFKGYLGCQAL | gcattctccagagtgaagactttttttcagatgaaagaccagctcgataatct |
| | SEMIQFYLEEVMPQAENQ | cctgctcaaagaaagtctactagaggatttcaagggctacctgggatgtca |
| | DPDIKAHVNSLGENLKTLR | ggccctgagcgaaatgatccagttttatttggaagaggtgatgccgcagg |
| | LRLRRCHRFLPCENKSKAV | ctgagaatcaagaccctgacattaaggcccacgtcaactctctgggtgag |
| | EQVKNAFNKLQEKGIYKA | aaccttaagacacttcggcttcgactgcggcgctgccatcgtttcttgccat |
| | MSEFDIFINYIEAYMTMKIR | gtgagaacaaatccaaagccgtagaacaggtgaagaatgcatttaacaaa |
| | N (SEQ ID NO: 48) | ttgcaggagaaagggatatataaggcaatgtcggaatttgacatcttcatca |
| | | actacattgaagcctacatgaccatgaagattagaaat (SEQ ID |
| | | NO: 75) |
| DR1067 | SPGQGTQSENSCTHFPGNL | agcccaggacagggaacacagtccgaaaattcatgtacccacttcccgg |
| | PNMLRDLRDAFSRVKTFFQ | ggaatctccccaatatgcttcgagacctgagggatgcgttctctagagttaa |
| | MKDQLDNLLLKESLLEDF | gaccttcttccagatgaaggatcagttggataatctgctgctgaaggagag |
| | KGYLGCQALSEMIQFYLEE | cctgctggaggattttaaagggtatttaggttgtcaagccctgtccgaaatg |
| | VMPQAENQDPDIKAHVNS | atccaattctaccttgaggaagtgatgcctcaggcagagaatcaagatccc |
| | LGENLKTLRLRLRRCHRFL | gacatcaaggctcacgtgaactccctaggtgagaacctgaagaccttaag |
| | PCENKSKAVEQVKNAFNK | gttgagattgcgccggtgccataggttcctcccttgtgaaaataagagcaa |
| | LQEKGIYKAMSEFDIFINYI | agctgtcgaacaggtgaaaaacgcgtttaacaaactgcaggaaaaaggc |
| | EAYMTMKIRNTKPEPMDQ | atctacaaggcaatgtctgagttcgacatcttcataaactacatcgaggctta |
| | YPSCTHFPGNLPNMLRDLR | tatgactatgaaaattagaaacacaaaacccgagcccatggatcagtatcc |
| | DAFSRVKTFFQMKDQLDN | ttcttgcacgcactttccgggcaacctcccaaacatgttacgcgatctgagg |
| | LLLKESLLEDFKGYLGCQA | gacgcatttagtcgcgtaaaaacctttttccagatgaaggaccagcttgaca |
| | LSEMIQFYLEEVMPQAENQ | atctcctgttgaaggagagtctgcttgaagattttaaaggatacctggggtg |
| | DPDIKAHVNSLGENLKTLR | ccaagccctctccgaaatgattcagttctatctggaagaggttatgccacag |
| | LRLRRCHRFLPCENKSKAV | gccgagaatcaagacccagacattaaggctcatgtgaatagcctcggcg |
| | EQVKNAFNKLQEKGIYKA | agaaccttaagacactgcgtctccggttgcgaagatgccatcggtttctac |
| | MSEFDIFINYIEAYMTMKIR | cttgtgaaaataaatcaaaggccgtggagcaggtcaagaacgccttcaac |
| | N (SEQ ID NO: 49) | aagctacaggagaaaggcatatataaagcaatgtcggagtttgacatcttt |
| | | attaattacatcgaagcctacatgactatgaagattcggaac (SEQ ID |
| | | NO: 76) |
| DR1068 | SPGQGTQSENSCTHFPGNL | tctccaggccagggcacgcaatctgaaaatagctgcacacattttcctggc |
| | PNMLRDLRDAFSRVKTFFQ | aacctccccaacatgcttagagacttaagggacgcatttagcagggttaag |
| | MKDQLDNLLLKESLLEDF | acattcttccagatgaaggatcagttggataatctcctcctgaaagagtccc |
| | KGYLGCQALSEMIQFYLEE | tactagaggactttaagggatacttaggttgtcaagccctgagcgaaatgat |
| | VMPQAENQDPDIKAHVNS | ccagttctacctggaagaagtgatgccccaggccgagaaccaagatcca |
| | LGENLKTLRLRLRRCHRFL | gatattaaagcgcacgtcaactccctgggagaaaacctcaaaactctgcg |
| | PCENKSKAVEQVKNAFNK | cctgaggcttagacgctgtcatcgattcctgccgtgcgagaataaatcgaa |
| | LQEKGIYKAMSEFDIFINYI | ggcggtggagcaagtgaaaaacgcttttaacaagttgcaggaaaaaggt |
| | EAYMTMKIRNNQMFDQK | atctacaaagctatgagcgagtttgacattttcataaactatatcgaggcttat |
| | YDDPSCTHFPGNLPNMLR | atgaccatgaagatccggaacaaccaaatgtttgaccagaaatatgacga |
| | DLRDAFSRVKTFFQMKDQ | cccatcctgtacccatttccctgggaatctccctaacatgctcagagacctg |
| | LDNLLLKESLLEDFKGYLG | cgggatgccttctcccgcgttaaaaccttttttcagatgaaggatcagttgg |
| | CQALSEMIQFYLEEVMPQA | ataatctgcttctcaaggagtctcttcttgaagacttcaagggatacctggg |
| | ENQDPDIKAHVNSLGENLK | gtgccaggccctgtcagaaatgattcagttctatctagaggaggtgatgcc |
| | TLRLRLRRCHRFLPCENKS | tcaggctgagaatcaggaccccgatattaaagcccacgtcaacagtctgg |
| | KAVEQVKNAFNKLQEKGI | gggaaaatctgaagacactgcggttaaggttgcggcgatgtcacagattct |
| | YKAMSEFDIFINYIEAYMT | tgccctgcgaaaataagtcaaaagcagtagagcaggtgaaaaacgcattt |
| | MKIRN (SEQ ID NO: 50) | aataagctgcaggaaaagggcatttacaaggccatgagtgagttcgatat |
| | | ctttataaattacatcgaggcatatatgactatgaagatccgtaat (SEQ |
| | | ID NO: 77) |
| | SENSCTHFPGNLPNMLRDL | tcagagaatagttgcacacactttcctggcaatctgcctaatatgctacgcg |
| | RDAFSRVKTFFQMKDQLD | atctcagggatgccttttctcgcgttaaaacttttttccaaatgaaggaccag |
| | NLLLKESLLEDFKGYLGCQ | cttgataacctgctactcaaagaaagccttttggaggattttaaggggtacct |
| | ALSEMIQFYLEEVMPQAEN | gggttgccaggccctgtcggaaatgattcagttttatttggaggaggtgatg |
| | QDPDIKAHVNSLGENLKTL | ccccaggcagaaaaccaagatcctgacatcaaggcacatgtaaattccct |
| | RLRLRRCHRFLPCENKSKA | tggagagaacctcaaaacactccgtcttaggctccgacgctgccatcgatt |
| | VEQVKNAFNKLQEKGIYK | cctgccttgcgagaacaagtccaaggcagtggagcaagtcaagaacgcc |
| | AMSEFDIFINYIEAYMTMKI | tttaataaattacaagagaagggcatctacaaagcgatgtccgagtttgaca |
| | RNTSPGQGTQSENSCTHFP | tcttcattaactacattgaagcttacatgaccatgaaaataaggaacactag |
| | GNLPNMLRDLRDAFSRVK | cccgggacagggcacccagagtgaaaactcttgtacgcacttccccggg |
| | TFFQMKDQLDNLLLKESLL | aatctgcccaacatgctgagagatctgcgggacgccttctcaagagtgaa |
| | EDFKGYLGCQALSEMIQFY | gaccttcttccaaatgaaagaccagttggacaatctgctgctgaaggaaag |
| | LEEVMPQAENQDPDIKAH | cttattagaggatttcaaaggctatctaggatgtcaggctctgtcagaaatg |
| | VNSLGENLKTLRLRLRRCH | atccagttctatctggaggaggtgatgccacaggccgagaatcaggaccc |
| | RFLPCENKSKAVEQVKNA | agacattaaagcgcacgtcaattctctgggagaaaacctcaagactctccg |
| | FNKLQEKGIYKAMSEFDIFI | gttgcggctgagacggtgtcatagattcttgccatgtgaaaacaagtccaa |
| | NYIEAYMTMKIRN (SEQ ID | agctgttgaacaggtgaaaaatgctttcaataagcttcaggaaaaggggat |
| | NO: 119) | ctacaaggccatgagtgagtttgacatatttatcaactacattgaggcatata |
| | | tgaccatgaagatcaggaac (SEQ ID NO: 120) |

The disclosure provides a fused IL10 polypeptide the formula of (hIL10A)-Lₙ-(hIL10B) having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to any one of the polypeptide sequences of Table 2. The disclosure provides a fused IL10 polypeptide the formula of (hIL10A)-Lₙ-(hIL10B) having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to any one of the polypeptide sequences of Table 2A, In some embodiments the fused IL10 polypeptide the formula of (hIL10A)-Lₙ-(hIL10B) is a polypeptide having at least alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to a polypeptide selected from the group consisting of SEQ ID NOS:157, 158, 159, 160, 161, 162, 163, 164, 165, 166, 167, 168, 169, 170 and 171. In some embodiments the fused IL10 polypeptide the formula of (hIL10A)-Lₙ-(hIL10B) is a polypeptide selected from the group consisting of SEQ ID NOS:157, 158, 159, 160, 161, 162, 163, 164, 165, 166, 167, 168, 169, 170 and 171. In some embodiments the fused IL10 polypeptide the formula of (hIL10A)-Lₙ-(hIL10B) is SEQ ID NO: 159. In some embodiments the fused IL10 polypeptide the formula of (hIL10A)-Lₙ-(hIL10B) is SEQ ID NO: 160. In some embodiments the fused IL10 polypeptide the formula of (hIL10A)-Lₙ-(hIL10B) is SEQ ID NO: 165. In some embodiments the fused IL10 polypeptide the formula of (hIL10A)-Lₙ-(hIL10B) is SEQ ID NO: 170.

TABLE 2A

| SEQ | | |
|---|---|---|
| ID# | Desc | hIL 10 Mutein Fused Dimer Sequences |
| 157 | hIL10 N21D | SPGQGTQSENSCTHFPGNLPDMLRDLRDAFSRVKTFFQMKDQL |
| | fused | DNLLLKESLLEDFKGYLGCQALSEMIQFYLEEVMPQAENQDPDI |
| | homodimer | KAHVNSLGENLKTLRLRLRRCHRFLPCENKSKAVEQVKNAFNK |
| | | LQEKGIYKAMSEFDIFINYIEAYMTMKIRNSPGQGTQSENSCTHF |
| | | PGNLPDMLRDLRDAFSRVKTFFQMKDQLDNLLLKESLLEDFKG |
| | | YLGCQALSEMIQFYLEEVMPQAENQDPDIKAHVNSLGENLKTLR |
| | | LRLRRCHRFLPCENKSKAVEQVKNAFNKLQEKGIYKAMSEFDIFI |
| | | NYIEAYMTMKIRN (SEQ ID NO: 157) |
| 158 | hIL10 N21E | SPGQGTQSENSCTHFPGNLPEMLRDLRDAFSRVKTFFQMKDQLD |
| | fused | NLLLKESLLEDFKGYLGCQALSEMIQFYLEEVMPQAENQDPDIK |
| | homodimer | AHVNSLGENLKTLRLRLRRCHRFLPCENKSKAVEQVKNAFNKL |
| | | QEKGIYKAMSEFDIFINYIEAYMTMKIRNSPGQGTQSENSCTHFP |
| | | GNLPEMLRDLRDAFSRVKTFFQMKDQLDNLLLKESLLEDFKGY |
| | | LGCQALSEMIQFYLEEVMPQAENQDPDIKAHVNSLGENLKTLRL |
| | | RLRRCHRFLPCENKSKAVEQVKNAFNKLQEKGIYKAMSEFDIFI |
| | | NYIEAYMTMKIRN (SEQ ID NO: 158) |
| 159 | hIL10 N21K | SPGQGTQSENSCTHFPGNLPKMLRDLRDAFSRVKTFFQMKDQL |
| | fused | DNLLLKESLLEDFKGYLGCQALSEMIQFYLEEVMPQAENQDPDI |
| | homodimer | KAHVNSLGENLKTLRLRLRRCHRFLPCENKSKAVEQVKNAFNK |
| | | LQEKGIYKAMSEFDIFINYIEAYMTMKIRNSPGQGTQSENSCTHF |
| | | PGNLPKMLRDLRDAFSRVKTFFQMKDQLDNLLLKESLLEDFKG |
| | | YLGCQALSEMIQFYLEEVMPQAENQDPDIKAHVNSLGENLKTLR |
| | | LRLRRCHRFLPCENKSKAVEQVKNAFNKLQEKGIYKAMSEFDIFI |
| | | NYIEAYMTMKIRN (SEQ ID NO: 159) |
| 160 | hIL10 M22A | SPGQGTQSENSCTHFPGNLPDALRDLRDAFSRVKTFFQMKDQLD |
| | fused | NLLLKESLLEDFKGYLGCQALSEMIQFYLEEVMPQAENQDPDIK |
| | homodimer | AHVNSLGENLKTLRLRLRRCHRFLPCENKSKAVEQVKNAFNKL |
| | | QEKGIYKAMSEFDIFINYIEAYMTMKIRNSPGQGTQSENSCTHFP |
| | | GNLPDALRDLRDAFSRVKTFFQMKDQLDNLLLKESLLEDFKGY |
| | | LGCQALSEMIQFYLEEVMPQAENQDPDIKAHVNSLGENLKTLRL |
| | | RLRRCHRFLPCENKSKAVEQVKNAFNKLQEKGIYKAMSEFDIFI |
| | | NYIEAYMTMKIRN (SEQ ID NO: 160) |
| 161 | hIL10 M22S | SPGQGTQSENSCTHFPGNLPDSLRDLRDAFSRVKTFFQMKDQLD |
| | fused | NLLLKESLLEDFKGYLGCQALSEMIQFYLEEVMPQAENQDPDIK |
| | homodimer | AHVNSLGENLKTLRLRLRRCHRFLPCENKSKAVEQVKNAFNKL |
| | | QEKGIYKAMSEFDIFINYIEAYMTMKIRNSPGQGTQSENSCTHFP |
| | | GNLPDSLRDLRDAFSRVKTFFQMKDQLDNLLLKESLLEDFKGYL |
| | | GCQALSEMIQFYLEEVMPQAENQDPDIKAHVNSLGENLKTLRLR |
| | | LRRCHRFLPCENKSKAVEQVKNAFNKLQEKGIYKAMSEFDIFIN |
| | | YIEAYMTMKIRN (SEQ ID NO: 161) |
| 162 | hIL10 M22T | SPGQGTQSENSCTHFPGNLPDTLRDLRDAFSRVKTFFQMKDQLD |
| | fused | NLLLKESLLEDFKGYLGCQALSEMIQFYLEEVMPQAENQDPDIK |
| | homodimer | AHVNSLGENLKTLRLRLRRCHRFLPCENKSKAVEQVKNAFNKL |
| | | QEKGIYKAMSEFDIFINYIEAYMTMKIRNSPGQGTQSENSCTHFP |
| | | GNLPDTLRDLRDAFSRVKTFFQMKDQLDNLLLKESLLEDFKGYL |
| | | GCQALSEMIQFYLEEVMPQAENQDPDIKAHVNSLGENLKTLRLR |
| | | LRRCHRFLPCENKSKAVEQVKNAFNKLQEKGIYKAMSEFDIFIN |
| | | YIEAYMTMKIRN (SEQ ID NO: 162) |
| 163 | hIL10 M22D | SPGQGTQSENSCTHFPGNLPDDLRDLRDAFSRVKTFFQMKDQLD |
| | fused | NLLLKESLLEDFKGYLGCQALSEMIQFYLEEVMPQAENQDPDIK |
| | homodimer | AHVNSLGENLKTLRLRLRRCHRFLPCENKSKAVEQVKNAFNKL |
| | | QEKGIYKAMSEFDIFINYIEAYMTMKIRNSPGQGTQSENSCTHFP |
| | | GNLPDDLRDLRDAFSRVKTFFQMKDQLDNLLLKESLLEDFKGY |
| | | LGCQALSEMIQFYLEEVMPQAENQDPDIKAHVNSLGENLKTLRL |
| | | RLRRCHRFLPCENKSKAVEQVKNAFNKLQEKGIYKAMSEFDIFI |
| | | NYIEAYMTMKIRN (SEQ ID NO: 163) |
| 164 | hIL10 M22W | SPGQGTQSENSCTHFPGNLPDWLRDLRDAFSRVKTFFQMKDQL |
| | fused | DNLLLKESLLEDFKGYLGCQALSEMIQFYLEEVMPQAENQDPDI |
| | homodimer | KAHVNSLGENLKTLRLRLRRCHRFLPCENKSKAVEQVKNAFNK |
| | | LQEKGIYKAMSEFDIFINYIEAYMTMKIRNSPGQGTQSENSCTHF |
| | | PGNLPDWLRDLRDAFSRVKTFFQMKDQLDNLLLKESLLEDFKG |
| | | YLGCQALSEMIQFYLEEVMPQAENQDPDIKAHVNSLGENLKTLR |
| | | LRLRRCHRFLPCENKSKAVEQVKNAFNKLQEKGIYKAMSEFDIFI |
| | | NYIEAYMTMKIRN (SEQ ID NO: 164) |
| 165 | hIL10 R24E | SPGQGTQSENSCTHFPGNLPNMLEDLRDAFSRVKTFFQMKDQLD |
| | fused | NLLLKESLLEDFKGYLGCQALSEMIQFYLEEVMPQAENQDPDIK |
| | homodimer | AHVNSLGENLKTLRLRLRRCHRFLPCENKSKAVEQVKNAFNKL |
| | | QEKGIYKAMSEFDIFINYIEAYMTMKIRNSPGQGTQSENSCTHFP |
| | | GNLPNMLEDLRDAFSRVKTFFQMKDQLDNLLLKESLLEDFKGY |
| | | LGCQALSEMIQFYLEEVMPQAENQDPDIKAHVNSLGENLKTLRL |
| | | RLRRCHRFLPCENKSKAVEQVKNAFNKLQEKGIYKAMSEFDIFI |
| | | NYIEAYMTMKIRN (SEQ ID NO: 165) |
| 166 | hIL10 D25K | SPGQGTQSENSCTHFPGNLPNMLRKLRDAFSRVKTFFQMKDQL |
| | fused | DNLLLKESLLEDFKGYLGCQALSEMIQFYLEEVMPQAENQDPDI |
| | homodimer | KAHVNSLGENLKTLRLRLRRCHRFLPCENKSKAVEQVKNAFNK |
| | | LQEKGIYKAMSEFDIFINYIEAYMTMKIRNSPGQGTQSENSCTHF |
| | | PGNLPNMLRKLRDAFSRVKTFFQMKDQLDNLLLKESLLEDFKG |
| | | YLGCQALSEMIQFYLEEVMPQAENQDPDIKAHVNSLGENLKTLR |
| | | LRLRRCHRFLPCENKSKAVEQVKNAFNKLQEKGIYKAMSEFDIFI |
| | | NYIEAYMTMKIRN (SEQ ID NO: 166) |
| 167 | hIL10 E96K | SPGQGTQSENSCTHFPGNLPNMLRDLRDAFSRVKTFFQMKDQL |
| | fused | DNLLLKESLLEDFKGYLGCQALSEMIQFYLEEVMPQAENQDPDI |
| | homodimer | KAHVNSLGKNLKTLRLRLRRCHRFLPCENKSKAVEQVKNAFNK |
| | | LQEKGIYKAMSEFDIFINYIEAYMTMKIRNSPGQGTQSENSCTHF |
| | | PGNLPNMLRDLRDAFSRVKTFFQMKDQLDNLLLKESLLEDFKG |
| | | YLGCQALSEMIQFYLEEVMPQAENQDPDIKAHVNSLGKNLKTL |
| | | RLRLRRCHRFLPCENKSKAVEQVKNAFNKLQEKGIYKAMSEFDI |
| | | FINYIEAYMTMKIRN (SEQ ID NO: 167) |
| 168 | hIL10 E96Q | SPGQGTQSENSCTHFPGNLPNMLRDLRDAFSRVKTFFQMKDQL |
| | fused | DNLLLKESLLEDFKGYLGCQALSEMIQFYLEEVMPQAENQDPDI |
| | homodimer | KAHVNSLGQNLKTLRLRLRRCHRFLPCENKSKAVEQVKNAFNK |
| | | LQEKGIYKAMSEFDIFINYIEAYMTMKIRNSPGQGTQSENSCTHF |
| | | PGNLPNMLRDLRDAFSRVKTFFQMKDQLDNLLLKESLLEDFKG |
| | | YLGCQALSEMIQFYLEEVMPQAENQDPDIKAHVNSLGQNLKTL |
| | | RLRLRRCHRFLPCENKSKAVEQVKNAFNKLQEKGIYKAMSEFDI |
| | | FINYIEAYMTMKIRN (SEQ ID NO: 168) |
| 169 | hIL10 T100E | SPGQGTQSENSCTHFPGNLPNMLRDLRDAFSRVKTFFQMKDQL |
| | fused | DNLLLKESLLEDFKGYLGCQALSEMIQFYLEEVMPQAENQDPDI |
| | homodimer | KAHVNSLGENLKELRLRLRRCHRFLPCENKSKAVEQVKNAFNK |
| | | LQEKGIYKAMSEFDIFINYIEAYMTMKIRNSPGQGTQSENSCTHF |
| | | PGNLPNMLRDLRDAFSRVKTFFQMKDQLDNLLLKESLLEDFKG |
| | | YLGCQALSEMIQFYLEEVMPQAENQDPDIKAHVNSLGENLKELR |
| | | LRLRRCHRFLPCENKSKAVEQVKNAFNKLQEKGIYKAMSEFDIFI |
| | | NYIEAYMTMKIRN (SEQ ID NO: 169) |
| 170 | hIL10 T100L | SPGQGTQSENSCTHFPGNLPNMLRDLRDAFSRVKTFFQMKDQL |
| | fused | DNLLLKESLLEDFKGYLGCQALSEMIQFYLEEVMPQAENQDPDI |
| | homodimer | KAHVNSLGENLKLLRLRLRRCHRFLPCENKSKAVEQVKNAFNK |
| | | LQEKGIYKAMSEFDIFINYIEAYMTMKIRNSPGQGTQSENSCTHF |
| | | PGNLPNMLRDLRDAFSRVKTFFQMKDQLDNLLLKESLLEDFKG |
| | | YLGCQALSEMIQFYLEEVMPQAENQDPDIKAHVNSLGENLKLLR |
| | | LRLRRCHRFLPCENKSKAVEQVKNAFNKLQEKGIYKAMSEFDIFI |
| | | NYIEAYMTMKIRN (SEQ ID NO: 170) |
| 171 | hIL10 T100C | SPGQGTQSENSCTHFPGNLPNMLRDLRDAFSRVKTFFQMKDQL |
| | fused | DNLLLKESLLEDFKGYLGCQALSEMIQFYLEEVMPQAENQDPDI |
| | homodimer | KAHVNSLGENLKCLRLRLRRCHRFLPCENKSKAVEQVKNAFNK |
| | | LQEKGIYKAMSEFDIFINYIEAYMTMKIRNSPGQGTQSENSCTHF |
| | | PGNLPNMLRDLRDAFSRVKTFFQMKDQLDNLLLKESLLEDFKG |
| | | YLGCQALSEMIQFYLEEVMPQAENQDPDIKAHVNSLGENLKCLR |
| | | LRLRRCHRFLPCENKSKAVEQVKNAFNKLQEKGIYKAMSEFDIFI |
| | | NYIEAYMTMKIRN (SEQ ID NO: 171) |

As discussed herein, the N-terminal, wherein the molecule is the fused hIL10 polypeptide is recombinantly produced in bacterial cells by direct expression (i.e. not as a fusion protein), the naturally occurring N-terminal serine may be deleted to provide for efficient cleavage of the N-terminal methionyl residue. The disclosure provides a fused IL10 polypeptide the formula of (hIL10A)-Lₙ-(hIL10B) having at least 90%, alternatively at least 910%, alternatively at least 920%, alternatively at least 93%, alternatively at least 940%, alternatively at least 9500, alternatively at least 9600, alternatively at least 970%, alternatively at least 98%, alternatively at least 99% or 100% sequence identity to any one of the polypeptide sequences of Table 2B. In some embodiments, the disclosure provides a fused IL10 polypeptide the formula of (hIL10A)-Lₙ-(hIL10B) having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to any one of the polypeptide sequences of Table 2B. In some embodiments the fused IL10 polypeptide the formula of (hIL10A)-Lₙ-(hIL10B) is a polypeptide having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to a polypeptide selected from the group consisting of SEQ ID NOS:172, 173, 174, 175, 176, 177, 178, 179, 180, 181, 182, 183 184, 185 and 186. In some embodiments the fused IL10 polypeptide the formula of (hIL10A)-Lₙ-(hIL10B) is SEQ ID NO: 174. In some embodiments the fused IL10 polypeptide the formula of (hIL10A)-Lₙ-(hIL10B) is SEQ ID NO: 175. In some embodiments the fused IL10 polypeptide the formula of (hIL10A)-Lₙ-(hIL10B) is SEQ ID NO: 180. In some embodiments the fused IL10 polypeptide the formula of (hIL10A)-Lₙ-(hIL10B) is SEQ ID NO: 185. In some embodiments the fused IL10 polypeptide the formula of (hIL10A)-Lₙ-(hIL10B) is selected from the group consisting of SEQ ID NOS:172, 173, 174, 175, 176, 177, 178, 179, 180, 181, 182, 183 184, 185 and 186.

TABLE 2B

| SEQ | | |
|---|---|---|
| ID# | Desc | des-SER1-hIL10 Mutein Fused Dimer Sequences |
| 172 | des-SER1 | PGQGTQSENSCTHFPGNLPDMLRDLRDAFSRVKTFFQMKDQLD |
| | hIL10 N21D/ | NLLLKESLLEDFKGYLGCQALSEMIQFYLEEVMPQAENQDPDIK |
| | hIL10 N21D | AHVNSLGENLKTLRLRLRRCHRFLPCENKSKAVEQVKNAFNKL |
| | fused | QEKGIYKAMSEFDIFINYIEAYMTMKIRNSPGQGTQSENSCTHFP |
| | heterodimer | GNLPDMLRDLRDAFSRVKTFFQMKDQLDNLLLKESLLEDFKGY |
| | | LGCQALSEMIQFYLEEVMPQAENQDPDIKAHVNSLGENLKTLRL |
| | | RLRRCHRFLPCENKSKAVEQVKNAFNKLQEKGIYKAMSEFDIFI |
| | | NYIEAYMTMKIRN (SEQ ID NO: 172) |
| 173 | des-SER1 | PGQGTQSENSCTHFPGNLPEMLRDLRDAFSRVKTFFQMKDQLD |
| | hIL10 N21E/ | NLLLKESLLEDFKGYLGCQALSEMIQFYLEEVMPQAENQDPDIK |
| | hIL10 N21E | AHVNSLGENLKTLRLRLRRCHRFLPCENKSKAVEQVKNAFNKL |
| | fused | QEKGIYKAMSEFDIFINYIEAYMTMKIRNSPGQGTQSENSCTHFP |
| | heterodimer | GNLPEMLRDLRDAFSRVKTFFQMKDQLDNLLLKESLLEDFKGY |
| | | LGCQALSEMIQFYLEEVMPQAENQDPDIKAHVNSLGENLKTLRL |
| | | RLRRCHRFLPCENKSKAVEQVKNAFNKLQEKGIYKAMSEFDIFI |
| | | NYIEAYMTMKIRN (SEQ ID NO: 173) |
| 174 | des-SER1 | PGQGTQSENSCTHFPGNLPKMLRDLRDAFSRVKTFFQMKDQLD |
| | hIL10 N21K/ | NLLLKESLLEDFKGYLGCQALSEMIQFYLEEVMPQAENQDPDIK |
| | hIL10 N21K | AHVNSLGENLKTLRLRLRRCHRFLPCENKSKAVEQVKNAFNKL |
| | fused | QEKGIYKAMSEFDIFINYIEAYMTMKIRNSPGQGTQSENSCTHFP |
| | heterodimer | GNLPKMLRDLRDAFSRVKTFFQMKDQLDNLLLKESLLEDFKGY |
| | | LGCQALSEMIQFYLEEVMPQAENQDPDIKAHVNSLGENLKTLRL |
| | | RLRRCHRFLPCENKSKAVEQVKNAFNKLQEKGIYKAMSEFDIFI |
| | | NYIEAYMTMKIRN (SEQ ID NO: 174) |
| 175 | des-SER1 | PGQGTQSENSCTHFPGNLPDALRDLRDAFSRVKTFFQMKDQLD |
| | hIL10 M22A/ | NLLLKESLLEDFKGYLGCQALSEMIQFYLEEVMPQAENQDPDIK |
| | hIL10 | AHVNSLGENLKTLRLRLRRCHRFLPCENKSKAVEQVKNAFNKL |
| | M22A | QEKGIYKAMSEFDIFINYIEAYMTMKIRNSPGQGTQSENSCTHFP |
| | fused | GNLPDALRDLRDAFSRVKTFFQMKDQLDNLLLKESLLEDFKGY |
| | heterodimer | LGCQALSEMIQFYLEEVMPQAENQDPDIKAHVNSLGENLKTLRL |
| | | RLRRCHRFLPCENKSKAVEQVKNAFNKLQEKGIYKAMSEFDIFI |
| | | NYIEAYMTMKIRN (SEQ ID NO: 175) |
| 176 | des-SER1 | PGQGTQSENSCTHFPGNLPDSLRDLRDAFSRVKTFFQMKDQLDN |
| | hIL10 M22S/ | LLLKESLLEDFKGYLGCQALSEMIQFYLEEVMPQAENQDPDIKA |
| | hIL10 M22S | HVNSLGENLKTLRLRLRRCHRFLPCENKSKAVEQVKNAFNKLQ |
| | fused | EKGIYKAMSEFDIFINYIEAYMTMKIRNSPGQGTQSENSCTHFPG |
| | heterodimer | NLPDSLRDLRDAFSRVKTFFQMKDQLDNLLLKESLLEDFKGYLG |
| | | CQALSEMIQFYLEEVMPQAENQDPDIKAHVNSLGENLKTLRLRL |
| | | RRCHRFLPCENKSKAVEQVKNAFNKLQEKGIYKAMSEFDIFINYI |
| | | EAYMTMKIRN (SEQ ID NO: 176) |
| 177 | des-SER1 | PGQGTQSENSCTHFPGNLPDTLRDLRDAFSRVKTFFQMKDQLDN |
| | hIL10 M22T/ | LLLKESLLEDFKGYLGCQALSEMIQFYLEEVMPQAENQDPDIKA |
| | hIL10 M22T | HVNSLGENLKTLRLRLRRCHRFLPCENKSKAVEQVKNAFNKLQ |
| | fused | EKGIYKAMSEFDIFINYIEAYMTMKIRNSPGQGTQSENSCTHFPG |
| | heterodimer | NLPDTLRDLRDAFSRVKTFFQMKDQLDNLLLKESLLEDFKGYLG |
| | | CQALSEMIQFYLEEVMPQAENQDPDIKAHVNSLGENLKTLRLRL |
| | | RRCHRFLPCENKSKAVEQVKNAFNKLQEKGIYKAMSEFDIFINYI |
| | | EAYMTMKIRN (SEQ ID NO: 177) |
| 178 | des-SER1 | PGQGTQSENSCTHFPGNLPDDLRDLRDAFSRVKTFFQMKDQLD |
| | hIL10 M22D/ | NLLLKESLLEDFKGYLGCQALSEMIQFYLEEVMPQAENQDPDIK |
| | hIL10M22D | AHVNSLGENLKTLRLRLRRCHRFLPCENKSKAVEQVKNAFNKL |
| | fused | QEKGIYKAMSEFDIFINYIEAYMTMKIRNSPGQGTQSENSCTHFP |
| | heterodimer | GNLPDDLRDLRDAFSRVKTFFQMKDQLDNLLLKESLLEDFKGY |
| | | LGCQALSEMIQFYLEEVMPQAENQDPDIKAHVNSLGENLKTLRL |
| | | RLRRCHRFLPCENKSKAVEQVKNAFNKLQEKGIYKAMSEFDIFI |
| | | NYIEAYMTMKIRN (SEQ ID NO: 178) |
| 179 | des-SER1 | PGQGTQSENSCTHFPGNLPDWLRDLRDAFSRVKTFFQMKDQLD |
| | hIL10 M22W/ | NLLLKESLLEDFKGYLGCQALSEMIQFYLEEVMPQAENQDPDIK |
| | hIL10 M22W | AHVNSLGENLKTLRLRLRRCHRFLPCENKSKAVEQVKNAFNKL |
| | fused | QEKGIYKAMSEFDIFINYIEAYMTMKIRNSPGQGTQSENSCTHFP |
| | heterodimer | GNLPDWLRDLRDAFSRVKTFFQMKDQLDNLLLKESLLEDFKGY |
| | | LGCQALSEMIQFYLEEVMPQAENQDPDIKAHVNSLGENLKTLRL |
| | | RLRRCHRFLPCENKSKAVEQVKNAFNKLQEKGIYKAMSEFDIFI |
| | | NYIEAYMTMKIRN (SEQ ID NO: 179) |
| 180 | des-SER1 | PGQGTQSENSCTHFPGNLPNMLEDLRDAFSRVKTFFQMKDQLD |
| | hIL10 R24E/ | NLLLKESLLEDFKGYLGCQALSEMIQFYLEEVMPQAENQDPDIK |
| | hIL10 R24E | AHVNSLGENLKTLRLRLRRCHRFLPCENKSKAVEQVKNAFNKL |
| | fused | QEKGIYKAMSEFDIFINYIEAYMTMKIRNSPGQGTQSENSCTHFP |
| | heterodimer | GNLPNMLEDLRDAFSRVKTFFQMKDQLDNLLLKESLLEDFKGY |
| | | LGCQALSEMIQFYLEEVMPQAENQDPDIKAHVNSLGENLKTLRL |
| | | RLRRCHRFLPCENKSKAVEQVKNAFNKLQEKGIYKAMSEFDIFI |
| | | NYIEAYMTMKIRN (SEQ ID NO: 180) |
| 181 | des-SER1 | PGQGTQSENSCTHFPGNLPNMLRKLRDAFSRVKTFFQMKDQLD |
| | hIL10 D25K/ | NLLLKESLLEDFKGYLGCQALSEMIQFYLEEVMPQAENQDPDIK |
| | hIL10 D25K | AHVNSLGENLKTLRLRLRRCHRFLPCENKSKAVEQVKNAFNKL |
| | fused | QEKGIYKAMSEFDIFINYIEAYMTMKIRNSPGQGTQSENSCTHFP |
| | heterodimer | GNLPNMLRKLRDAFSRVKTFFQMKDQLDNLLLKESLLEDFKGY |
| | | LGCQALSEMIQFYLEEVMPQAENQDPDIKAHVNSLGENLKTLRL |
| | | RLRRCHRFLPCENKSKAVEQVKNAFNKLQEKGIYKAMSEFDIFI |
| | | NYIEAYMTMKIRN (SEQ ID NO: 181) |
| 182 | des-SER1 | PGQGTQSENSCTHFPGNLPNMLRDLRDAFSRVKTFFQMKDQLD |
| | hIL10 E96K/ | NLLLKESLLEDFKGYLGCQALSEMIQFYLEEVMPQAENQDPDIK |
| | hIL10 E96K | AHVNSLGKNLKTLRLRLRRCHRFLPCENKSKAVEQVKNAFNKL |
| | fused | QEKGIYKAMSEFDIFINYIEAYMTMKIRNSPGQGTQSENSCTHFP |
| | heterodimer | GNLPNMLRDLRDAFSRVKTFFQMKDQLDNLLLKESLLEDFKGY |
| | | LGCQALSEMIQFYLEEVMPQAENQDPDIKAHVNSLGKNLKTLRL |
| | | RLRRCHRFLPCENKSKAVEQVKNAFNKLQEKGIYKAMSEFDIFI |
| | | NYIEAYMTMKIRN (SEQ ID NO: 182) |
| 183 | des-SER1 | PGQGTQSENSCTHFPGNLPNMLRDLRDAFSRVKTFFQMKDQLD |
| | hIL10 E96Q/ | NLLLKESLLEDFKGYLGCQALSEMIQFYLEEVMPQAENQDPDIK |
| | hIL10 E96Q | AHVNSLGQNLKTLRLRLRRCHRFLPCENKSKAVEQVKNAFNKL |
| | fused | QEKGIYKAMSEFDIFINYIEAYMTMKIRNSPGQGTQSENSCTHFP |
| | heterodimer | GNLPNMLRDLRDAFSRVKTFFQMKDQLDNLLLKESLLEDFKGY |
| | | LGCQALSEMIQFYLEEVMPQAENQDPDIKAHVNSLGQNLKTLRL |
| | | RLRRCHRFLPCENKSKAVEQVKNAFNKLQEKGIYKAMSEFDIFI |
| | | NYIEAYMTMKIRN (SEQ ID NO: 183) |
| 184 | des-SER1 | PGQGTQSENSCTHFPGNLPNMLRDLRDAFSRVKTFFQMKDQLD |
| | hIL10 T100E/ | NLLLKESLLEDFKGYLGCQALSEMIQFYLEEVMPQAENQDPDIK |
| | hIL10 | AHVNSLGENLKELRLRLRRCHRFLPCENKSKAVEQVKNAFNKL |
| | T100E | QEKGIYKAMSEFDIFINYIEAYMTMKIRNSPGQGTQSENSCTHFP |
| | fused | GNLPNMLRDLRDAFSRVKTFFQMKDQLDNLLLKESLLEDFKGY |
| | heterodimer | LGCQALSEMIQFYLEEVMPQAENQDPDIKAHVNSLGENLKELRL |
| | | RLRRCHRFLPCENKSKAVEQVKNAFNKLQEKGIYKAMSEFDIFI |
| | | NYIEAYMTMKIRN (SEQ ID NO: 184) |
| 185 | des-SER1 | PGQGTQSENSCTHFPGNLPNMLRDLRDAFSRVKTFFQMKDQLD |
| | hIL10 T100L/ | NLLLKESLLEDFKGYLGCQALSEMIQFYLEEVMPQAENQDPDIK |
| | hIL10 T100L | AHVNSLGENLKLLRLRLRRCHRFLPCENKSKAVEQVKNAFNKL |
| | fused | QEKGIYKAMSEFDIFINYIEAYMTMKIRNSPGQGTQSENSCTHFP |
| | heterodimer | GNLPNMLRDLRDAFSRVKTFFQMKDQLDNLLLKESLLEDFKGY |
| | | LGCQALSEMIQFYLEEVMPQAENQDPDIKAHVNSLGENLKLLRL |
| | | RLRRCHRFLPCENKSKAVEQVKNAFNKLQEKGIYKAMSEFDIFI |
| | | NYIEAYMTMKIRN (SEQ ID NO: 185) |
| 186 | des-SER1 | PGQGTQSENSCTHFPGNLPNMLRDLRDAFSRVKTFFQMKDQLD |
| | hIL10 T100C/ | NLLLKESLLEDFKGYLGCQALSEMIQFYLEEVMPQAENQDPDIK |
| | hIL10 T100C | AHVNSLGENLKCLRLRLRRCHRFLPCENKSKAVEQVKNAFNKL |
| | fused | QEKGIYKAMSEFDIFINYIEAYMTMKIRNSPGQGTQSENSCTHFP |
| | heterodimer | GNLPNMLRDLRDAFSRVKTFFQMKDQLDNLLLKESLLEDFKGY |
| | | LGCQALSEMIQFYLEEVMPQAENQDPDIKAHVNSLGENLKCLRL |
| | | RLRRCHRFLPCENKSKAVEQVKNAFNKLQEKGIYKAMSEFDIFI |
| | | NYIEAYMTMKIRN (SEQ ID NO: 186) |

Examples of fused mouse IL10 polypeptides are shown in the table below, in which the linker is in bold. In some embodiments, for each of the fused mIL10 polypeptide sequence listed below, a His-tag can be fused to the N-terminus of the sequence with or without a linker, i.e., GGSHHHHHHHH (SEQ ID NO:23) can be fused to the N-terminus of a fused mIL10 polypeptide listed below.

TABLE 3
Exemplary Mouse Fused IL10 Polypeptides and Nucleic Acids Encoding Same

| Name | Protein Sequence | DNA Sequence |
|---|---|---|
| TP0035 | QYSREDNNCTHFPVGQS | cagtacagcagagaagataataactgcacccacttccccgtagggcag |
| | HMLLELRTAFSQVKTFF | agccatatgctgctggagctgagaacagcctttagccaagtcaagacct |
| | QTKDQLDNILLTDSLMQ | tcttccaaacgaaggatcagcttgacaatatactgctgaccgatagcctg |
| | DFKGYLGCQALSEMIQF | atgcaagacttcaaaggttacctcggctgtcaagccctctccgagatgat |
| | YLVEVMPQAEKHGPEIK | ccaattttacctggtggaggtgatgccgcaagccgaaaaacatggccc |
| | EHLNSLGEKLKTLRMRL | cgagatcaaagaacatctgaacagtcttggtgaaaagctgaagaccctg |
| | RRCHRFLPCENKSKAVE | cgaatgcggctgcgcagatgtcacagatttctaccgtgcgagaataaga |
| | QVKSDFNKLQDQGVYK | gcaaggccgtggagcaagtgaagagcgacttcaataagctgcaagac |
| | AMNEFDIFINCIEAYMMI | caaggcgtttataaggcgatgaacgagttcgatatatttatcaactgcata |
| | KMKSSRGQYSREDNQYS | gaagcctatatgatgatcaagatgaagagcagcagagggcagtactcg |
| | REDNNCTHFPVGQSHML | agagaggataatcagtacagccgagaagacaacaactgtacccacttc |
| | LELRTAFSQVKTFFQTKD | cccgtgggtcaaagccacatgctgctagagctcagaacggccttcagc |
| | QLDNILLTDSLMQDFKG | caagtgaagactttctttcaaaccaaggaccaactggacaacatcctgtt |
| | YLGCQALSEMIQFYLVE | aaccgactcactaatgcaagactttaaggggtacctgggttgccaagcc |
| | VMPQAEKHGPEIKEHLN | ctgagcgagatgattcagttctacctcgtggaggtcatgcctcaagccg |
| | SLGEKLKTLRMRLRRCH | aaaagcacggccccgagattaaagagcacttaaacagtctaggggag |
| | RFLPCENKSKAVEQVKS | aaactgaagaccttgagaatgagattaagacggtgtcatagatttctccc |
| | DFNKLQDQGVYKAMNE | ctgtgagaacaaaagcaaagcggtggagcaagtgaagagcgattttaa |
| | FDIFINCIEAYMMIKMKS | caagctccaagatcaaggcgtatataaagcgatgaatgagttcgatatct |
| | (SEQ ID NO: 78) | tcattaactgcatcgaagcctatatgatgattaaaatgaaaagcgg |
| | | (SEQ ID NO: 88) |
| TP0040 | QYSREDNNCTHFPVGQS | cagtacagcagagaagataataactgcacccacttccccgtagggcag |
| | HMLLELRTAFSQVKTFF | agccatatgctgctggagctgagaacagcctttagccaagtcaagacct |
| | QTKDQLDNILLTDSLMQ | tcttccaaacgaaggatcagcttgacaatatactgctgaccgatagcctg |
| | DFKGYLGCQALSEMIQF | atgcaagacttcaaaggttacctcggctgtcaagccctctccgagatgat |
| | YLVEVMPQAEKHGPEIK | ccaattttacctggtggaggtgatgccgcaagccgaaaaacatggccc |
| | EHLNSLGEKLKLLRMRL | cgagatcaaagaacatctgaacagtcttggtgaaaagctgaagctgctg |
| | RRCHRFLPCENKSKAVE | cgaatgcggctgcgcagatgtcacagatttctaccgtgcgagaataaga |
| | QVKSDFNKLQDQGVYK | gcaaggccgtggagcaagtgaagagcgacttcaataagctgcaagac |
| | AMNEFDIFINCIEAYMMI | caaggcgtttataaggcgatgaacgagttcgatatatttatcaactgcata |
| | KMKSSRGQYSREDNQYS | gaagcctatatgatgatcaagatgaagagcagcagagggcagtactcg |
| | REDNNCTHFPVGQSHML | agagaggataatcagtacagccgagaagacaacaactgtacccacttc |
| | LELRTAFSQVKTFFQTKD | cccgtgggtcaaagccacatgctgctagagctcagaacggccttcagc |
| | QLDNILLTDSLMQDFKG | caagtgaagactttctttcaaaccaaggaccaactggacaacatcctgtt |
| | YLGCQALSEMIQFYLVE | aaccgactcactaatgcaagactttaaggggtacctgggttgccaagcc |
| | VMPQAEKHGPEIKEHLN | ctgagcgagatgattcagttctacctcgtggaggtcatgcctcaagccg |
| | S | aaaagcacggccccgagattaaagagcacttaaacagtctaggggag |
| | LGEKLKLLRMRLRRCHR | aaactgaagctgttgagaatgagattaagacggtgtcatagatttctccc |
| | FLPCENKSKAVEQVKSD | ctgtgagaacaaaagcaaagcggtggagcaagtgaagagcgattttaa |
| | FNKLQDQGVYKAMNEF | caagctccaagatcaaggcgtatataaagcgatgaatgagttcgatatct |
| | DIFINCIEAYMMIKMKS | tcattaactgcatcgaagcctatatgatgattaaaatgaaaagcgg |
| | (SEQ ID NO: 79) | SEQ ID NO: 89) |
| TP0054 | QYSREDNNCTHFPVGQS | cagtacagcagagaagataataactgcacccacttccccgtagggcag |
| | HMLLELRTAFSQVKTFF | tcacacatgctgctggagctgcggactgcctttagccaagtcaagacctt |
| | QTKDQLDNILLTDSLMQ | cttccaaacgaaggatcagcttgacaatatactgctgactgacagcctta |
| | DFKGYLGCQALSEMIQF | tgcaagacttcaaaggttacctcggctgtcaagccctctccgagatgatc |
| | YLVEVMPQAEKHGPEIK | caattttacctggtggaggtgatgccgcaagccgagaaacacggaccg |
| | EHLNSLGEKLKLLRMRL | gagatcaaagaacatctgaacagtcttggtgaaaagctgaagctgctga |
| | RRCHRFLPCENKSKAVE | gaatgcgcctgagaagatgccatagattcctgccctgcgagaacaagt |
| | QVKSDFNKLQDQGVYK | ccaaagcggttgaacaagtgaagagcgacttcaataagctgcaagacc |
| | AMNEFDIFINCIEAYMMI | aaggcgtgtataaggctatgaacgagttcgacatctttataaattgtatcg |
| | KMKSSRGQYSREDNQYS | aggcgtacatgatgataaaaatgaaaagcagcagagggcagtactcga |
| | REDNNCTHFPVGQSHML | gagaggataatcaatacagcagagaagacaacaactgcactcactttcc |
| | LELRTAFSQVKTFFQTKD | cgtgggtcagtcacatatgctcctggaactgagaaccgccttctcgcaa |
| | QLDNILLTDSLMQDFKG | gtgaagactttcttccaaaccaaggatcagctggacaacatcttgctcac |
| | YLGCQALSEMIQFYLVE | tgacagcctgatgcaagacttcaaaggctaccttggctgccaagccctg |
| | VMPQAEKHGPEIKEHLN | agcgaaatgattcagttctacctagttgaagtgatgcctcaagccgaaaa |
| | SLGEKLKTLRMRLRRCH | gcacggccccgaaataaaggaacacctgaacagcttaggcgagaagc |
| | RFLPCENKSKAVEQVKS | tgaagacattgagaatgagattaagaagatgccacagattcttaccctgt |
| | DFNKLQDQGVYKAMNE | gaaaataaaagcaaggcggtggagcaagtgaagagcgattttaacaa |
| | FDIFINCIEAYMMIKMKS | gctccaagaccaaggcgtatataaagcgatgaacgagttcgacatcttc |
| | (SEQ ID NO: 80) | attaactgcatcgaagcatacatgatgattaagatgaagagcgg |
| | | (SEQ ID NO: 90) |
| DR2467 | QYSREDNSCTHFPVGQS | caatactcaagagaagataacagttgcacacatttccccgtagggcaaa |
| | HMLLELRTAFSQVKTFF | gtcacatgctgttggaattgcgcactgcattttctcaggtaaaaacctttttc |
| | QTKDQLDNILLTDSLMQ | caaaccaaagaccagcttgataacattcttctgaccgactcacttatgca |
| | DFKGYLGCQALSEMIQF | ggacttcaaaggctaccttggctgccaagccctttctgaaatgatacaatt |
| | YLVEVMPQAEKHGPEIK | ctacttggtcgaggtcatgcctcaggcagaaaagcatggtccagaaatc |
| | EHLNSLGEKLKTLRMRL | aaagaacatttgaactccctgggagagaagcttaaaactcttaggatgc |
| | RRCHRFLPCENKSKAVE | gccttcggagatgccatcgctttcttccatgcgagaataaaagcaaggc |
| | QVKSDFNKLQDQGVYK | cgtcgaacaagtaaagtccgactttaataagctgcaagaccaaggtgtc |
| | AMNEFDIFINCIEAYMMI | tataaagcaatgaacgaatttgacatctttatcaattgtattgaggcctatat |
| | KMKSSRGQYSREDNQYS | gatgatcaaaatgaaaagttcccgcgggcaatactcacgggaggacaa |
| | REDNSCTHFPVGQSHML | tcaatattcccgagaagataattcttgtacccacttccccgtcggtcaaag |
| | LELRTAFSQVKTFFQTKD | ccacatgcttcttgagctcagaactgcattctcccaagtcaagacttttttc |
| | QLDNILLTDSLMQDFKG | caaacaaaagatcaacttgacaacatcctcttgactgactccttgatgca |
| | YLGCQALSEMIQFYLVE | agacttcaaaggttacctcggctgtcaagctctcagtgagatgattcaatt |
| | VMPQAEKHGPEIKEHLN | ctatttggtagaggttatgcctcaagccgagaagcatgggccagaaatc |
| | SLGEKLKTLRMRLRRCH | aaagagcaccttaactccctgggtgaaaaactcaagacccttcggatga |
| | RFLPCENKSKAVEQVKS | ggcttcgcagatgtcaccggttcctcccatgcgagaataagtccaaggc |
| | DFNKLQDQGVYKAMNE | tgttgagcaagtgaaatcagacttcaacaaattgcaagaccaaggagttt |
| | FDIFINCIEAYMMIKMKS | acaaagcaatgaatgagttcgatatctttattaactgtatcgaagcatacat |
| | (SEQ ID NO: 81) | gatgattaaaatgaagagc (SEQ ID NO: 91) |
| WC163 | GNCTHFPVGQSHMLLEL | ggcaactgtactcatttcccagtggggcaaagccatatgttgttggaattg |
| | RTAFSQVKTFFQTKDQL | cgcactgctttctctcaggtcaaaacattttttcagacaaaagaccaattg |
| | DNILLTDSLMQDFKGYL | gacaacattttattgacagatagtctgatgcaggattttaaggggtacttg |
| | GCQALSEMIQFYLVEVM | ggttgtcaagctctttctgaaatgatccagttttatctggttgaagttatgcc |
| | PQAEKHGPEIKEHLNSLG | acaggcggaaaagcacggaccggagatcaaggaacatttaaattcact |
| | EKLKTLRMRLRRCHRFL | gggcgaaaaacttaagactctgcgcatgcgcctgcgccgctgtcaccg |
| | PCENKSKAVEQVKSDFN | ttttctgccatgtgaaaataagtccaaggccgtagaacaggtgaaatctg |
| | KLQDQGVYKAMNEFDIF | acttcaacaaactgcaggaccagggggtgtacaaagcaatgaatgagt |
| | INCIEAYMMIKMKSSRG | ttgatatttttatcaattgtattgaagcctatatgatgatcaagatgaaatcat |
| | QYSREDNQYSREDNSCT | ctcgcggtcaatacagccgcgaagacaaccagtattcacgtgaggaca |
| | HFPVGQSHMLLELRTAFS | attcgtgcacccacttcccgggggccaatcgcatatgctgctggagtta |
| | QVKTFFQTKDQLDNILLT | cgcacagccttctcgcaggtgaagactttttttcagactaaggatcagct |
| | DSLMQDFKGYLGCQALS | ggataatatcctgttgaccgattcattaatgcaagatttcaagggttacctg |
| | EMIQFYLVEVMPQAEKH | ggttgccaggcgttatccgagatgattcaattctacttagtagaagtgatg |
| | GPEIKEHLNSLGEKLKTL | ccccaggctgaaaaacacggtcctgagattaaagagcacttgaacagtt |
| | RMRLRRCHRFLPCENKS | tgggagaaaagctgaagactcttcgtatgcgtctgcgccgctgccatcg |
| | KAVEQVKSDFNKLQDQG | ctttttgccctgtgagaataaatccaaggccgtcgagcaggttaagtctg |
| | VYKAMNEFDIFINCIEAY | atttcaacaaattacaggatcaaggggtttacaaagcgatgaatgagttt |
| | MMIKMKS (SEQ ID | gacatctttattaattgtatcgaggcttatatgatgattaagatgaagagtg |
| | NO: 82) | g (SEQ ID NO: 92) |
| SS0051 | GNCTHFPVGQSHMLLEL | ggcaactgtactcatttcccagtggggcaaagccatatgttgttggaattg |
| | RTAFSQVKTFFQTKDQL | cgcactgctttctctcaggtcaaaacattttttcagacaaaagaccaattg |
| | DNILLTDSLMQDFKGYL | gacaacattttattgacagatagtctgatgcaggattttaaggggtacttg |
| | GCQALSEMIQFYLVEVM | ggttgtcaagctctttctgaaatgatccagttttatctggttgaagttatgcc |
| | PQAEKHGPEIKEHLNSLG | acaggcggaaaagcacggaccggagatcaaggaacatttaaattcact |
| | EKLKLLRMRLRRCHRFL | gggcgaaaaacttaagctactgcgcatgcgcctgcgccgctgtcaccg |
| | PCENKSKAVEQVKSDEN | ttttctgccatgtgaaaataagtccaaggccgtagaacaggtgaaatctg |
| | KLQDQGVYKAMNEFDIF | acttcaacaaactgcaggaccagggggtgtacaaagcaatgaatgagt |
| | INCIEAYMMIKMKSSRG | ttgatatttttatcaattgtattgaagcctatatgatgatcaagatgaaatcat |
| | QYSREDNQYSREDNSCT | ctcgcggtcaatacagccgcgaagacaaccagtattcacgtgaggaca |
| | HFPVGQSHMLLELRTAFS | attcgtgcacccacttcccggtgggccaatcgcatatgctgctggagtta |
| | QVKTFFQTKDQLDNILLT | cgcacagccttctcgcaggtgaagactttttttcagactaaggatcagct |
| | DSLMQDFKGYLGCQALS | ggataatatcctgttgaccgattcattaatgcaagatttcaagggttacctg |
| | EMIQFYLVEVMPQAEKH | ggttgccaggcgttatccgagatgattcaattctacttagtagaagtgatg |
| | GPEIKEHLNSLGEKLKLL | ccccaggctgaaaaacacggtcctgagattaaagagcacttgaacagtt |
| | RMRLRRCHRFLPCENKS | tgggagaaaagctgaagctccttcgtatgcgtctgcgccgctgccatcg |
| | KAVEQVKSDFNKLQDQG | ctttttgccctgtgagaataaatccaaggccgtcgagcaggttaagtctg |
| | VYKAMNEFDIFINCIEAY | atttcaacaaattacaggatcaaggggtttacaaagcgatgaatgagttt |
| | MMIKMKS (SEQ ID | gacatctttattaattgtatcgaggcttatatgatgattaagatgaagagt |
| | NO: 83) | (SEQ ID NO: 93) |
| SS0060 | SRGQYSREDNNCTHFPV | agcagaggccagtacagcagagaagataataactgcacccacttcccc |
| | GQSHMLLELRTAFSQVK | gtagggcagagccatatgctgctggagctgagaacagcctttagccaa |
| | TFFQTKDQLDNILLTDSL | gtcaagaccttcttccaaacgaaggatcagcttgacaatatactgctgac |
| | MQDFKGYLGCQALSEMI | cgatagcctgatgcaagacttcaaaggttacctcggctgtcaagccctct |
| | QFYLVEVMPQAEKHGPE | ccgagatgatccaattttacctggtggaggtgatgccgcaagccgaaaa |
| | IKEHLNSLGEKLKTLRMR | acatggccccgagatcaaagaacatctgaacagtcttggtgaaaagctg |
| | LRRCHRFLPCENKSKAV | aagaccctgcgaatgcggctgcgcagatgtcacagatttctaccgtgcg |
| | EQVKSDFNKLQDQGVYK | agaataagagcaaggccgtggagcaagtgaagagcgacttcaataag |
| | AMNEFDIFINCIEAYMMI | ctgcaagaccaaggcgtttataaggcgatgaacgagttcgatatatttat |
| | KMKSSRGQYSREDNQYS | caactgcatagaagcctatatgatgatcaagatgaagagcagcagagg |
| | REDNNCTHFPVGQSHML | gcagtactcgagagaggataatcagtacagccgagaagacaacaact |
| | LELRTAFSQVKTFFQTKD | gtacccacttccccgtgggtcaaagccacatgctgctagagctcagaac |
| | QLDNILLTDSLMQDFKG | ggccttcagccaagtgaagactttctttcaaaccaaggaccaactggac |
| | YLGCQALSEMIQFYLVE | aacatcctgttaaccgactcactaatgcaagactttaaggggtacctggg |
| | VMPQAEKHGPEIKEHLN | ttgccaagccctgagcgagatgattcagttctacctcgtggaggtcatgc |
| | SLGEKLKTLRMRLRRCH | ctcaagccgaaaagcacggccccgagattaaagagcacttaaacagtc |
| | RFLPCENKSKAVEQVKS | taggggagaaactgaagaccttgagaatgagattaagacggtgtcata |
| | DFNKLQDQGVYKAMNE | gatttctcccctgtgagaacaaaagcaaagcggtggagcaagtgaaga |
| | FDIFINCIEAYMMIKMKS | gcgattttaacaagctccaagatcaaggcgtatataaagcgatgaatga |
| | (SEQ ID NO: 84) | gttcgatatcttcattaactgcatcgaagcctatatgatgattaaaatgaaa |
| | | agcgg (SEQ ID NO: 94) |
| SS0061 | SRGQYSREDNNCTHFPV | agcagaggccagtacagcagagaagataataactgcacccacttcccc |
| | GQSHMLLELRTAFSQVK | gtagggcagagccatatgctgctggagctgagaacagcctttagccaa |
| | TFFQTKDQLDNILLTDSL | gtcaagaccttcttccaaacgaaggatcagcttgacaatatactgctgac |
| | MQDFKGYLGCQALSEMI | cgatagcctgatgcaagacttcaaaggttacctcggctgtcaagccctct |
| | QFYLVEVMPQAEKHGPE | ccgagatgatccaattttacctggtggaggtgatgccgcaagccgaaaa |
| | IKEHLNSLGEKLKLLRMR | acatggccccgagatcaaagaacatctgaacagtcttggtgaaaagctg |
| | LRRCHRFLPCENKSKAV | aagctgctgcgaatgcggctgcgcagatgtcacagatttctaccgtgcg |
| | EQVKSDFNKLQDQGVYK | agaataagagcaaggccgtggagcaagtgaagagcgacttcaataag |
| | AMNEFDIFINCIEAYMMI | ctgcaagaccaaggcgtttataaggcgatgaacgagttcgatatatttat |
| | KMKSSRGQYSREDNQYS | caactgcatagaagcctatatgatgatcaagatgaagagcagcagagg |
| | REDNNCTHFPVGQSHML | gcagtactcgagagaggataatcagtacagccgagaagacaacaact |
| | LELRTAFSQVKTFFQTKD | gtacccacttccccgtgggtcaaagccacatgctgctagagctcagaac |
| | QLDNILLTDSLMQDFKG | ggccttcagccaagtgaagactttctttcaaaccaaggaccaactggac |
| | YLGCQALSEMIQFYLVE | aacatcctgttaaccgactcactaatgcaagactttaaggggtacctggg |
| | VMPQAEKHGPEIKEHLN | ttgccaagccctgagcgagatgattcagttctacctcgtggaggtcatgc |
| | SLGEKLKLLRMRLRRCH | ctcaagccgaaaagcacggccccgagattaaagagcacttaaacagtc |
| | RFLPCENKSKAVEQVKS | taggggagaaactgaagctgttgagaatgagattaagacggtgtcatag |
| | DFNKLQDQGVYKAMNE | atttctcccctgtgagaacaaaagcaaagcggtggagcaagtgaagag |
| | FDIFINCIEAYMMIKMKS | cgattttaacaagctccaagatcaaggcgtatataaagcgatgaatgagt |
| | (SEQ ID NO: 85) | tcgatatcttcattaactgcatcgaagcctatatgatgattaaaatgaaaag |
| | | cgg (SEQ ID NO: 95) |
| DR2677 | EYSREDNNCTHFPVGQS | gagtactccagagaagataataactgtacccactttcctgtgggccaga |
| | HMLLELRTAFSQVKTFF | gtcacatgctcctggagttgcgtaccgccttttcacaggtcaagaccttttt |
| | QTKDQLDNILLTDSLMQ | ccagactaaggaccagctggacaatatcctcctgaccgattccctgatg |
| | DFKGYLGCQALSEMIQF | caggactttaagggctatctgggctgccaagccctgtccgagatgatcc |
| | YLVEVMPQAEKHGPEIK | agttctatctggtcgaagttatgcctcaagcagagaaacacggacctga |
| | EHLNSLGEKLKTLRMRL | gatcaaggaacatttgaacagcctcggggaaaagctgaagactcttcgt |
| | RRCHRFLPCENKSKAVE | atgcgccttcgcagatgccatcggtttctcccgtgtgagaataagtccaa |
| | QVKSDFNKLQDQGVYK | ggcggtggagcaggtgaagagcgacttcaacaagttgcaggaccagg |
| | AMNEFDIFINCIEAYMMI | gcgtttacaaagctatgaatgagttcgacatcttcatcaactgcatcgagg |
| | KMKSSRGQYSREDNNCT | cttacatgatgattaagatgaaaagctctcgcggtcagtacagccgcga |
| | HFPVGQSHMLLELRTAFS | ggataataattgcacacatttccccgtcggccaatcacacatgcttctgg |
| | QVKTFFQTKDQLDNILLT | agcttcggactgctttttcccaggtcaagactttctttcagacaaaggacc |
| | DSLMQDFKGYLGCQALS | aactggacaacattctcttgaccgatagcctgatgcaggactttaagggg |
| | EMIQFYLVEVMPQAEKH | tatctgggttgtcaagcactgtccgagatgatccagttctacctcgtggag |
| | GPEIKEHLNSLGEKLKTL | gtgatgcctcaagctgagaagcatggccccgagattaaggaacacctc |
| | RMRLRRCHRFLPCENKS | aattcccttggcgagaagctgaagactctgcgcatgaggctgagaagg |
| | KAVEQVKSDFNKLQDQG | tgccaccgttttttgccttgtgagaacaagtctaaggccgtggaacaggt |
| | VYKAMNEFDIFINCIEAY | gaagagcgactttaacaagctccaggatcagggcgtatataaggccat |
| | MMIKMKS (SEQ ID | gaacgagttcgatatttttataaattgcatcgaggcttacatgatgattaaa |
| | NO: 86) | atgaagagc (SEQ ID NO: 96) |
| DR2678 | EYSREDNNCTHFPVGQS | gagtattctcgggaggacaataattgcacccacttccctgtcggccaga |
| | HMLLELRTAFSQVKTFF | gccacatgcttctggagcttagaactgccttttcccaggttaagacattctt |
| | QTKDQLDNILLTDSLMQ | tcaaaccaaggatcagctggacaacatcttgctgactgactctctgatgc |
| | DFKGYLGCQALSEMIQF | aggacttcaaaggctatcttggttgccaggccctgtccgagatgatcca |
| | YLVEVMPQAEKHGPEIK | gttctatctggtcgaagtgatgccacaggcagaaaagcatggccctgag |
| | EHLNSLGEKLKLLRMRL | atcaaggagcacctcaattccctgggcgaaaagctgaagctcctgcgg |
| | RRCHRFLPCENKSKAVE | atgcgtctgcgcagatgccatagattcctgccgtgtgagaacaagtctaa |
| | QVKSDFNKLQDQGVYK | agccgtggagcaggtcaagagcgatttcaacaagctccaggaccagg |
| | AMNEFDIFINCIEAYMMI | gggtttacaaggccatgaacgagttcgatatcttcattaactgcatcgaa |
| | KMKSSRGQYSREDNNCT | gcctacatgatgattaagatgaagtctagtagaggacagtacagcagag |
| | HFPVGQSHMLLELRTAFS | aggacaataactgcacacacttcccggtgggccagagtcacatgctgtt |
| | QVKTFFQTKDQLDNILLT | ggagctgcgcacggccttttcccaggtgaaaacttttttccagacgaag |
| | DSLMQDFKGYLGCQALS | gaccagctcgataatatcctcttgaccgacagcctcatgcaggactttaa |
| | EMIQFYLVEVMPQAEKH | aggctacctgggctgtcaggctctctccgagatgatccagttctacctcg |
| | GPEIKEHLNSLGEKLKLL | tcgaggtgatgccccaggcggagaagcacggtcctgagatcaaggag |
| | RMRLRRCHRFLPCENKS | cacctgaactccctgggcgagaagctgaaactcttgcgtatgcgcctgc |
| | KAVEQVKSDFNKLQDQG | ggcgttgtcacagatttctcccttgtgaaaacaagagcaaggctgttgaa |
| | VYKAMNEFDIFINCIEAY | caggtgaaatccgacttcaacaaacttcaagaccagggcgtctacaag |
| | MMIKMKS (SEQ ID | gccatgaacgaatttgacatcttcataaattgcattgaagcctatatgatga |
| | NO: 87) | ttaagatgaagtcag (SEQ ID NO: 97) |

Further, hIL10 muteins and fused hIL10 polypeptides containing hIL10 muteins of the disclosure may exhibit differential expression or thermostability depending on the particular mutation incorporated into the hIL10A or hIL10B subunit. hIL10 muteins and fused hIL10 polypeptides containing hIL10 muteins, where the hIL10 mutein includes a T100L, N21K, M22A, or R24E mutation may exhibit increased expression or thermostability as compared to hIL10 muteins having substitution mutations at other amino acid positions or having a different amino acid substitution at the same position.

### Biased Activity

The inflammatory response is a series of biological events in a mammal initiated in response to an infectious and/or injurious stimulus that mitigates the potential for systemic infection. Typically, the mammalian inflammatory response mediated by myeloid cells, particularly macrophages which are activated by foreign stimuli, for example components of bacterial cell walls such as the lipopolysaccharide of gram-negative bacteria (“LPS”). The activated myeloid cells act as the harbingers of infection and/or injury by secreting various pro-inflammatory signaling molecules including but not limited to interleukin-6 (IL6), interleukin-1 (IL1, especially IL1(3) and tumor necrosis factor alpha (TNFα) that initiate and/or mediate the multiple biological processes associated with the inflammatory response.

Although the inflammatory response is essential for protecting the mammalian subject against infection, excessive and/or chronic activation of immune cells, particularly myeloid cells, is associated with tissue damage, organ malfunction, and autoimmune disease. A wide variety of human diseases are associated with excessive and/or chronic inflammation including but not limited to inflammatory bowel disease (IBD), rheumatoid arthritis (RA), alzheimer's disease, asthma, type 1 and type 2 diabetes, and cancer). The subject produces certain molecules, e.g., IL10, that act (in additional to other activities) to dampen the inflammatory response to prevent the deleterious effects associated with excess inflammation. Genetic loss of IL10 in both mice and humans is associated with severe inflammatory bowel disease (IBD). Expression and secretion of IL10 is correlated with suppression of the inflammatory response in immune cells including but not limited to inhibition of expression and/or secretion of proinflammatory cytokines and antigen presentation by activated myeloid cells. Despite a central role in the suppression of the inflammatory response, IL10 has also associated with pro-inflammatory activity particularly in activated CD8+ T cells. The contacting of activated CD8+ T cells with IL10 is observed to result in enhanced secretion of the pro-inflammatory cytokine interferon-gamma (IFNγ) as well the release of cytolytic factors such as granzyme A and granzyme B. These competing pro- and anti-inflammatory effects present a challenge to the therapeutic use of IL10 in the treatment of inflammatory disease in mammalian subjects.

In some embodiments, a fused hIL10 polypeptide of the present disclosure exhibits cell type biased activity relative to wild-type IL10. As used herein the term “biased” when used in the in the context of fused IL10 polypeptide is used to indicated that a fused IL10 polypeptide exhibits a greater fraction of the level of wild-type IL10 activity in a first cell type than the level of wild-type IL10 activity in second cell type relative to the wild-type IL10 species from which the fused IL10 polypeptide was derived. In one embodiment, the first cell type is a cell of myeloid origin, including a myeloid cell. In some embodiments, the myeloid cell is selected from a myelocyte, granulocyte, (e.g. neutrophil, eosinophil, or basophil), mast cell, or monocyte. In some embodiments, the monocyte is a macrophage or dendritic cell. In some embodiments, the macrophage is a Kupffer cell. In one embodiment, the first cell type is an activated myeloid cell. In one embodiment, the first cell type is a LPS activated human myeloid cell. In some embodiments, the second cell type is an T cell.

In some embodiments, a fused IL10 polypeptide of the present disclosure inhibits pro-inflammatory responses and/or STAT3-mediated signaling in a cell-type dependent manner, such that inflammatory macrophage activation is inhibited without substantially promoting the production of inflammatory cytokines such as interferon-γ by T cells. In some embodiments, a fused hIL10 polypeptide of the present disclosure retains the immunosuppressive functions of wild-type hIL10, such as inhibiting the production of inflammatory cytokines, while decreasing the immunostimulatory functions of wild-type hIL10, such as the production of IFNγ by CD8⁺ T cells. For example, in some embodiments a fused hIL10 polypeptide of the present disclosure retains activity comparable to wild-type hIL10 to suppress myeloid cell activation (e.g., as evaluated by increased STAT3-mediated signaling in myeloid cells), but possess substantially reduced activation (e.g., as evaluated by decreased production of IFNγ) in PBMCs, T cells, B cells and NK cells.

In some embodiments, the fused IL10 polypeptides of the present disclosure are hIL10 partial agonists.

### Relative STAT3 Induction

As previously noted, the interaction of IL10 with the IL10 receptor results in the intracellular signaling characterized by the enhanced intracellular production of phosphorylated STAT3 (phosphor-STAT3). Consequently, one measure of IL10 activity may be evaluated using a cell expressing the IL10 receptor (comprised of IL10Ra and IL10Rb) is the intracellular production of phospho-STAT3.

In one embodiment, a fused hIL10 polypeptide is biased hIL10 partial agonist, the first cell type is an activated human myeloid cell and the second cell type is an activated human T cell wherein the level of IL10 activity is measured by intracellular production of phospho-STAT3. In one embodiment, a fused hIL10 polypeptide is a biased hIL10 partial agonist that retains a greater fraction of hIL10 activity on activated human monocytes than activated human CD8+ T cells wherein the level of IL10 activity is measured by intracellular production of phospho-STAT3. In some embodiments, the relative activation of STAT3 signaling of a fused hIL10 polypeptide described herein in a first cell type versus a second cell type is different from the relative activation of STAT3 signaling of a wild-type human or murine IL10 in the first cell type versus the second cell type. In some embodiments, the level of intracellular phospho-STAT3 induced in a human myeloid cell in response to contacting the myeloid cell with an effective amount of a fused hIL10 polypeptide is at least 10 fold, alternatively at least 100 fold, alternatively at least 1000 fold, greater than the level of intracellular phospho-STAT3 induced in a human lymphocyte cell in response to contacting the human lymphocyte cell with the same amount of the fused hIL10 polypeptide. In one embodiment, the ratio of the level of STAT3 signaling induced in a myeloid cell in response to contacting a myeloid cell with a fused hIL10 polypeptide relative to the level of STAT3 signaling induced in a lymphocyte cell in response to contacting a lymphocyte with the fused hIL10 polypeptide is different than (greater than or lesser than) the ratio of the level of STAT3 signaling induced in the myeloid cell in response to contacting the myeloid cell with wild-type hIL10 relative to the level of STAT3 signaling induced in the lymphocyte in response to contacting the lymphocyte with wild-type hIL10. In some embodiments, the ratio of the activity (as determined by the level of intracellular phospho-STAT3) of a fused hIL10 polypeptide in human myeloid cells relative to human lymphocytes is greater than the ratio of activity of wild-type human IL10 in human myeloid cells relative to human lymphocytes. In some embodiments, the myeloid cell is a neutrophil, eosinophil, mast cell, basophil or monocyte. In some embodiments, the monocyte is a macrophage or a dendritic cell. In some embodiments, the macrophage is a Kupffer cell. In some embodiments, the lymphocyte is a CD8+ T cell, a CD4+ T cell, a B cell or an NK cell.

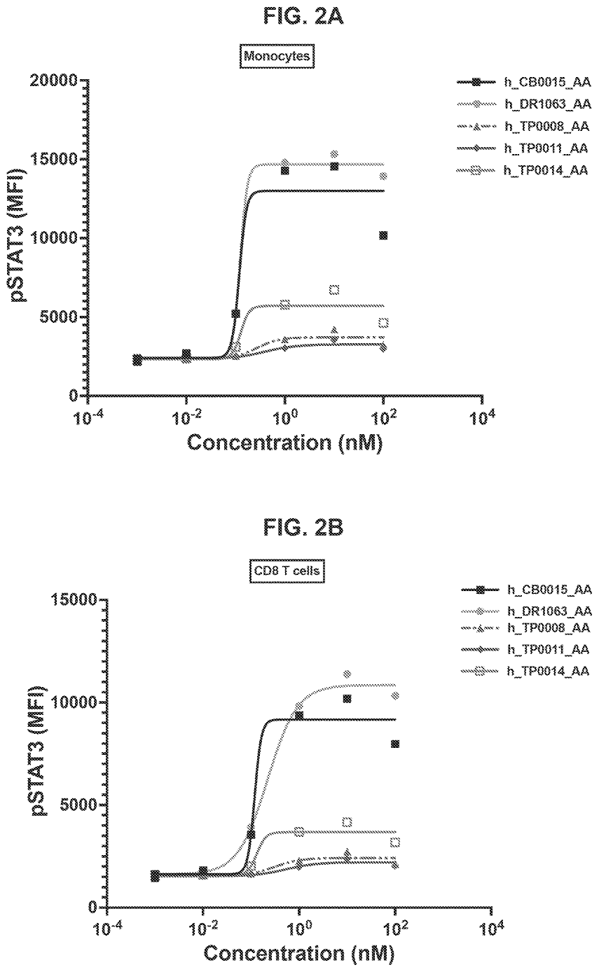

In some embodiments, a fused hIL10 polypeptide of the present disclosure have a pSTAT3 Eₘₐₓ of greater than 20%, greater than 30%, greater than 40%, greater than 50%, greater than 60%, or greater than 70% of the pSTAT3 Eₘₐₓ of wild-type hIL10 in myeloid cells (See, for example FIG. 2A). In some embodiments, a fused hIL10 polypeptide of the present disclosure exhibits decreased STAT3-mediated signaling in lymphocytes such as T cells, B cells or NK cells compared to wild-type hIL10. In some embodiments, a fused hIL10 polypeptide of the present disclosure has a pSTAT3 Eₘₐₓ in a lymphocyte less than 70%, less than 60%, less than 50%, less than 40%, or less than 30%, of the pSTAT3 Eₘₐₓ of a wild-type hIL10 in lymphocytes. In some embodiments, a fused hIL10 polypeptide thereof result in a pSTAT3 Eₘₐₓ in a lymphocyte less than 70% (e.g., less than 70%, less than 60%, less than 50%, less than 40%, or less than 30%) but greater than 20% of the pSTAT3 Eₘₐₓ of a wild-type or parental IL10 polypeptide in the lymphocyte. In some embodiments, the lymphocyte is selected from a CD8+ T cell, a CD4+ T cell, a B cell or an NK cell.

### Relative Pro-Inflammatory and Anti-inflammatory Activity

In some embodiments, a fused IL10 polypeptide: (a) exhibits a significant level of at least one anti-inflammatory property of wild-type IL10 and (b) exhibits a significantly reduced level of at least one pro-inflammatory property of wild-type IL10. In some embodiments, “a significant level of at least one anti-inflammatory property” means that the Eₘₐₓ of the fused IL10 polypeptide with respect to such anti-inflammatory property is greater than 10%, alternatively greater than 20%, alternatively greater than 30%, alternatively greater than 40%, alternatively greater than 50%, alternatively greater than 60%, alternatively greater than 70%, alternatively greater than 80%, alternatively greater than 90% of the Eₘₐₓ level of such anti-inflammatory property exhibited by wild-type IL10 as determined in a test system. Examples of anti-inflammatory properties that may be measured in a test system include but are not limited to: (a) the suppression of expression and/or secretion of ILP by activated human myeloid cells, (b) the suppression of expression and/or secretion of IL6 by activated human myeloid cells, and (c) the suppression of expression and/or secretion of TNFα by activated human myeloid cells. In some embodiments, activated human myeloid cells are obtained by isolating human monocytes from the buffy coat of a centrifuged anticoagulated human blood sample, and activating the isolated monocytes by contacting the isolated monocytes with lipopolysaccharide [LPS] in accordance with procedures well known in the art. The levels of ILP, IL6, and TNFα expressed and/or secreted by the activated monocytes may be determined by immunoassay or flow-cytometry methods in accordance with procedures well known in the art. Protocols for the evaluation of the suppression of expression and/or secretion of ILP, IL6, and TNFα by LPS activated human monocytes is provided in Examples herein.

In some embodiments, “a significantly reduced level of at least one pro-inflammatory property” means that the Eₘₐₓ of the fused IL10 polypeptide with respect to such pro-inflammatory property that is less than 90%, alternatively less than 80%, alternatively less than 70%, alternatively less than 60%, alternatively less than 50%, alternatively less than 40%, alternatively less than 30%, alternatively less than 20%, alternatively less than 10% of the Emax of that pro-inflammatory property of wild-type IL10 as determined in a test system. Examples of pro-inflammatory properties include but are not limited to: (a) the suppression of expression and/or secretion of IFNγ by activated human CD8+ T cells, (b) the suppression of expression and/or secretion of granzyme A by activated human CD8+ T cells, and (c) the suppression of expression and/or secretion of granzyme A by activated human CD8+ T cells. In some embodiments, activated human T cells are obtained by isolating CD8+ T cells human whole blood and activated by contacting the isolated CD8+ cells with anti-CD3 and anti-CD28 antibodies in accordance with procedures well known in the art. The levels of IFNγ, granzyme A, and granzyme B expressed and/or secreted by the isolated CD8+ T cells may be determined by immunoassay or flow-cytometry methods in accordance with procedures well known in the art. Protocols for the evaluation of the expression and/or secretion of IFNγ, granzyme A, and granzyme B expressed and/or secreted by CD3/CD28 activated CD8+ T cells are provided in Examples.

In some embodiments, the fused hIL10 exhibits a significant level of at least one anti-inflammatory property of wild-type hIL10 and exhibits a significantly reduced level of at least one pro-inflammatory property of wild-type hIL10, wherein: (a) the significant level of at least one anti-inflammatory property of wild-type hIL10 is an Emax of at least one anti-inflammatory property greater than 30% of the Emax of such anti-inflammatory property exhibited by wild-type hIL10 wherein the at least one anti-inflammatory property is selected from the group consisting of (i) the suppression of expression and/or secretion of hILb in LPS activated human monocytes, (ii) the suppression of expression and/or secretion of hIL6 in LPS activated human monocytes, or (iii) the suppression of expression and/or secretion of h TNFα in LPS activated human monocytes; and (b) the significantly reduced level of at least one pro-inflammatory property of wild-type hIL10 is an Eₘₐₓ of at least one anti-inflammatory property less than 30% of the Eₘₐₓ of such anti-inflammatory property exhibited by wild-type hIL10 wherein the at least one pro-inflammatory property is selected from the group consisting of (i) the suppression of expression and/or secretion of IFNγ by activated human CD8+ T cells, (i) the suppression of expression and/or secretion of granzyme A by activated human CD8+ T cells, and (iii) the suppression of expression and/or secretion of granzyme A by activated human CD8+ T cells.

In some embodiments, the fused hIL10 polypeptide of the present disclosure comprising a polypeptide of formula (1) or formula (2) is a biased hIL10 partial agonist that exhibits a significant level of at least one anti-inflammatory property of wild-type hIL10 and exhibits a significantly reduced level of at least one pro-inflammatory property of wild-type IL10, wherein: the significant level of at least one anti-inflammatory property of wild-type hIL10 is an Emax of at least one anti-inflammatory property greater than 30% of the Emax of such anti-inflammatory property exhibited by wild-type hIL10 wherein the at least one anti-inflammatory property is selected from the group consisting of (i) the suppression of expression and/or secretion of hILb in LPS activated human monocytes, (ii) the suppression of expression and/or secretion of hIL6 in LPS activated human monocytes, or (iii) the suppression of expression and/or secretion of hTNFα in LPS activated human monocytes; and (b) the significantly reduced level of at least one pro-inflammatory property of wild-type hIL10 is an Emax of at least one anti-inflammatory property less than 30% of the Emax of such anti-inflammatory property exhibited by wild-type hIL10 wherein the at least one pro-inflammatory property is selected from the group consisting of (i) the suppression of expression and/or secretion of IFNγ by activated human CD8+ T cells, (i) the suppression of expression and/or secretion of granzyme A by activated human CD8+ T cells, and (iii) the suppression of expression and/or secretion of granzyme A by activated human CD8+ T cells.

In some embodiments, the fused hIL10 polypeptide is a partial agonist wherein: (a) the Emax of the fused IL10 polypeptide is greater than 30% of the Emax of wild-type hIL10 in an assay of anti-inflammatory activity selected from the group consisting of (i) the suppression of expression and/or secretion of hILβ in LPS activated human monocytes, (ii) the suppression of expression and/or secretion of hIL6 in LPS activated human monocytes, or (iii) the suppression of expression and/or secretion of hTNFα in LPS activated human monocytes; and (b) the Emax of the fused IL10 polypeptide is less than 10% of the Emax of wild-type hIL10 in an assay of pro-inflammatory activity selected from the group consisting of (i) the suppression of expression and/or secretion of IFNγ by activated human CD8+ T cells, (i) the suppression of expression and/or secretion of granzyme A by activated human CD8+ T cells, and (iii) the suppression of expression and/or secretion of granzyme A by activated human CD8+ T cells.

In some embodiments, a fused hIL10 polypeptide is a partial agonist wherein: (a) the Emax of the fused hIL10 polypeptide is greater than 50% of the Emax of wild-type hIL10 in an assay of anti-inflammatory activity selected from the group consisting of (i) the suppression of expression and/or secretion of hILβ in LPS activated human monocytes, (ii) the suppression of expression and/or secretion of hIL6 in LPS activated human monocytes, or (iii) the suppression of expression and/or secretion of hTNFα in LPS activated human monocytes; and (b) the Emax of the fused hIL10 polypeptide is less than 20% of the Emax of wild-type hIL10 in an assay of pro-inflammatory activity selected from the group consisting of (i) the suppression of expression and/or secretion of IFNγ by activated human CD8+ T cells, (i) the suppression of expression and/or secretion of granzyme A by activated human CD8+ T cells, and (iii) the suppression of expression and/or secretion of granzyme A by activated human CD8+ T cells.

In some embodiments, a fused hIL10 polypeptide is a partial agonist wherein: (a) the Emax of the fused hIL10 polypeptide is greater than 50% of the Emax of wild-type hIL10 in an assay of anti-inflammatory activity selected from the group consisting of (i) the suppression of expression and/or secretion of hILβ in LPS activated human monocytes, (ii) the suppression of expression and/or secretion of hIL6 in LPS activated human monocytes, or (iii) the suppression of expression and/or secretion of hTNFα in LPS activated human monocytes; and (b) the Emax of the fused hIL10 polypeptide is less than 10% of the Emax of wild-type hIL10 in an assay of pro-inflammatory activity selected from the group consisting of (i) the suppression of expression and/or secretion of IFNγ by activated human CD8+ T cells, (i) the suppression of expression and/or secretion of granzyme A by activated human CD8+ T cells, and (iii) the suppression of expression and/or secretion of granzyme A by activated human CD8+ T cells.

### Modifications to Provide Additional Functions

In some embodiments, the hIL10 mutein may comprise a functional domain of a chimeric polypeptide. Human IL10 mutein fusion proteins of the present disclosure may be readily produced by recombinant DNA methodology by techniques known in the art by constructing a recombinant vector comprising a nucleic acid sequence comprising a nucleic acid sequence encoding the hIL10 mutein in frame with a nucleic acid sequence encoding the fusion partner either at the N-terminus or C-terminus of the hIL10 muteins, the sequence optionally further comprising a nucleic acid sequence in frame encoding a linker or spacer polypeptide.

In other embodiments, the hIL10 mutein can be modified to include an additional polypeptide sequence that functions as an antigenic tag, such as a FLAG sequence. FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the binding molecule further comprises a C-terminal c-myc epitope tag.

In some embodiments, the hIL10 mutein is conjugated to a molecule (“targeting domain”) to facilitate selective binding to particular cell type or tissue expressing a cell surface molecule that specifically binds to such targeting domain, optionally incorporating a linker molecule of from 1-40 (alternatively 2-20, alternatively 5-20, alternatively 10-20) amino acids between the hIL10 mutein sequence and the sequence of the targeting domain of the fusion protein.

In other embodiments, a chimeric polypeptide including a hIL10 mutein and an antibody or antigen-binding portion thereof can be generated. For example, it can be used to localize the chimeric protein to a particular subset of cells or target molecule. The antibody or antigen-binding component of the chimeric protein can serve as a targeting moiety. In some embodiments, the targeting domain is an antibody. As used herein, the term “antibody” means any form of antibody (also known as an immunoglobulin (Ig)) that exhibits the desired biological activity of binding to an antigen epitope, as described herein. The term “antibody” specifically covers, but is not limited to, polyclonal antibodies, monoclonal antibodies (including full length monoclonal antibodies comprising two light chains and two heavy chains), multispecific antibodies (e.g., bispecific antibodies that bind to two or more antigens or antigen epitopes on a single antigen), fully human antibodies (huAb), humanized antibodies (hzAb), chimeric antibodies, single chain variable fragment antibodies (scFv), single-domain antibodies (sdAb), variable heavy (VH) domain antibodies, diabodies (dAb), and antigen-binding fragments of heavy chain only antibodies (VHH), comprising the amino acid sequences of the variable region, as described herein. As used herein, the term “antibody” refers collectively to: (a) glycosylated and non-glycosylated immunoglobulins (including but not limited to mammalian immunoglobulin classes IgG1, IgG2, IgG3, and IgG4) that specifically bind to a target molecule, such as an antigen, and (b) immunoglobulin derivatives including but not limited to IgG(1-4)deltaC_{H}2, F(ab′)₂, Fab, ScFv, V_{H}, V_{L}, tetrabodies, triabodies, diabodies, dsFv, F(ab′)₃, scFv-Fc and (scFv)₂ that compete with the immunoglobulin from which it was derived for binding to the target molecule. The term antibody is not restricted to immunoglobulins derived from any particular mammalian species and includes murine, human, equine, camelids, and human antibodies. The term antibody includes “heavy chain antibodies,” and single-domain antibodies (sdAbs) such as “VHHs” as typically obtained from immunization of camelids (including camels, llamas, and alpacas, such as described by e.g., Hamers-Casterman et al. 1993. Nature. 363:446-448, as described in greater detail below in the definition of “VHH.” The term “antibody” encompasses antibodies isolatable from natural sources or from animals following immunization with an antigen, as well as engineered antibodies including monoclonal antibodies, bispecific antibodies, tri-specific, chimeric antibodies, humanized antibodies, human antibodies, CDR-grafted, veneered, or deimmunized (e.g., to remove B and/or T-cell epitopes) antibodies. In some embodiments the targeting domain specifically binds to a cell surface marker of pro-inflammatory cell such as activated immune cell. In some embodiments, the targeting domain is an antibody that selectively binds to a cell surface marker including but not limited to the IL1R1 receptor, IL-1 receptor accessory protein, the IL6 receptor subunit (IL6R), HLA-DR, HLA-DR α-chain, HLA-DR β-chain the TNFR1, TNFR2, CD4, CD8, F4/80, CCR2, CD169, CX3CR1, CD206, CD163, and Lyvel. Methods of generating cytokine-antibody chimeric polypeptides are described, for example, in U.S. Pat. No. 6,617,135.

Association with Carrier Molecules to Increase Duration of Action

The fused hIL10 polypeptides described herein can be modified to provide for an extended lifetime in vivo and/or extended duration of action in a subject. In some embodiments, the fused hIL10 polypeptide is conjugated to one or more carrier molecules to provide desired pharmacological properties such as an extended half-life. In some embodiments, a fused hIL10 polypeptide is covalently linked to the Fc domain of an IgG, albumin, water soluble polymers, or other molecules to extend its half-life, e.g. glycosylation, acylation and the like as known in the art. In some embodiments, the fused hIL10 polypeptide modified to provide an extended duration of action in a mammalian subject has a half-life in a mammalian of greater than 4 hours, alternatively greater than 5 hours, alternatively greater than 6 hours, alternatively greater than 7 hours, alternatively greater than 8 hours, alternatively greater than 9 hours, alternatively greater than 10 hours, alternatively greater than 12 hours, alternatively greater than 18 hours, alternatively greater than 24 hours, alternatively greater than 2 days, alternatively greater than 3 days, alternatively greater than 4 days, alternatively greater than 5 days, alternatively greater than 6 days, alternatively greater than 7 days, alternatively greater than 10 days, alternatively greater than 14 days, alternatively greater than 21 days, or alternatively greater than 30 days.

Modifications of the fused hIL10 polypeptide to provide an extended duration of action in a mammalian subject include (but are not limited to);
conjugation of the fused hIL10 polypeptide to one or more protein carrier molecules,
conjugation of the fused hIL10 polypeptide to protein carrier molecules, optionally in the form of a fusion protein with additional polypeptide sequences (e.g, fused hIL10 polypeptide-Fc fusions) and
conjugation to polymers, (e.g. water soluble polymers to provide a PEGylated IL10 polypeptide).

It should be noted that the more than one type of modification that provides for an extended duration of action in a mammalian subject may be employed with respect to a given fused hIL10 polypeptide. For example, a fused hIL10 polypeptide of the present disclosure may comprise both amino acid substitutions that provide for an extended duration of action as well as conjugation to a carrier molecule such as a polyethylene glycol (PEG) molecule.

Examples of protein carrier molecules which may be covalently attached to the fused hIL10 polypeptide to provide an extended duration of action in vivo include, but are not limited to albumins, antibodies and antibody fragments such and Fc domains of IgG molecules.

### Fc Fusions

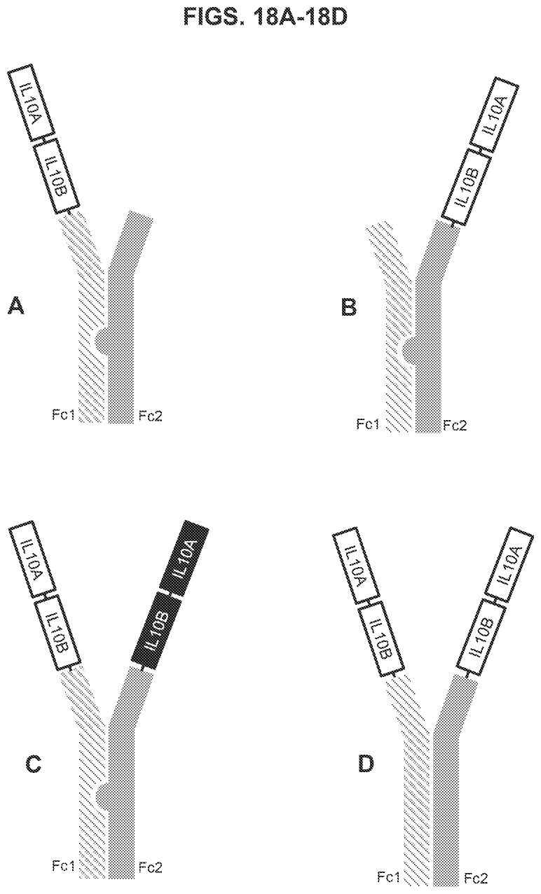

In some embodiments, a fused IL10 polypeptide can be conjugated to an Fc polypeptide. In some embodiments, a fused IL10 polypeptide is conjugated to one Fc polypeptide of an Fc dimer. In some embodiments, a fused IL10 polypeptide is conjugated to both Fc polypeptides of an Fc dimer. Schematic representataions of four exemplary configurations of Fc conjugated fused IL10 polypeptides are provided in FIGS. 18A and 18B of the attached drawings. As illustrated in FIGS. 18A and 18B, the IL10 fused polypeptide may be covalently linked to the N-terminus of one Fc polypeptide of an Fc dimer modified to promote heterodimerization employing knob-into-hole (KiH) modifications to the Fc polypeptides wherein the IL10 fused polypeptide may be attached to either the Fc polypeptide containing the “hole” amino acid substitutions (FIG. 18A) or the “knob” amino acid substitutions (FIG. 18B). Additionally, as illustrated in FIG. 18B, two different IL10 fused polypeptides may each be attached to a Fc polypeptide of a Fc dimer wherein the Fc domain is modified to promote heterodimerization. Alternatively, as illustrated in FIG. 18D, two of the same IL10 fused polypeptides may each be attached to the Fc polypeptides of a Fc dimer which is not modified to promote heterodimerization.

The Fc polypeptide can be an Fc domain from hIgG1, hIgG2, hIgG3, or hIgG4, or a variant thereof. In some embodiments, the Fc polypeptide comprises a sequence that is modified from a wild-type Fc polypeptide sequence to reduce effector function. In some embodiments, the Fc polypeptides of the Fc dimer may be modified to promote heterodimerization.

The “Fc region” useful in the preparation of Fc fusions can be a naturally occurring or synthetic polypeptide that is homologous to an IgG C-terminal domain produced by digestion of IgG with papain. The binding molecule described herein can be conjugated to the entire Fc region, or a smaller portion that retains the ability to extend the circulating half-life of a chimeric polypeptide of which it is a part. In addition, full-length or fragmented Fc regions can be variants of the wild-type molecule.

As indicated, the linkage of the fused hIL10 polypeptide to the Fc subunit may incorporate a linker molecule between the fused hIL10 polypeptide and Fc subunit. In some embodiments, the fused hIL10 polypeptide is expressed as a fusion protein with the Fc domain incorporating an amino acid sequence of a hinge region of an IgG antibody. The Fc domains engineered in accordance with the foregoing may be derived from IgG1, IgG2, IgG3 and IgG4 mammalian IgG species. In some embodiments, the Fc domains may be derived from human IgG1, IgG2, IgG3 and IgG4 IgG species. In some embodiments, the hinge region is the hinge region of an IgG1. In one particular embodiment, the fused hIL10 polypeptide is linked to an Fc domain using a human IgG1 hinge domain.

In some embodiments, the linker is a chemical linker. Examples of chemical linkers include aryl acetylene, ethylene glycol oligomers containing 2-10 monomer units, diamines, diacids, amino acids, or combinations thereof. In some embodiments, the linker is a peptide linker. A peptide linker can include between 1 and 50 amino acids (e.g., between 2 and 50, between 5 and 50, between 10 and 50, between 15 and 50, between 20 and 50, between 25 and 50, between 30 and 50, between 35 and 50, between 40 and 50, between 45 and 50, between 2 and 45, between 2 and 40, between 2 and 35, between 2 and 30, between 2 and 25, between 2 and 20, between 2 and 15, between 2 and 10, between 2 and 5 amino acids). Glycine and glycine-serine polymers are relatively unstructured, and therefore may serve as a neutral tether between components. Examples of glycine polymers include (G)n, glycine-alanine polymers, alanine-serine polymers, glycine-serine polymers (for example, (GmSo)n (SEQ ID NO:129), (GSGGS)n (SEQ ID NO:130), (GmSoGm)n (SEQ ID NO:131), (GmSoGmSoGm)n (SEQ ID NO:132), (GSGGSm)n (SEQ ID NO:133), (GSGSmG)n (SEQ ID NO:134) and (GGGSm)n (SEQ ID NO:135), and combinations thereof, where m, n, and o are each independently selected from an integer of at least 1 to 20, e.g., 1-18, 216, 3-14, 4-12, 5-10, 1, 2, 3, 4, 5, 6, 7, 8, 9, or 10), and other flexible linkers.

In some embodiments the amino acid sequence of the Fc polypeptide conjugated to the fused hIL10 polypeptide can be modified to reduce effector function. In some embodiments, the Fc polypeptide may be modified to substantially reduce binding to Fc receptors (FcyR and FcR) which reduces or abolishes antibody directed cytotoxicity (ADCC) effector function. Modification of Fc polypeptides to reduce effector function are well known in the art. See, e.g., Wang, et al. (2018) IgG Fc engineering to modulate antibody effector functions, Protein Cell 9(1):63-73. For example, mutation of the lysine residue at position 235 (EU numbering) from leucine (L) to glutamic acid (E) is known to reduce effector function by reducing FcgR and C1q binding. Alegre, et al. (1992) J. Immunology 148:3461-3468. Additionally, substitution of the two leucine (L) residues at positions 234 and 235 (EU numbering) in the IgG1 hinge region with alanine (A), i.e., L234A and L235A, results in decreased complement dependent cytotoxicity (CDC) and antibody dependent cellular cytotoxicity (ADCC). Hezereh et al., (2001) J. Virol 75(24):12161-68. Furthermore, mutation of the proline at position 329 (EU numbering) to alanine (P329A) or glycine, (P329G) mitigates effector function and may be combined with the L234A and L235A substitutions. In some embodiments, the Fc polypeptides may comprise the amino acid substitutions L234A/L235A/P329A (EU numbering) referred to as the “LALAPA” substitutions or L234A/L235A/P329G (EU numbering) referred to as the “LALAPG” substitutions. In some embodiments, the Fc polypeptides may comprises the amino acid substitutions E233P/L234V/L235A/AG237 (referred to in the scientific literature as the PVAdelG mutation).

In some embodiments, the Fc polypeptide is derived from hIgG4. It has been shown that glycosylation at position 297 (EU numbering) contributes to effector function in IgG4. Edelman, et al (1969) PNAS (USA) 63:78-85 Examples of modifications at N297 to eliminate glycosylation sites and effector functions in the Fc domain of hIgG4 include the amino acid substitutions selected from N297Q and N297G (EU numbering).

In some embodiments, the amino acid sequence of the Fc polypeptide can be further modified to incorporate amino acid substitutions which extend the duration of action of the molecule and prevent clearance. In some embodiments, such modifications to the Fc polypeptide include the amino acid substitutions M428L and N434S (EU numbering) referred to as the “LS” modification. The LS modification may optionally be combined with amino acid substitutions to reduce effector function and provide for disulfide bonds between the Fc domains.

In some embodiments, the amino acid sequence of the Fc polypeptide conjugated to the fused hIL10 polypeptide can be further modified to eliminate N-linked or O-linked glycosylation sites. Aglycosylated variants of Fc polypeptides, particularly of the IgG1 subclass are known to be poor mediators of effector function. Jefferies et al. 1998, Immol. Rev., vol. 163, 50-76). It has been shown that glycosylation at position 297 (EU numbering) contributes to effector function. Edelman, et al (1969) PNAS (USA) 63:78-85. In some embodiments, the Fc polypeptides comprises one or modifications to eliminate N- or O linked glycosylation sites. Examples of modifications at N297 to eliminate glycosylation sites in the Fc polypeptide include the amino acid substitutions N297Q and N297G. In some embodiments, the Fc polypeptide is an hIgG4 Fc comprising an amino acid substitution selected from the group consisting of N297Q and N297G.

In some embodiments, a fused hIL10 Fc polypeptide may be further modified to extend its duration of action in vivo. In some embodiments, conjugation of the PEG moiety may be accomplished via a sulfhydryl (—SH) group of a cysteine residue. In some embodiments, the PEGylation of the fused hIL10 polypeptide is provided the naturally occurring cysteine residues at position 220 (C220, EU Numbering) of the upper hinge region of the Fc polypeptide.

In some embodiments, the fused hIL10 polypeptide may be conjugated to a single Fc polypeptide of an Fc dimer wherein the first and second Fc polypeptides of the Fc dimer are modified to promote heterodimerization (FIGS. 18A and 18B). In some embodiments, the fused hIL10 polypeptide may be conjugated to both of the Fc polypeptides of an Fc dimer wherein the first and second Fc polypeptides of the Fc dimer are modified to promote heterodimerization, wherein each of the fused hIL10 polypeptides are the same or different (FIG. 18C). A variety of techniques are established for the promotion of heterodimerization of Fc domains. See, e.g., Kim, et al., U.S. Pat. No. 11,087,249, issued Aug. 3, 2021. In some embodiments, the modifications to promoter heterodimerization of the first and second Fc polypeptides are the 1F-TA mutations and the HA-TF mutations as described in Moore, et al (2011) mAbs 3(6):546-557. The HF-TA method employs the S364H/T394F substitutions on one Fc monomer and the Y349T/F405A substitutions on the complementary Fc polypeptide. The (HA-TF) method employs the S364H/F405A substitutions on one Fc polypeptide and the Y349T/T394F substitutions on the complementary Fc polypeptide. Alternatively, the first and second Fc polypeptides are modified to promote heterodimerization by the ZW1 heterodimerization method which employs the T350V/L351Y/F405A/Y407V substitutions on one Fc polypeptide and the T350V/T366L/K392L/T394W substitutions on the complementary Fc polypeptide. Von Kreudenstein, et al (2013) mAbs, 5(5):646-654. Alternatively, the first and second Fc polypeptides are modified to promote heterodimerization by the EW-RVT heterodimerization method which employs the K360E/K409W substitutions on one Fc polypeptide and the Q347R/D399V/F405T substitutions on the complementary Fc polypeptide. Choi, et al (2015) Molecular Immunology 65(2):377-83.

In one embodiment, first and second Fc polypeptides are modified to promote heterodimerization by the employment of the “knob-into-hole” (abbreviated KiH) modification as exemplified herein. The KiH modification comprises one or more amino acid substitutions in a first Fc polypeptide that creates a bulky “knob” domain on a first Fc polypeptide and one or more amino acid substitutions on a second Fc polypeptide that create a complementary pocket or “hole” to receive the “knob” of the first Fc monomer. A variety of amino acid substitutions have been established for the creation of complementary knob and hole Fc monomers. See, e.g., Ridgway, et al (1996) Protein Engineering 9(7):617-921; Atwell, et al (1997) J. Mol. Biol. 270:26-35; Carter, et al. U.S. Pat. No. 5,807,706 issued Sep. 15, 1998; Carter, et al., U.S. Pat. No. 7,695,936 issued Apr. 13, 2010; Zhao et al. “A new approach to produce IgG4-like bispecific antibodies,” Scientific Reports 11: 18630 (2021); Cao et al. “Characterization and Monitoring of a Novel Light-heavy-light Chain Mispair in a Therapeutic Bispecific Antibody,” and Liu et al. “Fc Engineering for Developing Therapeutic Bispecific Antibodies and Novel Scaffolds”. Frontiers in Immunology 8: 38, 2017. In some embodiments, the Fc dimer comprises two Fc polypeptides wherein the CH3 domain of a first Fc polypeptides wherein the threonine at (EU numbering) position 366 is modified with a bulky residue (e.g. a T366W) create a “knob” and the substitution, and a second Fc polypeptides comprising one or more substitutions in complementary residues of the CH3 domain of the second Fc polypeptide to create a pocket or “hole” to receive the bulky residue, for example by amino acid substitutions such as T366S, L368A, and/or Y407V. n one embodiment, the Fc1 monomer of formula 1 is a “knob” modified Fc monomer comprising the amino acid substitution T366W and the Fc2 monomer of formula 2 is a “hole” modified Fc comprising the set of amino acid substitutions T366S/L368A/Y407V. In one embodiment the first Fc polypeptide of the Fc dimer is a “hole” modified Fc monomer comprising the set of amino acid substitutions T366S/L368A/Y407V and the second Fc polypeptide of the Fc dimer is a “knob” modified Fc monomer comprising the amino acid substitution T366W.

In some embodiments, the fused hIL10 polypeptide Fc conjugates of the present disclosure are provided as a complementary heterodimeric pair of fused hIL10 Fc polypeptides wherein the first and second fused hIL10 polypeptide Fc polypeptides are linked by at least one disulfide bond. In some embodiments, the incorporation of a disulfide bond between the first and second fused hIL10 polypeptide Fc polypeptides may be achieved by cysteine substitutions at particular points within the first and second Fc polypeptides. In one embodiment, the Fc polypeptide of the first fused hIL10 Fc polypeptide is derived from the Fc domain of hIgG1 comprising an amino acid substitution S354C (EU numbering) and the second fused hIL10 Fc polypeptide domain is derived from the Fc domain of hIgG1 comprising an amino acid substitution Y349C (EU numbering) to provide a disulfide bond between the S354C of the first Fc polypeptide and Y349C of the second Fc polypeptide. Alternatively, the first Fc polypeptide is derived from the Fc domain of hIgG1 comprising an amino acid substitution Y349C (EU numbering) and the second Fc polypeptide is derived from the Fc domain of hIgG1 comprising an amino acid substitution S354C (EU numbering) to provide a disulfide bond between the S354C of the first Fc polypeptide and Y349C of second Fc polypeptide.

In some embodiments, the present disclosure provides a heterodimeric fused IL10 polypeptide Fc, the heterodimeric fused IL10 polypeptide Fc comprising a first polypeptide of the formula #1:

IL10FP-L1ₐ-UH1Fc1 [1]

and a second polypeptide of the formula #2:

UH2-Fc2 [2]
wherein:
   hIL10FP is a fused hIL10 polypeptide of the present disclosure (e.g., a fused hIL10 polypeptide of formula (hIL10A)-Lₙ-(hIL10B));
   L1 is a linker and a is independently selected from 0 (absent) or 1 (present);
   UH1 and UH2 are each an upper hinge domain of human immunoglobulin independently selected from the group consisting of the IgG1, IgG2, IgG3 and IgG4 upper hinge domains, optionally comprising the amino acid substitution C220S (EU numbering);
   Fc1 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc2, and
   Fc2 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc1,
      and optionally wherein the polypeptide of formula [1] and the polypeptide of formula [2] are linked by at least one interchain disulfide bond.

A heterodimeric fused IL10 polypeptide Fc of the present disclosure is a heterodimer comprising polypeptides of the formulae [1] and [2] that each incorporate an upper hinge region of a human immunoglobulin molecule. The term “upper hinge” or “UH” refers to an amino acid sequence corresponding to amino acid residues 216-220 (EU numbering) of a human immunoglobulin molecule. In some embodiments, the upper hinge region is a naturally occurring upper hinge region of a human immunoglobulin selected from the human IgG1, human IgG2, human IgG3 and human IgG4 upper hinge domains. In some embodiments, the upper hinge region is the upper hinge region of a human IgG1 immunoglobulin. In some embodiments, the upper hinge region is the upper hinge region of a human IgG1 immunoglobulin comprising the pentameric amino acid sequence: EPKSC (SEQ ID NO:121).

In some embodiments, the upper hinge region contains an unpaired cysteine residue at position 220 (EU numbering) that typically, in a complete immunoglobulin molecule, binds to a cysteine on a light chain. When only the Fc domain is used comprising the hinge domain, the unpaired cysteine in the hinge domain creates the potential of the formation of improper disulfide bonds. Consequently, in some embodiments the cysteine at position 220 (C220, numbered in accordance with EU numbering) is substituted with an amino acid that does not promote disulfide bonding. In some embodiments, the Fc domain comprises a C220S mutation having the amino acid sequence EPKSS (SEQ ID NO: 122).

### Fc1 and Fc2

The heterodimeric fused IL10 polypeptide Fc of the present disclosure are heterodimers comprising polypeptides of the formulae [1] and [2], which each incorporate an Fc region (Fc1 and Fc2) of a human immunoglobulin molecule modified to promote heterodimerization.

As used herein the term “Fc” and “Fc monomer” and “Fc polypeptide” are used interchangeably herein to designate the monomeric polypeptide subunit of an Fc dimer. An Fc comprises an amino acid sequence (from amino to carboxy terminal) comprising a lower hinge domain and the CH2 and CH3 domains of a human immunoglobulin molecule. In some embodiments, the Fc monomer is a polypeptide comprising the lower hinge domain and the CH2 and CH3 domains of a human immunoglobulin molecule domains of human IgG1, human IgG2, human IgG3 and human IgG4 hinge domains. The CH2 domain of hIgG1 corresponds to amino acid residues 231-340 (EU numbering) and is provided as SEQ ID NO:128. The CH3 domain of hIgG1 corresponds to amino acid residues 341-447 (EU numbering) and is provided as SEQ ID NO:128.

The polypeptides of the formulae [1] and [2] each incorporate a lower hinge region of a human immunoglobulin. As used herein, the term “lower hinge” or “LH” refers to an amino acid sequence corresponding to amino acid residues 221-229 (EU numbering) of a human immunoglobulin molecule. In some embodiments, the lower hinge region is a naturally occurring lower hinge region of a human immunoglobulin selected from the LH regions of IgG1, IgG2, IgG3 and IgG4 lower hinge domains. In some embodiments, the lower hinge region is the lower hinge region of a human IgG1 immunoglobulin. In some embodiments, the lower hinge region is the lower hinge region of a human IgG1 immunoglobulin comprising the decameric amino acid sequence: DKTHTCPPCP (SEQ ID NO:127).

In some embodiments, Fc1 and Fc2 are derived from a polypeptide corresponding to amino acids 221-447 (EU numbering) of the human IgG1 immunoglobulin as shown below:

```
(SEQ ID NO: 128)
              230 240 250 260 270
DKTHTCPPCP APELLGGPSV FLFPPKPKDT LMISRTPEVT CVVVDVSHED
              280. 290. 300 310 320
PEVKFNWYVD GVEVHNAKTK PREEQYNSTY RVVSVLTVLH QDWLNGKEYK
              330 340 350 360 370
CKVSNKALPA PIEKTISKAK GQPREPQVYT LPPSRDELTK NQVSLTCLVK
              380 390 400 410 420
GFYPSDIAVE WESNGQPENN YKTTPPVLDS DGSFFLYSKL TVDKSRWQQG
              430 440 447
NVFSCSVMHE ALHNHYTQKS LSLSPGK
```

As indicated in above sequence, the C-terminal residue of the wild-type form of the IgG1 Fc domain is a lysine, referred to as K447 in accordance with EU numbering. The K447 is inconsistently removed by the producer cell during recombinant production. As a result, the population of recombinant Fc monomers may be heterogenous in that some fraction of the recombinantly produced Fc monomers will contain K447 and others will not. Such inconsistent proteolytic processing by producer cells may therefore result in a heterogenous population of hIL10 Fcs. Typically, particularly in the context of human pharmaceutical agents, such heterogeneity of the active pharmaceutical ingredient is to be avoided. Consequently, in addition to modifications to the Fc monomer sequence promote heterodimerization, the present disclosure provides Fc monomers that further comprising a deletion of the C-terminal K447 or a deletion of G446 and K447 and nucleic acid sequences encoding Fc monomers comprising a: (a) a deletion of the lysine residue at position 447 (K447,EU numbering, abbreviated as AK447 or des-K447), or (b) deletion of both the glycine at position 456 (G446 EU numbering, abbreviated as des-G446) and K447 (this double deletion of G446 and K447 being referred to herein as des-G446/des-K447 or AG446/AK447).

As provided in formulae [1] and [2] above, the Fc1 and Fc2 monomers of the dimeric Fc contain amino acid substitutions that promote heterodimerization between Fc1 and Fc2.

In some embodiments, the fused hIL10 polypeptide Fc conjugates of the present disclosure are covalently linked via one or more, optionally two or more optionally three or more disulfide bonds, optionally four or more disulfide bonds between the side chains of the following groups of cystine pairs: (a) C96 of the hP35 and C199 of the hP40M; (b) between C226 of the first Fc monomer and the C226 of the second Fc monomer; (c) between C229 of the first Fc monomer and the C229 of the second Fc monomer; and (d) between S354C of the first Fc domain comprising a S354C amino acid substitution and Y349C of the second Fc domain comprising a Y349C amino acid substitution.

### Albumin Carrier Molecules

In some embodiments, a fused hIL10 polypeptide is conjugated to an albumin molecule (e.g., human serum albumin) which is known in the art to facilitate extended exposure in vivo. In some embodiments, the fused hIL10 polypeptide is conjugated to albumin via chemical linkage or expressed as a fusion protein with an albumin molecule (referred to herein as a “fused hIL10 polypeptide albumin fusion”). The term “albumin” as used in the context fused hIL10 polypeptide-albumin fusions includes albumins such as human serum albumin (HSA), cyno serum albumin, and bovine serum albumin (BSA). In some embodiments, the HSA comprises a C34S or K573P amino acid substitution relative to the wild-type HSA sequence. According to the present disclosure, albumin can be conjugated to a fused hIL10 polypeptide at the carboxyl terminus, the amino terminus, both the carboxyl and amino termini, and internally (see, e.g., U.S. Pat. Nos. 5,876,969 and 7,056,701). In the HAS-fused hIL10 polypeptide contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising a fused hIL10 polypeptide fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. As an alternative to chemical linkage between the fused hIL10 polypeptide and the albumin molecule, the fused hIL10 polypeptide-albumin complex may be provided as a fusion protein comprising an albumin polypeptide sequence and a fused hIL10 polypeptide recombinantly expressed in a host cell as a single polypeptide chain, optionally comprising a linker molecule between the albumin and fused hIL10 polypeptide. Such fusion proteins may be readily prepared through recombinant technology to those of ordinary skill in the art. Nucleic acid sequences encoding such fusion proteins may be ordered from any of a variety of commercial sources. The nucleic acid sequence encoding the fusion protein is incorporated into an expression vector operably linked to one or more expression control elements, the vector introduced into a suitable host cell and the fusion protein isolated from the host cell culture by techniques well known in the art.

### Polymeric Carriers

In some embodiments, extended in vivo duration of action of the fused hIL10 polypeptide may be achieved by conjugation to one or more polymeric carrier molecules such as XTEN polymers or water soluble polymers.

### XTEN Conjugates

The fused hIL10 polypeptide may further comprise an XTEN polymer. The XTEN polymer conjugated (either chemically or as a fusion protein) to a fused hIL10 polypeptide provides extended duration akin to PEGylation and may be produced as a recombinant fusion protein in E. coli. XTEN polymers suitable for use in conjunction with the fused hIL10 polypeptide are provided in Podust, et al. (2016) “Extension of in vivo half-life of biologically active molecules by XTEN protein polymers”, J Controlled Release 240:52-66 and Haeckel et al. (2016) “XTEN as Biological Alternative to PEGylation Allows Complete Expression of a Protease-Activatable Killin-Based Cytostatic” PLOS ONE DOI:10.1371/journal.pone.0157193 Jun. 13, 2016. The XTEN polymer fusion protein may incorporate a protease sensitive cleavage site between the XTEN polypeptide and the hIL2 mutein such as an MMP-2 cleavage site.

### Water Soluble Polymers

In some embodiments, the fused hIL10 polypeptide can be conjugated to one or more water-soluble polymers. Examples of water soluble polymers useful in the practice of the present disclosure include polyethylene glycol (PEG), poly-propylene glycol (PPG), polysaccharides (polyvinylpyrrolidone, copolymers of ethylene glycol and propylene glycol, poly(oxyethylated polyol), polyolefinic alcohol,), polysaccharides), poly-alpha-hydroxy acid), polyvinyl alcohol (PVA), polyphosphazene, polyoxazolines (POZ), poly(N-acryloylmorpholine), or a combination thereof.

### PEGylation

In some embodiments, the fused hIL10 polypeptide can be conjugated to one or more polyethylene glycol molecules or “PEGylated.” Although the method or site of PEG attachment to the binding molecule may vary, in certain embodiments the PEGylation does not alter, or only minimally alters, the activity of the binding molecule.

PEGs suitable for conjugation to a polypeptide sequence are generally soluble in water at room temperature, and have the general formula:

R(O—CH₂—CH₂)ₙO—R,

where R is hydrogen or a protective group such as an alkyl or an alkanol group, and where n is an integer from 1 to 1000. When R is a protective group, it generally has from 1 to 8 carbons. The PEG can be linear or branched. Branched PEG derivatives, “star-PEGs” and multi-armed PEGs are contemplated by the present disclosure.

In some embodiments, selective PEGylation of the fused hIL10 polypeptide, for example, by the incorporation of non-natural amino acids having side chains to facilitate selective PEG conjugation, may be employed. Specific PEGylation sites can be chosen such that PEGylation of the binding molecule does not affect its binding to the target receptors.

In certain embodiments, the increase in half-life is greater than any decrease in biological activity. PEGs suitable for conjugation to a polypeptide sequence are generally soluble in water at room temperature and have the general formula R(O—CH2-CH2)nO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

A molecular weight of the PEG used in the present disclosure is not restricted to any particular range. The PEG component of the binding molecule can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa, or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

In some embodiments, the present disclosure provides a fused human IL10 (hIL10) polypeptide of the formula:

PEG-L2ₘ-[(hIL10A)-Lₙ-(hIL10B)],
wherein:
PEG is a linear or branched polyethylene glycol molecule having a molecular weight of from about 10 kD to about 80 kD;
L2 is a polypeptide or chemical linker and m=0 (absent) or 1 (present);
hIL10A is a human IL10 mutein comprising an amino acid substitution at a position selected from the group consisting of T100, H14, N18, N21, M22, R24, D25, D28, R32, E74, H90, N92, 593, E96, and R104 (e.g., selected from the group consisting of T100, H14, N18, N21, M22, D25, R32, S93, and E96) of numbered in accordance with SEQ ID NO:4, optionally comprising an N-terminal deletion of one or more amino acid selected from the group consisting of Ser1, Ser1-Pro2, Ser1-Pro2-Gly3, Ser1-Pro2-Gly3-Gln4 (SEQ ID NO:141), Ser1-Pro2-Gly3-Gln4-Gly5 (SEQ ID NO:142), Ser1-Pro2-Gly3-Gln4-Gly5-Thr6 (SEQ ID NO:143), Ser1-Pro2-Gly3-Gln4-Gly5-Thr6-Gln7 (SEQ ID NO:22), Ser1-Pro2-Gly3-Gln4-Gly5-Thr6-Gln7-Ser8 (SEQ ID NO:144), Ser1-Pro2-Gly3-Gln4-Gly5-Thr6-Gln7-Ser8-Glu9 (SEQ ID NO:145) and Ser1-Pro2-Gly3-Gln4-Gly5-Thr6-Gln7-Ser8-Glu9-Asn10 (SEQ ID NO:21), optionally comprising an additional N-terminal methionine residue;
hIL10B is a human IL10 mutein comprising an amino acid substitution at a position selected from the group consisting of T100, H14, N18, N21, M22, R24, D25, D28, R32, E74, H90, N92, S93, E96, and R104 (e.g., selected from the group consisting of T100, H14, N18, N21, M22, D25, R32, S93, and E96) of numbered in accordance with SEQ ID NO:4, optionally comprising an N-terminal deletion of one or more amino acid selected from the group consisting of Ser1, Ser1-Pro2, Ser1-Pro2-Gly3, Ser1-Pro2-Gly3-Gln4 (SEQ ID NO:141), Ser1-Pro2-Gly3-Gln4-Gly5 (SEQ ID NO:142), Ser1-Pro2-Gly3-Gln4-Gly5-Thr6 (SEQ ID NO:143), Ser1-Pro2-Gly3-Gln4-Gly5-Thr6-Gln7 (SEQ ID NO:22), Ser1-Pro2-Gly3-Gln4-Gly5-Thr6-Gln7-Ser8 (SEQ ID NO:144), Ser1-Pro2-Gly3-Gln4-Gly5-Thr6-Gln7-Ser8-Glu9 (SEQ ID NO:145) and Ser1-Pro2-Gly3-Gln4-Gly5-Thr6-Gln7-Ser8-Glu9-Asn10 (SEQ ID NO: 21); and
L is a polypeptide linker of from 1-30 amino acids, and n=0 (absent) or 1 (present).

In one embodiment, the present disclosure provides a PEGylated fused hIL10 polypeptide of the structure:

PEG-[hIL10A-Lₙ-hIL10B],
wherein:
   a) PEG is a 40 kD branched PEG molecule comprising two 20 kD arms covalently bonded to the N-terminus of hIL10A, optionally via an aldehyde linker;
   b) hIL10A is a hIL10 mutein comprising one or more amino acid substitutions at positions corresponding to residues T100, H14, N18, N21, M22, R24, D25, D28, R32, E74, H90, N92, S93, E96, and R104 (e.g., corresponding to residues T100, H14, N18, N21, M22, D25, R32, S93, and E96) of SEQ ID NO:4 and optionally comprising an N-terminal deletion corresponding to residues 1, 1-2, 1-3, 1-4, 1-5, 1-6, 1-7, 1-8, 1-9, or 1-10 of SEQ ID NO:4; optionally comprising an additional N-terminal methionine residue;
   c) hIL10B is a hIL10 mutein comprising one or more amino acid substitutions at positions corresponding to residues T100, H14, N18, N21, M22, R24, D25, D28, R32, E74, H90, N92, S93, E96, and R104 (e.g., corresponding to residues T100, H14, N18, N21, M22, D25, R32, S93, and E96) of SEQ ID NO:4 and optionally comprising an N-terminal deletion corresponding to residues 1, 1-2, 1-3, 1-4, 1-5, 1-6, 1-7, 1-8, 1-9, or 1-10 of SEQ ID NO:4;
   d) L is a polypeptide linker and n=0 (absent) or 1 (present).

In one embodiment, the present disclosure provides a PEGylated fused hIL10 polypeptide of the structure:

PEG-[hIL10A-Lₙ-hIL10B],
wherein:
   a) PEG is a 40 kD branched PEG molecule comprising two 20 kD arms covalently bonded to the N-terminus of hIL10A, optionally via an aldehyde linker;
   b) hIL10A is a hIL10 mutein selected from the group consisting of SEQ ID NOS:6, 7, 8, 9, 10, 11, 12, 13, 14, 15, 16, 17, 18, 148, 149, 150, 151, 152, 153 154, 155, 156, 191, 192, 193, 194, 195, 196, 197, 198, 199, 200, 201, 202, 203, 204, and 205 and optionally comprising an N-terminal deletion corresponding to residues 1, 1-2, 1-3, 1-4, 1-5, 1-6, 1-7, 1-8, 1-9, or 1-10 of SEQ ID NO:4; optionally comprising an additional N-terminal methionine residue;
   c) hIL10B is a hIL10 mutein selected from the group consisting of SEQ ID NOS:6, 7, 8, 9, 10, 11, 12, 13, 14, 15, 16, 17, 18, 148, 149, 150, 151, 152, 153 154, 155, 156, 191, 192, 193, 194, 195, 196, 197, 198, 199, 200, 201, 202, 203, 204, and 205 and optionally comprising an N-terminal deletion corresponding to residues 1, 1-2, 1-3, I-4, 1-5, I-6, 1-7, 1-8, 1-9, or 1-10 of SEQ ID NO:4;
   d) L is a polypeptide linker and n=0 (absent) or 1 (present).

In one embodiment, the present disclosure provides a PEGylated fused hIL10 polypeptide of the structure:

PEG-[hIL10A-Lₙ-hIL10B],
wherein:
(a) PEG is a 40 kD branched PEG molecule comprising two 20 kD arms covalently bonded to the N-terminus of hIL10A, optionally via an aldehyde linker;
(b) hIL10A is a hIL10 mutein selected from the group consisting of SEQ ID NOS:6, 7, 8, 9, 10, 11, 12, 13, 14, 15, 16, 17, 18, 148, 149, 150, 151, 152, 153 154, 155, 156, 191, 192, 193, 194, 195, 196, 197, 198, 199, 200, 201, 202, 203, 204, and 205; optionally comprising an additional N-terminal methionine residue;
(c) hIL10B is a hIL10 mutein selected from the group consisting of SEQ ID NOS:6, 7, 8, 9, 10, 11, 12, 13, 14, 15, 16, 17, 18, 148, 149, 150, 151, 152, 153 154, 155, 156, 191, 192, 193, 194, 195, 196, 197, 198, 199, 200, 201, 202, 203, 204, and 205;
(d) L is a polypeptide linker and n=0 (absent) or 1 (present).

In one embodiment, the present disclosure provides a PEGylated fused hIL10 polypeptide of the structure:

PEG-[hIL10A-Lₙ-hIL10B],
wherein:
(a) PEG is a 40 kD branched PEG molecule comprising two 20 kD arms covalently bonded to the N-terminus of hIL10A, optionally via an aldehyde linker; and
(b) the polypeptide of the formula [hIL10A-Lₙ-hIL10B] is a polypeptide is selected from the group consisting of SEQ ID NOS:24, 25, 26, 27, 28, 29, 30, 31, 32, 33, 34, 35, 36, 37, 38, 39, 40, 41, 42, 43, 44, 45, 46, 47, 48, 49, 50, 119, 157, 158, 159, 160, 161, 162, 163, 164, 165, 166, 167, 168, 169, 170, 171, 172, 173, 174, 175, 176, 177, 178, 179, 180, 181, 182, 183 184, 185 and 186.

In one embodiment, the present disclosure provides a PEGylated fused hIL10 polypeptide of the structure:

PEG-[hIL10A-Lₙ-hIL10B],
wherein:
   (a) PEG is a 40 kD branched PEG molecule comprising two 20 kD arms covalently bonded to the N-terminus of hIL10A, optionally via an aldehyde linker; and
   (b) the polypeptide of the formula [hIL10A-Lₙ-hIL10B] is a polypeptide is selected from the group consisting of SEQ ID NOS:36, 37, 38, 42, and 44.

In one embodiment, the present disclosure provides a PEGylated fused hIL10 polypeptide of the structure:

PEG-[hIL10A-Lₙ-hIL10B],
wherein:
(a) PEG is a 40 kD branched PEG molecule comprising two 20 kD arms of the formula:

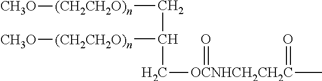

covalently bonded to the N-terminus of hIL10A, optionally via a linker; and
(b) the polypeptide of the formula [hIL10A-Lₙ-hIL10B] is a polypeptide is a polypeptide is selected from the group consisting of SEQ ID NOS:24, 25, 26, 27, 28, 29, 30, 31, 32, 33, 34, 35, 36, 37, 38, 39, 40, 41, 42, 43, 44, 45, 46, 47, 48, 49, 50, 119, 157, 158, 159, 160, 161, 162, 163, 164, 165, 166, 167, 168, 169, 170, 171, 172, 173, 174, 175, 176, 177, 178, 179, 180, 181, 182, 183 184, 185 and 186.

In one embodiment, the present disclosure provides a PEGylated fused hIL10 polypeptide of the structure:

PEG-[hIL10A-Lₙ-hIL10B],
wherein:
PEG is a 40 kD branched PEG molecule comprising two 20 kD arms of the formula:

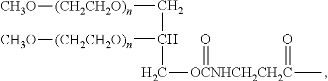

covalently bonded to the N-terminus of hIL10A, optionally via a linker;
hIL10A is a polypeptide selected from Table 1B;
hIL10B is a polypeptide selected from Table 1A; and
L is a polypeptide linker and n=0 (absent) or 1 (present).

In one embodiment, the present disclosure provides a PEGylated fused hIL10 polypeptide of the structure:

PEG-[hIL10A-Lₙ-hIL10B],
wherein:
   (a) PEG is a 40 kD branched PEG molecule comprising two 20 kD arms of the formula:

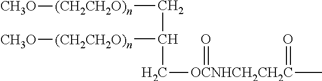

covalently bonded to the N-terminus of hIL10A, optionally via a linker; and
(b) the polypeptide of the formula [hIL10A-Lₙ-hIL10B] is a polypeptide of the sequence:

(SEQ ID NO: 44)
SPGQGTQSENSCTHFPGNLPNMLRDLRDAFSRVKTFFQMKDQLDNLLLKE
SLLEDFKGYLGCQALSEMIQFYLEEVMPQAENQDPDIKAHVNSLGENLKL
LRLRLRRCHRFLPCENKSKAVEQVKNAFNKLQEKGIYKAMSEFDIFINYI
EAYMTMKIRNSPGQGTQSENSCTHFPGNLPNMLRDLRDAFSRVKTFFQMK
DQLDNLLLKESLLEDFKGYLGCQALSEMIQFYLEEVMPQAENQDPDIKAH
VNSLGENLKLLRLRLRRCHRFLPCENKSKAVEQVKNAFNKLQEKGIYKAM
SEFDIFINYIEAYMTMKIRN.

In one embodiment, the present disclosure provides a PEGylated fused hIL10 polypeptide of the structure:

PEG-[hIL10A-Lₙ-hIL10B],
wherein:
   (a) PEG is a 40 kd branched PEG molecule comprising two 20 kD arms of the formula:

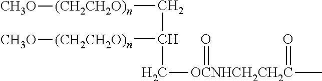

covalently bonded to the N-terminus of hIL10A, optionally via a linker; and
(b) the polypeptide of the formula [hIL10A-Lₙ-hIL10B] is a polypeptide of the sequence:

(SEQ ID NO: 123)
MSPGQGTQSENSCTHFPGNLPNMLRDLRDAFSRVKTFFQMKDQLDNLLLK
ESLLEDFKGYLGCQALSEMIQFYLEEVMPQAENQDPDIKAHVNSLGENLK
LLRLRLRRCHRFLPCENKSKAVEQVKNAFNKLQEKGIYKAMSEFDIFINY
IEAYMTMKIRNSPGQGTQSENSCTHFPGNLPNMLRDLRDAFSRVKTFFQM
KDQLDNLLLKESLLEDFKGYLGCQALSEMIQFYLEEVMPQAENQDPDIKA
HVNSLGENLKLLRLRLRRCHRFLPCENKSKAVEQVKNAFNKLQEKGIYKA
MSEFDIFINYIEAYMTMKIRN.

In one embodiment, the present disclosure provides a PEGylated fused hIL10 polypeptide of the structure:

PEG-[hIL10A-Lₙ-hIL10B],
wherein:
   (a) PEG is a 40 kd branched PEG molecule comprising two 20 kD arms of the formula:

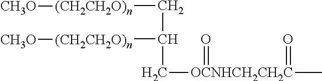

covalently bonded to the N-terminus of hIL10A, optionally via a linker; and
(b) the polypeptide of the formula [hIL10A-Lₙ-hIL10B] is a polypeptide of the sequence:

(SEQ ID NO: 124)
PGQGTQSENSCTHFPGNLPNMLRDLRDAFSRVKTFFQMKDQLDNLLLKES
LLEDFKGYLGCQALSEMIQFYLEEVMPQAENQDPDIKAHVNSLGENLKLL
RLRLRRCHRFLPCENKSKAVEQVKNAFNKLQEKGIYKAMSEFDIFINYIE
AYMTMKIRNSPGQGTQSENSCTHFPGNLPNMLRDLRDAFSRVKTFFQMKD
QLDNLLLKESLLEDFKGYLGCQALSEMIQFYLEEVMPQAENQDPDIKAHV
NSLGENLKLLRLRLRRCHRFLPCENKSKAVEQVKNAFNKLQEKGIYKAMS
EFDIFINYIEAYMTMKIRN.

In one embodiment, the present disclosure provides a PEGylated fused hIL10 polypeptide of the structure:

PEG-[hIL10A-Lₙ-hIL10B],
wherein:
   (a) PEG is a 40 kD branched PEG molecule comprising two 20 kD arms of the formula:

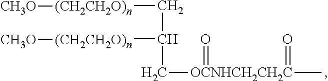

covalently bonded to the N-terminus of hIL10A, optionally via a linker; and
(b) the polypeptide of the formula [hIL10A-Lₙ-hIL10B] is a polypeptide of the sequence:

(SEQ ID NO: 185)
PGQGTQSENSCTHFPGNLPNMLRDLRDAFSRVKTFFQMKDQLDNLLLKES
LLEDFKGYLGCQALSEMIQFYLEEVMPQAENQDPDIKAHVNSLGENLKLL
RLRLRRCHRFLPCENKSKAVEQVKNAFNKLQEKGIYKAMSEFDIFINYIE
AYMTMKIRNSPGQGTQSENSCTHFPGNLPNMLRDLRDAFSRVKTFFQMKD
QLDNLLLKESLLEDFKGYLGCQALSEMIQFYLEEVMPQAENQDPDIKAHV
NSLGENLKLLRLRLRRCHRFLPCENKSKAVEQVKNAFNKLQEKGIYKAMS
EFDIFINYIEAYMTMKIRN.

In one embodiment, the present disclosure provides a PEGylated fused hIL10 polypeptide of the structure:

PEG-[hIL10A-Lₙ-hIL10B],
wherein:
   (a) PEG is a 40 kD branched PEG molecule comprising two 20 kD arms of the formula:

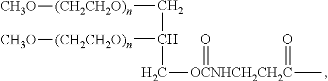

covalently bonded to the N-terminus of hIL10A, optionally via a linker; and
(b) the polypeptide of the formula [hIL10A-Lₙ-hIL10B] is a polypeptide of the sequence:

(SEQ ID NO: 170)
SPGQGTQSENSCTHFPGNLPNMLRDLRDAFSRVKTFFQMKDQLDNLLLKE
SLLEDFKGYLGCQALSEMIQFYLEEVMPQAENQDPDIKAHVNSLGENLKL
LRLRLRRCHRFLPCENKSKAVEQVKNAFNKLQEKGIYKAMSEFDIFINYI
EAYMTMKIRNSPGQGTQSENSCTHFPGNLPNMLRDLRDAFSRVKTFFQMK
DQLDNLLLKESLLEDFKGYLGCQALSEMIQFYLEEVMPQAENQDPDIKAH
VNSLGENLKLLRLRLRRCHRFLPCENKSKAVEQVKNAFNKLQEKGIYKAM
SEFDIFINYIEAYMTMKIRN.

In some embodiments, the present disclosure provides a “monoPEGylated” fused hIL10 polypeptide.

In one embodiment, the present disclosure provides a PEGylated fused hIL10 polypeptide of the structure:

PEG-hIL10A-Ln-hIL10B,
wherein:
   (a) PEG is a 40 kD branched PEG molecule attached to the N-terminus of the hIL10A optionally via a polypeptide or chemical linker;
   (b) hIL10A and hIL10B are hIL10 muteins wherein hIL10A and hIL10B are independently selected from hIL10 muteins comprising one or more amino acid substitutions at positions corresponding to residues T100, H14, N18, N21, M22, R24, D25, D28, R32, E74, H90, N92, S93, E96, and R104 (e.g., corresponding to residues T100, H14, N18, N21, M22, D25, R32, S93, and E96) of SEQ ID NO:4, and
   (c) L is a polypeptide linker and n=0 (absent) or 1 (present).

In one embodiment, the present disclosure provides a PEGylated fused hIL10 polypeptide of the structure:

PEG-hIL10A-Ln-hIL10B,
wherein:
   (a) PEG is a 40 kD branched PEG molecule attached to the N-terminus of the hIL10A optionally via a polypeptide or chemical linker;
   (b) hIL10A and hIL10B are hIL10 muteins wherein hIL10A and hIL10B are independently selected from hIL10 muteins comprising one or more amino acid substitutions at positions corresponding to residues T100, H14, N18, N21, M22, R24, D25, D28, R32, E74, H90, N92, 593, E96, and R104 (e.g., corresponding to residues T100, H14, N18, N21, M22, D25, R32, S93, and E96) of SEQ ID NO:4 wherein the one or more amino acid substitutions is selected from the group consisting of N21D, N21E, N21K, M22A, M22S, M22T, M22D, M22W, R24E, D25K, E96K, E96Q, T100E, T100L, and T100C and
   (c) L is a polypeptide linker and n=0 (absent) or 1 (present).

In one embodiment, the present disclosure provides a PEGylated fused hIL10 polypeptide of the structure:

PEG-hIL10 T100L-hIL10 T100L.

In one embodiment, the present disclosure provides a PEGylated fused hIL10 polypeptide of the structure:

PEG-[hIL10A-hIL10B]
wherein:
(a) PEG is a 40 kD branched PEG molecule attached to the N-terminus of the hIL10A optionally via a polypeptide or chemical linker;
(b) “—” is a peptide bond; and
(c) [hIL10A-hIL10B] is a polypeptide selected from the group consisting of [hIL10 N21D-hIL10 N21D], [ΔSer1 hIL10 N21D hIL10 N21D], [hIL10 N21E hIL10 N21E], [ΔSer1 hIL10 N21E-hIL10 N21E], {hIL10 N21K-hIL10 N21K], [ΔSer1hIL10 N21K-hIL10 N21K], [hIL10 M22A-hIL10 M22A], [ΔSer1 hIL10 M22A-hIL10 M22A], [hIL10 M22S-hIL10 M22S], [ΔSer1 hIL10 M22S-hIL10 M22S], [hIL10 M22T-hIL10 M22T], [ΔSer1 hIL10 M22T-hIL10 M22T], [hIL10 M22D-hIL10 M22D], [ΔSer1 hIL10 M22D-hIL10 M22D], [hIL10 M22W-hIL10 M22W], [ΔSer1 hIL10 M22W-hIL10 M22W], [hIL10 R24E-hIL10 R24E], [ΔSer1 hIL10 R24E-hIL10 R24E], [hIL10 D25K-hIL10 D25K], [ΔSer1 hIL10 D25K-hIL10 D25K], [hIL10 E96K-hIL10 E96K], [ΔSer1 hIL10 E96K-hIL10 E96K], [hIL10 E96Q-hIL10 E96Q], [ΔSer1 hIL10 E96Q-hIL10 E96Q], [hIL10 T100E-hIL10 T100E], [ΔSer1 hIL10 T100E-hIL10 T100E], [hIL10 T100L-hIL10 T100L], [ΔSer1 hIL10 T100L-hIL10 T100L], [hIL10 T100C-hIL10 T100C] and [ΔSer1 hIL10 T100C-hIL10 T100C].

In some instances, the fused hIL10 polypeptides of the present disclosure possess an N-terminal glutamine (“1Q”) residue. N-terminal glutamine residues have been observed to spontaneously cyclize to form pyroglutamate (pE) at or near physiological conditions. (See e.g., Liu, et al (2011) J. Biol. Chem. 286(13): 11211-11217). In some embodiments, the formation of pyroglutamate prevents N-terminal PEG conjugation particularly when aldehyde chemistry is used for N-terminal PEGylation. Consequently, when PEGylating the IL-10 agonist compounds of the present disclosure, particularly when aldehyde chemistry is to be employed, the IL-10 agonist compounds possessing an amino acid at position 1 (e.g., 1Q) are substituted at position 1 with an alternative amino acid or are deleted at position 1 (e.g., des-1Q). In some embodiments, the IL-10 agonist compounds of the present disclosure comprise an amino acid substitution selected from the group Q1E and Q1D.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons.

Two widely used first generation activated monomethoxy PEGs (mPEGs) are succinimdyl carbonate PEG (SC-PEG; see, e.g., Zalipsky, et al. (1992) Biotehnol. Appl. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

Pegylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General PEGylation strategies known in the art can be applied herein.

The PEG can be bound to a binding molecule of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the PEGylation of the binding molecules is facilitated by the incorporation of non-natural amino acids bearing unique side chains to facilitate site specific PEGylation. The incorporation of non-natural amino acids into polypeptides to provide functional moieties to achieve site specific PEGylation of such polypeptides is known in the art. See e.g., Ptacin et al., PCT International Application No. PCT/US2018/045257 filed Aug. 3, 2018 and published Feb. 7, 2019 as International Publication Number WO 2019/028419A1.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present disclosure include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g., Sunbright® ME-200AL, NOF), a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10 kDA linear PEG molecules (e.g., Sunbright® GL2-200AL3, NOF), a 20 kDa 2-arm branched PEG-NHS ester the 20 kDA PEG-NHS ester comprising two 10 kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20 kDA linear PEG molecules (e.g., Sunbright® GL2-400AL3), a 40 kDa 2-arm branched PEG-NHS ester the 40 kDA PEG-NHS ester comprising two 20 kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

In some embodiments, a linker can be used to join the fused hIL10 polypeptide and the PEG molecule. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of flexible linkers are described in Section IV. Further, a multimer (e.g., 1, 2, 3, 4, 5, 6, 7, 8, 9, 10, 10-20, 20-30, or 30-50) of these linker sequences may be linked together to provide flexible linkers that may be used to conjugate two molecules. Alternative to a polypeptide linker, the linker can be a chemical linker, e.g., a PEG-aldehyde linker. In some embodiments, the binding molecule is acetylated at the N-terminus by enzymatic reaction with N-terminal acetyltransferase and, for example, acetyl CoA. Alternatively, or in addition to N-terminal acetylation, the binding molecule can be acetylated at one or more lysine residues, e.g., by enzymatic reaction with a lysine acetyltransferase. See, for example Choudhary et al. (2009) Science 325 (5942):834-840.

In some embodiments, the present disclosure provides a fused hIL10 polypeptide that is PEGylated, wherein the PEG is conjugated to the fused hIL10 polypeptide and the PEG is a linear or branched PEG molecule having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

### Fatty Acid Carriers

In some embodiments a fused hIL10 polypeptide having an extended duration of action in a mammalian subject and useful in the practice of the present disclosure is achieved by covalent attachment of the fused hIL10 polypeptide to a fatty acid molecule as described in Resh (2016) Progress in Lipid Research 63: 120-131. Examples of fatty acids that may be conjugated include myristate, palmitate and palmitoleic acid. Myristoylate is typically linked to an N-terminal glycine but lysines may also be myristoylated. Palmitoylation is typically achieved by enzymatic modification of free cysteine —SH groups such as DHHC proteins catalyze S-palmitoylation. Palmitoleylation of serine and threonine residues is typically achieved enzymatically using PORCN enzymes. In some embodiments, the fused hIL10 polypeptide is acetylated at the N-terminus by enzymatic reaction with N-terminal acetyltransferase and, for example, acetyl CoA. Alternatively, or in addition to N-terminal acetylation, the fused hIL10 polypeptide is acetylated at one or more lysine residues, e.g., by enzymatic reaction with a lysine acetyltransferase. See, for example Choudhary et al. (2009) Science 325 (5942):834L2 ortho840.

### Nucleic Acid Sequences

In some embodiments, the fused hIL10 polypeptide is produced by recombinant methods using a nucleic acid sequence encoding the fused hIL10 polypeptide. The nucleic acid sequence encoding the desired the fused hIL10 polypeptide can be synthesized by chemical means using an oligonucleotide synthesizer.

The nucleic acid molecules are not limited to sequences that encode polypeptides; some or all of the non-coding sequences that lie upstream or downstream from a coding sequence can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The nucleic acid molecules encoding the fused hlL10 polypeptide may contain naturally occurring sequences or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

Nucleic acid sequences may be obtained from various commercial sources that provide custom made nucleic acid sequences. Amino acid sequence variants of the fused hIL10 polypeptide of the present disclosure are prepared by introducing appropriate nucleotide changes into the coding sequence based on the genetic code which is well known in the art. Such variants represent insertions, substitutions, and/or specified deletions of, residues as noted. Any combination of insertion, substitution, and/or specified deletion is made to arrive at the final construct, provided that the final construct possesses the desired biological activity as defined herein.

Methods for constructing DNA sequences and expressing those sequences in a suitably transformed host include, but are not limited to, using a PCR-assisted mutagenesis technique. Mutations that consist of deletions or additions of amino acid residues can also be made with standard recombinant techniques. In the event of a deletion or addition, the nucleic acid molecule is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

A fused hIL10 polypeptide of the present disclosure may be produced recombinantly not only directly, but also as a fusion polypeptide with a heterologous polypeptide, e.g. a signal sequence or other polypeptide having a specific cleavage site at the N-terminus or C-terminus of the mature fused hIL10 polypeptide. In some embodiments, the nucleic acid molecule further comprises a nucleic acid sequence encoding a signal peptide In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. The inclusion of a signal sequence depends on whether it is desired to secrete the molecule from the recombinant cells in which it is made. If the chosen cells are prokaryotic, it generally is preferred that the DNA sequence not encode a signal sequence. When the recombinant host cell is a yeast cell such as Saccharomyces cerevisiae, the alpha mating factor secretion signal sequence may be employed to achieve extracellular secretion of the molecule into the culture medium as described in Singh, U.S. Pat. No. 7,198,919 B1 issued Apr. 3, 2007. In some embodiments, the signal peptide comprises an endogenous or wild-type signal peptide. In some embodiments, the signal peptide comprises the amino acid sequence of the human IL10 polypeptide: MHSSALLCCLVLLTGVRA (SEQ ID NO:98). In some embodiments, the signal peptide comprises the amino acid sequence of murine IL10 polypeptide: MPGSALLCCLLLLTGMRI (SEQ ID NO:99).

In the event the fused hIL10 polypeptide to be expressed is to be expressed as a chimera (e.g., a fusion protein comprising a fused hIL10 polypeptide and a heterologous polypeptide sequence), the chimeric protein can be encoded by a hybrid nucleic acid molecule comprising a first sequence that encodes all or part of fused hIL10 polypeptide and a second sequence that encodes all or part of the heterologous polypeptide. For example, a molecule may be fused to a hexa-/octa-histidine tag (“H“and” HHHHHHHH” disclosed as SEQ ID NOS 147 and 146, respectively) to facilitate purification of bacterially expressed protein, or to a hemagglutinin tag to facilitate purification of protein expressed in eukaryotic cells. By first and second, it should not be understood as limiting to the orientation of the elements of the fusion protein and a heterologous polypeptide can be linked at either the N-terminus and/or C-terminus of the molecule. For example, the N-terminus may be linked to a targeting domain and the C-terminus linked to a hexa-histidine tag (SEQ ID NO:147) purification handle.

The complete amino acid sequence of the polypeptide (or fusion/chimera) to be expressed can be used to construct a back-translated gene. A DNA oligomer containing a nucleotide sequence coding a fused hIL10 polypeptide can be synthesized. For example, several small oligonucleotides coding for portions of the desired polypeptide can be synthesized and then ligated. The individual oligonucleotides typically contain 5′ or 3′ overhangs for complementary assembly.

### Codon Optimization

In some embodiments, the nucleic acid sequence encoding an amino acid molecule may be “codon optimized” to facilitate expression in a particular host cell type. Techniques for codon optimization in a wide variety of expression systems, including mammalian, yeast and bacterial host cells, are well known in the and there are online tools to provide for a codon optimized sequences for expression in a variety of host cell types. See e.g., Hawash, et al., (2017) 9:46-53 and Mauro and Chappell in Recombinant Protein Expression in Mammalian Cells: Methods and Protocols, edited by David Hacker (Human Press New York). Additionally, there are a variety of web based on-line software packages that are freely available to assist in the preparation of codon optimized nucleic acid sequences.

### Recombinant Production

In some embodiments, the fused hIL10 polypeptides of the present disclosure are produced by recombinant DNA technology. In the typical practice of recombinant production of polypeptides, a nucleic acid sequence encoding the desired polypeptide is incorporated into an expression vector suitable for the host cell in which expression will be accomplished, the nucleic acid sequence being operably linked to one or more expression control sequences encoded by the vector and functional in the target host cell. The recombinant protein may be recovered through disruption of the host cell or from the cell medium if a secretion leader sequence (signal peptide) is incorporated into the polypeptide.

In certain embodiments, a fused hIL10 polypeptide can contain amino acid substitutions which provide enhanced recombinant expression relative to the expression of wild-type hIL10 not containing such substitution. In some embodiments, the hIL10 mutein in a fused hIL10 polypeptide having increased expression in a transfected or recombinant cell comprises an amino acid substitution at the position corresponding to residue H14 of SEQ ID NO:4. In some embodiments, the amino acid substitution at the position corresponding to H14 of SEQ ID NO:4 is selected from the group consisting of H14A, H14D, H14E, H14I, H14K, H14L, H14M, H14N, H14Q, H14R, H14S, H14T, H14Y, and H14V. In some embodiments, the amino acid substitution at the position corresponding to H14 of SEQ ID NO:4 is selected from H14D, H14C, H14G, H14P, H14F, and H14W. In some embodiments, the amino acid substitutions at the position corresponding to H14 of SEQ ID NO:4 result in substantial increases in yield yet retain STAT3 signaling.

### Expression Vectors

Once assembled (by synthesis, site-directed mutagenesis or another method), the nucleic acid sequence encoding an amino acid molecule can be inserted into an expression vector. A variety of expression vectors for uses in various host cells are available and are typically selected based on the host cell for expression. An expression vector typically includes, but is not limited to, one or more of the following: an origin of replication, one or more marker genes, an enhancer element, a promoter, and a transcription termination sequence. Vectors include viral vectors, plasmid vectors, integrating vectors, and the like. Plasmids are examples of non-viral vectors.

In some embodiments, the vector can comprise a sequence operably linked to an expression control element (e.g. a promoter). To facilitate efficient expression of the recombinant polypeptide, the nucleic acid sequence encoding the polypeptide sequence to be expressed is operably linked to transcriptional and translational regulatory control sequences that are functional in the chosen expression host.

### Selectable Marker

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

### Regulatory Control Sequences

Expression vectors can contain a regulatory sequence that is recognized by the host organism and is operably linked to a nucleic acid sequence encoding the amino acid molecule. The terms “regulatory control sequence,” “regulatory sequence” or “expression control sequence” are used interchangeably herein to refer to promoters, enhancers, and other expression control elements (e.g., polyadenylation signals). See, for example, Goeddel (1990) in Gene Expression Technology: Methods in Enzymology 185 (Academic Press, San Diego CA USA Regulatory sequences include those that direct constitute expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. In selecting an expression control sequence, a variety of factors understood by one of skill in the art are to be considered. These include, for example, the relative strength of the sequence, its controllability, and its compatibility with the actual DNA sequence encoding the amino acid molecule, particularly as regards potential secondary structures.

In some embodiments, the regulatory sequence is a promoter, which is selected based on, for example, the cell type in which expression is sought. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

Transcription from vectors in mammalian host cells may be controlled, for example, by promoters obtained from the genomes of viruses such as polyoma virus, fowlpox virus, adenovirus (such as human adenovirus serotype 5), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter. Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Additional examples of marker or reporter genes include beta-lactamase, chloramphenicol acetyltransferase (CAT), adenosine deaminase (ADA), dihydrofolate reductase (DHFR), hygromycin-B-phosphotransferase (HPH), thymidine kinase (TK), lacZ (encoding beta-galactosidase), and xanthine guanine phosphoribosyltransferase (XGPRT). Those of skill in the art can readily determine whether a given regulatory element or selectable marker is suitable for use in a particular experimental context.

Proper assembly of the expression vector can be confirmed by nucleotide sequencing, restriction mapping, and expression of a biologically active polypeptide in a suitable host.

### Host Cells

The present disclosure further provides prokaryotic or eukaryotic cells that contain and express one or more nucleic acid molecules that encoding a fused hIL10 polypeptide. A cell of the present disclosure is a transfected cell, i.e., a cell into which a nucleic acid molecule, for example a nucleic acid molecule encoding a fused hIL10 polypeptide, has been introduced by means of recombinant DNA techniques.

In some embodiments, the recombinantly modified cell comprises a vector, the vector comprising a nucleic acid sequence encoding the amino acid molecule. In some embodiments, the recombinantly modified cell is a prokaryotic cell, such as a bacterial cell. In some embodiments, the recombinantly modified cell is a eukaryotic cell, such as a mammalian cell.

Host cells are typically selected in accordance with their compatibility with the chosen expression vector, the toxicity of the product coded for by the DNA sequences of this invention, their secretion characteristics, their ability to fold the polypeptides correctly, their fermentation or culture requirements, and the ease of purification of the products coded for by the DNA sequences. Suitable host cells for cloning or expressing the DNA in the vectors herein are the prokaryote, yeast, or higher eukaryote cells.

In some embodiments the recombinant fused hIL10 polypeptide or mutein can also be made in eukaryotes, such as yeast or human cells. Suitable eukaryotic host cells include insect cells (examples of baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerenvisiae include pYepSecl (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)).

Examples of useful mammalian host cell lines are mouse L cells (L-M[TK-], ATCC #CRL-2648), monkey kidney CV1 line transformed by SV40 (COS-7, ATCC CRL 1651); human embryonic kidney line (HEK293 or HEK293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2). In mammalian cells, the expression vector's control functions are often provided by viral regulatory elements. For example, commonly used promoters are derived from polyoma, Adenovirus 2, cytomegalovirus, and Simian Virus 40.

The fused hIL10 polypeptide may be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

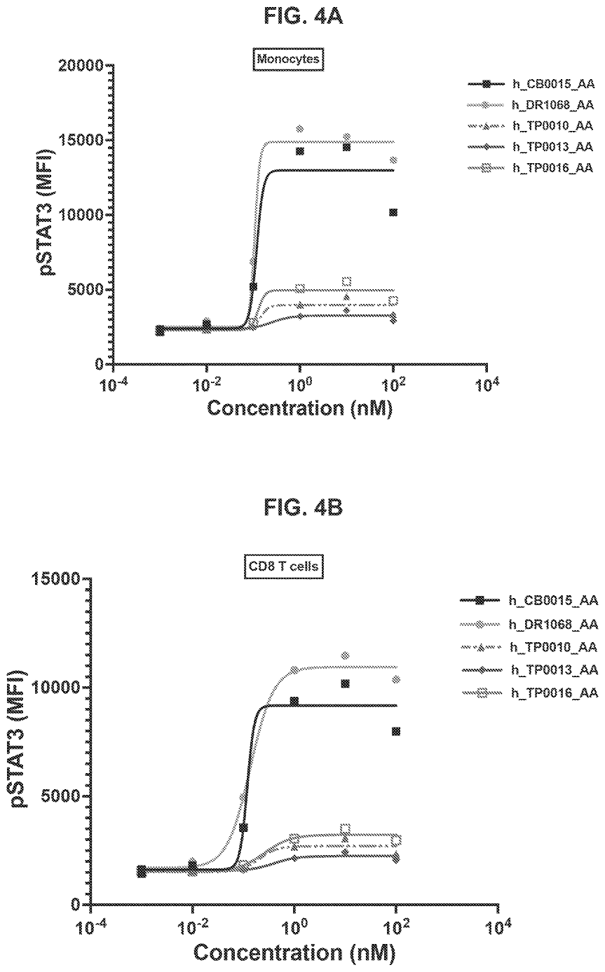

When wild-type hIL10 is expressed endogenously in mammalian cells, it is expressed as a pre-protein comprising a signal peptide which is efficiently cleaved in mammalian cells resulting in the N-terminal amino acid of the mature wild-type hIL10 polypeptide being a serine residue (Ser1). In an alternative to recombinant expression in mammalian cells, the fused hIL10 polypeptides may also be recombinantly expressed in bacterial cells. Direct expression (i.e., not as a N-terminal fusion protein) of a fused hIL10 polypeptides results in the addition of a N-terminal methionine residue to the fused hIL10 polypeptide (i.e., an N-terminal sequence of the beginning with Met-Ser-Pro- . . . ). If the Ser1 characteristic of the native N-terminal sequence of the wild-type IL10 sequence is retained at the N-terminus of the hIL10A of the fused (hIL10A-hIL10B) hIL10 polypeptide, this will result in a proline (P) residue at the +2 position relative to N-terminal methionine of the fused hIL10 polypeptide. When a proline is present at the +2 position relative to the N-terminal methionine of a polypeptide, the endogenous bacterial methionyl amino peptidase (MAP) of the bacterial host cell frequently does not efficiently cleave the N terminal methionine. (See e.g., FIG. 4B of Frottin, et al. (2019) The Proteomics of N-terminal Methionine Cleavage, Molecular & Cellular Proteomics 5(12):2336-2349). Consequently, bacterial direct expression of the fused hIL10 polypeptides comprising the native N-terminal sequence may result in a mixture of fused hIL10 polypeptide species, one fraction having an N-terminal methionine residue and another species lacking the N-terminal methionine. Such a mixture of fused hIL10 polypeptide species may be difficult to resolve by typical manufacturing procedures which may result in increased processing, loss of product and other difficulties when attempting to conjugate the molecules to N-terminus of the fused hIL10 polypeptide such as a targeting molecules or carrier molecules such as a PEG molecule. However, by deleting Ser1 (des-Ser1) from the fused hIL10 polypeptide, the residue in the +2 position relative to the N-terminal methionine is a glycine residue (G3) which provides for efficient cleavage of the N-terminal methionine and facilitates bacterial production of the fused hIL10 polypeptide and provides a more uniform fused hIL10 polypeptide product. In some embodiments, the present disclosure, provides fused hIL10 polypeptide comprising a deletion of the serine at position 1 (des-Ser1), numbered in accordance with hIL10) of the N-terminal hIL10 (hIL10A) of the fused hIL10 polypeptide. Alternatively, it has been shown that deletion of 1, 2, 3, 4, 5, 6, 7, 8, 9 or 10 amino acids of the N-terminus of the hIL10 molecule results in a hIL10 molecule substantially retaining the activity of hIL10. In some embodiments, the hIL10A of the fused hIL10 polypeptide comprises a deletion of 1, 2, 3, 4, 5, 6, 7, 8, 9 or 10 amino acids of the N-terminus of the hIL10A of the fused hIL10 polypeptide.

In some embodiments, a fused hIL10 polypeptide or mutein obtained will be glycosylated or unglycosylated depending on the host organism used to produce the mutein. If bacteria are chosen as the host then the molecule produced will be unglycosylated. Eukaryotic cells, on the other hand, will typically result in glycosylation of the molecule.

In some embodiments, it is possible that an amino acid of a fused hIL10 polypeptide may contain a glycosylation motif, particularly an N-linked glycosylation motif of the sequence Asn-X-Ser (N-X-S) or Asn-X-Thr (N-X-T), wherein X is any amino acid except for proline. In such instances, it is desirable to eliminate such N-linked glycosylation motifs by modifying the sequence of the N-linked glycosylation motif to prevent glycosylation. In some embodiments, the N-linked glycosylation motif is disrupted by the incorporation of conservative amino acid substitution of the Asn (N) residue of the N-linked glycosylation motif. For example, residues N116/K117/S118 of the wild-type sequence of human IL10 define a potential NXS N-linked glycosylation motif. While hIL10 produced in mammals is not observed to be glycosylated, in some embodiments, the hIL10A and/or IL10B of the fused hIL10 polypeptide may comprise a conservative amino acid amino acid substitution at one or more of positions N116, K117, and/or S118 to preclude glycosylation at this putative N-linked glycosylation motif.

For other additional expression systems for both prokaryotic and eukaryotic cells, see Chapters 16 and 17 of Sambrook et al. (1989) Molecular Cloning: A Laboratory Manual (2nd ed., Cold Spring Harbor Laboratory Press, Plainview, N.Y.). See, Goeddel (1990) in Gene Expression Technology: Methods in Enzymology 185 (Academic Press, San Diego, Calif.).

### Transfection

The expression constructs of the can be introduced into host cells to thereby produce a fused hIL10 polypeptide or mutein disclosed herein. The expression vector comprising a nucleic acid sequence encoding amino acid molecule is introduced into the prokaryotic or eukaryotic host cells via conventional transformation or transfection techniques. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals. To facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, and magnetic fields (electroporation).

### Cell Culture

Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

### Recovery of Recombinant Proteins

Recombinantly produced molecules can be recovered from the culture medium as a secreted polypeptide if a secretion leader sequence is employed. Alternatively, the molecules can also be recovered from host cell lysates. A protease inhibitor, such as phenyl methyl sulfonyl fluoride (PMSF) may be employed during the recovery phase from cell lysates to inhibit proteolytic degradation during purification, and antibiotics may be included to prevent the growth of adventitious contaminants.

Various purification steps are known in the art and find use, e.g., affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural specific binding of one molecular species to separate and purify a second species from a mixture. Antibodies are commonly used in affinity chromatography. Size selection steps may also be used, e.g. gel filtration chromatography (also known as size-exclusion chromatography or molecular sieve chromatography) is used to separate proteins according to their size. In gel filtration, a protein solution is passed through a column that is packed with semipermeable porous resin. The semipermeable resin has a range of pore sizes that determines the size of proteins that can be separated with the column.

A recombinant molecule expressed by the transformed host can be purified according to any suitable method. Recombinant molecules can be isolated from inclusion bodies generated in E. coli, or from conditioned medium from either mammalian or yeast cultures producing a given mutein using cation exchange, gel filtration, and or reverse phase liquid chromatography. The substantially purified forms of the recombinant molecules can be purified from the expression system using routine biochemical procedures, and can be used, e.g., as therapeutic agents, as described herein.

In some embodiments, where the molecule is expressed with a purification tag as discussed above, this purification handle may be used for isolation of the molecule from the cell lysate or cell medium. Where the purification tag is a chelating peptide, methods for the isolation of such molecules using immobilized metal affinity chromatography are well known in the art. See, e.g., Smith, et al. U.S. Pat. No. 4,569,794. In some embodiments, the chelating peptide is a poly-histidine polypeptide sequence comprising 4, 5, 6. 7. 8, 9, or 10 histidine residues. Such polyhistidine chelating peptides are also referred to as “His tags” in the scientific literature. In some embodiments, the fused hIL10 polypeptide is modified to comprise a chelating peptide at the N-terminus optionally wherein the chelating peptide is covalently linked to the fused hIL10 polypeptide via a GS linker. In some embodiments, the fused hIL10 polypeptide is modified to comprise a chelating peptide at the C-terminus, optionally wherein the chelating peptide is covalently linked to the fused hIL10 polypeptide via a linker.

The biological activity of the molecule recovered can be assayed for activating by any suitable method known in the art and may be evaluated as substantially purified forms or as part of the cell lysate or cell medium when secretion leader sequences are employed for expression.

### Pharmaceutical Formulations

In some embodiments, the fused hIL10 polypeptides (and/or nucleic acids encoding the fused hIL10 polypeptide or recombinant cells incorporating a nucleic acid sequence and modified to express the fused hIL10 polypeptide) can be incorporated into compositions, including pharmaceutical compositions. Such compositions typically include the polypeptide or nucleic acid molecule and a pharmaceutically acceptable carrier. A pharmaceutical composition is formulated to be compatible with its intended route of administration and is compatible with the therapeutic use for which the fused hIL10 polypeptide is to be administered to the subject in need of treatment or prophyaxis.

### Carriers:

Carriers include a sterile diluent such as water for injection, saline solution, fixed oils, polyethylene glycols, glycerine, propylene glycol or other synthetic solvents. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. For intravenous administration, suitable carriers include physiological saline, bacteriostatic water, Cremophor EL™ (BASF, Parsippany, N.J.) or phosphate buffered saline (PBS).

### Buffers:

The term buffers includes buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5).

### Dispersions:

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### Preservatives:

The pharmaceutical formulations for parenteral administration to a subject should be sterile and should be fluid to facilitate easy syringability. It should be stable under the conditions of manufacture and storage and are preserved against the contamination. Prevention of the action of microorganisms can be achieved by various antibacterial and antifungal agents, for example, agents such as benzyl alcohol or methyl parabens; antioxidants such as ascorbic acid or sodium bisulfite; chelating agents such as ethylenediaminetetraacetic acid, parabens, chlorobutanol, phenol, ascorbic acid, thimerosal, and the like. Sterile solutions can be prepared by incorporating the active compound in the required amount in an appropriate solvent with one or a combination of ingredients enumerated above, as required, followed by filtered sterilization.

### Tonicity Agents:

In many cases, it will be preferable to include isotonic agents, for example, sugars, polyalcohols such as mannitol, sorbitol, sodium chloride in the composition.

### Routes of Administration:

In some embodiments of the therapeutic methods of the present disclosure involve the administration of a pharmaceutical formulation comprising a fused hIL10 polypeptide (and/or nucleic acids encoding the fused hIL10 polypeptide or recombinantly modified host cells expressing the fused hIL10 polypeptide) to a subject in need of treatment. The pharmaceutical formulation comprising a fused hIL10 polypeptide of the present disclosure may be administered to a subject in need of treatment or prophyaxis by a variety of routes of administration, including parenteral administration, oral, topical, or inhalation routes.

### Parenteral Administration:

In some embodiments, the methods of the present disclosure involve the parenteral administration of a pharmaceutical formulation comprising a fused hIL10 polypeptide (and/or nucleic acids encoding the fused hIL10 polypeptide or recombinantly modified host cells expressing the fused hIL10 polypeptide) to a subject in need of treatment. Examples of parenteral routes of administration include, for example, intravenous, intradermal, subcutaneous, transdermal (topical), transmucosal, and rectal administration. Parenteral formulations comprise solutions or suspensions used for parenteral application can include vehicles the carriers and buffers. Pharmaceutical formulations for parenteral administration include sterile aqueous solutions (where water soluble) or dispersions and sterile powders for the extemporaneous preparation of sterile injectable solutions or dispersion. The parenteral preparation can be enclosed in ampoules, disposable syringes or multiple dose vials made of glass or plastic. In one embodiment, the formulation is provided in a prefilled syringe for

### Oral Administration:

In some embodiments, the methods of the present disclosure involve the oral administration of a pharmaceutical formulation comprising a fused hIL10 polypeptide (and/or nucleic acids encoding the fused hIL10 polypeptide or recombinantly modified host cells expressing the fused hIL10 polypeptide) to a subject in need of treatment. Oral compositions, if used, generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

### Inhalation Formulations:

In some embodiments, the methods of the present disclosure involve the inhaled administration of a pharmaceutical formulation comprising a fused hIL10 polypeptide (and/or nucleic acids encoding the fused hIL10 polypeptide or recombinantly modified host cells expressing the fused hIL10 polypeptide) to a subject in need of treatment. In the event of administration by inhalation, subject fused hIL10 polypeptides, or the nucleic acids encoding them, are delivered in the form of an aerosol spray from pressured container or dispenser which contains a suitable propellant, e.g., a gas such as carbon dioxide, or a nebulizer. Such methods include those described in U.S. Pat. No. 6,468,798.

### Mucosal and Transdermal Formulations:

In some embodiments, the methods of the present disclosure involve the mucosal or transdermal administration of a pharmaceutical formulation comprising a fused hIL10 polypeptide (and/or nucleic acids encoding the fused hIL10 polypeptide or recombinantly modified host cells expressing the fused hIL10 polypeptide) to a subject in need of treatment. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories (e.g., with conventional suppository bases such as cocoa butter and other glycerides) or retention enemas for rectal delivery. For transdermal administration, the active compounds are formulated into ointments, salves, gels, or creams as generally known in the art and may incorporate permeation enhancers such as ethanol or lanolin.

### Extended Release and Depot Formulations:

In some embodiments of the method of the present disclosure, the fused hIL10 polypeptide is administered to a subject in need of treatment in a formulation to provide extended release of the fused hIL10 polypeptide. Examples of extended release formulations of the injectable compositions can be brought about by including in the composition an agent which delays absorption, for example, aluminum monostearate and gelatin. In one embodiment, the subject fused hIL10 polypeptide or nucleic acids are prepared with carriers that will protect the fused hIL10 polypeptide against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Administration of Nucleic Acids Encoding the Fused hIL10 Polypeptide:

In some embodiments of the method of the present disclosure, delivery of the fused hIL10 polypeptide to a subject in need of treatment is achieved by the administration of a nucleic acid encoding the fused hIL10 polypeptide. Methods for the administration nucleic acid encoding the fused hIL10 polypeptide to a subject is achieved by transfection or infection using methods known in the art, including but not limited to the methods described in McCaffrey et al. (Nature (2002) 418:6893), Xia et al. (Nature Biotechnol. (2002) 20:1006-1010), or Putnam (Am. J. Health Syst. Pharm. (1996) 53: 151-160 erratum at Am. J. Health Syst. Pharm. (1996) 53:325). In some embodiments, the fused hIL10 polypeptide is administered to a subject by the administration of a pharmaceutically acceptable formulation of recombinant expression vector comprising a nucleic acid sequence encoding the fused hIL10 polypeptide operably linked to one or more expression control sequences operable in a mammalian subject. In some embodiments, the expression control sequence may be selected that is operable in a limited range of cell types (or single cell type) to facilitate the selective expression of the fused hIL10 polypeptide in a particular target cell type. In one embodiment, the recombinant expression vector is a viral vector. In some embodiments, the recombinant vector is a recombinant viral vector. In some embodiments the recombinant viral vector is a recombinant adenoassociated virus (rAAV) or recombinant adenovirus (rAd), in particular a replication deficient adenovirus derived from human adenovirus serotypes 3 and/or 5. In some embodiments, the replication deficient adenovirus has one or more modifications to the E1 region which interfere with the ability of the virus to initiate the cell cycle and/or apoptotic pathways in a human cell. The replication deficient adenoviral vector may optionally comprise deletions in the E3 domain. In some embodiments the adenovirus is a replication competent adenovirus. In some embodiments the adenovirus is a replication competent recombinant virus engineered to selectively replicate in the target cell type.

In some embodiments, particularly for administration of fused hIL10 polypeptides to the subject, particular for treatment of diseases of the intestinal tract or bacterial infections in a subject, the nucleic acid encoding the fused hIL10 polypeptide may be delivered to the subject by the administration of a recombinantly modified bacteriophage vector encoding the fused hIL10 polypeptide. As used herein, the terms ‘procaryotic virus,” “bacteriophage” and “phage” are used interchangeably hereinto describe any of a variety of bacterial viruses that infect and replicate within a bacterium. Bacteriophage selectively infect procaryotic cells, restricting the expression of the fused hIL10 polypeptide to procaryotic cells in the subject while avoiding expression in mammalian cells. A wide variety of bacteriophages capable of selection a broad range of bacterial cells have been identified and characterized extensively in the scientific literature. In some embodiments, the phage is modified to remove adjacent motifs (PAM). Elimination of Cas9 sequences from the phage genome reduces ability of the Cas9 endonuclease of the target procaryotic cell to neutralize the invading phage encoding the fused hIL10 polypeptide.

Administration of Recombinantly Modified Cells Expressing the Fused hIL10 Polypeptide

In some embodiments of the method of the present disclosure, delivery of the fused hIL10 polypeptide to a subject in need of treatment is achieved by the administration of recombinant host cells modified to express the fused hIL10 polypeptide may be administered in the therapeutic and prophylactic applications described herein. In some embodiments, the recombinant host cells are mammalian cells, e.g., human cells. In some embodiments, the recombinant host cells are procaryotic cells, e.g., bacterial cells associated with the intestinal flora such as E. coli or Lactobacillus lacti.

In some embodiments, the nucleic acid sequence encoding the fused hIL10 polypeptide (or vectors comprising same) may be maintained extrachromosomally in the recombinantly modified host cell for administration. In other embodiments, the nucleic acid sequence encoding the fused hIL10 polypeptide may be incorporated into the genome of the host cell to be administered using at least one endonuclease to facilitate incorporate insertion of a nucleic acid sequence into the genomic sequence of the cell. As used herein, the term “endonuclease” is used to refer to a wild-type or variant enzyme capable of catalyzing the cleavage of bonds between nucleic acids within a DNA or RNA molecule, preferably a DNA molecule. Endonucleases are referred to as “rare-cutting” endonucleases when such endonucleases have a polynucleotide recognition site greater than about 12 base pairs (bp) in length, more preferably of 14-55 bp. Rare-cutting endonucleases can be used for inactivating genes at a locus or to integrate transgenes by homologous recombination (HR) i.e. by inducing DNA double-strand breaks (DSBs) at a locus and insertion of exogenous DNA at this locus by gene repair mechanism. Examples of rare-cutting endonucleases include homing endonucleases (Grizot, et al (2009) Nucleic Acids Research 37(16):5405-5419), chimeric Zinc-Finger nucleases (ZFN) resulting from the fusion of engineered zinc-finger domains (Porteus M and Carroll D., Gene targeting using zinc finger nucleases (2005) Nature Biotechnology 23(3):967-973, a TALEN-nuclease, a Cas9 endonuclease from CRISPR system as or a modified restriction endonuclease to extended sequence specificity (Eisenschmidt, et al. 2005; 33(22): 7039-7047).

In some embodiments, particularly for administration of fused hIL10 polypeptide to the intestinal tract, the fused hIL10 polypeptide may be delivered to the subject by a recombinantly modified procaryotic cell (e.g., Lactobacillus lacti). The use of engineered procaryotic cells for the delivery of recombinant proteins to the intestinal tract are known in the art. See, e.g. Lin, et al. (2017) Microb Cell Fact 16:148. In some embodiments, the engineered bacterial cell expressing the fused hIL10 polypeptide may be administered orally, typically in aqueous suspension, or rectally (e.g. enema).

### Methods of Use

The present disclosure further provides methods of treating a subject suffering from a disease disorder or condition by the administration of a therapeutically effective amount of: (a) a fused hIL10 polypeptide, (b) a pharmaceutically acceptable formulation containing as an active ingredient a fused hIL10 polypeptide; (c) oa recombinant non-viral or eucaryotic viral or bacteriophage vectors comprising a nucleic acid sequence encoding an a fused hIL10 polypeptide thereof operably linked to one or more expression control sequences, (d) a recombinant procaryotic or mammalian cells genomically modified to comprise a nucleic acid sequence encoding a fused hIL10 polypeptide operably linked to one or more expression control sequences, or (e) a recombinant procaryotic or mammalian cell comprising a nucleic acid encoding a fused hIL10 polypeptide operably linked to one or more expression control sequences.

### Inflammatory and Autoimmune Disorders

Disorders amenable to treatment with a fused hIL10 polypeptide (including pharmaceutically acceptable formulations comprising a fused hIL10 polypeptide and/or the nucleic acid molecules that encode them including recombinant viruses encoding such a fused hIL10 polypeptide) of the present disclosure include inflammatory or autoimmune diseases including but not limited to, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes including Type 1 or type 2 diabetes, inflammation, autoimmune disease, atopic diseases, paraneoplastic autoimmune diseases, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome (Seronegativity Enthesopathy Arthropathy Syndrome), juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome,SEA Syndrome (Seronegativity, Enthesopathy, Arthropathy Syndrome).

### Ulcerative Colitis

In some embodiments, the present disclosure provides a method of treating a mammalian subject suffering from ulcerative colitis, the method comprising the step of administering to the subject a fused hIL10 polypeptide (or pharmaceutical formulation comprising a fused hIL10 polypeptide) of the present disclosure, the administering providing an improvement in one or more symptoms of the ulcerative colitis. In one embodiment, the present disclosure provides a method of treating a mammalian subject suffering from ulcerative colitis the method comprising the step of administering to the subject a fused hIL10 polypeptide having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to a polypeptide selected from the group consisting of SEQ ID NOS:24, 25, 26, 27, 28, 29, 30, 31, 32, 33, 34, 35, 36, 37, 38, 39, 40, 41, 42, 43, 44, 45, 46, 47, 48, 49, 50, 119, 157, 158, 159, 160, 161, 162, 163, 164, 165, 166, 167, 168, 169, 170, 171, 172, 173, 174, 175, 176, 177, 178, 179, 180, 181, 182, 183 184, 185 and 186, wherein the administering providing an improvement in one or more symptoms of the ulcerative colitis.

### IFNγ Induced Anemia:

In some embodiments, the present disclosure provides a method of treating and/or preventing IFNγ induced anemia in a subject. the method comprising the step of administering to the subject a therapeutically effective amount of a fused IL10 dimer of the present disclosure wherein the administering results in an amelioration of one or more symptoms of the IFNγ induced anaemia and/or a reduction in serum IFNγ levels in the subject. IFNγ induced anaemia is a frequent complication in patients with chronic inflammatory diseases. Chronic inflammation may arise from a variety of conditions such as infectious disease, AIDS, malignancies and/or autoimmune diseases such as inflammatory bowel disease, rheumatoic arthritis. Activated T cells secrete IFNγ and interleukin 2 (IL-2). IFNγ has been observed to increase the cytotoxic effects of TNFα. The compositions of the present disclosure were evaluated in a murine model of IFNγ induced anemia (Example 32). As illustrated in FIG. 44, the administration of the wild-type mIL10 surrogate molecule exacerbated IFNγ induced anemia whereas the administration of a fused dimer of the present disclosure did not. In one embodiment, the present disclosure provides a method of treating and/or preventing IFNγ induced anemia in a subject the method comprising the step of administering to the subject a fused hIL10 polypeptide having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to a polypeptide selected from the group consisting of SEQ ID NOS: 24, 25, 26, 27, 28, 29, 30, 31, 32, 33, 34, 35, 36, 37, 38, 39, 40, 41, 42, 43, 44, 45, 46, 47, 48, 49, 50, 119, 157, 158, 159, 160, 161, 162, 163, 164, 165, 166, 167, 168, 169, 170, 171, 172, 173, 174, 175, 176, 177, 178, 179, 180, 181, 182, 183 184, 185 and 186, wherein the administering providing an improvement in one or more symptoms of the IFNγ induced anemia.

### Macrophage Activation Syndrome

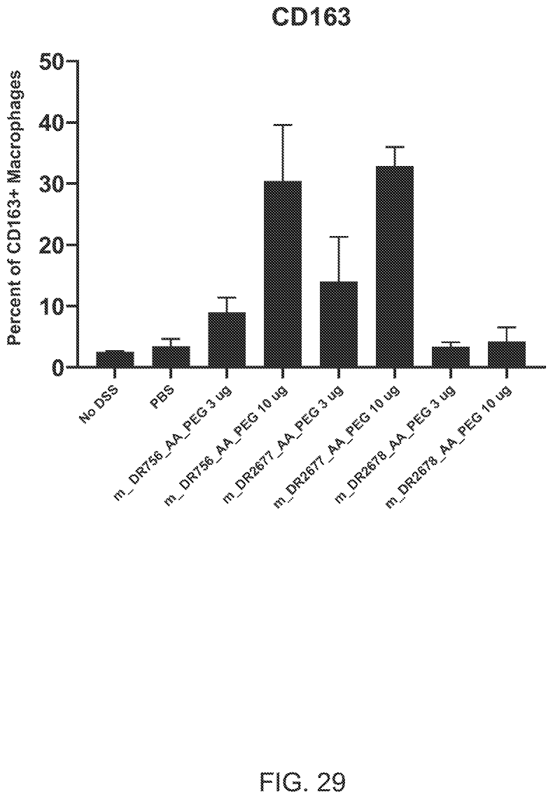

In some embodiments, the present disclosure provides a method of treating and/or preventing macrophage activation syndrome (MAS) in a subject. the method comprising the step of administering to the subject a therapeutically effective amount of a fused IL10 dimer of the present disclosure. Macrophage activation syndrome (MAS) is a potentially life-threatening complication of systemic inflammatory disorders, including but not limited to systemic juvenile idiopathic arthritis (sJIA), Kawasaki disease, systemic lupus erythematosus (SLE) and infections, particularly Epstein-Barr Virus (EBV) infection, malignancy, and primary immunodeficiencies. Elevated CD163 is associated with MAS. As shown in FIG. 29, treatment with wild-type IL-10 induced CD163 expression on Day 15 PECs whereas the fused IL-10 dimer comprising hIL10 muteins did not induce CD163. In some embodiments, the present disclosure provides a method of treating and/or preventing macrophage activation syndrome (MAS) in a subject. the method comprising the step of administering to the subject a therapeutically effective amount of a fused IL10 dimer of the present disclosure, wherein the administering results in an amelioration of one or more symptoms of the macrophage activation syndrome. In one embodiment, the present disclosure provides a method of treating and/or preventing macrophage activation syndrome in a subject the method comprising the step of administering to the subject a fused hIL10 polypeptide having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to a polypeptide selected from the group consisting of SEQ ID NOS: 24, 25, 26, 27, 28, 29, 30, 31, 32, 33, 34, 35, 36, 37, 38, 39, 40, 41, 42, 43, 44, 45, 46, 47, 48, 49, 50, 119, 157, 158, 159, 160, 161, 162, 163, 164, 165, 166, 167, 168, 169, 170, 171, 172, 173, 174, 175, 176, 177, 178, 179, 180, 181, 182, 183 184, 185 and 186, wherein the administering providing an improvement in one or more symptoms of the macrophage activation syndrome.

Other examples of proliferative and/or differentiative disorders amenable to treatment with fused hIL10 polypeptide (including pharmaceutically acceptable formulations comprising fused hIL10 polypeptide and/or the nucleic acid molecules that encode them including recombinant viruses encoding such fused hIL10 polypeptide) of the present disclosure include, but are not limited to, skin disorders. The skin disorder may involve the aberrant activity of a cell or a group of cells or layers in the dermal, epidermal, or hypodermal layer, or an abnormality in the dermal-epidermal junction. For example, the skin disorder may involve aberrant activity of keratinocytes (e.g., hyperproliferative basal and immediately suprabasal keratinocytes), melanocytes, Langerhans cells, Merkel cells, immune cell, and other cells found in one or more of the epidermal layers, e.g., the stratum basale (stratum germinativum), stratum spinosum, stratum granulosum, stratum lucidum or stratum corneum. In other embodiments, the disorder may involve aberrant activity of a dermal cell, for example, a dermal endothelial, fibroblast, immune cell (e.g., mast cell or macrophage) found in a dermal layer, for example, the papillary layer or the reticular layer.

Examples of inflammatory or autoimmune skin disorders include psoriasis, psoriatic arthritis, dermatitis (eczema), for example, exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitises such as seborrheic dermatitis and solar dermatitis; keratoses such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis. The skin disorder can be dermatitis, e.g., atopic dermatitis or allergic dermatitis, or psoriasis.

The compositions of the present disclosure (including pharmaceutically acceptable formulations comprising fused hIL10 polypeptide and/or the nucleic acid molecules that encode them including recombinant viruses encoding such fused hIL10 polypeptide) can also be administered to a patient who is suffering from (or may suffer from) psoriasis or psoriatic disorders. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis. Examples of psoriatic disorders include chronic stationary psoriasis, plaque psoriasis, moderate to severe plaque psoriasis, psoriasis vulgaris, eruptive psoriasis, psoriatic erythroderma, generalized pustular psoriasis, annular pustular psoriasis, or localized pustular psoriasis.

In some embodiments, the present disclosure provides a method of treating cancer the method comprising administering to the subject a therapeutically effective amount of a fused IL10 polypeptide of the present disclosure wherein the administering results in the amelioration of one or more symptoms of the cancer. As previously discussed, chronic inflammation is associated with the development of a wide variety of cancers. See. e.g. Coussens and Werb (2002) Nature 420: 860-867. Consequently, compositions of the inverted of the present disclosure may be used in the treatment and/or prevention of cancers arising from chronic inflammation. In one embodiment, the cancer associated with chronic inflammation is selected from the group consisting of colon cancer, colorectal cancer, pancreatic cancer and liver cancer. In one embodiment, the present disclosure provides a method of preventing the development of cancer associated with chronic inflammation in a human subject by the administration to the subject of a prophylactically effective amount of a composition comprising, vector encoding or recombinant cell expressing a fused IL10 polypeptide of the present disclosure.

### Dosing

The fused hIL10 polypeptides of the present disclosure exhibit a broadened therapeutic window as compared to wild-type IL10. The “therapeutic window” refers to the range of dosing of the fused hIL10 polypeptide that provides a concentration of the fused hIL10 polypeptide in the subject sufficient to provide substantial level of an IL10 activity (e.g., STAT3 signaling) in monocytes but that is less than the dose that results in a concentration in the subject sufficient to induce a substantial level of the IL10 activity signaling in T cells. Consequently, in some embodiments the fused hIL10 polypeptide is dosed to a subject at a level that results in a concentration in the subject sufficient to provide substantial level STAT3 signaling in monocytes but that is less than the dose that results in a concentration in the subject sufficient to induce a substantial level of STAT3 signaling in T cells. In some embodiments, the present disclosure provides a method of treating a subject suffering from an inflammatory or autoimmune disease, disorder or condition characterized by the administration of a therapeutically effective amount of an fused hIL10 polypeptide (or a pharmaceutically acceptable formulation containing as an active ingredient a fused hIL10 polypeptide) wherein the therapeutically effective amount is that amount that results in a concentration in the subject sufficient to provide substantial level STAT3 signaling in monocytes but that is less than the dose that results in a concentration in the subject sufficient to induce a substantial level of STAT3 signaling in T cells.

In one embodiment, the present disclosure provides a method of treating a subject suffering from an inflammatory or autoimmune disease, disorder, or condition by the administration of a therapeutically effective amount of a fused hIL10 polypeptide (or a pharmaceutically acceptable formulation containing as an active ingredient a fused hIL10 polypeptide) at a dose level that monomer that maximizes the ratio of the Emax of an IL10 activity on monocytes relative to the Emax of such IL10 activity on T cells. In some embodiments, the present disclosure provides a methods of treating a subject suffering from a disease disorder or condition by the administration of a therapeutically effective amount of z fused hIL10 polypeptide (or a pharmaceutically acceptable formulation containing as an active ingredient z fused hIL10 polypeptide) wherein the therapeutically effective amount is a dose to provide a concentration that is: (a) greater than the STAT3 EC₂₀ on activated human monocytes, alternatively greater than the STAT3 EC₃₀ on activated human monocytes, alternatively greater than the STAT3 EC₄₀ on activated human monocytes, alternatively greater than the STAT3 EC₅₀ on activated human monocytes, alternatively greater than the STAT3 EC₆₀ on activated human monocytes, STAT3 EC₇₀ on activated human monocytes, alternatively greater than the STAT3 EC₈₀ on activated human monocytes, alternatively greater than the STAT3 EC₉₀ on activated human monocytes, alternatively greater than the Emax (EC₁₀₀) on activated human monocytes; and (b) less than the STAT3 EC₄₀ on activated human T cells, alternatively less than the STAT3 EC₃₀ on activated human T cells, alternatively less than the STAT3 EC₂₀ on activated human T cells, alternatively less than the STAT3 EC₁₀ on activated human T cells, alternatively less than the STAT3 EC₅ on activated human T cells. In some embodiments, the present disclosure provides a methods of treating a subject suffering from a disease disorder or condition by the administration of a therapeutically effective amount of za fused hIL10 polypeptide (or a pharmaceutically acceptable formulation containing a fused hIL10 polypeptide) wherein the therapeutically effective amount is a dose to provide a concentration that is: greater than the STAT3 EC₂₀ on activated human monocytes but less than the STAT3 EC₃₀ on activated human T cells; alternatively greater than the STAT3 EC₅₀ on activated human monocytes but less than the STAT3 EC₂₀ on activated human T cells; alternatively greater than the STAT3 EC₇₀ on activated human monocytes but less than the STAT3 EC₂₀ on activated human T cells; alternatively greater than the STAT3 EC₁₀ on activated human monocytes but less than the STAT3 EC₂₀ on activated human T cells; alternatively greater than the STAT3 Eₘₐₓ on activated human monocytes but less than the STAT3 EC₃₀ on activated human T cells, or alternatively greater than the STAT3 EC₅₀ on activated human monocytes but less than the STAT3 EC₁₀ on activated human T cells.

### Methods for Modulating IL10-Mediated Signaling

In another aspect, the disclosure provides methods for modulating IL10-mediated signaling in a subject. In some embodiments, the method comprises administering to the subject an effective amount of a pharmaceutical composition to the subject, where the pharmaceutical composition comprises a fused hIL10 polypeptide described herein, a nucleic acid molecule encoding a fused hIL10 polypeptide described herein, a recombinantly modified cell comprising a nucleic acid molecule encoding a fused hIL10 polypeptide described herein. In some embodiments, the pharmaceutical composition comprises a pharmaceutically acceptable carrier.

In some embodiments, the method for modulating IL10-mediated signaling in a subject comprises determining STAT3-mediated signaling in one or more cells obtained from the subject. In some embodiments, the STAT3-mediated signaling is determined by an assay selected from the group consisting of a gene expression assay, a phospho-flow signaling assay, and an enzyme-linked immunosorbent assay (ELISA). In some embodiments, the STAT3-mediated signaling in the subject is reduced by about 20% to about 100% compared to a reference level. In some embodiments, the administered composition results in a reduced capacity to induce expression of a pro-inflammatory gene selected from IFN-7, granzyme B, granzyme A, perforin, TNF-α, GM-CSF, and MIP1α in the subject.

### Kits

Also provided are kits comprising the fused hIL10 polypeptides of the disclosure. In some embodiments, the kit comprises one or more components for modulating IL10-mediated signaling in a subject, or treating a health condition in a subject in need thereof, wherein the components are selected from a fused hIL10 polypeptide described herein, a nucleic acid molecule encoding a fused hIL10 polypeptide described herein, a recombinantly modified cell comprising a nucleic acid molecule encoding a fused hIL10 polypeptide described herein, or a pharmaceutical composition comprising one of more the components. In some embodiments, the pharmaceutical composition of the kit comprises a pharmaceutically acceptable carrier.

### EXAMPLES

The following Examples are provided to illustrate, but not to limit the claimed invention.

### Example 1: Methods

### Protein Expression and Purification

Protein-coding DNA sequences were inserted into pEXSyn 2.0 between EcoRI and NotI restriction sites as C-terminal His-tag fusions with either the wild-type hIL10 signal peptide (AA seq: MHSSALLCCLVLLTGVRA (SEQ ID NO:98); DNA seq: ATGCACAGCAGCGCCCTGCTGTGCTGCCTGGTGCTGCTGACCGGCGTGAGAGCC (SEQ ID NO:125)) or the mouse IgG heavy chain signal peptide (AA seq: MGWSCIILFLVATATGVHS (SEQ ID NO:100) DNA seq: ATGGGATGGTCCTGCATCATCCTGTTTCTGGTGGCTACCGCCACCGGTGTGCACA GC (SEQ ID NO:126)) for mouse molecules.

The proteins were expressed in expi293 cells by transient transfection. The polypeptides containing the C-terminal His-tag were purified from harvested cell culture supernatants using Ni-affinity chromatography. Culture supernatants were supplemented with 5 mM Imidazole and were purified on gravity columns containing Ni-Excel (Cytiva) resin, equilibrated in PBS containing 5 mM Imidazole. Washes and elutions were carried out in PBS supplemented with 5 mM and 250 mM Imidazole, respectively. Proteins were dialyzed into PBS before storage and testing. Protein purity and quality was evaluated using SDS-PAGE, LCMS and analytical SEC.

### Protein PEGylation

Purified proteins were concentrated to 2-3 mg/ml in a 10 k Amicon (Millipore) in 100 mM Sodium Phosphate buffer pH 6.3, 100 mM NaCl. Protein was PEGylated at the N-terminus via aldehyde chemistry using a 20 kDa linear PEG (Sunbright® ME-200AL, NOF) for wild-type IL10 and a 40 kDa branched PEG (Sunbright® GL2-400AL3) for the fused molecules. The PEGylation reaction was carried out at room temperature with end-to-end rotation for 24-48 hours with addition of 5-fold molar excess of PEG over protein and 10 mM sodium cyanoborohydride (NaCNBH3, Sigma). PEGylation progress was evaluated by analytical SEC. The PEGylation reaction mixture was diluted 10-fold and purified over a cation exchange column (SP HP, Cytiva) with step-elution using 20 mM Sodium Phosphate pH 6.3, 0.7M NaCl to separate the protein from free PEG and NaCNBH3. The eluted protein contains a mix of unPEGylated, mono and multi-PEGylated protein species and was further purified by size exclusion chromatography with a Superose 6 increase column (Cytiva) equilibrated in Phosphate-buffered Saline (PBS). Elution peaks containing the PEGylated protein were pooled and concentrated to 0.5-2 mg/ml and sterile-filtered before use.

### Surface Plasmon Resonance

All experiments were conducted in 10 mM Hepes, 150 mM NaCl, 0.05% (v/v) Polysorbate 20 (PS20) and 3 mM EDTA (HBS-EP+ buffer) on a Biacore® T200 instrument equipped with Anti-human capture (AHC)-CM5 chip (Cytiva).

Human or mouse IL10RαFc (human: R&D systems #9044-RI; mouse: Sino biologicals #51298-MO2H) were captured on an AHC surface. Since IL10 analytes can be bivalent, ligand capture density was set to 12-25 RU to restrict Rmax to <8.5 RU (determined in a separate experiment) to measure binding kinetics under non-avid conditions. IL10 analytes were injected at 0.74, 2.22, 6.66, 20, 60, 180 nM in high performance mode (90 second association, 360 second dissocation) followed by surface regeneration with 3M MgCl2 (40 seconds, 30 L/min). Buffer-subtracted sensograms were processed with Biacore T200 Evaluation Software and globally fit with a 1:1 Langmuir binding model to extract kinetics and affinity constants (kₒₙ, k_{off}, K_{D}). Rₘₐₓ ranged from 4 to 8.5 RU, indicating surface density supporting true binding kinetics measurements in the absence of avidity. Affinities for all the molecules to IL10Rα were comparable to the native WT molecules, indicating that the T100L mutation or fusion did not affect binding to IL10Rα.

Binding to human or mouse IL10Rβ is not reported since the affinity of IL10 for IL10Rβ is expected to be weak (M to mM) and not detectable under these conditions.

### Thermal Stability Measurements

Proteins were formulated at −1 mg/mL in PBS and 0.1 m-filtered to remove aggregates. Each protein was loaded in 4 capillaries and dynamic light scattering (DLS) and turbidity were measured over a temperature ramp from 25 to 95 °C at 1 °C/min on a Prometheus Panta instrument. Unfolding data via differential scanning fluorimetry was not analyzed due to lack of tryptophans in IL10 molecules, which resulted in poor signal. The data indicate that the engineered molecules display equivalent thermal stability to the IL10 WT with aggregation onset (DLS) at ~52 °C.

### pH Stability Measurements

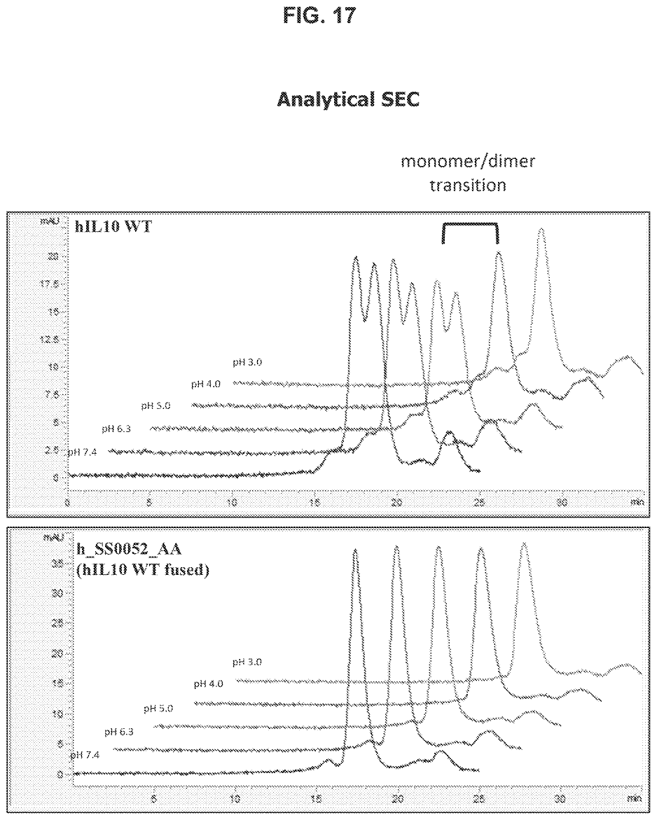

Proteins at ~1 mg/mL were dialyzed for 24 hours into 10 mM HEPES 150 mM NaCl pH 7.4 or 10 mM Sodium Acetate pH 3.0, 4.0, 5.0, or 6.3 with 150 mM NaCl. Following dialysis, proteins were tested by analytical SEC on a Superdex 200 Increase 10/300 GL column. The data in FIG. 17 indicate that the native IL10 molecule dissociated into monomers at pH<5.0 while the fused proteins remained stable.

TABLE 4
Human Fused IL10 Polypeptide Biacore Binding Data

| | | | | | | | Calc. | |
|---|---|---|---|---|---|---|---|---|
| | | k_{ON} | k_{OFF} | Affinity | Rmax | Load | Rmax | |
| Ligand | Analyte | (1/Ms) | (1/s) | (nM) | (RU) | (RU) | (RU) | Activity |
| hIL10RαFc | hIL10 WT | 2.42E+06 | 1.51E−04 | 0.06 | 7.6 | 22.5 | 12.5 | 61% |
| (R&D | hIL10 T100L | 1.22E+06 | 3.16E−04 | 0.26 | 7.5 | 23.4 | 13.0 | 58% |
| 9044-RI) | h_SS0052_AA | 2.32E+06 | 2.61E−04 | 0.11 | 8.5 | 23.7 | 13.2 | 64% |
| | (hIL10 WT | | | | | | | |
| | fused) | | | | | | | |
| | h_SS0053_AA | 3.10E+06 | 2.27E−04 | 0.07 | 8.5 | 23.6 | 13.1 | 65% |
| | (hIL10 T100L | | | | | | | |
| | fused) | | | | | | | |

TABLE 5
Mouse Fused IL10 Polypeptide Biacore Binding Data

| | | | | | | | Calc. | |
|---|---|---|---|---|---|---|---|---|
| | | k_{ON} | k_{OFF} | Affinity | Rmax | Load | Rmax | |
| Ligand | Analyte | (1/Ms) | (1/s) | (nM) | (RU) | (RU) | (RU) | Activity |
| mIL10RαFc | mIL10 WT | 1.44E+07 | 1.16E−02 | 0.81 | 5.4 | 12.8 | 7.1 | 76% |
| (Sino 51298- | mIL10 T100L | 4.78E+06 | 9.20E−03 | 1.92 | 5.5 | 13.1 | 7.3 | 75% |
| M02H) | WC163_AA | 4.70E+06 | 4.04E−03 | 0.86 | 5.3 | 13.3 | 7.4 | 72% |
| | (mIL10WT fused) | | | | | | | |
| | SS0051_AA (mIL10 | 7.85E+06 | 5.75E−03 | 0.73 | 6 | 13.3 | 7.4 | 81% |
| | T100L fused) | | | | | | | |

### Example 2: pSTAT3 Flow Cytometric Assay

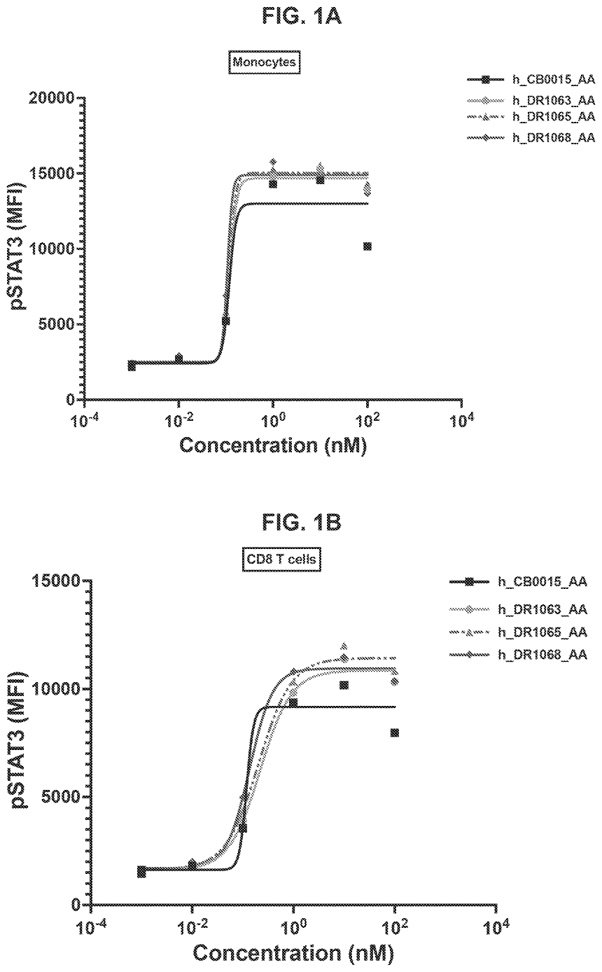

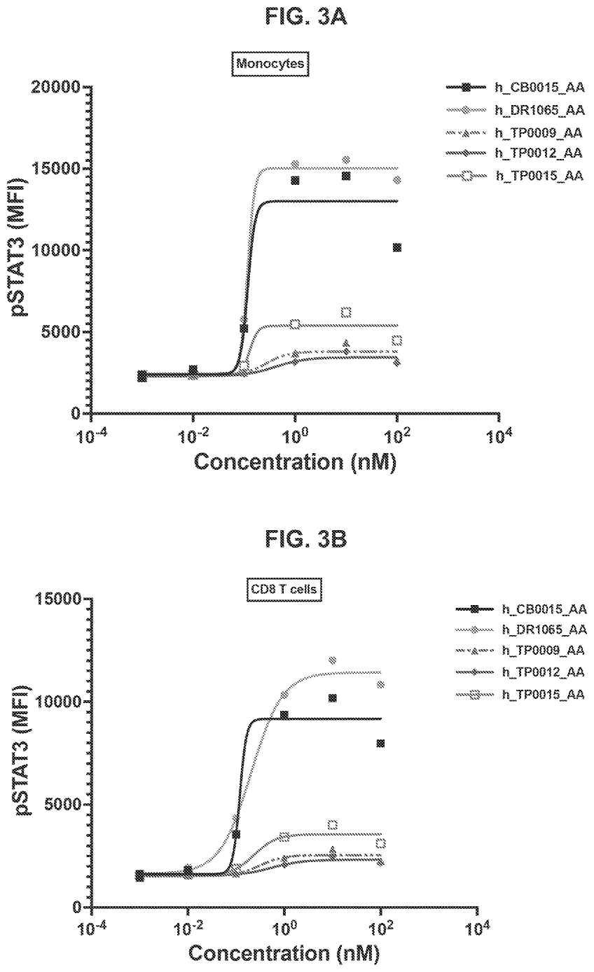

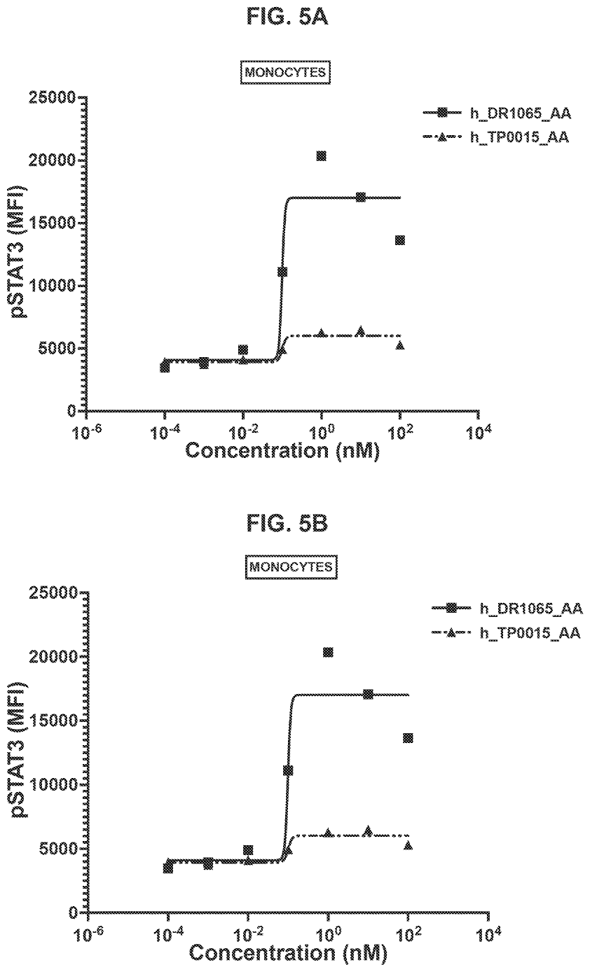

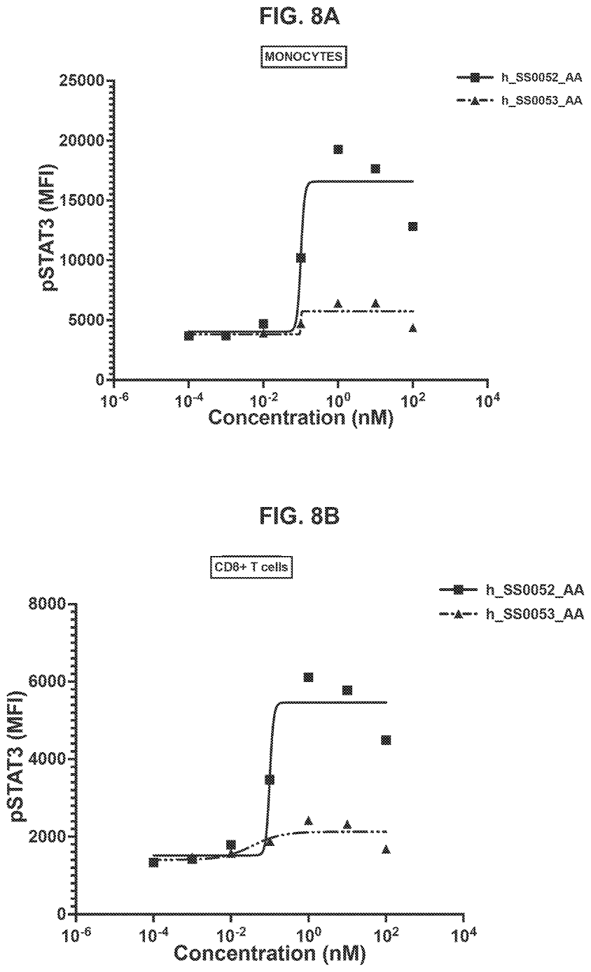

PBMCs were purified from healthy non-smoking donor blood collected in leukoreduction system (LRS) chambers using the human Miltenyi MACSprep™ PBMC isolation kit. The purified PBMCs (500,000 cells per well) were stimulated with IL10 proteins at concentrations ranging from 0.1 pM-100 nM for 20 min at 37 °C. The cells were fixed with BD Phosflow™ Fix Buffer I (BD Biosciences, Catalog No. 557870) for 15 mins at 37 °C. The cells were then washed and permeabilized with chilled BD Phosflow™ Perm Buffer III (Catalog No. 558050) overnight at −20 °C. The cells were washed to remove the permeabilization buffer and blocked with Human TruStain FcX (Biolegend, Catalog No. 422301) and mouse serum for 5 minutes at room temperature. The cells were then treated with the antibody cocktail (listed in Table 6 below) for 1 hour at room temperature. Following antibody staining, the cells were washed, fixed and ran on the Cytek® Aurora Spectral flow cytometer. The data was analyzed using the FlowJo software (www.flowjo.com). The various cell lineages were gated using their lineage markers and the geometric mean fluorescence intensity of pSTAT3 expression was calculated on FlowJo. Fusion WT molecules have a slightly higher pSTAT3 Emax as compared to the native WT IL10 in both monocytes and CD8 T cells. The data for pSTAT flow cytometric assay are shown in FIGS. 1-5 and 8.

TABLE 6

| | | Phosphor- | | | |
|---|---|---|---|---|---|
| Fluoro- | Anti- | ylation | | | |
| chrome | body | Site | Clone | Catalog# | Vendor |
| BV480 | CD4 | | L200 | 566148 | BD |
| | | | | | Biosciences |
| BV605 | CD8a | | RPA-T8 | 301040 | Biolegend |
| BV786 | CD56 | | 5.1H11 | 362550 | Biolegend |
| AF488 | pSTAT3 | Tyr705 | D3A7 | 4323S | CST |
| PE-Cy5 | CD3 | | UCHT1 | 555334 | BD |
| | | | | | Biosciences |
| PerCP-Cy | CD14 | | M5E2 | 301824 | Biolegend |
| 5.5 | | | | | |
| APC | CD20 | | 1412 | 340516 | Biolegend |

### Example 3: Monocyte IL10 Activity Assay

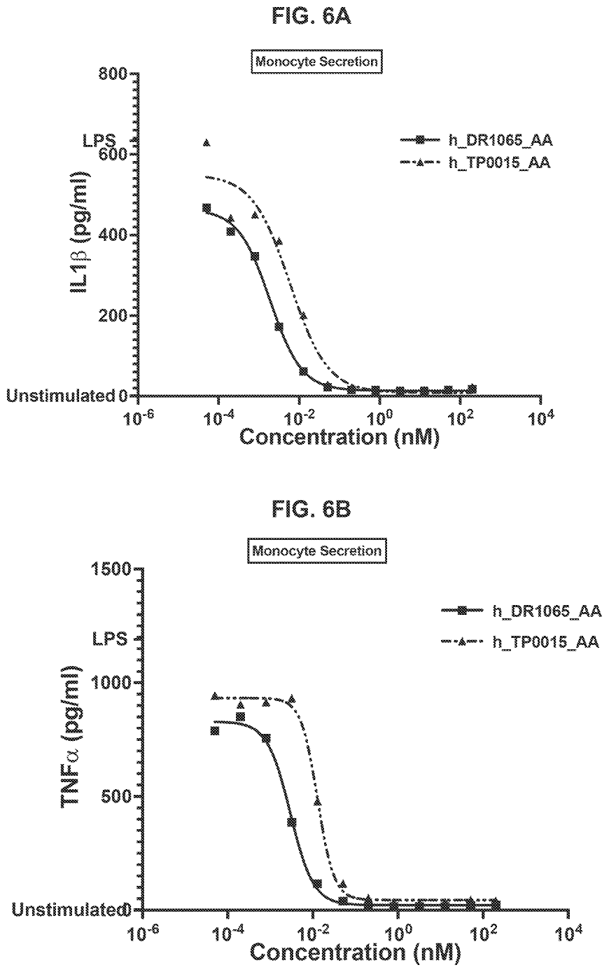

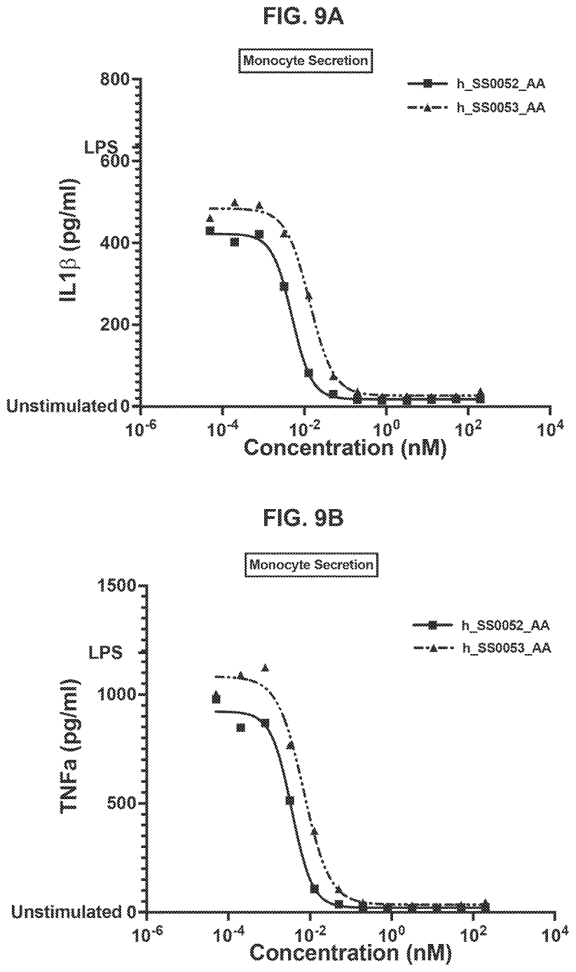

Human PBMCs were isolated from LRS chambers obtained from non-smoking human donors by Ficoll® gradient purification. Human monocytes were purified from the PBMCs by positive selection using Human CD14 MicroBeads (Miltenyi Biotech, Catalog No. 130-050-201) following manufacturer's protocol. Monocytes were treated with 1 ng/ml of LPS (Millipore Sigma, Catalog No. L2630) in the presence of varying concentrations of the IL10 proteins (0.05 pM-200 nM) for 48 hours at 37 °C. Post-treatment, cells were centrifuged at 400 g for 5 min and supernatants were collected. Supernatants were assessed for pro-inflammatory cytokines using the MSD kit (Meso Scale Discovery, Catalog No. K151A9H-4) in substantial accordance with the manufacturer's protocol. The data was graphed using Prism software (www.prismsoftware.com). The cytokine levels under unstimulated and LPS-alone treated conditions are marked on the Y-axis as unstimulated and LPS. The data for monocyte IL10 activity assay are shown in FIGS. 6A and 6B, and 9A and 9B. As illustrated by the data provided the fused molecule with the T100L mutation inhibits LPS-induced secretion of IL-1β and TNF-α by monocytes.

### Example 4: CD8 T Cell Blasts IL10 Activity Assay

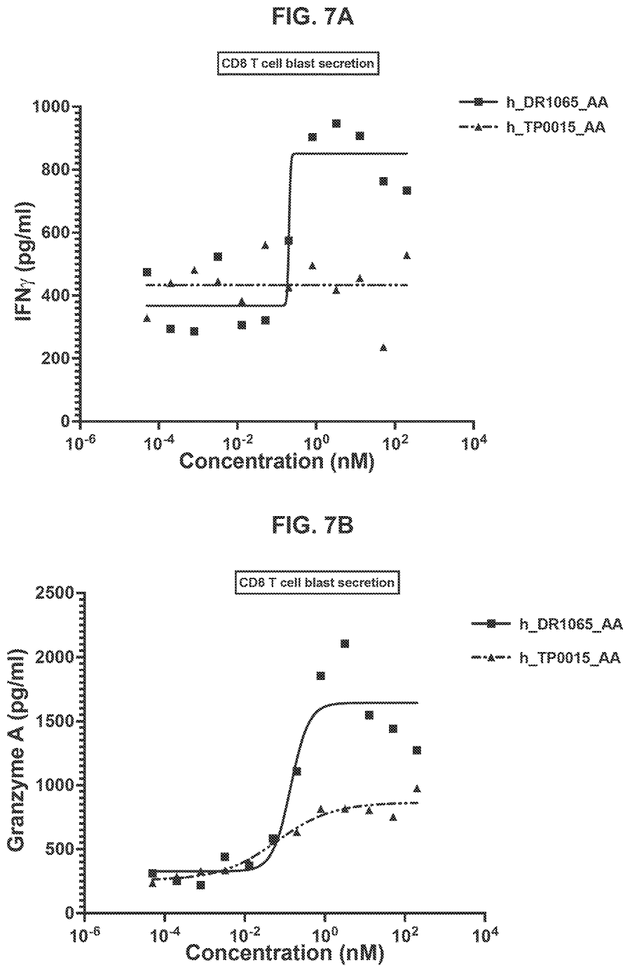

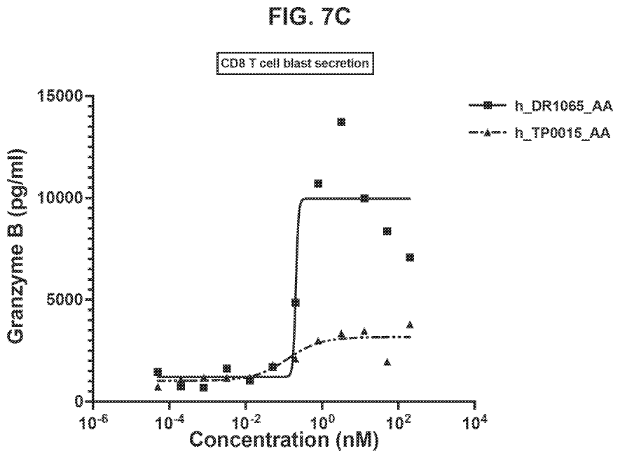

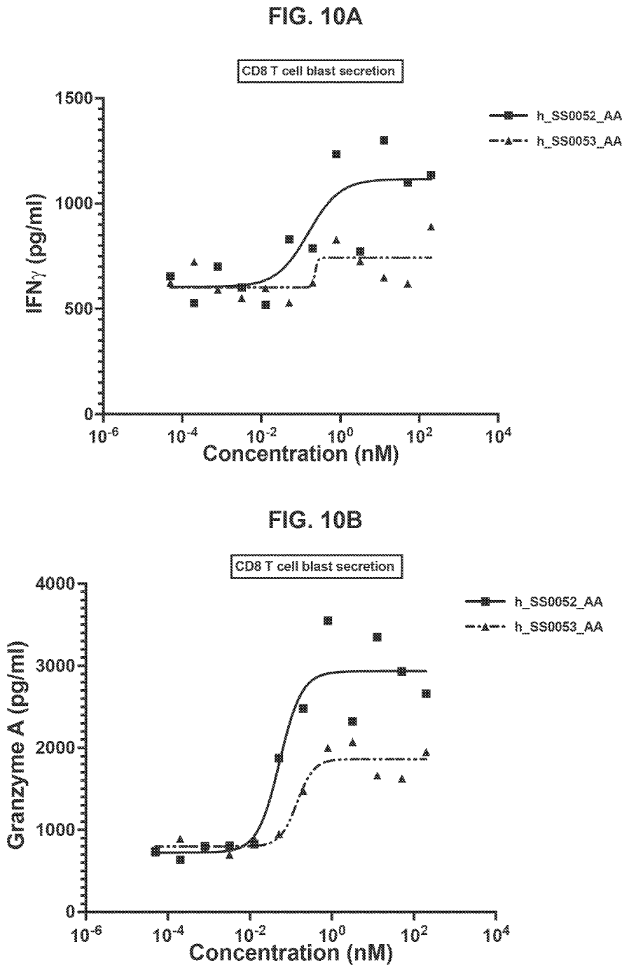

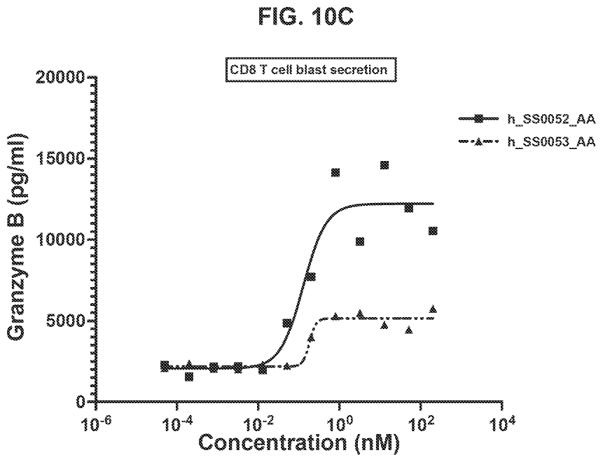

Human PBMCs were isolated from LRS chambers obtained from non-smoking human donors by Ficoll gradient purification. Human CD8 T cells were purified from the PBMCs by negative selection using the CD8+ T Cell Isolation Kit (Miltenyi Biotech-130-096-495) in substantial accordance with the manufacturer's protocol. Isolated CD8 T cells were activated using the human T Cell Activation/Expansion Kit (Miltenyi Biotech, Catalog No. 130-091-441) in the presence of 100 pM human IL2 for 72 hours at 37 °C. The activated CD8 T cells were washed and the beads were removed magnetically following which the cells were treated with varying concentrations of the IL10 proteins (0.05 pM-200 nM) for 72 hours at 37 °C. Post-treatment, cells were centrifuged at 400 g for 5 min and the supernatants were collected. Supernatants were assessed for the cytokines IFNγ, Granzyme A and Granzyme B using the MSD kit (Meso Scale Discovery, Catalog No. K151AEL-4) in substantial accordance with the manufacturer's protocol. The data was graphed using Prism software. The data for CD8 T cell blasts IL10 activity assay are shown in FIGS. 7A-7C and 10A-10C. As illustrated in the Figures, the fused molecule with the T100L mutation is weaker at inducing IFNγ, Granzyme A and Granzyme B by CD8 T cell blasts and does not achieve Emax levels equivalent to the Fused-WT up to 100 nM concentration.

### Example 5: Mouse pSTAT3 Flow Cytometric Assay

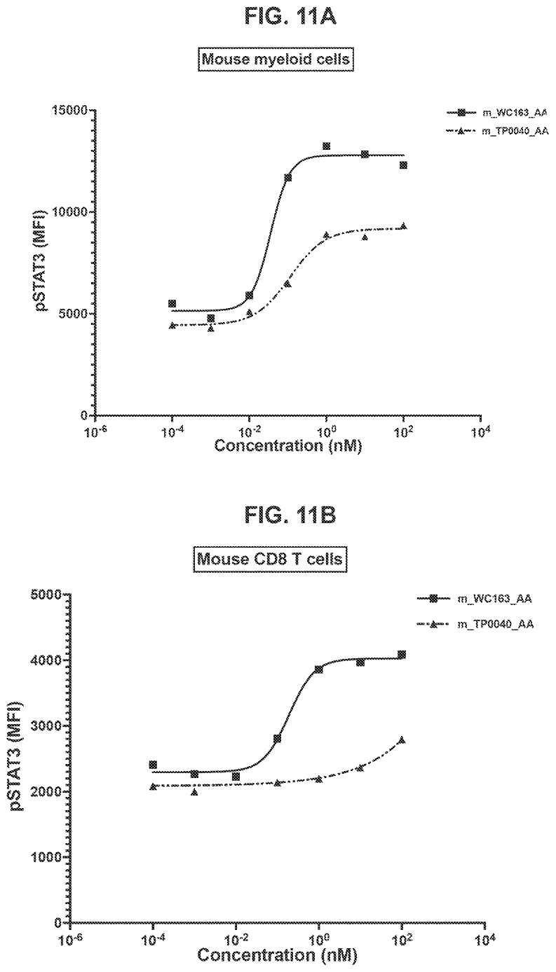

Spleens were removed from naïve C57BL/6 mice. Splenocytes were isolated by crushing the spleens with the plunger end of a syringe in complete RPMI medium (Gibco RPMI 1640+10% FBS+1% penicillin-streptomycin) and straining through a 70 μM strainer. The cells were centrifuged at 400 g for 5 min and the supernatant was discarded. The cell pellet was resuspended in 10 ml ammonium-chloride-potassium (ACK) lysis buffer (ThermoFisher) and allowed to sit at 37 °C for 2 min. The cells were washed and resuspended in complete RPMI. The purified splenocytes (500,000 cells per well) were stimulated with IL10 proteins at concentrations ranging from 0.1 pM-100 nM for 20 min at 37 °C. The cells were fixed with BD Phosflow™ Fix Buffer I (BD Biosciences, Catalog No. 557870) for 15 mins at 37 °C. The cells were then washed and permeabilized with chilled BD Phosflow™ Perm Buffer III (BD Biosciences, Catalog No. 558050) overnight at −20 °C. The cells were washed to remove the permeabilization buffer and blocked with Mouse Trustain Fcx (Biolegend, Catalog No. 101320) for 5 minutes at room temperature. The cells were then treated with the antibody cocktail (listed in Table 7 below) for 1 hour at room temperature. Following antibody staining, the cells were washed, fixed and ran on the Cytek® Aurora Spectral flow cytometer. The data was analyzed using the FlowJo software. The various cell lineages were gated using their lineage markers and the geometric mean fluorescence intensity of pSTAT3 expression was calculated on FlowJo. The data for mouse pSTAT3 flow cytometric assay are shown in FIGS. 11A and 11B.

TABLE 7

| | | Phosphor- | | | |
|---|---|---|---|---|---|
| Fluoro- | Anti- | ylation | | | |
| chrome | body | Site | Clone | Catalog# | Vendor |
| BV480 | CD4 | | RM4-5 | 565634 | BD Biosciences |
| BV711 | CD8a | | 53-6.7 | 563046 | BD Biosciences |
| BV750 | CD11b | | M1/70 | 746910 | BD Biosciences |
| BV786 | CD3 | | 17A2 | 564010 | BD Biosciences |
| AF488 | pSTAT3 | Tyr705 | D3A7 | 4323S | CST |

### Example 6: Mouse Splenocyte LPS Assay

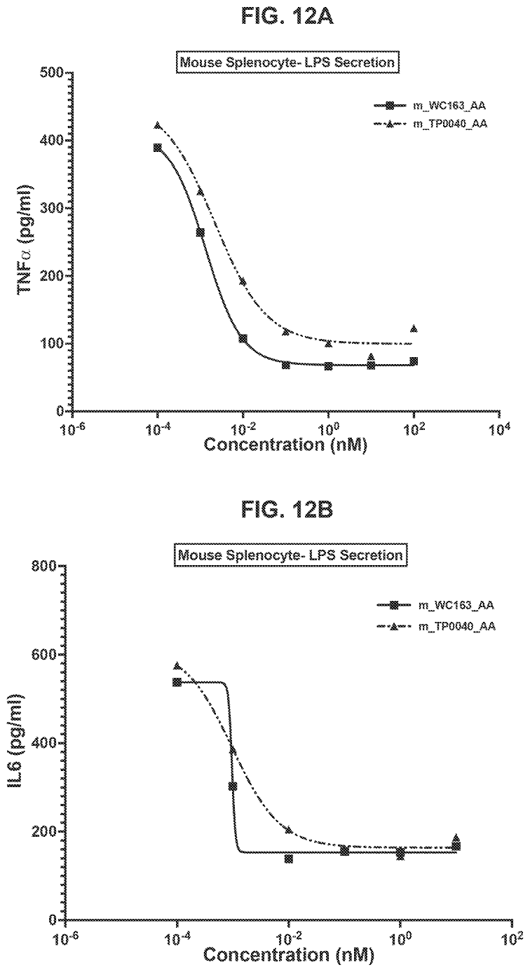

Spleens were removed from naïve C57BL/6 mice. Splenocytes were isolated by crushing the spleens with the plunger end of a syringe in complete RPMI medium (Gibco RPMI 1640 medium+10% FBS+1% penicillin-streptomycin) and straining through a 70 μM strainer. The cells were centrifuged at 400 g for 5 min and the supernatant was discarded. The cell pellet was resuspended in 10 ml ACK lysis buffer and allowed to sit at 37 °C for 2 min. The cells were washed and resuspended in complete RPMI. The purified splenocytes (500,000 cells per well) were treated with 1 ng/ml of LPS (Millipore Sigma, Catalog No. L2630) in the presence of varying concentrations of the IL10 proteins (0.1 pM-100 nM) for 48 hours at 37 °C. Post-treatment, cells were centrifuged at 400 g for 5 min and supernatants were collected. Supernatants were assessed for pro-inflammatory cytokines using the MSD kit (Meso Scale Discovery, Catalog No. K152A0H-2) in substantial accordance with the manufacturer's protocol. The data as shown in FIGS. 12A and 12B was graphed using Prism software.

### Example 7: Mouse CD8 T Cell Blast Assay

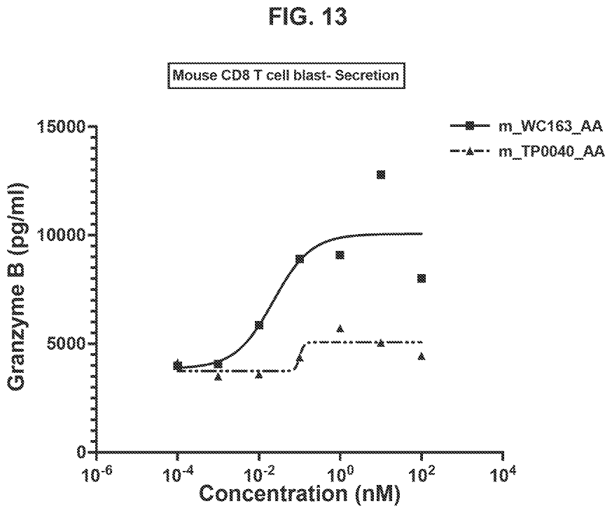

Spleens were removed from naïve C57BL/6 mice. Splenocytes were isolated by crushing the spleens with the plunger end of a syringe in complete RPMI medium (Gibco RPMI 1640+10% FBS+1% penicillin-streptomycin) and straining through a 70 μM strainer. The cells were centrifuged at 400 g for 5 min and the supernatant was discarded. The cell pellet was resuspended in 10 ml ACK lysis buffer and allowed to sit at 37 °C for 2 min. The cells were washed and resuspended in complete RPMI. Mouse CD8 T cells were purified from the PBMCs by negative selection using a kit (Miltenyi Biotech, Catalog No. 130-104-075) following manufacturer's protocol. Isolated CD8 T cells were activate using the Mouse T cell Activation and Expansion Kit (Miltenyi Biotech-130-093-627) in the presence of 100 pM mouse IL2 for 72 hours at 37 °C. The blasted CD8 T cells were washed, the beads were removed magnetically following which the cells were treated with varying concentrations of the IL10 proteins (0.1 pM-100 nM) in the presence of 100 pM mouse IL2 for 72 hours at 37 °C. Post-treatment, cells were centrifuged at 400 g for 5 min and supernatants were collected. Supernatants were assessed for cytokines (Granzyme B) using the MSD kit (Meso Scale Discovery, Catalog No. K1523XR-2) in substantial accordance with the manufacturer's protocol. The data was graphed using Prism software. The data for mouse CD8 T cell blasy assay are shown in FIG. 13. The fused molecule with the T100L mutation is weaker at inducing IFNγ, Granzyme A and Granzyme B by CD8 T cell blasts and does not achieve Emax levels equivalent to the Fused-WT up to 100 nM concentration.

### Example 8: Mouse CD8 T Cell Blast Survival Assay

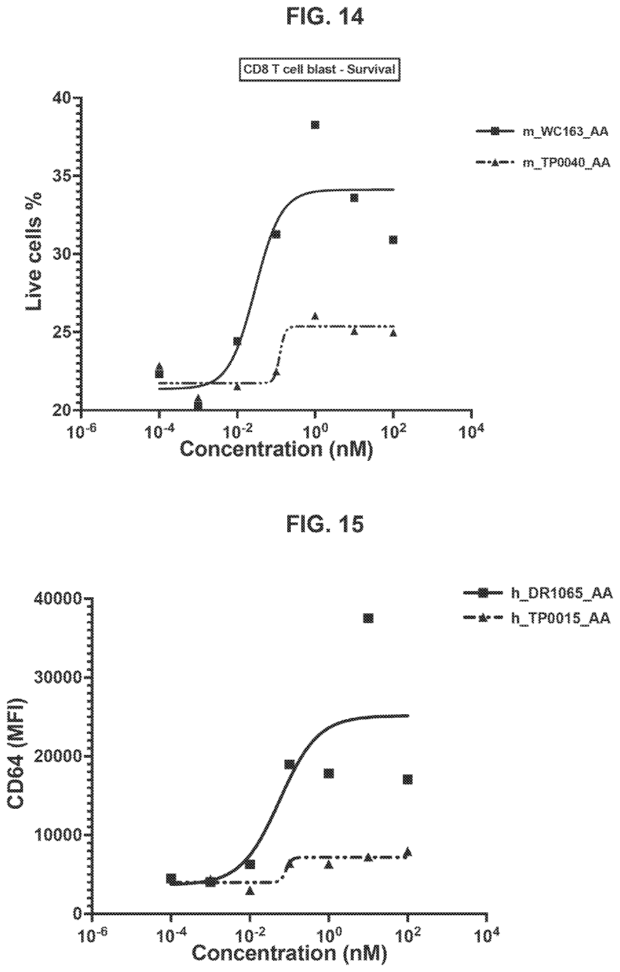

Spleens were removed from naïve C57BL/6 mice. Splenocytes were isolated by crushing the spleens with the plunger end of a syringe in complete RPMI medium (RPMI+10% FBS+1% Pennicillin-Streptomycin) and straining through a 70 μM strainer. The cells were centrifuged at 400 g for 5 min and the supernatant was discarded. The cell pellet was resuspended in 10 ml ACK lysis buffer and allowed to sit at 37 °C for 2 min. The cells were washed and resuspended in complete RPMI. Mouse CD8 T cells were purified from the PBMCs by negative selection using a kit (Miltenyi Biotech, Catalog No. 130-104-075) in substantial accordance with the manufacturer's protocol. 200,000 CD8 T cells were added to each well of a 96-well plate pre-coated with 2.5 μg/ml of anti mouse CD3E antibody (Biolegend 100302). The cells were then treated with IL10 proteins ranging in concentration from 0.1 pM to 100 nM in the presence of 5 μg/ml of anti mouse CD28 antibody (Biolegend 102102) for 72 hours at 37 °C. The cells were then centrifuged at 400 g for 5 min and the supernatant was discarded. Cells were stained for Annexin V and Propidium Iodide using the Dead Cell Apoptosis Kits with Annexin V for Flow Cytometry (Invitrogen, Catalog No. V13241) in substantial accordance with the manufacturer's protocol. The data was analyzed using the FlowJo software. Following staining, the cells were run on the Cytek Aurora Spectral flow cytometer. The data was analyzed using the FlowJo software. The data as shown in FIG. 14 was graphed using Prism software. Mouse fused-T100L mutation is weaker at inducing survival of CD8 T cell blasts and does not achieve Emax levels equivalent to the fused-WT up to 100 nM concentration.

### Example 9: Monocyte Cell Surface CD64 Staining

Human PBMCs were isolated from LRS chambers obtained from non-smoking human donors by Ficoll gradient purification. Human Monocytes were purified from the PBMCs by positive selection using Human CD14 Microbeads (Miltenyi Biotech, Catalog No. 130-050-201) following manufacturer's protocol. Monocytes were treated with varying concentrations of the fused IL10 polypeptides (0.1 pM-100 nM) for 48 hours at 37 °C. Post-treatment, cells were spun down at 400 g for 5 min. The cells were then washed and blocked with Human Trustain Fcx (Biolegend, Catalog No. 422301) and mouse serum for 15 minutes at 4 °C. The cells were then treated with CD64 antibody (Biolegend, Catalog No. 305006) for 30 min at 4 °C. Following antibody staining, the cells were washed, fixed and ran on the Cytek Aurora Spectral flow cytometer. The data was analyzed using the FlowJo software. The data was graphed using Prism software. The fused polypeptide with T100L mutation (TP0015) exhibited in lower induction of cell surface CD64 on monocytes as compared to the native dimeric IL10 molecule (FIG. 15).

### Example 10: PK Study

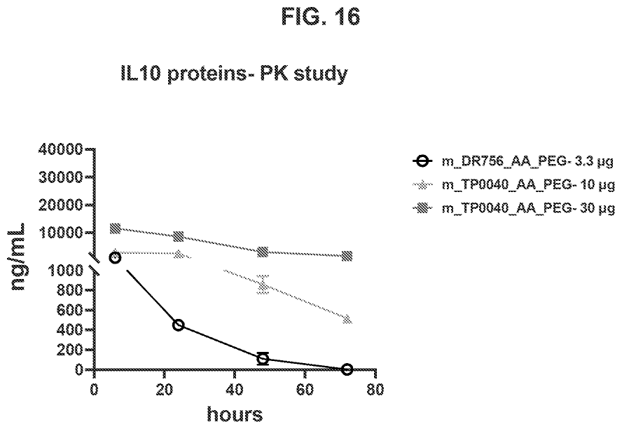

Fused IL-10 polypeptides produced in substantial accordance with the teaching of Example 9 were subcutaneously injected into 10 week old C57BL/6J female mice (RRID:IMSR_JAX:000664) at indicated doses. Blood was collected via submandibular bleeds and serum was prepared using serum microtubes (Sarstedt, 41.1378.005). Serum IL-10 concentration was measured using the MSD kit (Meso Scale Discovery, K15069M). The concentrations were interpolated using four parameter (4PL) model in GraphPad Prism 9.3.1 software. The data for the PK study are shown in FIG. 16. The fused-T100L had a better exposure compared to native WT IL-10 upto 72 hours post a single dose injection of the molecules.

### Example 11: Evaluation of Species Cross-Reactivity, Mouse Splenocyte LPS Assay

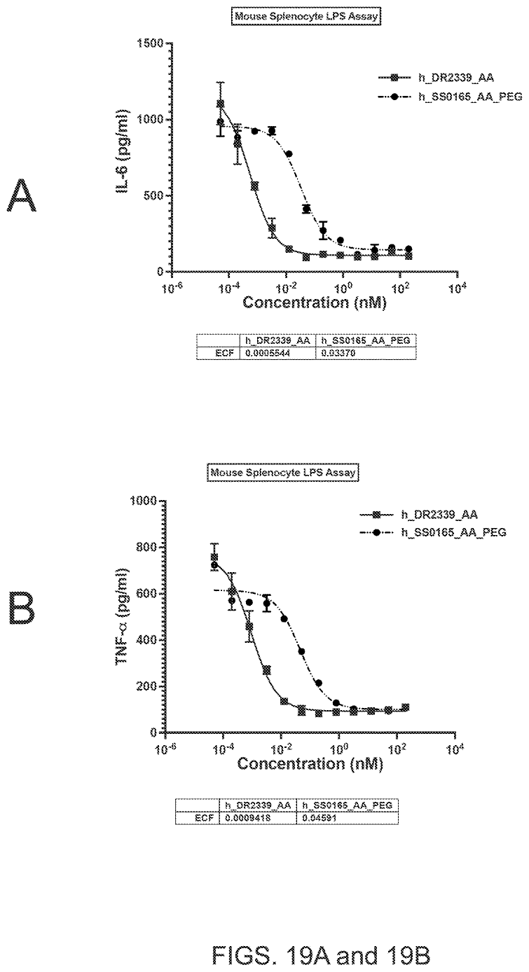

To evaluate species cross reactivity of the human IL10 molecules in mice, spleens were removed from naïve C57BL/6 mice. Splenocytes were isolated by mashing the spleens with the plunger end of a syringe in complete RPMI medium (RPMI+10% FBS+1% Pennicillin-Streptomycin) and straining through a 70 μM strainer. The cells were spun down at 400 g for 5 min and the supernatant was discarded. The cell pellet was resuspended in 10 ml ACK lysis buffer and allowed to sit at 37 °C for 2 min. The cells were washed and resuspended in complete RPMI. The purified splenocytes (500,000 cells per well) were treated with 1 ng/ml of LPS (Millipore Sigma: L2630) in the presence of varying concentrations of the IL10 proteins (0.05 pM-200 nM) for 48 hours at 37 °C. Post-treatment, cells were spun down at 400 g for 5 min and supernatants were collected. Supernatants were assessed for pro-inflammatory cytokines using the MSD kit (Meso Scale Discovery) following manufacturer's protocol. The data was graphed using Prism software. The data is presented in FIGS. 19A and 19B of the attached drawings. Human STK-030 (h_SS0165_AA_PEG; SEQ ID NO:123) shows good cross-species reactivity to mouse as assessed by inhibition of LPS-induced secretion of IL-6 and TNF-α.

### Example 12: Evaluation of Granzyme B Secretion from Mouse CD8 T Cell Blasts

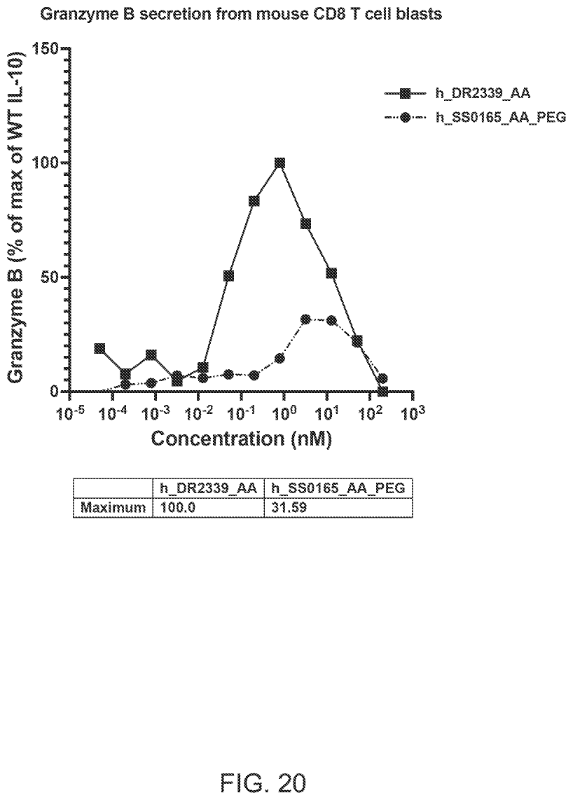

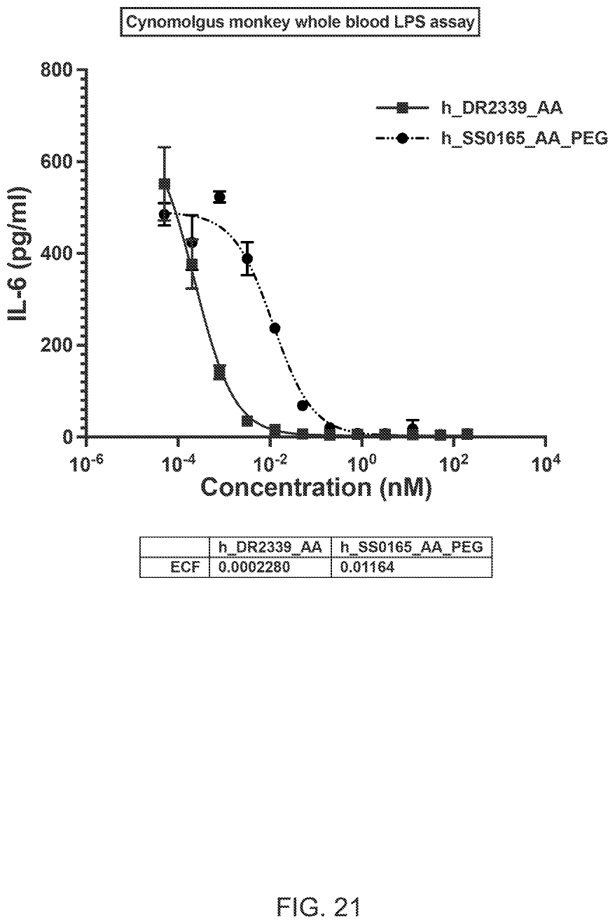

Spleens were removed from Naïve C57BL/6 mice. Splenocytes were isolated by mashing the spleens with the plunger end of a syringe in complete RPMI medium (RPMI+10% FBS+1% Pennicillin-Streptomycin) and straining through a 70 μM strainer. The cells were spun down at 400 g for 5 min and the supernatant was discarded. The cell pellet was resuspended in 10 ml ACK lysis buffer and allowed to sit at 37 °C for 2 min. The cells were washed and resuspended in complete RPMI. Mouse CD8 T cells were purified from the PBMCs by negative selection using a kit (Miltenyi Biotech-130-104-075) following manufacturer's protocol. Isolated CD8 T cells were blasted using the Mouse T cell activation and expansion kit (Miltenyi Biotech-130-093-627) in the presence of 100 pM mouse IL2 for 72 hours at 37 °C. The blasted CD8 T cells were washed and the cells were treated with varying concentrations of the IL10 proteins (0.05 pM-200 nM) in the presence of 100 pM mouse IL2 for 72 hours at 37 °C. Post-treatment, cells were spun down at 400 g for 5 min and supernatants were collected. Supernatants were assessed for cytokines (Granzyme B) using the MSD kit (Meso Scale Discovery: K1523XR-2) following manufacturer's protocol. The data was graphed using Prism software. The data is presented in FIG. 20 of the attached drawings. Data is expressed as percentage of maximum secretion by WT IL-10 (h_DR2339_AA). Human STK-030 (h_SS0165_AA_PEG; SEQ ID NO:123) is weaker at inducing Granzyme B secretion, reaching only a maximum of 32% Granzyme B secretion achieved by that of WT IL-10. hSTK-030 shows a myeloid-bias in function in mouse assays, similar to that observed in human cell assays.

### Example 13: Evaluation of Species Cross Reactivity (CYNO)

100 μl of Li-Heparin blood from Cynomolgus monkey was treated with 1 ng/ml of LPS (Millipore Sigma: L2630) in the presence of varying concentrations of the IL10 proteins (0.05 pM-200 nM) for 48 hours at 37 °C. Post-treatment, samples were spun down at 400 g for 5 min and supernatants were collected. Supernatants were assessed for pro-inflammatory cytokines using the MSD kit (Meso Scale Discovery:) following manufacturer's protocol. The data was graphed using Prism software. The data is provided in FIG. 21 of the attached drawings. Human STK-030 (h_SS0165_AA_PEG; SEQ ID NO:123) shows good cross-species reactivity to cynomolgus monkey as assessed by inhibition of LPS-induced secretion of IL-6.

### Example 14: In Vivo Evaluation In DSS Model of Ulcerative Colitis

The IL10 variants of the present disclosure were evaluated for therapeutic efficacy in a murine DSS model of ulcerative colitis. The murine DSS-induced colitis model is widely used and possess many similarities with human ulcerative colitis. Chassaing, B., et al. (2014) Current Protocols in Immunology 104:15.25.1-15.25.14. CD163

The canonical sequence of mature (absent the endogenous 18 amino acid signal peptide) wild-type murine IL10 molecule (UniProt Reference P18893) is a 160 amino acid polypeptide having the amino acid sequence:

(SEQ ID NO: 187)
SRGQYSREDNNCTHFPVGQSHMLLELRTAFSQVKTFFQTKDQLDNILLTD
SLMQDFKGYLGCQALSEMIQFYLVEVMPQAEKHGPEIKEHLNSLGEKLKT
LRMRLRRCHRFLPCENKSKAVEQVKSDFNKLQDQGVYKAMNEFDIFINCI
EAYMMIKMK

When referencing amino acid substitutions or deletions in the murine IL10 variant molecules described herein are made in reference to the canonical wild-type mature mIL10 polypeptide (SEQ ID NO:187).

To perform in vivo experimentation in the mouse DSS model, murine surrogates of human IL10 variants were used. The following Table 8 describes the pegylated murine IL10 (mIL10) test articles were used in the DSS study. m_DR756_AA was used as the murine surrogate for the wild-type molecule and exhibits substantially equivalent activity to the wild-type mIL10. m_DR2677_AA is a fused mIL10 dimer comprising substantially mIL10 wild-type sequences with the exception of the deletion of the 3 N-terminal amino acids (ΔS1/ΔR2/ΔG3) and a Q4E amino acid substitution to avoid pyroglutamate formation and facilitate N-terminal PEGylation, m_DR2678_AA is a fused mIL10 dimer comprising IL10 variants having the T100L mutation, again comprising a deletion of the 3 N-terminal amino acids (ΔS1/ΔR2/ΔG3) and a Q4E amino acid substitution to avoid pyroglutamate formation and facilitate N-terminal PEGylation.

TABLE 8
DSS Study Murine Test Articles

| Name | Polypeptide | PEGylation |
|---|---|---|
| m_DR756_AA_PEG | m_DR756_AA | N-terminal 20 kDa |
| | | Linear PEG |
| m_DR2677_AA_PEG | m_DR2677_AA | N-terminal 40 kDa |
| | | PEG branched |
| | | (2 × 20) PEG |
| m_DR2677_AA_PEG | m_DR2678_AA | N-terminal 40 kDa |
| | | PEG branched |
| | | (2 × 20) PEG |

The amino acid sequences of the parent polypeptides of the m_DR756_AA_PEG, m_DR2677_AA_PEG, m_DR267_AA_PEG test articles are provided in Table 9 below:

TABLE 9
Parent Polypeptides of PEGylated Test Articles

| SEQ ID# | Name | Description | hIL10 Mutein Monomer Sequences |
|---|---|---|---|
| 188 | m_DR756_AA: | Alanine; 8xHis chelating | AHHHHHHHHGSQYSREDNNCTHFP |
| | (WT IL-10) | peptide (SEQ ID NO: | VGQSHMLLELRTAFSQVKTFFQTKD |
| | | 146); Gly-Ser linker; | QLDNILLTDSLMQDFKGYLGCQALS |
| | | ΔS1/ΔR2/ΔG3 mIL10 | EMIQFYLVEVMPQAEKHGPEIKEHL |
| | | | NSLGEKLKTLRMRLRRCHRFLPCEN |
| | | | KSKAVEQVKSDFNKLQDQGVYKAM |
| | | | NEFDIFINCIEAYMMIKMKS (SEQ ID |
| | | | NO: 188) |
| 189 | m_DR2677_AA: | [ΔS1/ΔR2/ΔG3/Q4E | EYSREDNNCTHFPVGQSHMLLELRT |
| | (Fused WT IL-10) | mIL10]-[wt mIL10 1- | AFSQVKTFFQTKDQLDNILLTDSLM |
| | | 160]-GGS linker-His8 | QDFKGYLGCQALSEMIQFYLVEVMP |
| | | | QAEKHGPEIKEHLNSLGEKLKTLRM |
| | | | RLRRCHRFLPCENKSKAVEQVKSDF |
| | | | NKLQDQGVYKAMNEFDIFINCIEAY |
| | | | MMIKMKSSRGQYSREDNNCTHFPV |
| | | | GQSHMLLELRTAFSQVKTFFQTKDQ |
| | | | LDNILLTDSLMQDFKGYLGCQALSE |
| | | | MIQFYLVEVMPQAEKHGPEIKEHLN |
| | | | SLGEKLKTLRMRLRRCHRFLPCENK |
| | | | SKAVEQVKSDFNKLQDQGVYKAMN |
| | | | EFDIFINCIEAYMMIKMKSGGSHHHH |
| | | | HHHH (SEQ ID NO: 189) |
| 190 | m_DR2678_AA: | [ΔS1/ΔR2/ΔG3/Q4E/ | EYSREDNNCTHFPVGQSHMLLELRT |
| | (Fused T100L) | T100L mIL10]-[T100L | AFSQVKTFFQTKDQLDNILLTDSLM |
| | | mIL10 1-160]-GGS | QDFKGYLGCQALSEMIQFYLVEVMP |
| | | linker; 8xHis chelating | QAEKHGPEIKEHLNSLGEKLKLLRM |
| | | peptide (SEQ ID NO: | RLRRCHRFLPCENKSKAVEQVKSDF |
| | | 146) | NKLQDQGVYKAMNEFDIFINCIEAY |
| | | | MMIKMKSSRGQYSREDNNCTHFPV |
| | | | GQSHMLLELRTAFSQVKTFFQTKDQ |
| | | | LDNILLTDSLMQDFKGYLGCQALSE |
| | | | MIQFYLVEVMPQAEKHGPEIKEHLN |
| | | | SLGEKLKLLRMRLRRCHRFLPCENK |
| | | | SKAVEQVKSDFNKLQDQGVYKAMN |
| | | | EFDIFINCIEAYMMIKMKSGGSHHHH |
| | | | HHHH (SEQ ID NO: 190) |

Briefly, the DSS study was conducted as follows. Female C571/6 mice (Jackson Laboratories) were administered 2.500 DSS, ad libitum, in drinking water on study days 0-6. Mice given normal water served as a negative disease control. Mice were treated with either PBS, m_DR2677_AA-PEG (3 or 10 micrograms administered subcutaneously every other day) or m_DR2678_AA-PEG (3 or 10 micrograms administered subcutaneously every other day) in accordance with the following Table 10.

TABLE 10
DSS Study Treatment Groups

| Group | DSS | Treatment | Dose [ug] | Schedule |
|---|---|---|---|---|
| A | Water | PBS | — | — |
| B | 2.5% | PBS | — | D 0, D 2, D 4, |
| | | | | D 6, D 8 |
| C | 2.5% | m_DR756_AA_PEG | 3 | D 0, D 2, D 4, |
| | | | | D 6, D 8 |
| D | 2.5% | m_DR756_AA_PEG | 10 | D 0, D 2, D 4, |
| | | | | D 6, D 8 |
| E | 2.5% | m_DR2677_AA_PEG | 3* | D 0, D 2, D 4, |
| | | | | D 6, D 8 |
| F | 2.5% | m_DR2677_AA_PEG | 10** | D 0, D 2, D 4, |
| | | | | D 6, D 8 |
| G | 2.5% | m_DR2678_AA_PEG | 3 | D 0, D 2, D 4, |
| | | | | D 6, D 8 |
| H | 2.5% | m_DR2678_AA_PEG | 10 | D 0, D 2, D 4, |
| | | | | D 6, D 8 |

*On Day 0, mice in Treatment Group E received a dose of 2.5 ug of m_DR2677_AA_PEG.
**On Day 0, mice in Treatment Group F received a dose of 8.5 ug of m_DR2677_AA_PEG and mice 2, 3 and 4 did not receive a 5^{th} dose.

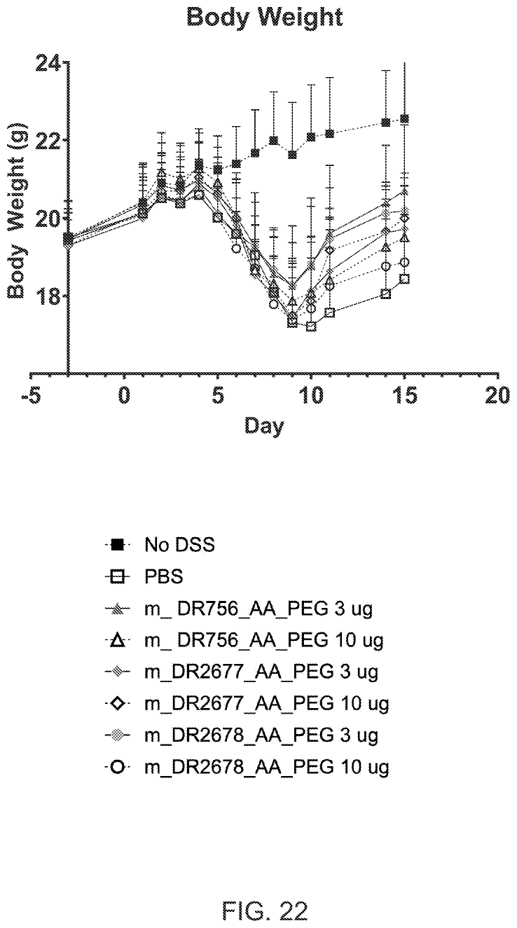

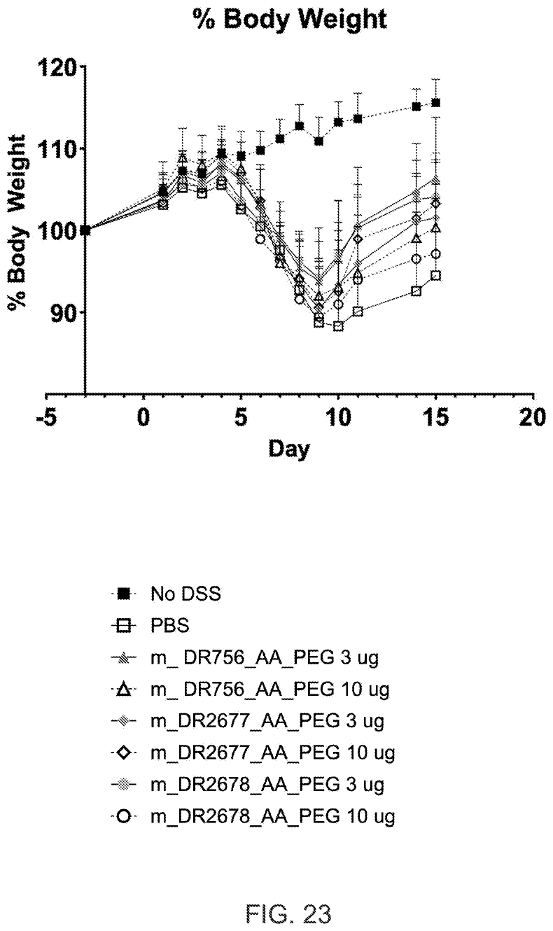

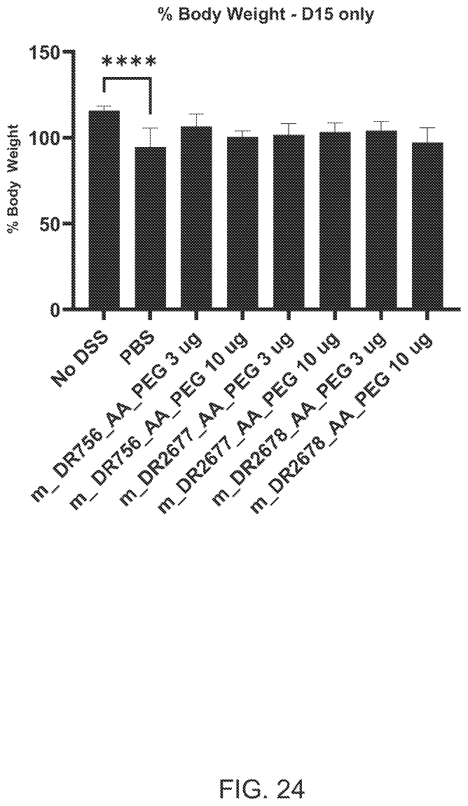

The bodyweights of the mice were evaluated throughout the study. Mice were weighed three days prior to initiation of DSS (Study Day −3) and after DSS administration and test article treatment. The data relating to bodyweight for each treatment group is provided in FIGS. 22 and 23 of the attached drawings. Both average body weight and average 00 body weight+/−SEM are shown. Percent body weight was calculated as =(Body weight on Study Day X—Baseline body weight on Study Day −3)/(Baseline body weight on Study Day −3). Terminal bodyweight of the mice was evaluated on Study Day 15 and the average % body weight+/−SEM on day 15 is shown in FIG. 24 of the attached drawings. Significance was determined by One-Way ANOVA with Holm-Šídík's multiple comparisons test. ****p<0.0001. As shown by the data in FIGS. 22, 23 and 24 the fused IL10 polypeptides protect against DSS associated weight loss.

On Study Day 15, terminal blood was harvested via cardiac puncture and collected into serum collection tubes. In-life and terminal serum was isolated by centrifuging clotted blood for 5 minutes at 10,000 RPM. Serum was collected after centrifugation and placed on ice temporarily before freezing for later analysis.

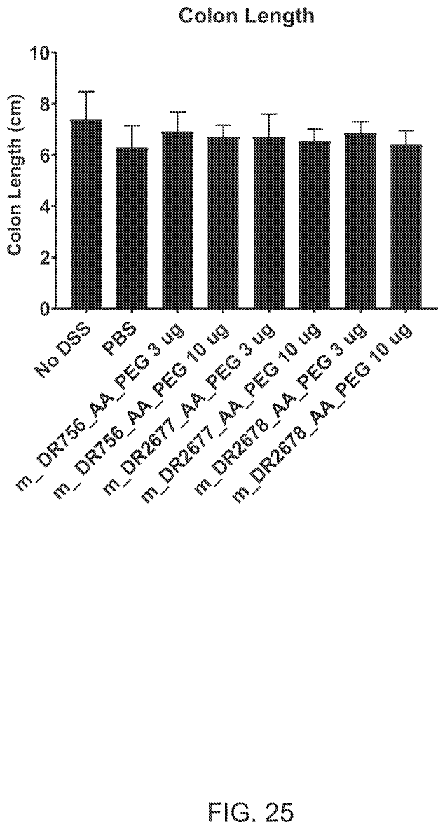

Mice were terminated on day 15 by C02 asphyxiation. Colons were harvested, cleared of feces, and measured for length. The data relating to colon length in response to the various treatments is provided in FIG. 25 and reflects average colon length+/−SEM. As shown by the data presented in FIG. 25, m_DR756_AA_PEG, m_DR2677_AA_PEG, and m_DR2678_AA_PEG show evidence of protection against DSS-associated outcomes and pathology.

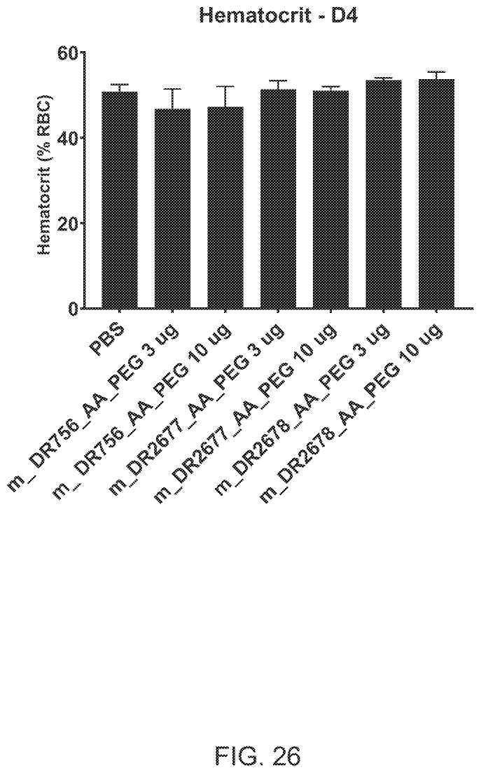

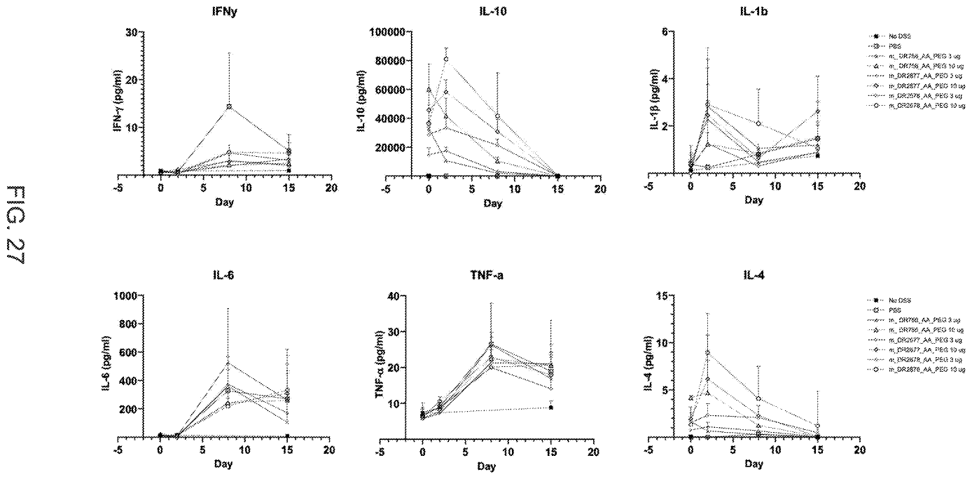

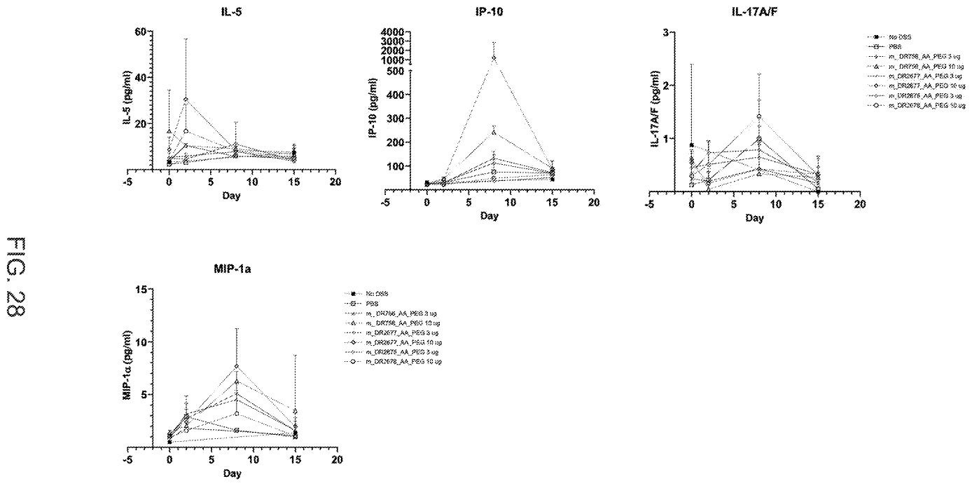

Hemocrit levels of were evaluated on Study Day 4 (D4). Blood samples collected via submandibular blood collection on Study Day 4 were placed into serum collection tubes and quantified using a Hemata Stat II hemaotocrit microcentrifuge (EKF Diagnostics, Inc.) in substantial accordance with the manufacturer's instructions. The data relating to hemocrit levels is provided in FIG. 26 of the attached drawings and illustrates average hematocrit+/−SEM. RBC=red blood cells. As illustrated, a putative reduction in HCT may be observed on Day 4 with m_DR756_AA_PEG.

Serum was collected at various timepoints during the study and serum cytokines (IFNγ, IL10, IL-1B, IL6, TNF-α, IL-4, IL-5, IP10, IL17A/F, MIP1α) levels a were measured. Serum cytokines were assessed using Meso Scale Discovery multiplex kits (Meso Scale Discovery) in substantial accordance with the manufacturer's instructions. The data relating to the levels of these cytokines for for the various treatment groups is provided in FIGS. 27 and 28 of the attached drawings and reflects average analyte concentrations (pg/mL)+/−SEM. As illustrated in FIG. 27, treatment with m_DR2677_AA_PEG and m_DR2678_AA_PEG associates with elevated serum IL-4 and IL-5 (T_{H}2 cytokines)m_DR756_AA_PEG and fused IL10 muteins show reduced systemic IFNγ and TNFα and potentially elevated serum IL-4 (a TH2 cytokine). As illustrated in FIG. 28, Treatment with m_DR756_AA_PEG and m_DR2677_AA_PEG show elevated serum IP-10 and MIP1a not seen with m_DR2678_AA_PEG.

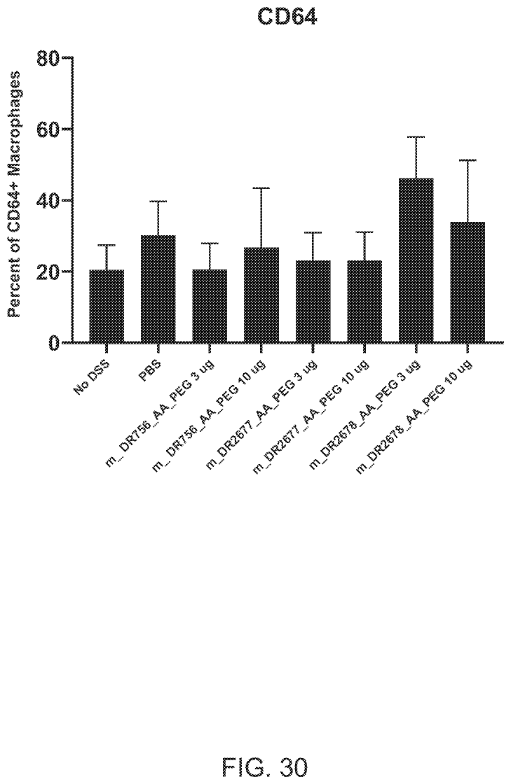

Peritoneal exudate cells were collected following sacrifice on D15 and CD163 or CD64 expression on CD45+CD11b+F4/80+ macrophages was detected by flow cytometry. The data is provided in FIGS. 29 and 30. Average percent of macrophages positive for CD163 or CD64+/−SEM is illustrated on the vertical axis with respect to each of the treatment groups. m_DR756_AA_PEG m_DR2677_AA_PEG induce greater CD163 expression than observed with _DR2678_AA_PEG. MCD163 upregulation on macrophages was not seen with m_DR2678_AA_PEG.

Following evaluation for colon length, the isolated colons were then placed in formalin for 24 hours at room temperature before processing and embedding into FFPE blocks using standardized procedure on a tissue processor. FFPE blocks were sectioned at 5 m thickness and placed on charged glass slides.

Multiplex immunofluorescence analysis was performed on the harvested tissues for the presence of the following markers: RORγ, CD11b & CD3e. Primary antibodies used for immunohistochemical analysis were: anti-mouse: RORγ (Abcam, catalog ab207802) at a working concentration 1:750; anti-CD11b (Abcam, catalog ab216445) at a working concentration 1:3000; and anti-CD3e (Thermo/Invitrogen, catalog MA1-90582) at a working concentration 1:200. All antibodies were incubated at room temperature for 60 minutes.

Slides were stained with the antibodies on Leica Bond Rx (Leica Biosystems) automated staining system. The automated staining system was programmed to perform the following steps: (a) heat-induced epitope retrieval (HIER) and de-paraffinization was performed for 20 minutes at 97 °C in the presence of BOND-PRIME Epitope Retrieval Solution 2 (ER2) (Leica Biosystems); (b) peroxidase quenching was performed; (c) incubation with primary antibody for 60 minutes; (d) incubation with secondary antibody polymer for 30 minutes; and (e) detection fluorophores for 10 minutes at room temperature. Fluorophores used to visualize the signal included Opal 520, Opal 620, and Opal 690 fluorophores (Akoya Biosciences, Marlborough MA).

For labeling, the staining was performed in a sequential manner by including a second antigen retrieval step between the two procedures to strip out any unbound reagents from the previous markers using BOND-PRIME Epitope Retrieval Solution 1 (ER1) (Leica Biosystems). Appropriate cross-reactivity controls were included for quality check.

Upon completion of fluorescence staining, tissues were counterstained with Akoya spectral DAPI (Akoya Biosciences) and cover slipped using ProLong™ Gold Antifade Mountant (ThermoFisher Scientifid/Invitrogen, catalog number: P36930) aqueous mounting medium. Whole slide scanning and signal quantification was performed using Akoya Vectra multispectral imaging system (Akoya Biosciences) and Akoya Vectra InForm analysis software suite (Akoya Biosciences). Digital quantification of specific staining signal was performed, and data was collated and aggregated from all images by using Akoya Phenoptr Reports software package (Akoya Biosciences).

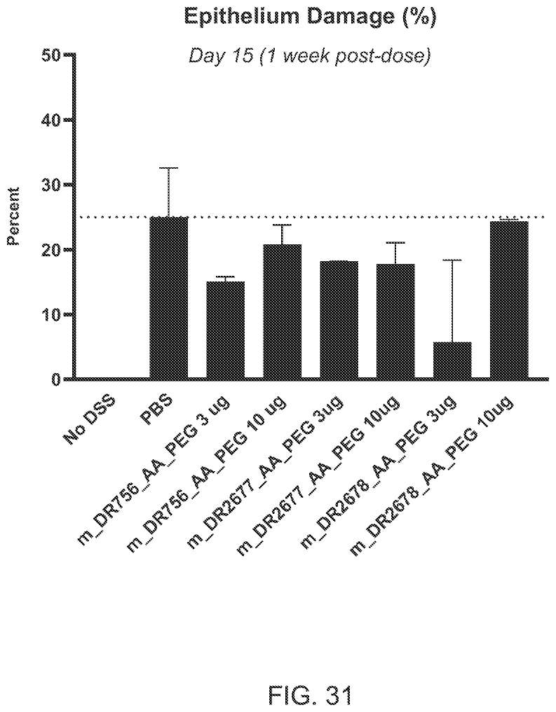

The tissues were evaluated for colon epithelial damage. Whole slide scanned hematoxylin and eosin (H&E) stained mouse colon sections were evaluated with Visiopharm software (Visiopharm A/S, DK 2970-Horsholm, Denmark) for intact epithelium layer. The data for the various treatment groups is provided in FIG. 31 of the attached drawings and reflects the average percent colon epithelial damage. As illustrated, the test articles m_DR756_AA, m_DR2677_AA_PEG, and m_DR2678_AA_PEG protect against DSS-associated epithelial damage.

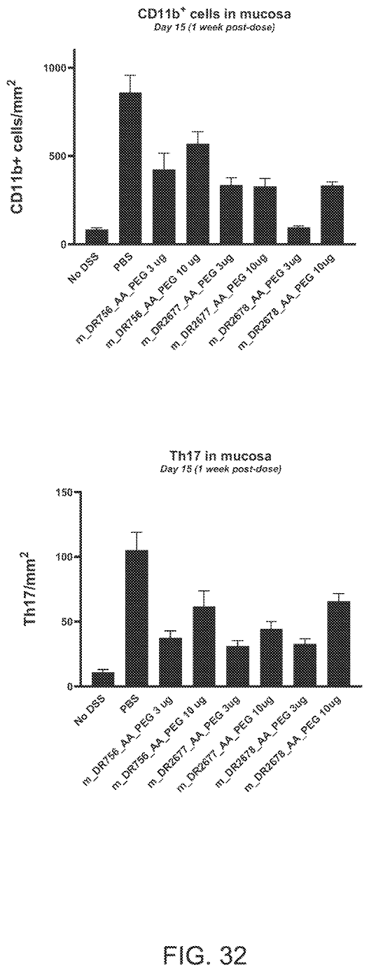

Whole slide scanned mutiplex immunofluorescence stained mouse colon sections were evaluated with in Form image analysis software (Akoya Biosciences) for the presence of CD11b and CD4+ RorG T cells. The data for the various treatment groups is provided in FIG. 32 of the attached drawings and reflects the average number of cells/mm².

### Example 15: PSTAT3 Induction in Myeloid Cells Over Time

Induction of pSTAT3 in isolated myeloid cells was evaluated by flow cytometry. Immune cell populations were identified by lineage marker-based immune cell subsetting. Whole blood was collected into K2-EDTA tubes and 100 μl of blood per mouse was treated with 2 ml prewarmed 1× Lyse/Fix Phosphoflow solution (BD #558049) for 10 minutes at 37 °C. Cells were pelleted by centrifuging at 500 g for 5 mins, followed by wash with FACS buffer (PBS+2% FBS). Cells were permeabilized by adding 1 mL of chilled Perm Buffer III (BD #558050) and incubated at −20 °C overnight. Cells were washed with FACS buffer to remove the permeabilization buffer and blocked with Mouse FcX (Biolegend #101320) and rat serum for 5 minutes at room temperature. The cells were then treated with the antibody cocktail listed in Table 11 below for 1 hour at room temperature.

TABLE 11
Antibody Cocktail Components

| Fluoro- | | | | |
|---|---|---|---|---|
| chrome | Antibody | Clone | Catalog # | Vendor |
| BV421 | CD25 | PC61 | 102034 | Biolegend |
| BV480 | CD4 | RM4-5 | 565634 | BD Biosciences |
| BV711 | CD8a | 53-6.7 | 563046 | BD Biosciences |
| BV750 | CD11b | M1/70 | 101267 | Biolegend |
| BV786 | CD3 | UCHT1 | 565491 | BD Biosciences |
| AF488 | pSTAT3 | D3A7 | 4323S | Cell Signaling |
| | | | | Technology |
| PE | pSTAT1 | REA159 | 130-129-183 | Miltenyi Biotec |
| PerCP-Cy 5.5 | B220 | RA3-6B2 | 552771 | BD Biosciences |
| AF647 | pSTAT5 | 47 | 562076 | BD Biosciences |
| APC/Fire750 | NK1.1 | PK136 | 108752 | Biolegend |

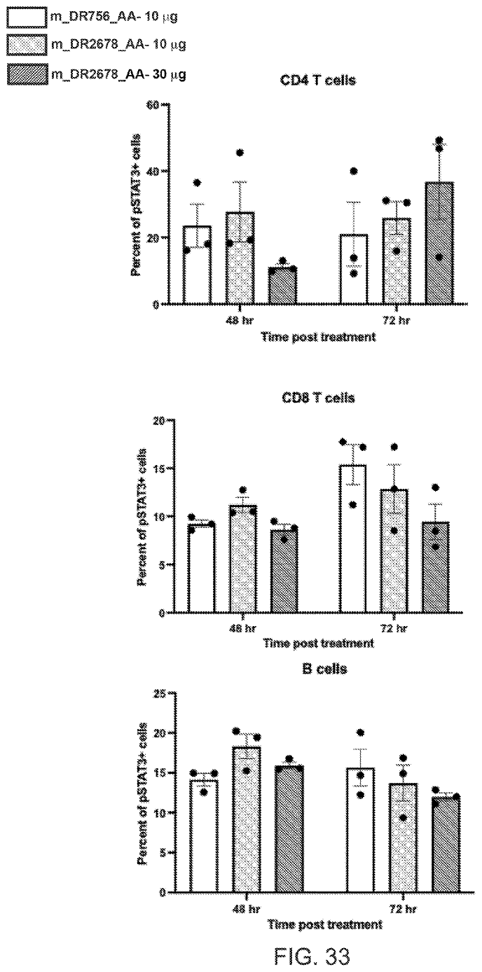

Following antibody staining, the cells were washed, fixed and ran on the Cytek Aurora Spectral flow cytometer. The data was analyzed using the Cell engine software. The various cell lineages were gated for using their lineage markers and the median fluorescence intensity of pSTAT3 expression was calculated on Cell engine. CD4 T cells were identified as live CD3+CD4+ cells; CD8 T cells were identified as live CD3+CD8+ cells; B cells were identified as live CD19+ cells. Individual and average percent of cells positive for pSTAT3+/−SEM are shown. The data is provided in FIG. 33 of the attached drawings. As illustrated in FIG. 33, m_DR2678_AA_PEG exhibited evidence of pSTAT induction in PBMCs at 48 and 72 hours after administration.

### Example 16: In Vivo Evaluation of Pharmacokinetic Parameters

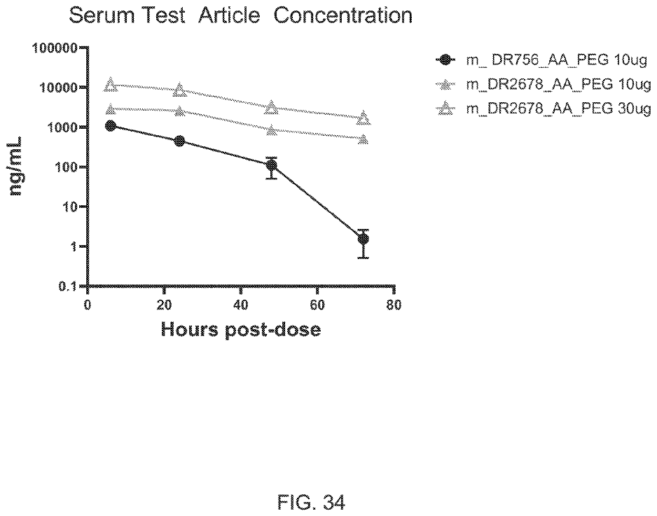

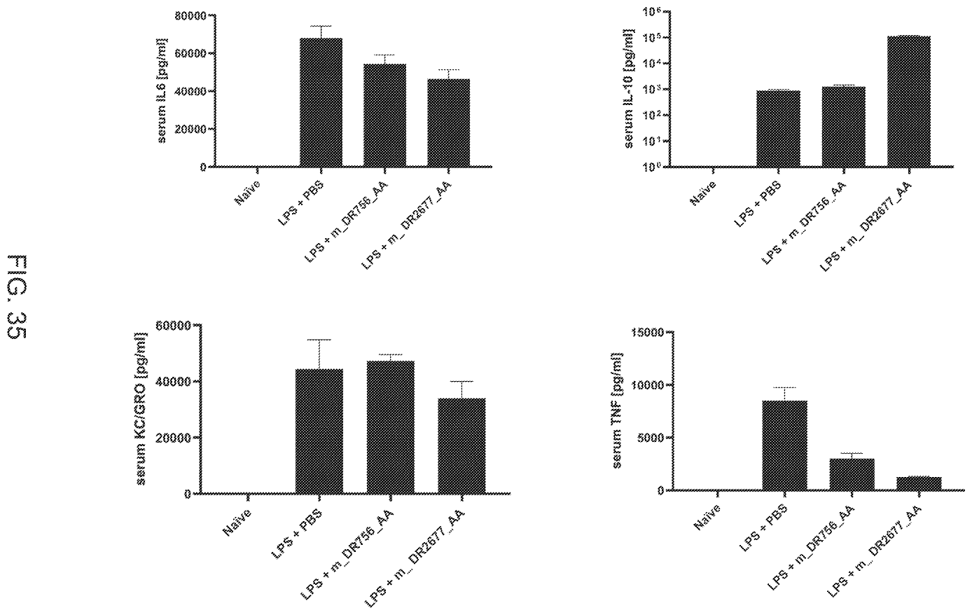

Pharmacokinetic parameters of the m_DR756_AA_PEG and m_DR2678_AA_PEG test articles were evaluated in an in vivo study in C57Bl1/6 mice. Briefly, on Study Day 0 (DO), female C57Bl1/6 mice received a single subcutaneous administration of 10 micrograms of m_DR756_AA_PEG, or a single subcutaneous administration of 10 or 30 micrograms m_DR2678_AA_PEG. In-life blood samples were collected at various timepoints via submandibular blood collection into serum collection tubes. Terminal blood was harvested via cardiac puncture and collected into serum collection tubes. In-life and terminal serum was isolated by centrifuging clotted blood for 5 minutes at 10,000 RPM. Serum was collected after centrifugation and placed on ice temporarily before freezing for later analysis. Test articles in serum were quantified using a mouse IL-10 MSD U-plex kit (Meso Scale Discovery) in substantial accordance with the manufacturer's instructions. Each individual test article was used as a standard for detection. Serum was collected over time and serum concentrations of test articles were quantified over time and the data provided in FIG. 34 of the attached drawings reflecting the average test article concentrations (ng/mL)+/−SEM. As illustrated in FIG. 34, m_DR756_AA_PEG, m_DR2677_AA_PEG, and m_DR2678_AA_PEG show high sustained exposure.

### Example 17: Evaluation of IL-10 Variants in LPS Shock Model

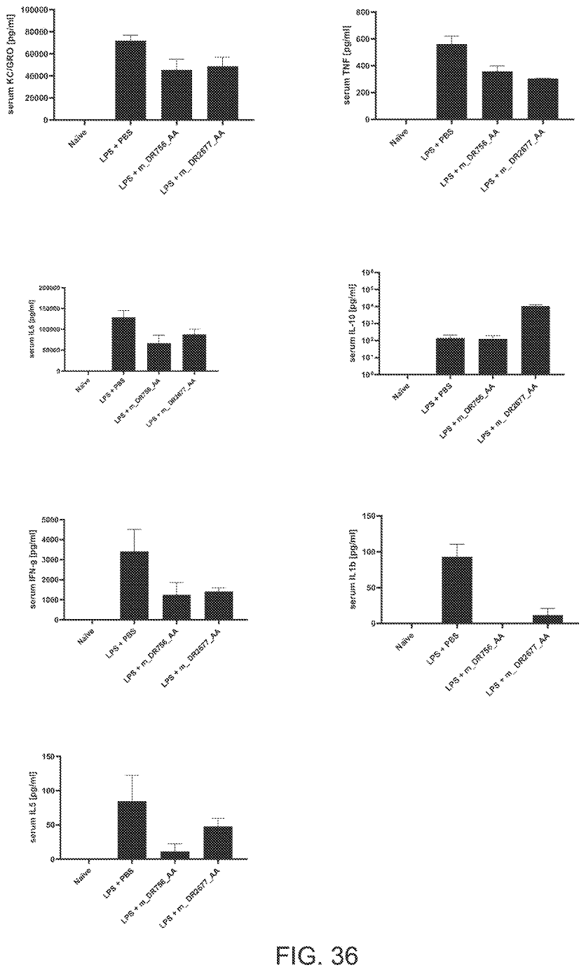

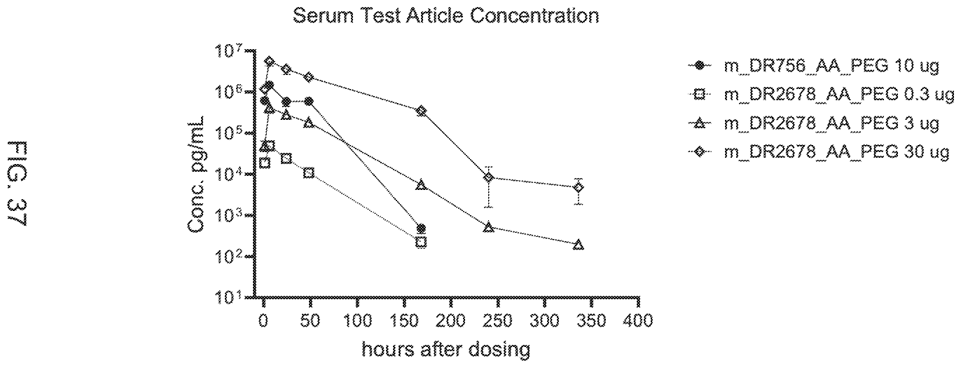

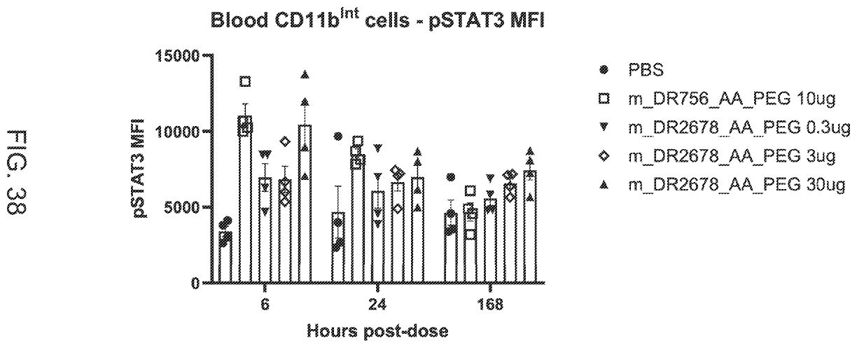

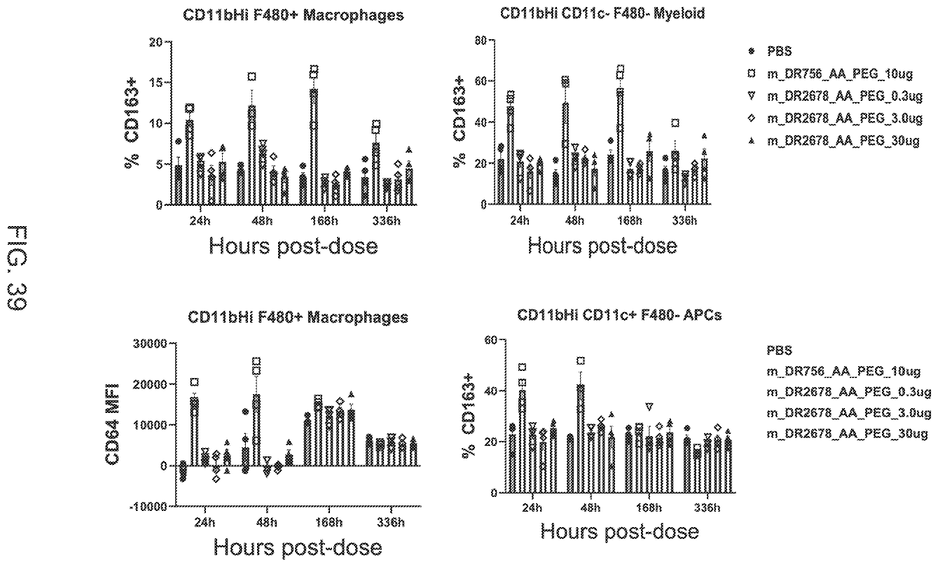

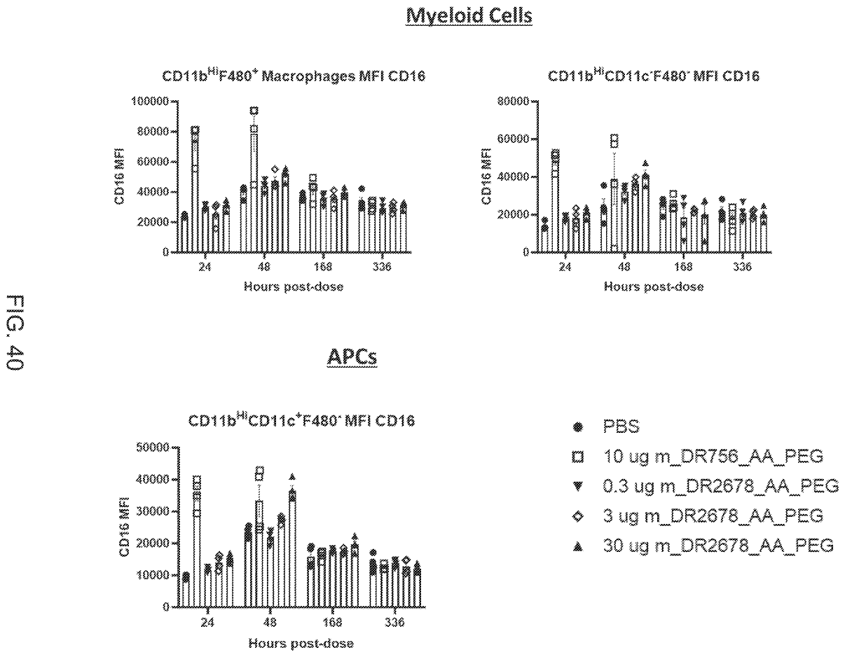

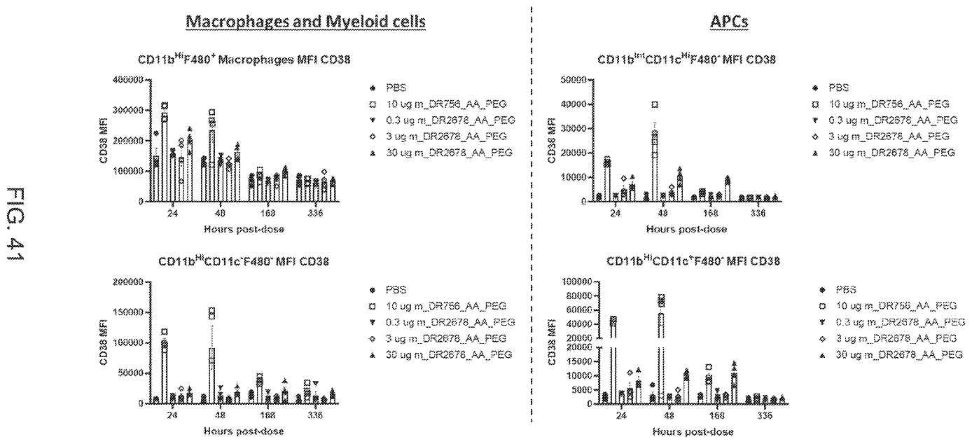

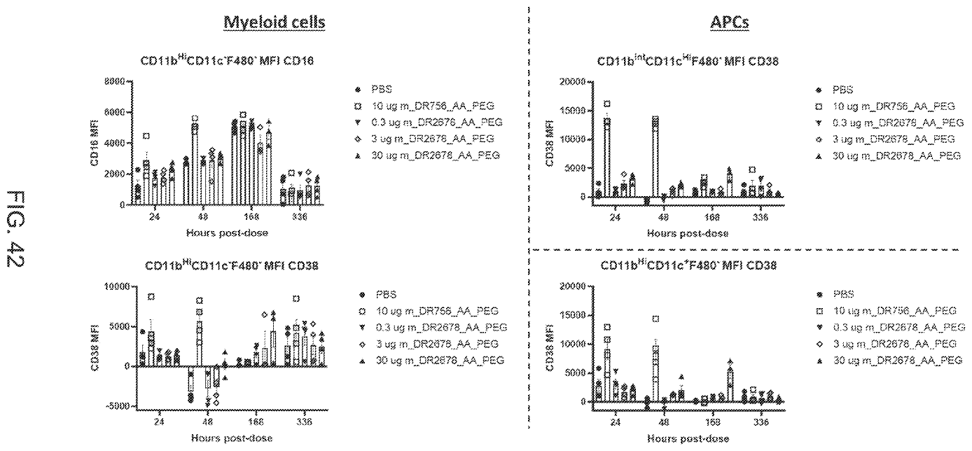

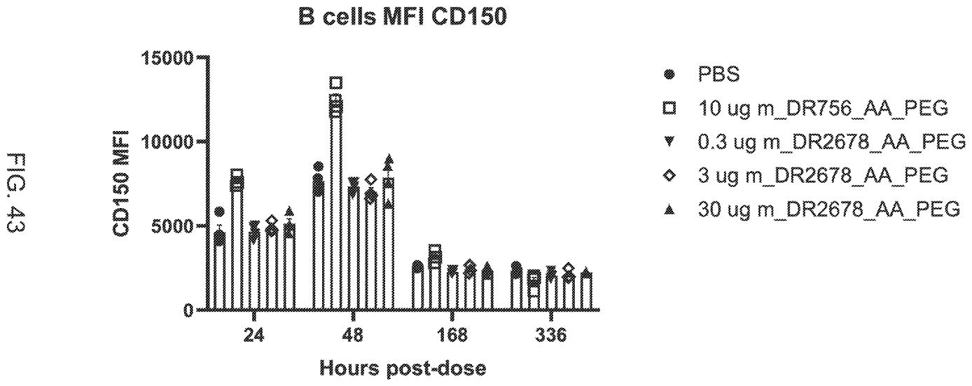

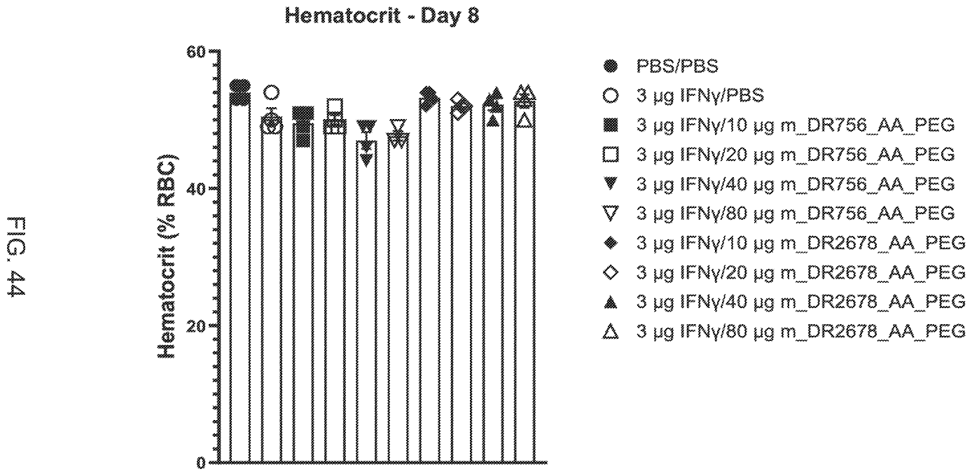

The LPS shock model is used as a method of evaluating the potential utility of therapeutic agents in the treatment of sepsis. Garrido, et al. (2004) “Experimental models of sepsis and septic shock: an overview.” Acta Cirúrgica Brasileira 19.2; Poli-de-Figueiredo, et al, (2008) Experimental models of sepsis and their clinical relevance. Shock, 30(7):53-59. The following LPS shock model was used to evaluate the efficacy of the test articles in the treatment of sepsis. Female BALB/c mice were challenged with a single 100 g dose of LPS administered intraperitoneally. Immediately following LPS challenge, mice were treated with either 0.5 g m_DR756_AA or 10 g m_DR2677_AA. Blood samples were collected at 1.5 and 6 hour timepoints following LPS administration via submandibular blood collection and terminal cardiac puncture, respectively, into serum collection tubes. Serum was isolated by centrifuging clotted blood for 5 minutes at 10,000 RPM. Serum was collected after centrifugation and placed on ice temporarily before freezing for later analysis. Serum cytokines (IL10, TNFα, IL6 and KC/GRO) were assessed using Meso Scale Discovery multiplex kits following manufacturer's recommendations. The data relating to 1.5 hour serum cytokine levels is provided in FIG. 35 of the attached drawings. The data relating to 6 hour serum cytokine levels is provided in FIG. 36 of the attached drawings. Average analyte concentrations (pg/mL)+/−SEM are shown.

### Example 18: Evaluation of Single Dose Pharmacodynamic and Pharmacokinetic Parameters

To determine the pharmacokinetic properties of the test articles, female C57Bl/6 mice were treated with a single dose of either m_DR756_AA_PEG (10 g administered subcutaneously on day 0 (DO)) or m_DR2678_AA_PEG (0.3, 3, or 30 g administered subcutaneously on DO). In-life blood samples were collected at various timepoints via submandibular blood collection into serum collection tubes. Terminal blood was harvested via cardiac puncture and collected into serum collection tubes. In-life and terminal serum was isolated by centrifuging clotted blood for 5 minutes at 10,000 RPM. Serum was collected after centrifugation and placed on ice temporarily before freezing for later analysis. Test articles in serum were quantified using MSD-based detection (Meso Scale Discovery). Each individual test article was used as a standard for detection. Serum was collected at various time points and serum concentrations of test articles were quantified over time. The data is provided in FIG. 37 of the attached drawings. Average test article concentrations (ng/mL)+/−SEM are shown. As illustrated in FIG. 37, m_DR2678_AA_PEG showed improved systemic exposure compared to m_DR756_AA_PEG

### Example 19: Evaluation of Myeloid Cell pSTAT3 Induction Induction Over Time

The effect of the test articles was evaluated for their ability to induce pSTAT3 in myeloid cells over time as follows. In-life or terminal whole blood from the experiments described in Example 18 above was collected into EDTA tubes for pSTAT or surface immunophenotyping flow cytometry. 100-200 μL of whole blood that was collected into EDTA tubes was transferred to 96 well deep well plates. BD Phosflow Lyse/Fix Buffer (cat #558049) was added to a 2 ml total volume and incubated at 37 °C for 10 mins. Cells were centrifuged at 600 g for 7 mins, supernatant removed, and washed twice with 1 ml PBS/2% FBS per well. Cell pellets were vortexed to resuspend and 1 ml prechilled BD Perm Buffer III (cat #558050) was added and cells stored at −80 °C. Following conclusion of the in-life sample collection, cells were thawed and washed three times to remove permeabilization buffer. Cells were blocked with 1:25 TruStain FcX (Biolegend #101320) and 1:50 rat serum for 10 mins on ice then incubated with a cocktail of antibodies for 30 mins at 4 °C (see Table 12 below). Cells were washed twice and resuspended in PBS/2% FBS and ran on a Cytek Aurora Spectral cytometer. Data was analyzed using Cell Engine. Cells were gated using lineage markers and the median fluorescent intensity of pSTAT 1 and pSTAT3 was calculated.

TABLE 12
pSTAT staining panel

| Fluoro- | | | | |
|---|---|---|---|---|
| chrome | Antibody | Clone | Source | Catalog# |
| PE | pSTAT-1 (Y701) | REA159 | Miltenyi Biotec | 130-129-183 |
| AF488 | pSTAT-3 (Y705) | C3A7 | Cell Signaling | 4323S |
| | | | Tech | |
| BV786 | CD3 | 17A2 | BD Biosciences | 564010 |
| APC | CD4 | RM4-5 | Biolegend | 100516 |
| BV711 | CD8 | 53-6.7 | BD Biosciences | 563046 |
| PacBlue | B220 | RA3-6B2 | BD Biosciences | 558108 |
| BV750 | cd11b | M1/70 | Biolegend | 101267 |
| | Trustain FcX | | Biolegend | 101320 |

To evaluate myeloid cell pSTAT3 induction overtime, peripheral blood was collected over time and pSTAT3 was detected by flow cytometry. Data from CD11b^{Int} (CD3⁻B220⁻CD11b^{Intermediate}) is shown below. The data is provided in FIG. 38 of the attached drawings. Individual and average median fluorescence intensity+/−SEM are also shown.

### Example 20: Immunophenotyping Methods

With respect to the evaluations performed in Examples 27-31, the following immunophenotyping methods were employed. Isolated cells were stained in 96 well deep well plates. First, cells were stained with Zombie NIR Fixable Viability Kit (Biolegend #423105) following the manufacturer's protocol. Cells were washed twice, resuspended in 50 ul residual volume and blocking cocktail of anti-mouse CD32 (1:50) and rat serum (1:50) was added to the cells to block non-specific binding and incubated at 4 °C for 10 minutes. A master mix of 30 antibodies was added to the cells (see table below) and incubated at 4 °C for 30 minutes. Cells were washed twice then fixed and permeabilized using FoxP3/Transcription Staining Buffer Set (ThermoFisher #00-5523-00). Cells were blocked with anti-mouse CD32+ rat serum for 10 mins at 4 °C. Antibodies to FoxP3 and Granzyme B were added to the cells and incubated for 30 mins at 4 °C. Cells were washed twice, resuspended in PBS/2% FBS and run on the Cytek Aurora Spectral Cytometer. Analysis was done using Cell Engine. Cell subsets were identified based on positive staining of known defining markers and the lack of expression of other markers. Fluorescence-minus-one (FMO) controls were used for markers where the distinction between positive and negative was not bimodal. Subsequent analysis of median fluorescence intensity and percent positive cells were calculated within Cell Engine.

TABLE 13
Immunophentyping Staining Panel

| Fluorophore | Antibody | Clone | Source | Catalog # |
|---|---|---|---|---|
| BUV395 | F480 | T45-2342 | BD Biosciences | 565614 |
| BUV496 | MHC II (I-A/I-E) | M5/114.15.2 | BD Biosciences | 750281 |
| BUV563 | Ly6G | 1A8 | BD Biosciences | 612921 |
| BUV615 | CD163 | TNKUPJ | Invitrogen | 366-1631-82 |
| BUV661 | CD80 | 18-1OA1 | BD Biosciences | 741515 |
| BUV737 | Ly6C | HK1.4.rMAb | BD Biosciences | 755201 |
| BUV 805 | CD35 | 8C12 | BD Biosciences | 741960 |
| BV421 | CD38 | 90 | Biolegend | 102732 |
| SB436 | CD25 | PC61.5 | invitrogen | 62-0251-82 |
| PB | CD11b | M1/70 | Biolegend | 101224 |
| BV480 | CD62L | MEL-14 | BD Biosciences | 746726 |
| BV570 | NK1.1 | PK136 | Biolegend | 108733 |
| BV605 | CD19 | 6D5 | Biolegend | 115540 |
| V610 | CD4 | RM4-5 | Cytek | R7-20269 |
| BV650 | CD83 | Michel-19 | Biolegend | 121515 |
| BV711 | NKp46 | 29A1.4 | Invitrogwn | 407-3351-82 |
| BV750 | CD3 | 17A2 | Biolegend | 100249 |
| BB515 | ICOS | C398.44 | BD Biosciences | 585880 |
| FITC | CD16 | S17014E | Biolegend | 158008 |
| AF532 | CD45 | 30-F11 | Invitrogen | 58-0451-82 |
| PerCPCY5.5 | CD8a | 53-6.7 | Biolegend | 100734 |
| PerCP-Vio 700 | FoxP3 | FJK-16s | Invitrogen | 46-5773-82 |
| APC | TCRb | H57-597 | Biolegend | 109212 |
| AF647 | CD14 | Sa14-2 | Biolegend | 123328 |
| AF700 | CD44 | IM7 | Biolegend | 103026 |
| Zombie NIR | dead | n/a | Biolegend | 423105 |
| APCFire 750 | CD64 | X54-5/7.1 | Biolegend | 139334 |
| PE | CD150 | Q38-480 | BD Biosciences | 562651 |
| PE/Dazzle594 | CD86 | GL-1 | Biolegend | 105042 |
| PE/Fire640 | CD127 | S18006K | Biolegend | 158214 |
| PECy5 | PD-1 | 29F.1A12 | Biolegend | 135256 |
| PE/Fire 700 | CD11c | QA18A72 | Biolegend | 161108 |
| PECy7 | Granzyme b | NGZB | Invitrogen | 25-8898-92 |
| pur | CD32 | S17012B | Biolegend | 156402 |

### Example 21: Evaluation of Peritoneal Exudate Cell Scavenger CD163 and CD64 Receptor Expression Over Time

For peritoneal exudate cell harvesting, peritoneal lavages were performed on euthanized mice and the exudate was collected (peritoneal exudate cells, PEC). Peritoneal lavage fluid was centrifuged (1500 rpm, 5 mins), supernatant removed, and cell pellets were resuspended in 1 ml PBS and kept on ice. Blood was collected into EDTA containing tubes, transferred to 5 ml tubes, and red blood cells were lysed using ACK lysing buffer (ThernoFisher #A1059201) then centrifuged (1500 rpm, 5 mins). Supernatants were removed and cell pellets were resuspended in 1 ml PBS. Peritoneal exudate cells were collected over time and CD163 or CD64 was detected by flow cytometry. The data from live CD45⁺ immune subsets in FIG. 39 of the attached drawings. Individual and average median fluorescence intensity+/−SEM are also shown. APC=antigen presenting cell. CD163 upregulation on macrophages was not observed in response to m_DR2678_AA_PEG.

### Example 22: Evaluation of Peritoneal Exudate Cell Scavenger CD16 Expression Over Time

Isolation of peritoneal exudate cells was performed in substantial accordance with Example 20. Peritoneal exudate cells were collected overtime and CD16 was detected by flow cytometry. The data from live CD45⁺ immune subsets in FIG. 40 of the attached drawings. Individual and average median fluorescence intensity+/−SEM are also shown. APC=antigen presenting cell.

### Example 23: Evaluation of Peritoneal Exudate Cell CD38 Expression Over Time

Isolation of peritoneal exudate cells was performed in substantial accordance with Example 20. Peritoneal exudate cells were collected overtime and CD38 was detected by flow cytometry. The data from live CD45⁺ immune subsets in FIG. 41 of the attached drawings. Individual and average median fluorescence intensity+/−SEM are also shown. APC=antigen presenting cell

### Example 24. Evaluation of Peripheral Blood CD16 and CD38 Expression Over Time

Peripheral blood was collected over time and CD16 or CD38 was detected by flow cytometry in substantial accordance with Example 23 above. The data from live CD45⁺CD3⁻CD19⁺ B cells are provided in FIG. 42 of the attached drawings. Individual and average median fluorescence intensity+/−SEM are also shown.

### Example 25. Evaluation of Peripheral Blood Scavenger CD150 Receptor Expression Over Time

Peripheral blood was collected overtime and CD150 was detected by flow cytometry in substantial accordance with Example 20 above. The data from live CD45⁺CD3⁻CD19⁺ B cells are provided in FIG. 43 of the attached drawings. Individual and average median fluorescence intensity+/−SEM are also shown.

### Example 26: IFNγ-Induced Anemia Study

In order to evaluate the effects of the test articles with respect to IFNγ induced anemia the following study was performed. Female C57Bl/6 mice were treated daily with either PBS or 3 μg of mouse IFNγ (Shenandoah, catalog #200-16AF) administered subcutaneously starting on day 0 (DO). Mice were also co-treated with PBS, m_DR756_AA_PEG (10, 20, 40, or 80 g administered subcutaneously on DO), or m_DR2678_AA_PEG (10, 20, 40, or 80 g administered subcutaneously on DO) every three to four days. Mice were assessed for hematocrit levels on day 8 (24 hours post-third test article dose) study were quantified using a Hemata Stat II following manufacturer's recommendations. The results of this study are provided in FIG. 44 of the attached drawings. The average hematocrit+/−SEM is shown (RBC=Red blood cells). As illustrated in the data presented in FIG. 44, the wild-type IL10 surrogate molecule (m_DR756_AA_PEG) exacerbated IFNγ-induced anemia while the fused dimer comprising the T100L amino acid substitutions (m_DR2678_AA_PEG) did not.

### Example 27. Monocyte IL10 Activity Assay

Human PBMCs were isolated from Leukopaks obtained from a non-smoking human donor (#451) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the test articles listed in Table 14 below at concentrations ranging from 0.02 pM to 1000 nM for 48 hours at 37 °C. The h_DR2339_AA polypeptide was expressed by transient transfection in Expi293 cells using the MHSSALLCCLVLLTGVRA (SEQ ID NO:98) signal peptide which was removed by the host cell and chromatographically purified. The test article h_SS0165_AA was produced in E. coli. The test articles h_DR2339_AA_PEG and h_SS0165_AA_PEG were both N-terminally PEGylated with a 40 kD branched PEG comprising two 20 kD arms. PEGylation was performed in substantial accordance with the teaching of Example 1.

TABLE 14
Example 27 Test Articles

| SEQ | | | |
|---|---|---|---|
| ID NO | Name | Description | AA Sequence |
| 4 | h_DR2339_AA and | Wild-type | SPGQGTQSENSCTHFPGNLPNMLRDL |
| | h_DR2339_AA_PEG | hIL10; | RDAFSRVKTFFQMKDQLDNLLLKES |
| | | mammalian | LLEDFKGYLGCQALSEMIQFYLEEV |
| | | expression | MPQAENQDPDIKAHVNSLGENLKTL |
| | | | RLRLRRCHRFLPCENKSKAVEQVKN |
| | | | AFNKLQEKGIYKAMSEFDIFINYIEAY |
| | | | MTMKIRN (SEQ ID NO: 4) |
| 123 | h_SS0165_AA_PEG | T100L/T100L | MSPGQGTQSENSCTHFPGNLPNMLR |
| | | hIL10 fused | DLRDAFSRVKTFFQMKDQLDNLLLK |
| | | dimer; | ESLLEDFKGYLGCQALSEMIQFYLEE |
| | | E. coli | VMPQAENQDPDIKAHVNSLGENLKL |
| | | expression | LRLRLRRCHRFLPCENKSKAVEQVK |
| | | | NAFNKLQEKGIYKAMSEFDIFINYIEA |
| | | | YMTMKIRNSPGQGTQSENSCTHFPG |
| | | | NLPNMLRDLRDAFSRVKTFFQMKDQ |
| | | | LDNLLLKESLLEDFKGYLGCQALSE |
| | | | MIQFYLEEVMPQAENQDPDIKAHVN |
| | | | SLGENLKLLRLRLRRCHRFLPCENKS |
| | | | KAVEQVKNAFNKLQEKGIYKAMSEF |
| | | | DIFINYIEAYMTMKIRN (SEQ ID NO: |
| | | | 123) |

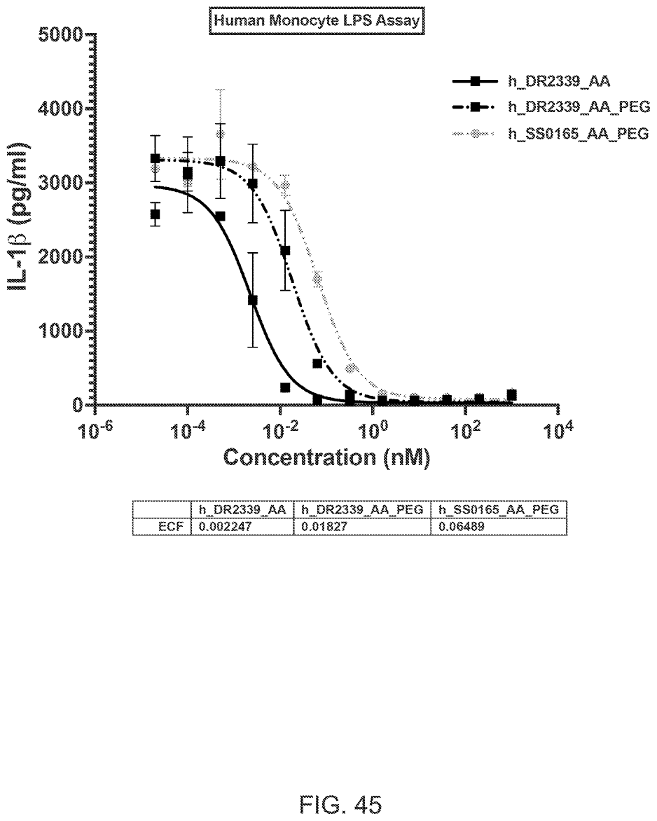

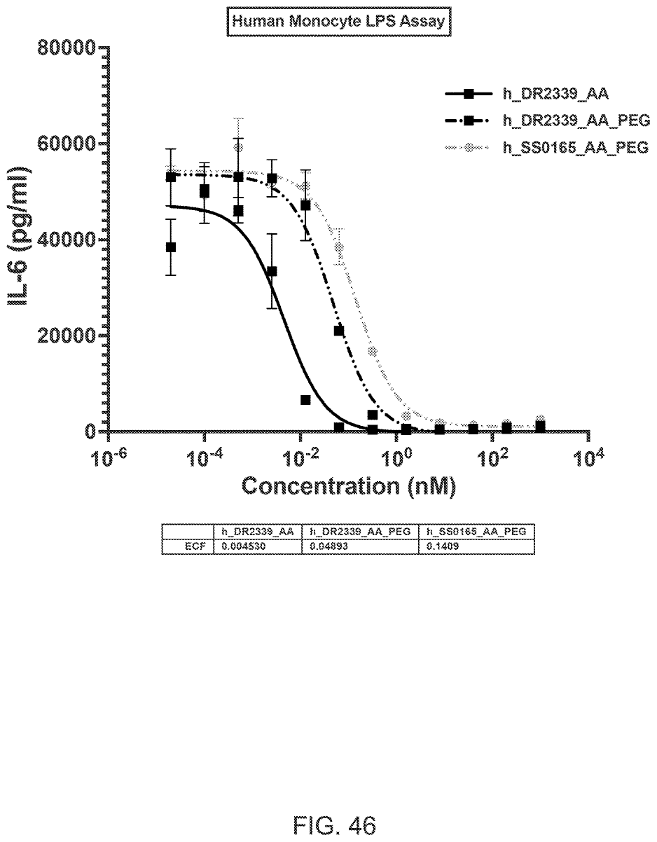

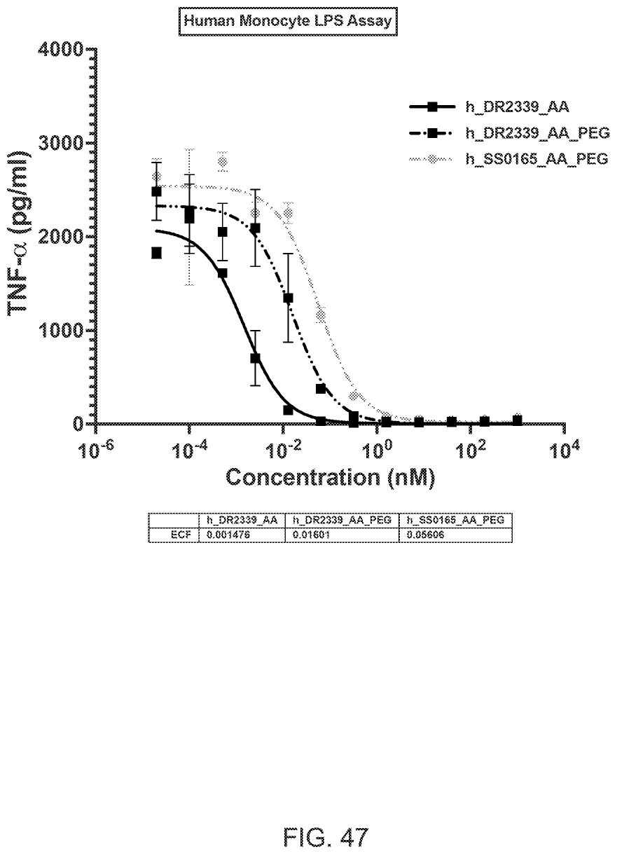

Post-treatment, cells were centrifuged at 400 g for 5 min and supernatants were collected. Supernatants were assessed for secretion of the pro-inflammatory cytokines IL1b (FIG. 45), IL6 (FIG. 46) and TNFα (FIG. 47) using an MSD kit (MesoScale Discovery; Catalog #K151A9H-4) in substantial accordance with the instructions provided by the manufacturer. The data was graphed using Prism software and provided as FIGS. 45-47. The tables under the drawings in FIGS. 45-47 provide data relating to EC50 (ECF) of the test articles in the assay. As shown in the FIGS. 45-47, the PEGylated fused hIL10 molecule with the T100L mutation (h_SS0165_AA_PEG) completely inhibits LPS-induced secretion of IL-10, IL-6 and TNF-α by monocytes, with approximately a 3-4 fold higher EC50 (ECF) compared to PEGylated WT IL-10 (h_DR2339_AA_PEG).

### Example 28. E. Coli and Mammalian Generated Proteins have Equivalent Potencies

Human PBMCs were isolated from Leukopaks obtained from a non-smoking human donor (#3323) by Ficoll® gradient purification. Human Monocytes were purified from the PBMCs by positive selection using Human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the proteins listed in Table 15 below at concentrations ranging from 0.1 pM to 100 nM for 48 hours at 37 °C. h_TP0015_AA was expressed by transient transfection in Expi293 cells as a precursor using the MHSSALLCCLVLLTGVRA (SEQ ID NO:98) signal peptide which was removed by the mammalian host cell to yield the sequence described in Table 15. h_SS0165_AA was expressed in E. coli strain B12. Proteins were purified using metal affinity chromatography.

TABLE 15
Example 28 Test Articles

| SEQ ID | | | |
|---|---|---|---|
| NO | Name | Description | AA Sequence |
| 206 | h_TP0015_AA | T100L/T/T100L | SPGQGTQSENSCTHFPGNLPNMLRDLRD |
| | | hIL10; | AFSRVKTFFQMKDQLDNLLLKESLLEDF |
| | | GGS-His8 tag | KGYLGCQALSEMIQFYLEEVMPQAENQ |
| | | (SEQ ID NO: 23); | DPDIKAHVNSLGENLKLLRLRLRRCHRF |
| | | Mammalian | LPCENKSKAVEQVKNAFNKLQEKGIYK |
| | | Expression | AMSEFDIFINYIEAYMTMKIRNTSPGQGT |
| | | | QSENSCTHFPGNLPNMLRDLRDAFSRVK |
| | | | TFFQMKDQLDNLLLKESLLEDFKGYLG |
| | | | CQALSEMIQFYLEEVMPQAENQDPDIKA |
| | | | HVNSLGENLKLLRLRLRRCHRFLPCENK |
| | | | SKAVEQVKNAFNKLQEKGIYKAMSEFDI |
| | | | FINYIEAYMTMKIRNGGSHHHHHHHH |
| | | | (SEQ ID NO: 206) |
| 207 | h_SS0165_AA | T100L/T/T100L | MSPGQGTQSENSCTHFPGNLPNMLRDL |
| | | hIL10; | RDAFSRVKTFFQMKDQLDNLLLKESLLE |
| | | GGS-His8 tag | DFKGYLGCQALSEMIQFYLEEVMPQAE |
| | | (SEQ ID NO: 23); | NQDPDIKAHVNSLGENLKLLRLRLRRCH |
| | | E. coli Expression | RFLPCENKSKAVEQVKNAFNKLQEKGIY |
| | | | KAMSEFDIFINYIEAYMTMKIRNSPGQG |
| | | | TQSENSCTHFPGNLPNMLRDLRDAFSRV |
| | | | KTFFQMKDQLDNLLLKESLLEDFKGYL |
| | | | GCQALSEMIQFYLEEVMPQAENQDPDIK |
| | | | AHVNSLGENLKLLRLRLRRCHRFLPCEN |
| | | | KSKAVEQVKNAFNKLQEKGIYKAMSEF |
| | | | DIFINYIEAYMTMKIRNGGSHHHHHHHH |
| | | | (SEQ ID NO: 207) |

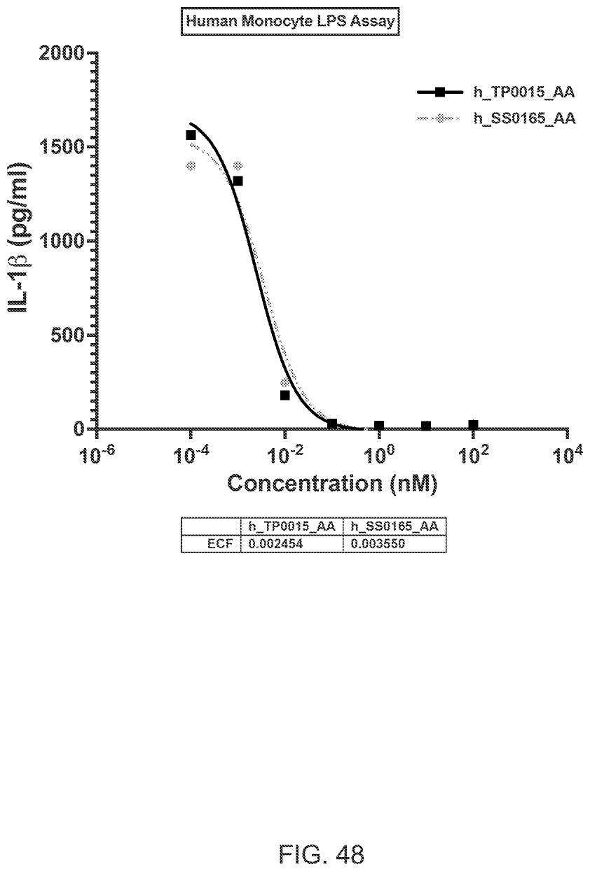

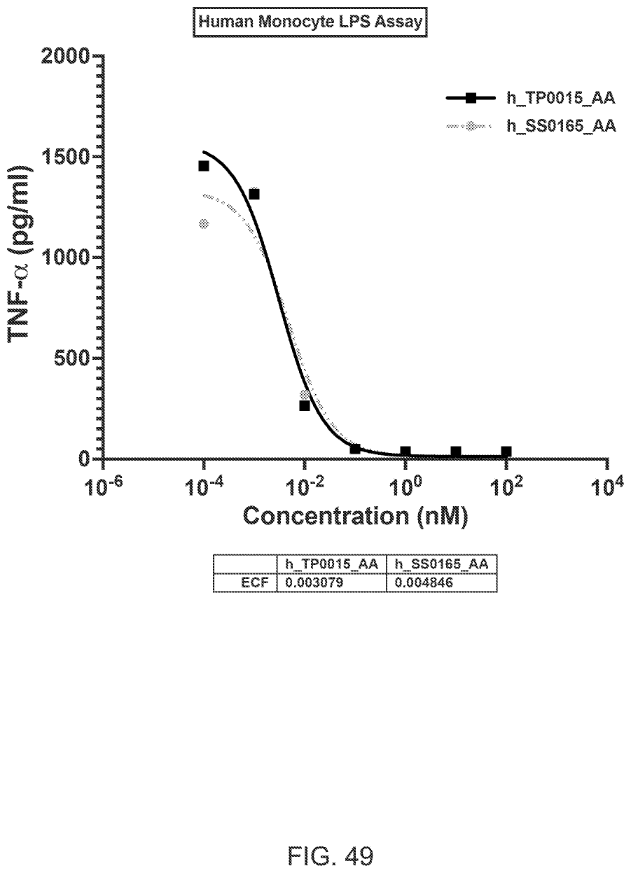

Post-treatment, cells were centrifuged at 400 g for 5 min and supernatants were collected. Supernatants were assessed for pro-inflammatory cytokines using an MSD kit (Meso Scale Discovery, Catalog #K151A9H-4) in substantial accordance with the instructions provided by the manufacturer. The data was graphed using Prism software and is provided in FIGS. 48 and 49 of the attached drawings. As illustrated in FIGS. 48 and 49, the fused hIL10 molecule comprising a T100L mutation obtained from expression in E. coli exhibited equivalent potency to the fused hIL10 molecule comprising T100L mutations to the mammalian generated Fused molecule with T100L mutation.

### Example 29. Activity of Various Single Point Mutations Introduced onto the Fused Molecule

Human PBMCs were isolated from Leukopaks obtained from non-smoking human donors by Ficoll® gradient purification. Human Monocytes were purified from the PBMCs by positive selection using Human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the test articles provided in Table 16 below at concentrations ranging from 0.02 pM to 1000 nM for 48 hours at 37 °C. The test article proteins were expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequences described in Table 16.

TABLE 16
Example 29 Test Articles

| SEQ | | | |
|---|---|---|---|
| ID | Name | Description | AA Sequence |
| 208 | h_DR2339 | hIL10-WT | SPGQGTQSENSCTHFPGNLPNMLRDLRDA |
| | | C-terminal GGS- | FSRVKTFFQMKDQLDNLLLKESLLEDFKG |
| | | His8 tag (SEQ ID | YLGCQALSEMIQFYLEEVMPQAENQDPDI |
| | | NO: 23); | KAHVNSLGENLKTLRLRLRRCHRFLPCEN |
| | | mammalian | KSKAVEQVKNAFNKLQEKGIYKAMSEFDI |
| | | expression | FINYIEAYMTMKIRNGGSHHHHHHHH |
| | | | (SEQ ID NO: 208) |
| 209 | h_SS0053 | T100L/T100L | SPGQGTQSENSCTHFPGNLPNMLRDLRDA |
| | | hIL10 fused; C- | FSRVKTFFQMKDQLDNLLLKESLLEDFKG |
| | | terminal GGS- | YLGCQALSEMIQFYLEEVMPQAENQDPDI |
| | | His8 tag (SEQ ID | KAHVNSLGENLKLLRLRLRRCHRFLPCEN |
| | | NO: 23); | KSKAVEQVKNAFNKLQEKGIYKAMSEFDI |
| | | mammalian | FINYIEAYMTMKIRNSPGQGTQSENSCTHF |
| | | expression | PGNLPNMLRDLRDAFSRVKTFFQMKDQL |
| | | | DNLLLKESLLEDFKGYLGCQALSEMIQFY |
| | | | LEEVMPQAENQDPDIKAHVNSLGENLKLL |
| | | | RLRLRRCHRFLPCENKSKAVEQVKNAFNK |
| | | | LQEKGIYKAMSEFDIFINYIEAYMTMKIRN |
| | | | GGSHHHHHHHH (SEQ ID NO: 209) |
| 210 | h_SS0258 | N21K/N21K | SPGQGTQSENSCTHFPGNLPKMLRDLRDA |
| | | hIL10 fused; C- | FSRVKTFFQMKDQLDNLLLKESLLEDFKG |
| | | terminal GGS- | YLGCQALSEMIQFYLEEVMPQAENQDPDI |
| | | His8 tag (SEQ ID | KAHVNSLGENLKTLRLRLRRCHRFLPCEN |
| | | NO: 23); | KSKAVEQVKNAFNKLQEKGIYKAMSEFDI |
| | | mammalian | FINYIEAYMTMKIRNSPGQGTQSENSCTHF |
| | | expression | PGNLPKMLRDLRDAFSRVKTFFQMKDQL |
| | | | DNLLLKESLLEDFKGYLGCQALSEMIQFY |
| | | | LEEVMPQAENQDPDIKAHVNSLGENLKTL |
| | | | RLRLRRCHRFLPCENKSKAVEQVKNAFNK |
| | | | LQEKGIYKAMSEFDIFINYIEAYMTMKIRN |
| | | | GGSHHHHHHHH (SEQ ID NO: 210) |
| 211 | h_SS0259 | R24E/R24E | SPGQGTQSENSCTHFPGNLPNMLEDLRDA |
| | | hIL10 fused; C- | FSRVKTFFQMKDQLDNLLLKESLLEDFKG |
| | | terminal GGS- | YLGCQALSEMIQFYLEEVMPQAENQDPDI |
| | | His8 tag (SEQ ID | KAHVNSLGENLKTLRLRLRRCHRFLPCEN |
| | | NO: 23); | KSKAVEQVKNAFNKLQEKGIYKAMSEFDI |
| | | mammalian | FINYIEAYMTMKIRNSPGQGTQSENSCTHF |
| | | expression | PGNLPNMLEDLRDAFSRVKTFFQMKDQL |
| | | | DNLLLKESLLEDFKGYLGCQALSEMIQFY |
| | | | LEEVMPQAENQDPDIKAHVNSLGENLKTL |
| | | | RLRLRRCHRFLPCENKSKAVEQVKNAFNK |
| | | | LQEKGIYKAMSEFDIFINYIEAYMTMKIRN |
| | | | GGSHHHHHHHH (SEQ ID NO: 211) |
| 212 | h_SS0260 | hIL10 | SPGQGTQSENSCTHFPGNLPNALRDLRDA |
| | | M22A/M22A | FSRVKTFFQMKDQLDNLLLKESLLEDFKG |
| | | fused; C-terminal | YLGCQALSEMIQFYLEEVMPQAENQDPDI |
| | | GGS-HIS8 tag | KAHVNSLGENLKTLRLRLRRCHRFLPCEN |
| | | (SEQ ID NO: | KSKAVEQVKNAFNKLQEKGIYKAMSEFDI |
| | | 23); mammalian | FINYIEAYMTMKIRNSPGQGTQSENSCTHF |
| | | expression | PGNLPNALRDLRDAFSRVKTFFQMKDQLD |
| | | | NLLLKESLLEDFKGYLGCQALSEMIQFYLE |
| | | | EVMPQAENQDPDIKAHVNSLGENLKTLRL |
| | | | RLRRCHRFLPCENKSKAVEQVKNAFNKLQ |
| | | | EKGIYKAMSEFDIFINYIEAYMTMKIRNGG |
| | | | SHHHHHHHH (SEQ ID NO: 212) |

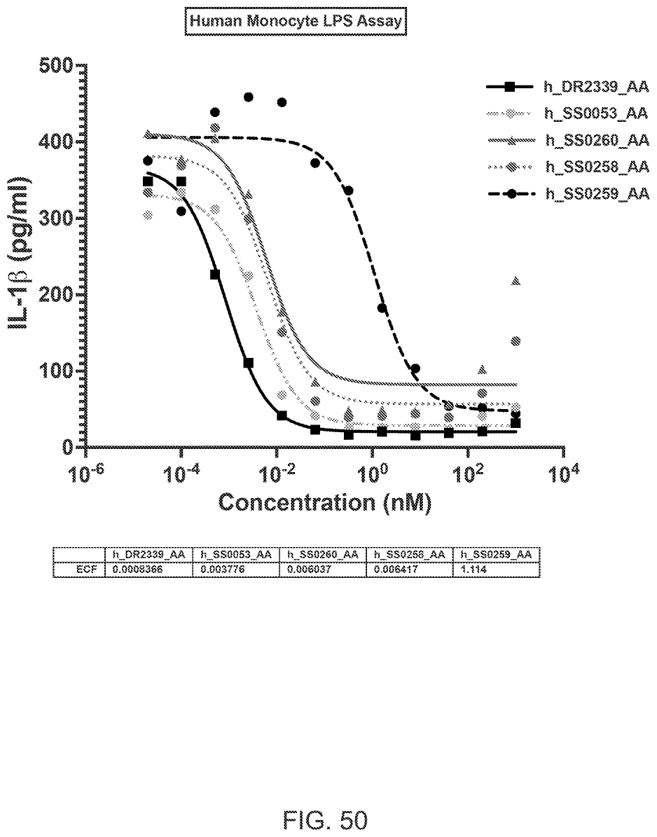

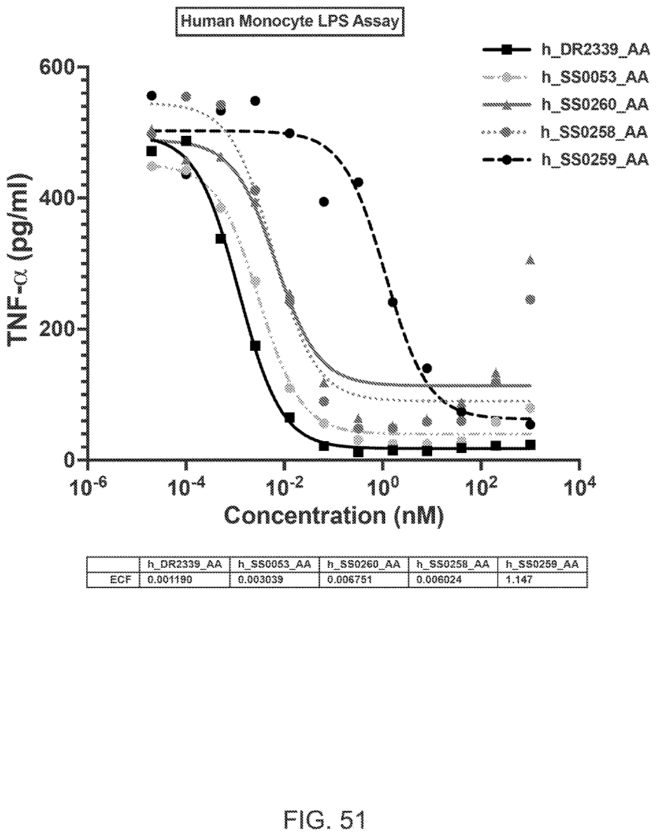

Post-treatment, cells were centrifuged at 400 g for 5 min and supernatants were collected. Supernatants were assessed for pro-inflammatory cytokines using an MSD kit (Meso Scale Discovery, Catalog #K151A9H-4) in substantial accordance with the instructions provided by the manufacturer. The data was graphed using Prism software and is provided in FIGS. 50 and 51 of the attached drawings. The various single point mutations introduced onto the hIL10 fused polypeptides affect myeloid assay potency to varying degrees.

### Example 30. Monocyte IL10 Activity Assay of CB0009 (E96K)

Human PBMCs were isolated from Leukopak obtained from a non-smoking human donor (Donor #072) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the h_DR2339_AA or h_CB0009_AA as described Table 17 below at concentrations ranging from 0.02 pM to 1000 nM for 48 hours at 37 °C. The description of h_DR2339_AA (SEQ ID NO:208) is provided in Example 29. The test article h_CB0009_AA was expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequence described in Table 17.

TABLE 17
h_CB0009_AA

| SEQ | | | |
|---|---|---|---|
| ID NO | Name | Description | AA Sequence |
| 213 | h_CB009_AA | E96K/E96K | SPGQGTQSENSCTHFPGNLPNMLRDL |
| | | hIL10; GGS- | RDAFSRVKTFFQMKDQLDNLLLKES |
| | | His8 (SEQ ID | LLEDFKGYLGCQALSEMIQFYLEEV |
| | | NO: 23) | MPQAENQDPDIKAHVNSLGKNLKTL |
| | | | RLRLRRCHRFLPCENKSKAVEQVKN |
| | | | AFNKLQEKGIYKAMSEFDIFINYIEAY |
| | | | MTMKIRNSPGQGTQSENSCTHFPGNL |
| | | | PNMLRDLRDAFSRVKTFFQMKDQLD |
| | | | NLLLKESLLEDFKGYLGCQALSEMIQ |
| | | | FYLEEVMPQAENQDPDIKAHVNSLG |
| | | | KNLKTLRLRLRRCHRFLPCENKSKA |
| | | | VEQVKNAFNKLQEKGIYKAMSEFDIF |
| | | | INYIEAYMTMKIRNGGSHHHHHHHH |
| | | | (SEQ ID NO: 213) |

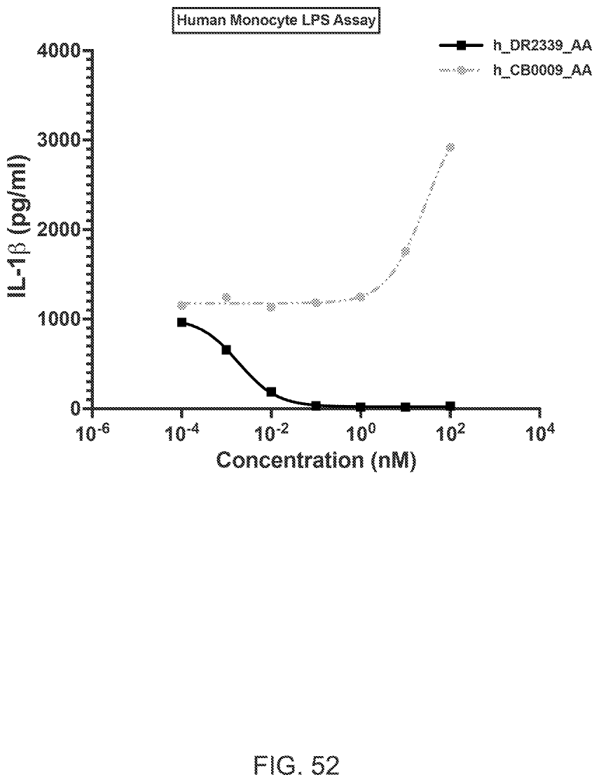

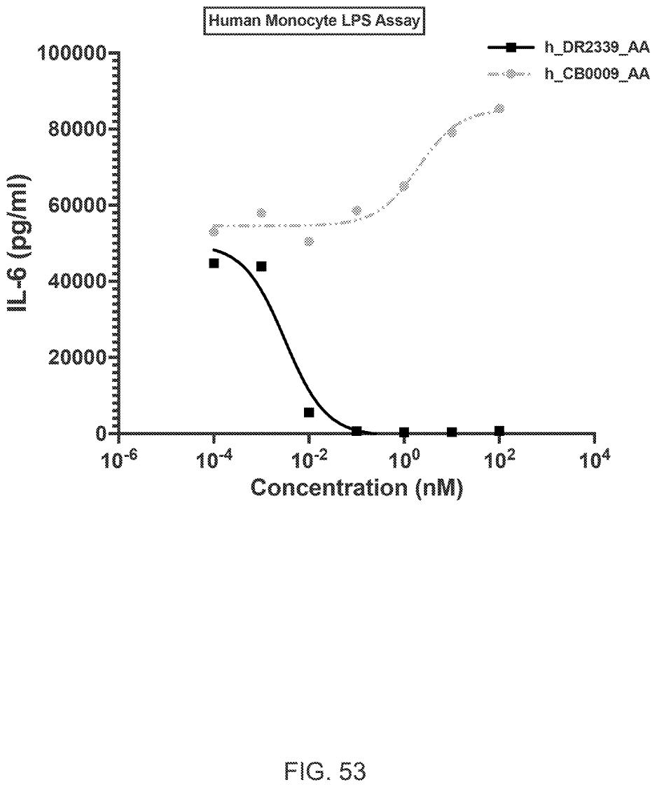

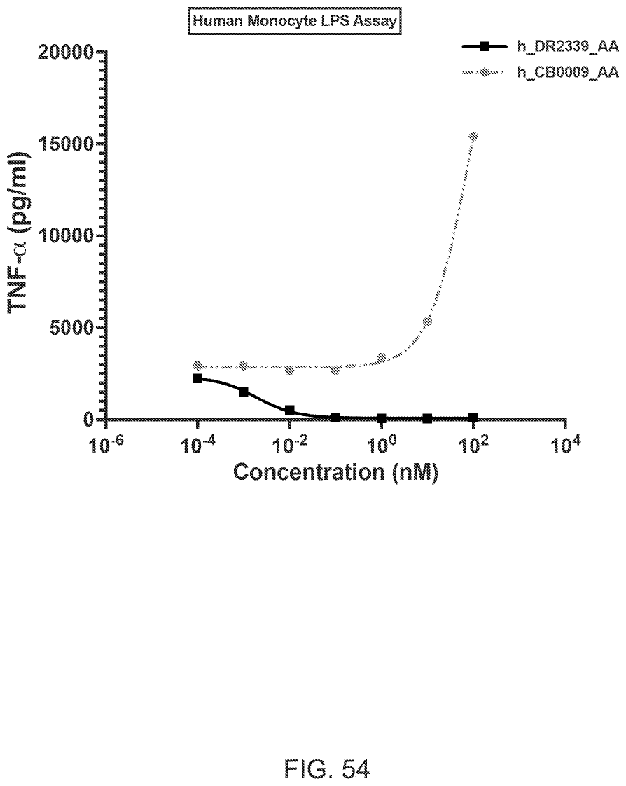

Post-treatment, cells were centrifuged at 400 g for 5 min and supernatants were collected. Supernatants were assessed for pro-inflammatory cytokines IL1b (FIG. 52), IL6 (FIG. 53) and TNFα (FIG. 54) MSD kit (Meso Scale Discovery, Catalog #K151A9H-4) in substantial accordance with the instructions provided by the manufacturer. The data was graphed using Prism software and is provided in FIGS. 52, 53 and 54 of the attached drawings. As illustrated in the data presented, introduction of the single point mutation E96K in the fused hIL10 format (h_CB0009_AA) reduces the activity of the molecule at inhibiting LPS-induced proinflammatory cytokine secretion by monocytes.

### Example 31. Monocyte IL10 Activity Assay of CB0010 (E96Q)

Human PBMCs were isolated from Leukopak obtained from a non-smoking human donor (Donor #072) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the h_DR2339_AA or h_CB0010_AA as described Table 18 below at concentrations ranging from 0.02 pM to 1000 nM for 48 hours at 37 °C. The description of h_DR2339_AA (SEQ ID NO:208) is provided in Example 29. The test article h_CB0010_AA was expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequence described in Table 18.

TABLE 18
h_CB0010_AA

| SEQ | | | |
|---|---|---|---|
| ID NO | Name | Description | AA Sequence |
| 214 | h_CB0010_AA | E96Q/E96Q | SPGQGTQSENSCTHFPGNLPNMLRDL |
| | | hIL10; GGS- | RDAFSRVKTFFQMKDQLDNLLLKES |
| | | His8 (SEQ ID | LLEDFKGYLGCQALSEMIQFYLEEV |
| | | NO: 23) | MPQAENQDPDIKAHVNSLGQNLKTL |
| | | | RLRLRRCHRFLPCENKSKAVEQVKN |
| | | | AFNKLQEKGIYKAMSEFDIFINYIEAY |
| | | | MTMKIRNSPGQGTQSENSCTHFPGNL |
| | | | PNMLRDLRDAFSRVKTFFQMKDQLD |
| | | | NLLLKESLLEDFKGYLGCQALSEMIQ |
| | | | FYLEEVMPQAENQDPDIKAHVNSLG |
| | | | QNLKTLRLRLRRCHRFLPCENKSKA |
| | | | VEQVKNAFNKLQEKGIYKAMSEFDIF |
| | | | INYIEAYMTMKIRNGGSHHHHHHHH |
| | | | (SEQ ID NO: 214) |

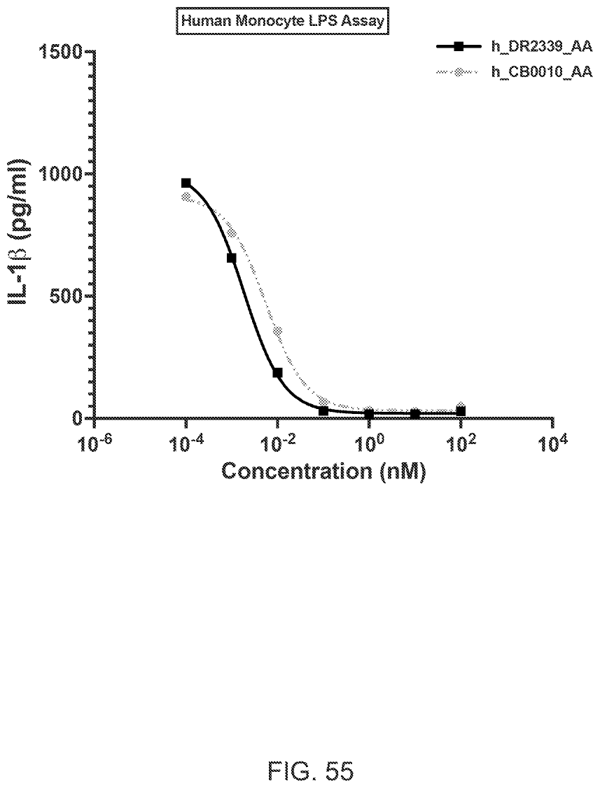

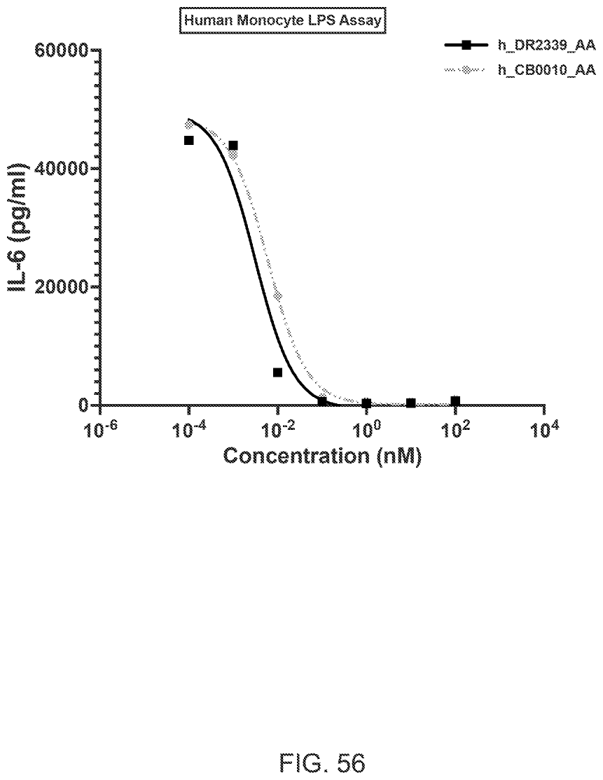

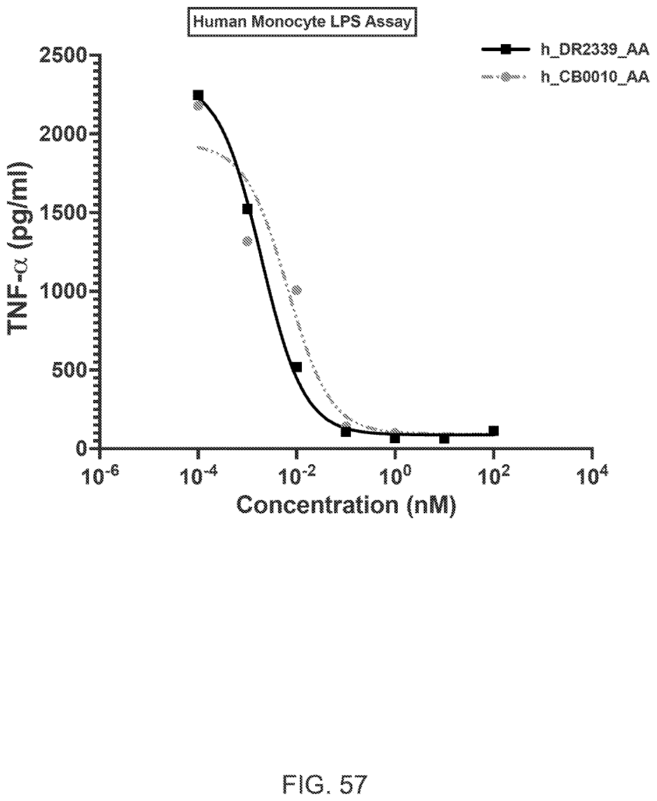

Post-treatment, cells were centrifuged at 400 g for 5 min and supernatants were collected. Supernatants were assessed for pro-inflammatory cytokines IL1b (FIG. 55), IL6 (FIG. 56) and TNFα (FIG. 57) using an MSD kit (Meso Scale Discovery, Catalog #K151A9H-4) in substantial accordance with the instructions provided by the manufacturer. The data was graphed using Prism software and is provided in FIGS. 55, 56 and 57 of the attached drawings. As illustrated in the data presented, the single point mutation E96Q introduced into the fused format (h_CB0010_AA) is potent at inhibiting LPS-induced proinflammatory cytokine secretion by monocytes with approximately 2-3 fold lower potency compared to fused_WT IL-10 (h_DR2339_AA).

### Example 32. Monocyte IL10 Activity Assay of CB0014 (R24E)

Human PBMCs were isolated from Leukopak obtained from a non-smoking human donor (Donor #072) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the h_DR2339_AA or h_CB0014_AA as described Table 19 below at concentrations ranging from 0.02 pM to 1000 nM for 48 hours at 37 °C. The description of h_DR2339_AA (SEQ ID NO:208) is provided in Example 29. The test article h_CB0014_AA was expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequence described in Table 19.

TABLE 19
h_CB0014_AA

| SEQ | | | |
|---|---|---|---|
| ID NO | Name | Description | AA Sequence |
| 215 | h_CB0014_AA | R24E/R24E | SPGQGTQSENSCTHFPGNLPNMLEDL |
| | | hIL10; GGS- | RDAFSRVKTFFQMKDQLDNLLLKES |
| | | His8 (SEQ ID | LLEDFKGYLGCQALSEMIQFYLEEV |
| | | NO: 23) | MPQAENQDPDIKAHVNSLGENLKTL |
| | | | RLRLRRCHRFLPCENKSKAVEQVKN |
| | | | AFNKLQEKGIYKAMSEFDIFINYIEAY |
| | | | MTMKIRNSPGQGTQSENSCTHFPGNL |
| | | | PNMLEDLRDAFSRVKTFFQMKDQLD |
| | | | NLLLKESLLEDFKGYLGCQALSEMIQ |
| | | | FYLEEVMPQAENQDPDIKAHVNSLG |
| | | | ENLKTLRLRLRRCHRFLPCENKSKAV |
| | | | EQVKNAFNKLQEKGIYKAMSEFDIFI |
| | | | NYIEAYMTMKIRNGGSHHHHHHHH |
| | | | (SEQ ID NO: 215) |

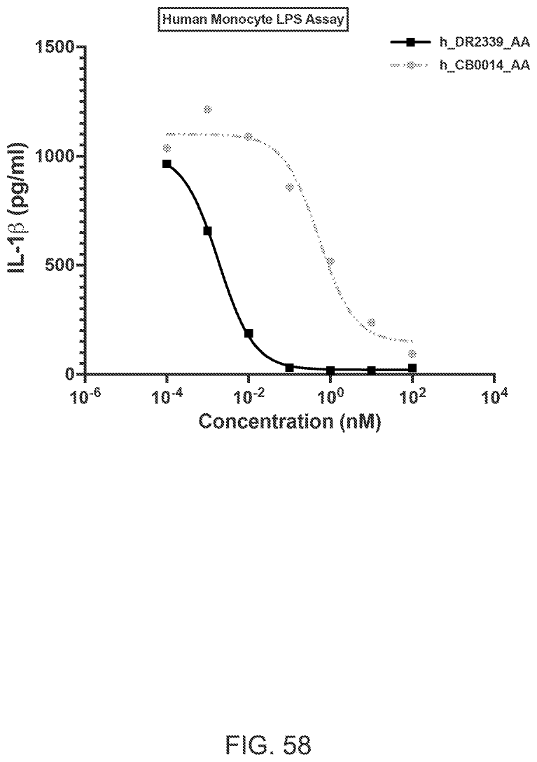

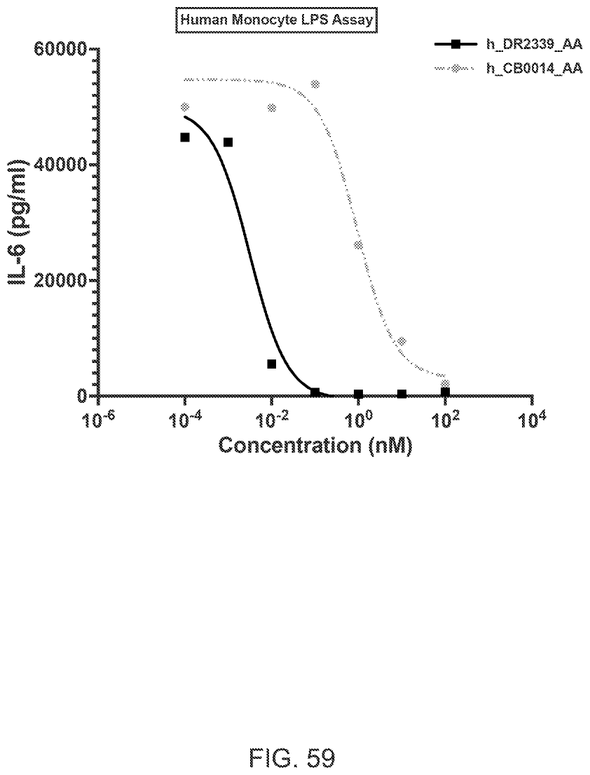

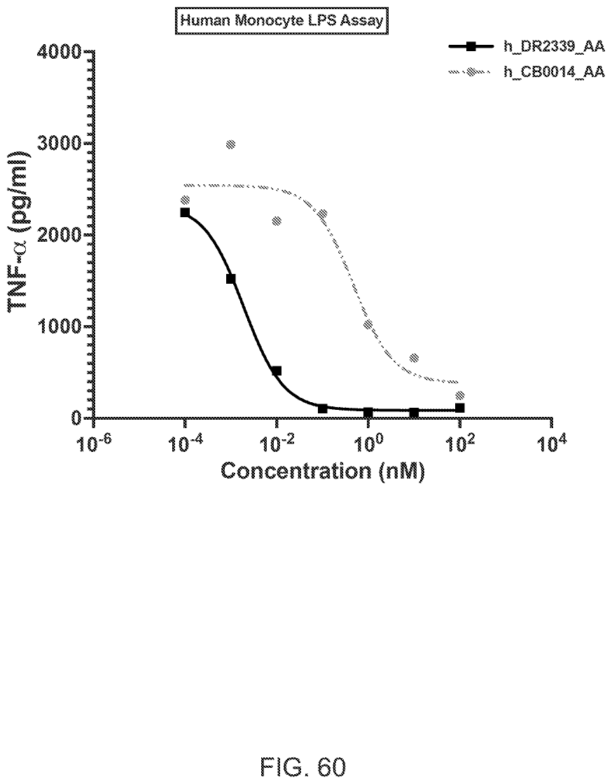

Post-treatment, cells were centrifuged at 400 g for 5 min and supernatants were collected. Supernatants were assessed for pro-inflammatory cytokines IL1b (FIG. 58), IL6 (FIG. 59) and TNFa (FIG. 60) using an MSD kit (Meso Scale Discovery, Catalog #K151A9H-4) in substantial accordance with the instructions provided by the manufacturer. The data was graphed using Prism software and is provided in FIGS. 58, 59, and 60 of the attached drawings. As illustrated in the data presented, the single point mutation R24E introduced into the fused format (h_CB0014_AA) inhibits LPS-induced proinflammatory cytokine secretion by monocytes, albeit with approximately 300 fold lower potency compared to fused_WT IL-10 (h_DR2339_AA).

### Example 33. Monocyte IL10 Activity Assay of CB0007 (E96K)

Human PBMCs were isolated from Leukopak obtained from a non-smoking human donor (Donor #072) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the h_DR2339_AA or h_CB0007_AA as described Table 20 below at concentrations ranging from 0.02 pM to 1000 nM for 48 hours at 37 °C. The description of h_DR2339_AA (SEQ ID NO:208) is provided in Example 29. The test article h_CB0007_AA was expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequence described in Table 20.

TABLE 20
h_CB0007_AA

| SEQ | | | |
|---|---|---|---|
| ID NO | Name | Description | AA Sequence |
| 216 | h_CB0007_AA | M22A/M22A | SPGQGTQSENSCTHFPGNLPDALRDL |
| | | hIL10; GGS- | RDAFSRVKTFFQMKDQLDNLLLKES |
| | | His8 (SEQ ID | LLEDFKGYLGCQALSEMIQFYLEEV |
| | | NO: 23) | MPQAENQDPDIKAHVNSLGENLKTL |
| | | | RLRLRRCHRFLPCENKSKAVEQVKN |
| | | | AFNKLQEKGIYKAMSEFDIFINYIEAY |
| | | | MTMKIRNSPGQGTQSENSCTHFPGNL |
| | | | PDALRDLRDAFSRVKTFFQMKDQLD |
| | | | NLLLKESLLEDFKGYLGCQALSEMIQ |
| | | | FYLEEVMPQAENQDPDIKAHVNSLG |
| | | | ENLKTLRLRLRRCHRFLPCENKSKAV |
| | | | EQVKNAFNKLQEKGIYKAMSEFDIFI |
| | | | NYIEAYMTMKIRNGGSHHHHHHHH |
| | | | (SEQ ID NO: 216) |

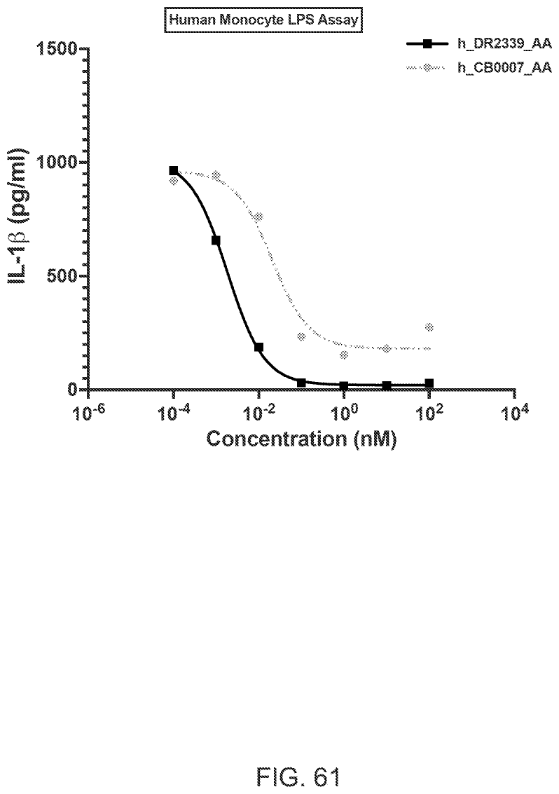

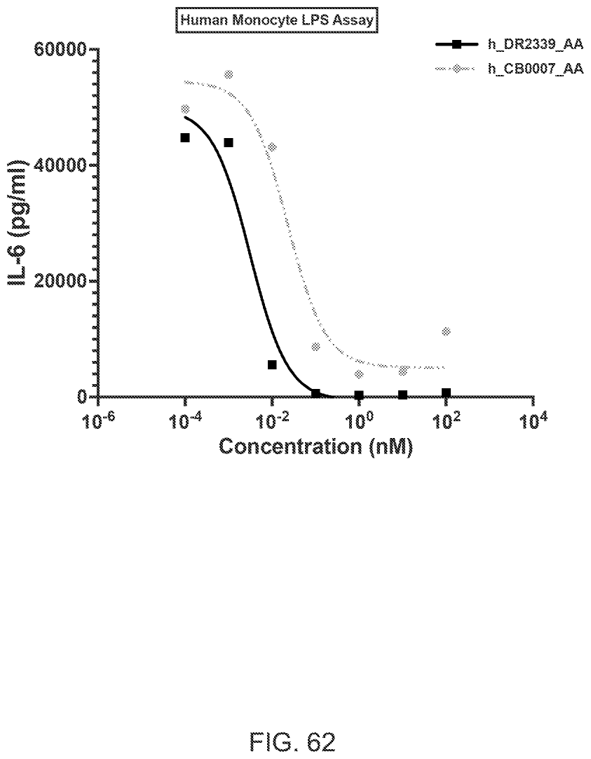

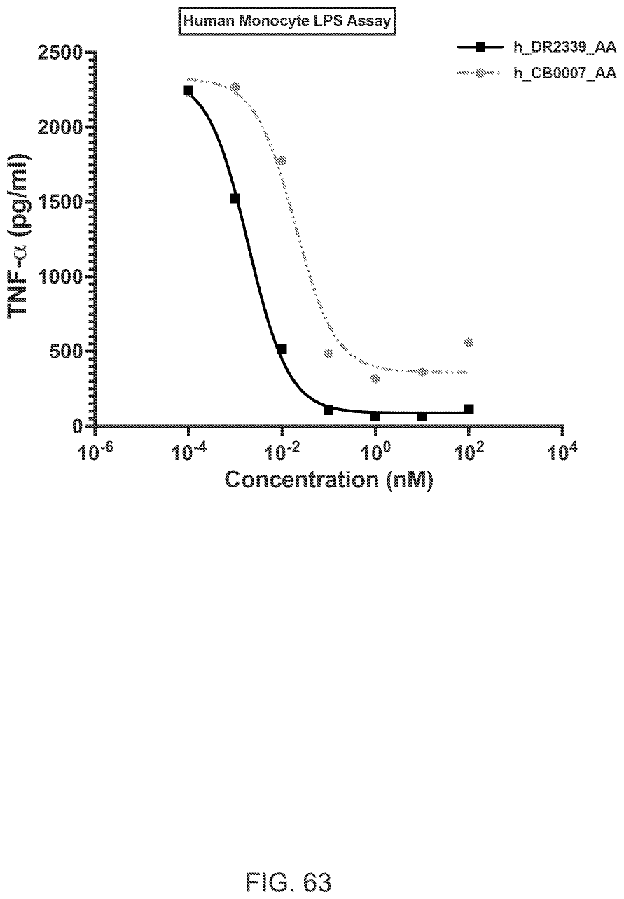

Post-treatment, cells were centrifuged at 400 g for 5 min and supernatants were collected. Supernatants were assessed for pro-inflammatory cytokines IL1b (FIG. 61), IL6 (FIG. 62) and TNFa (FIG. 63) using an MSD kit (Meso Scale Discovery, Catalog #K151A9H-4) in substantial accordance with the instructions provided by the manufacturer. The data was graphed using Prism software and is provided in FIGS. 61, 62 and 63 of the attached drawings.

As illustrated by the data presented in FIGS. 61, 62 and 63, the single point mutation M22A introduced into the fused format (h_CB0007_AA) inhibits LPS-induced proinflammatory cytokine secretion by monocytes, with approximately 10 fold lower potency compared to fused hWT IL-10 (h_DR2339_AA).

### Example 34. Monocyte IL10 Activity Assay of CB0005 (M22S)

Human PBMCs were isolated from Leukopak obtained from a non-smoking human donor (Donor #072) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the h_DR2339_AA or h_CB0005_AA as described Table 21 below at concentrations ranging from 0.02 pM to 1000 nM for 48 hours at 37 °C. The description of h_DR2339_AA (SEQ ID NO:208) is provided in Example 29. The test article h_CB0005_AA was expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequence described in Table 21.

TABLE 21
h_CB0005_AA

| SEQ | | | |
|---|---|---|---|
| ID NO | Name | Description | AA Sequence |
| 217 | h_CB0005_AA | M22S/M22S | SPGQGTQSENSCTHFPGNLPDSLRDL |
| | | hIL10; GGS- | RDAFSRVKTFFQMKDQLDNLLLKES |
| | | His8 (SEQ ID | LLEDFKGYLGCQALSEMIQFYLEEV |
| | | NO: 23) | MPQAENQDPDIKAHVNSLGENLKTL |
| | | | RLRLRRCHRFLPCENKSKAVEQVKN |
| | | | AFNKLQEKGIYKAMSEFDIFINYIEAY |
| | | | MTMKIRNSPGQGTQSENSCTHFPGNL |
| | | | PDSLRDLRDAFSRVKTFFQMKDQLD |
| | | | NLLLKESLLEDFKGYLGCQALSEMIQ |
| | | | FYLEEVMPQAENQDPDIKAHVNSLG |
| | | | ENLKTLRLRLRRCHRFLPCENKSKAV |
| | | | EQVKNAFNKLQEKGIYKAMSEFDIFI |
| | | | NYIEAYMTMKIRNGGSHHHHHHHH |
| | | | (SEQ ID NO: 217) |

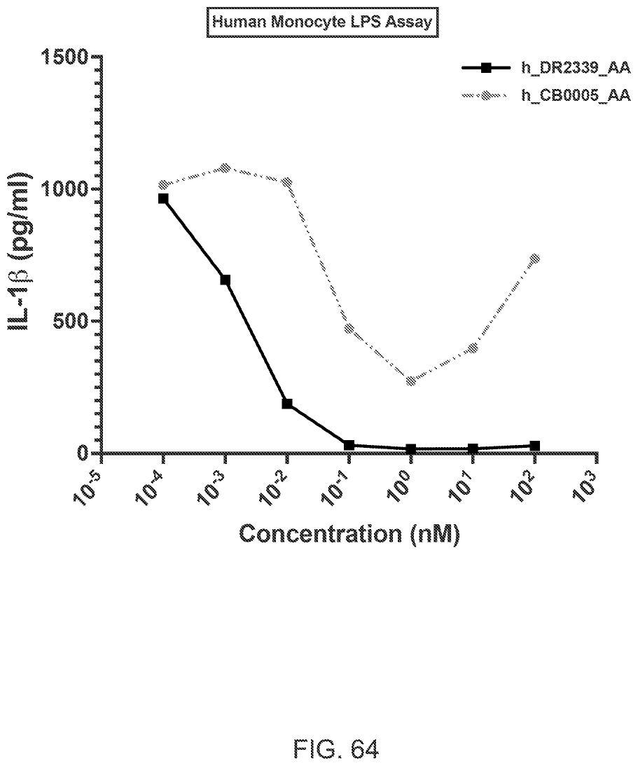

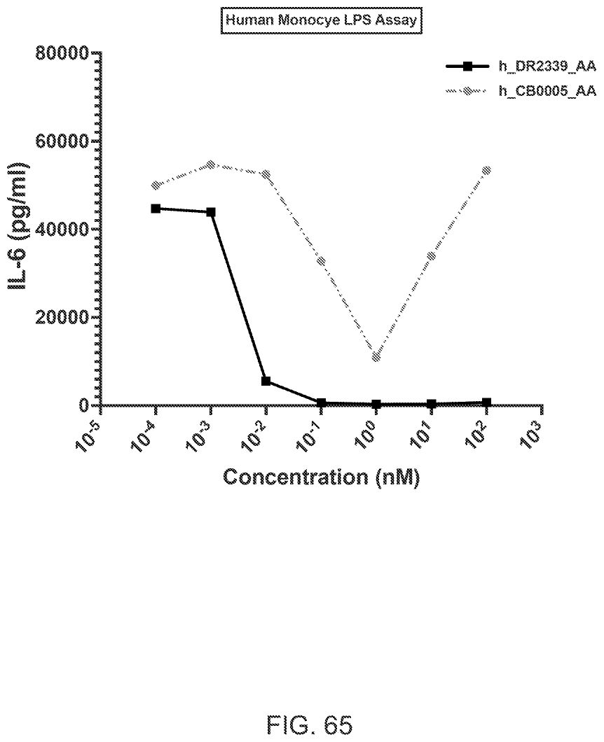

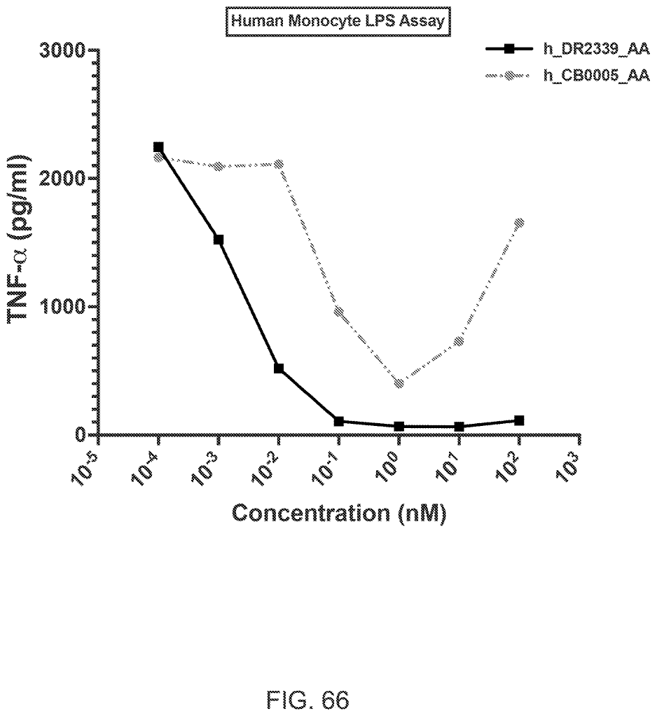

Post-treatment, cells were centrifuged at 400 g for 5 min and supernatants were collected. Supernatants were assessed for pro-inflammatory cytokines IL1b (FIG. 64), IL6 (FIG. 65) and TNFα (FIG. 66) using an MSD kit (Meso Scale Discovery, Catalog #K151A9H-4) in substantial accordance with the instructions provided by the manufacturer. The data was graphed using Prism software and is provided in FIGS. 64, 65 and 66 of the attached drawings. As illustrated by the presented, the single point mutation M22S introduced into the fused format (h_CB0005_AA) does not completely inhibit LPS-induced proinflammatory cytokine secretion by monocytes and has a blocking effect above 1 nM concentration.

### Example 35. Monocyte IL10 Activity Assay of CB0006 (M22T)

Human PBMCs were isolated from Leukopak obtained from a non-smoking human donor (Donor #072) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the h_DR2339_AA or h_CB0006_AA as described Table 22 below at concentrations ranging from 0.02 pM to 1000 nM for 48 hours at 37 °C. The description of h_DR2339_AA (SEQ ID NO:208) is provided in Example 29. The test article h_CB0006_AA was expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequence described in Table 22.

TABLE 22
h_CB0006AA

| SEQ | | | |
|---|---|---|---|
| ID NO | Name | Description | AA Sequence |
| 218 | h_CB0006_AA | M22T/M22T | SPGQGTQSENSCTHFPGNLPDTLRDL |
| | | hIL10; GGS- | RDAFSRVKTFFQMKDQLDNLLLKES |
| | | His8 (SEQ ID | LLEDFKGYLGCQALSEMIQFYLEEV |
| | | NO: 23) | MPQAENQDPDIKAHVNSLGENLKTL |
| | | | RLRLRRCHRFLPCENKSKAVEQVKN |
| | | | AFNKLQEKGIYKAMSEFDIFINYIEAY |
| | | | MTMKIRNSPGQGTQSENSCTHFPGNL |
| | | | PDTLRDLRDAFSRVKTFFQMKDQLD |
| | | | NLLLKESLLEDFKGYLGCQALSEMIQ |
| | | | FYLEEVMPQAENQDPDIKAHVNSLG |
| | | | ENLKTLRLRLRRCHRFLPCENKSKAV |
| | | | EQVKNAFNKLQEKGIYKAMSEFDIFI |
| | | | NYIEAYMTMKIRNGGSHHHHHHHH |
| | | | (SEQ ID NO: 218) |

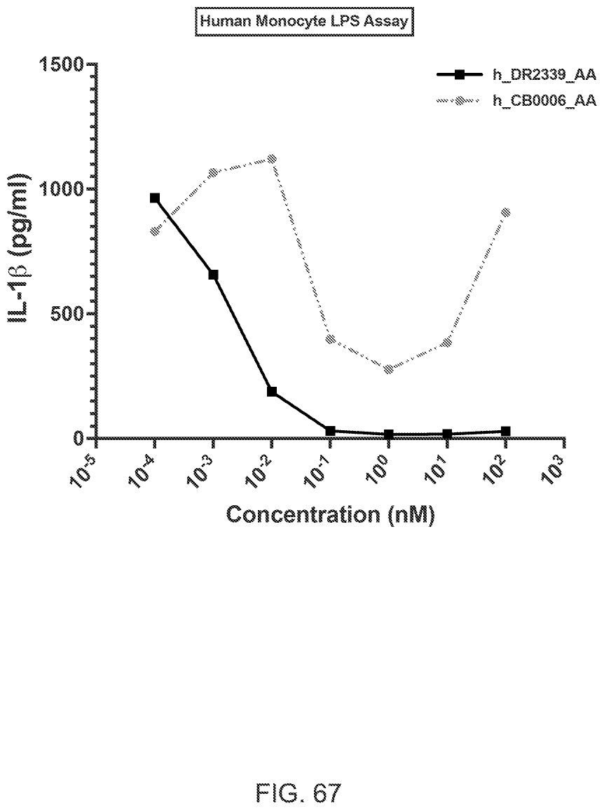

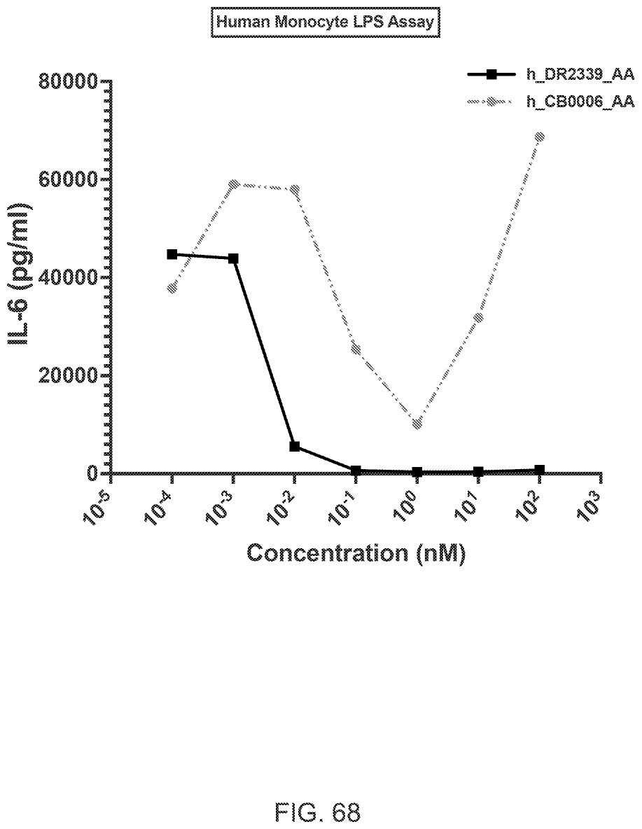

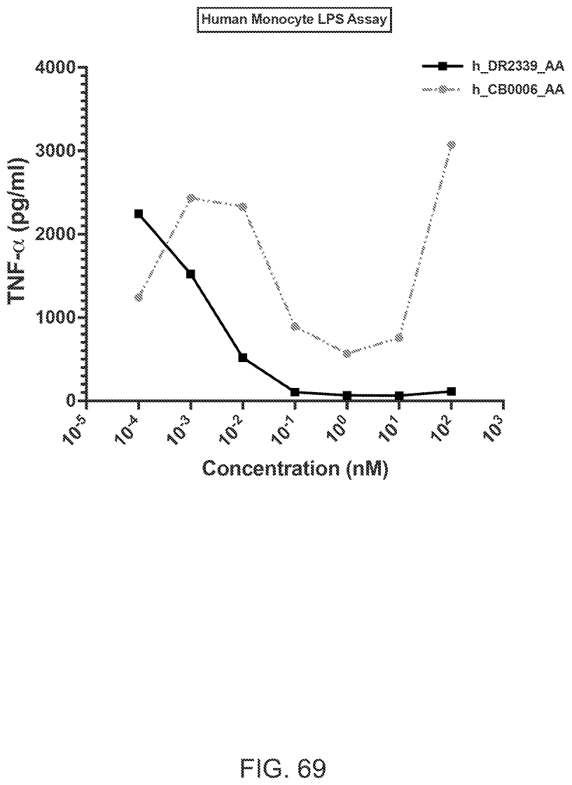

Post-treatment, cells were centrifuged at 400 g for 5 min and supernatants were collected. Supernatants were assessed for pro-inflammatory cytokines IL1b (FIG. 67), IL6 (FIG. 68) and TNFa (FIG. 69) using an MSD kit (Meso Scale Discovery, Catalog #K151A9H-4) in substantial accordance with the instructions provided by the manufacturer. The data was graphed using Prism software and is provided in FIGS. 67, 68, and 69 of the attached drawings. As illustrated by the presented, the single point mutation M22T introduced into the fused format (h_CB0006_AA) does not completely inhibit LPS-induced proinflammatory cytokine secretion by monocytes and has a blocking effect above 1 nM concentration.

### Example 36. Monocyte IL10 Activity Assay of CB0011 (T100E)

Human PBMCs were isolated from Leukopak obtained from a non-smoking human donor (Donor #072) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the h_DR2339_AA or h_CB0011_AA as described Table 23 below at concentrations ranging from 0.02 pM to 1000 nM for 48 hours at 37 °C. The description of h_DR2339_AA (SEQ ID NO:208) is provided in Example 29. The test article h_CB0011_AA was expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequence described in Table 23.

TABLE 23
h_CB0011_AA

| SEQ | | | |
|---|---|---|---|
| ID NO | Name | Description | AA Sequence |
| 219 | h_CB0011_AA | T100E/T100E | SPGQGTQSENSCTHFPGNLPNMLRDL |
| | | hIL10; GGS- | RDAFSRVKTFFQMKDQLDNLLLKES |
| | | His8 (SEQ ID | LLEDFKGYLGCQALSEMIQFYLEEV |
| | | NO: 23) | MPQAENQDPDIKAHVNSLGENLKEL |
| | | | RLRLRRCHRFLPCENKSKAVEQVKN |
| | | | AFNKLQEKGIYKAMSEFDIFINYIEAY |
| | | | MTMKIRNSPGQGTQSENSCTHFPGNL |
| | | | PNMLRDLRDAFSRVKTFFQMKDQLD |
| | | | NLLLKESLLEDFKGYLGCQALSEMIQ |
| | | | FYLEEVMPQAENQDPDIKAHVNSLG |
| | | | ENLKELRLRLRRCHRFLPCENKSKAV |
| | | | EQVKNAFNKLQEKGIYKAMSEFDIFI |
| | | | NYIEAYMTMKIRNGGSHHHHHHHH |
| | | | (SEQ ID NO: 219) |

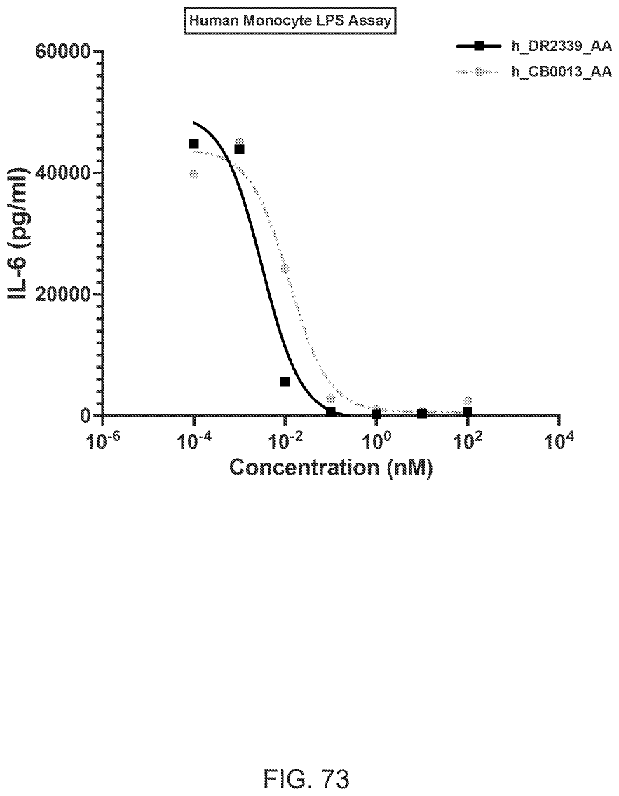

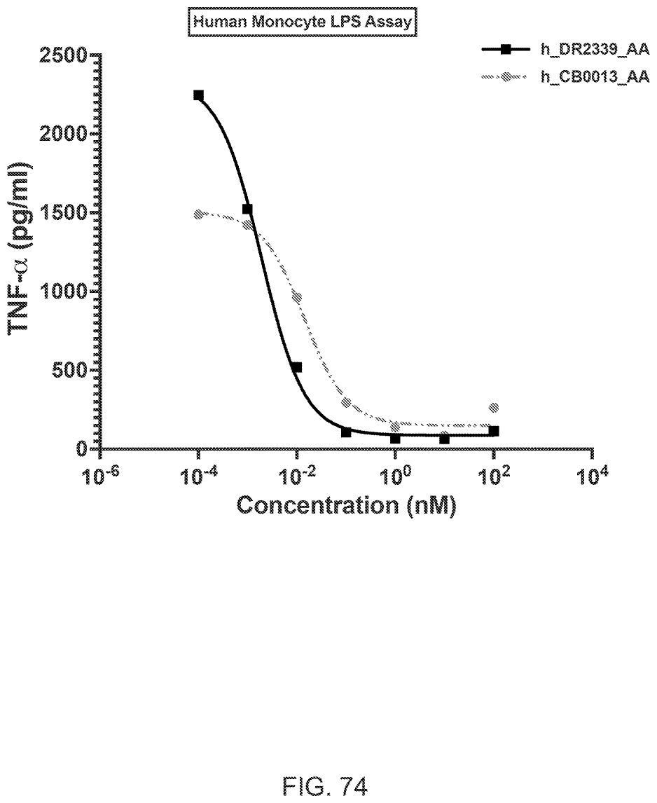

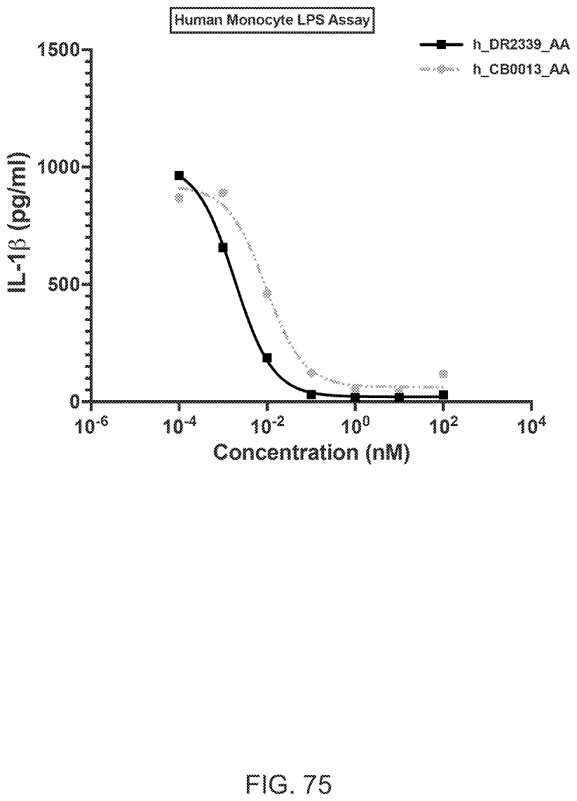

Post-treatment, cells were centrifuged at 400 g for 5 min and supernatants were collected. Supernatants were assessed for pro-inflammatory cytokines IL1b (FIG. 73), IL6 (FIG. 74) and TNFα (FIG. 75) using an MSD kit (Meso Scale Discovery, Catalog #K151A9H-4) in substantial accordance with the instructions provided by the manufacturer. The data was graphed using Prism software and is provided in FIGS. 73, 74 and 75 of the attached drawings. As illustrated by the data presented in FIGS. 73, 74 and 75, the single point mutation T100E introduced into the fused format (h_CB0011_AA) inhibits LPS-induced proinflammatory cytokine secretion by monocytes, with less than 3-fold lower potency drop compared to fused_WT IL-10 (h_DR2339_AA).

### Example 37. Monocyte IL10 Activity Assay of CB0013 (T100L)

Human PBMCs were isolated from Leukopak obtained from a non-smoking human donor (Donor #072) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the h_DR2339_AA or h_CB0013_AA as described Table 24 below at concentrations ranging from 0.02 pM to 1000 nM for 48 hours at 37 °C. The description of h_DR2339_AA (SEQ ID NO:208) is provided in Example 29. The test article_CB0013_AA was expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequence described in Table 24.

TABLE 24
h_CB0013_AA

| SEQ | | | |
|---|---|---|---|
| ID NO | Name | Description | AA Sequence |
| 220 | h_CB0013_AA | T100L/T100L | SPGQGTQSENSCTHFPGNLPNMLRDL |
| | | hIL10; GGS- | RDAFSRVKTFFQMKDQLDNLLLKES |
| | | His8 (SEQ ID | LLEDFKGYLGCQALSEMIQFYLEEV |
| | | NO: 23) | MPQAENQDPDIKAHVNSLGENLKLL |
| | | | RLRLRRCHRFLPCENKSKAVEQVKN |
| | | | AFNKLQEKGIYKAMSEFDIFINYIEAY |
| | | | MTMKIRNSPGQGTQSENSCTHFPGNL |
| | | | PNMLRDLRDAFSRVKTFFQMKDQLD |
| | | | NLLLKESLLEDFKGYLGCQALSEMIQ |
| | | | FYLEEVMPQAENQDPDIKAHVNSLG |
| | | | ENLKLLRLRLRRCHRFLPCENKSKAV |
| | | | EQVKNAFNKLQEKGIYKAMSEFDIFI |
| | | | NYIEAYMTMKIRNGGSHHHHHHHH |
| | | | (SEQ ID NO: 220) |

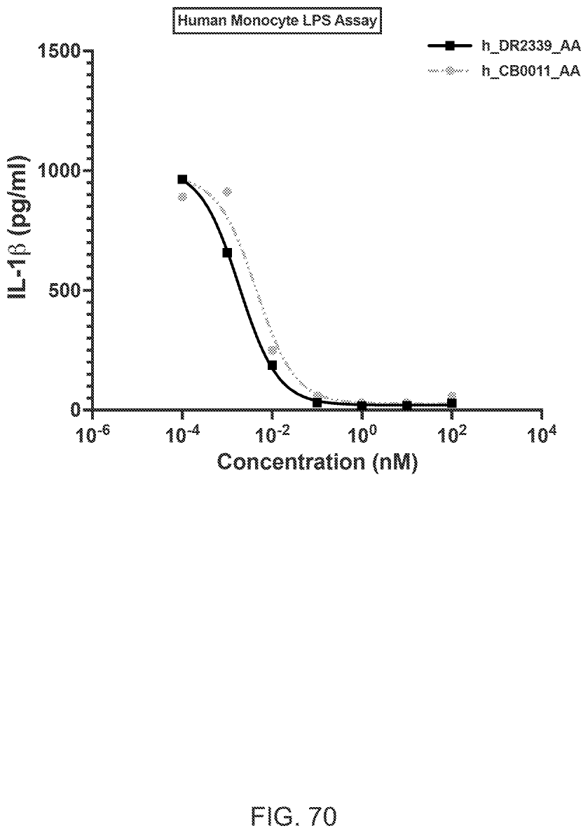

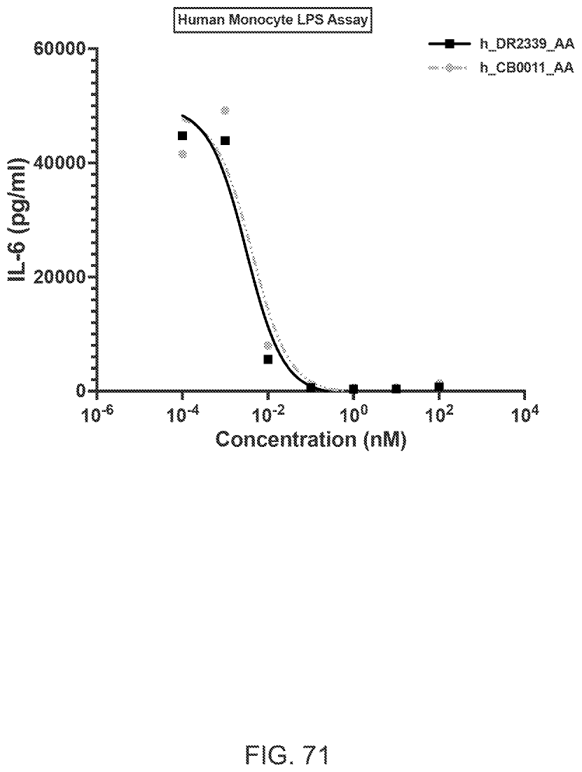

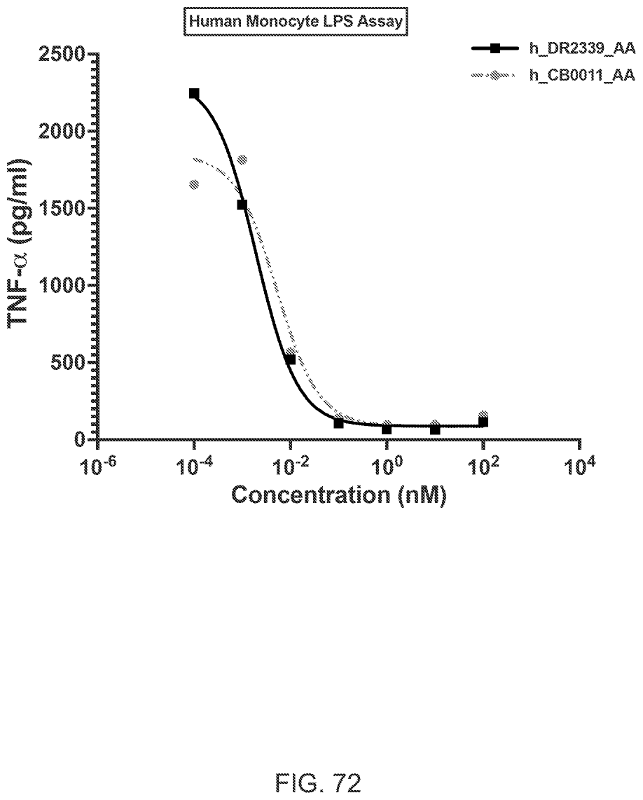

Post-treatment, cells were centrifuged at 400 g for 5 min and supernatants were collected. Supernatants were assessed for pro-inflammatory cytokines IL1b (FIG. 70), IL6 (FIG. 71) and TNFα (FIG. 72) using an MSD kit (Meso Scale Discovery, Catalog #K151A9H-4) in substantial accordance with the instructions provided by the manufacturer. The data was graphed using Prism software and is provided in FIGS. 73, 74 and 75 of the attached drawings. As illustrated by the data presented in provided in FIGS. 73, 74 and 75, the single point mutation T100L introduced into the fused format (h_CB0013_AA) inhibits LPS-induced proinflammatory cytokine secretion by monocytes, with 3-7 fold lower potency compared to fused_WT IL-10 (h_DR2339_AA)

### Example 38. Monocyte IL10 Activity Assay of CB0002 (N21D)

Human PBMCs were isolated from Leukopak obtained from a non-smoking human donor (Donor #072) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the h_DR2339_AA or h_CB0002_AA as described Table 25 below at concentrations ranging from 0.02 pM to 1000 nM for 48 hours at 37 °C. The description of h_DR2339_AA (SEQ ID NO:208) is provided in Example 29. The test article h_CB0002_AA was expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequence described in Table 25.

TABLE 25
h_CB0002_AA

| SEQ | | | |
|---|---|---|---|
| ID NO | Name | Description | AA Sequence |
| 221 | h_CB0002_AA | N21D/N21D | SPGQGTQSENSCTHFPGNLPDMLRDL |
| | | hIL10; GGS- | RDAFSRVKTFFQMKDQLDNLLLKES |
| | | His8 (SEQ ID | LLEDFKGYLGCQALSEMIQFYLEEV |
| | | NO: 23) | MPQAENQDPDIKAHVNSLGENLKTL |
| | | | RLRLRRCHRFLPCENKSKAVEQVKN |
| | | | AFNKLQEKGIYKAMSEFDIFINYIEAY |
| | | | MTMKIRNSPGQGTQSENSCTHFPGNL |
| | | | PDMLRDLRDAFSRVKTFFQMKDQLD |
| | | | NLLLKESLLEDFKGYLGCQALSEMIQ |
| | | | FYLEEVMPQAENQDPDIKAHVNSLG |
| | | | ENLKTLRLRLRRCHRFLPCENKSKAV |
| | | | EQVKNAFNKLQEKGIYKAMSEFDIFI |
| | | | NYIEAYMTMKIRNGGSHHHHHHHH |
| | | | (SEQ ID NO: 221) |

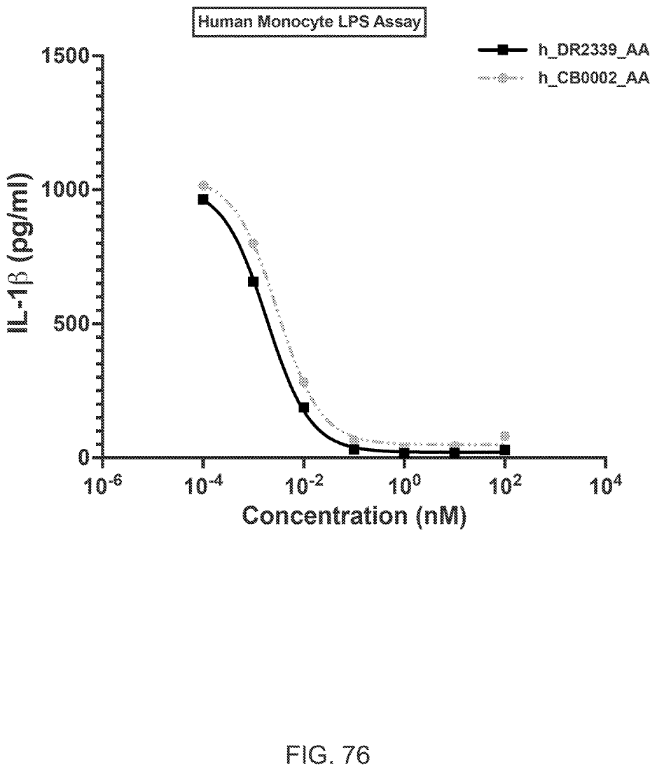

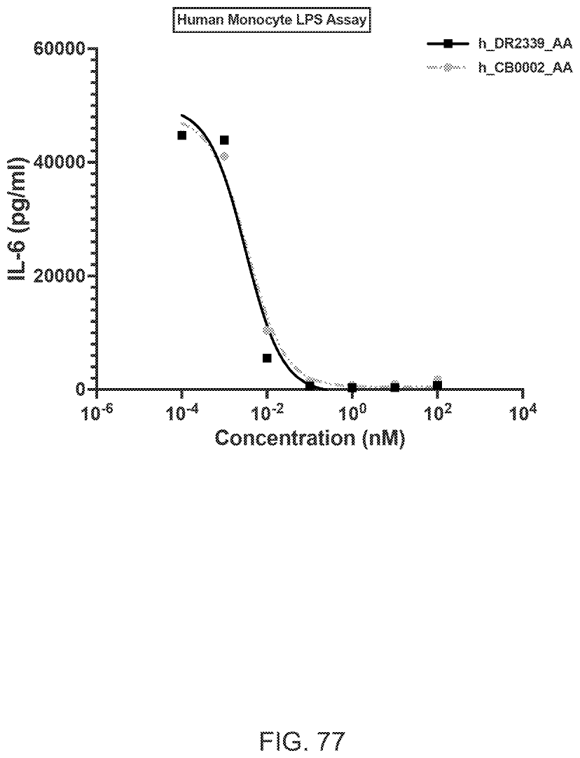

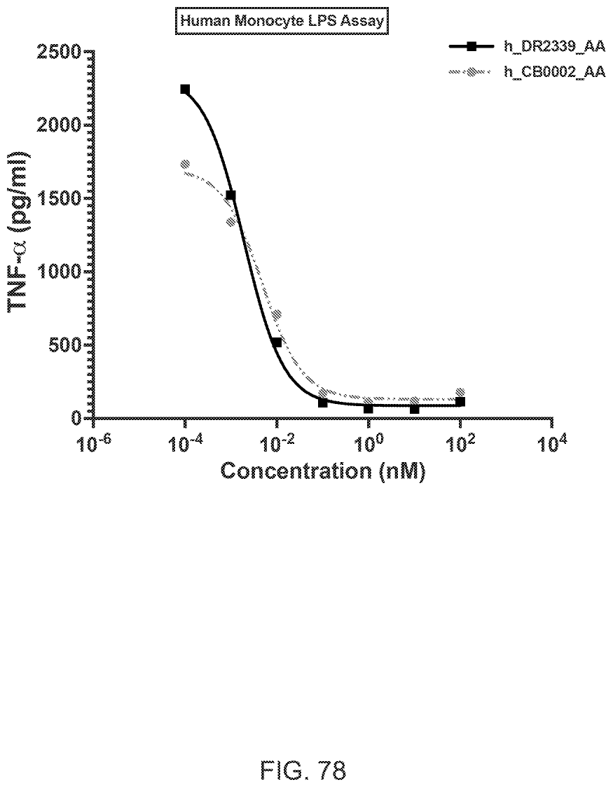

Post-treatment, cells were centrifuged at 400 g for 5 min and supernatants were collected. Supernatants were assessed for pro-inflammatory cytokines IL1b (FIG. 76), IL6 (FIG. 77) and TNFα (FIG. 78) using an MSD kit (Meso Scale Discovery, Catalog #K151A9H-4) in substantial accordance with the instructions provided by the manufacturer. The data was graphed using Prism software and is provided in FIGS. 76, 77 and 78 of the attached drawings. As illustrated by the data presented in provided in FIGS. 76, 77 and 78, the single point mutation N21D introduced into the fused format (h_CB0002_AA) inhibits LPS-induced proinflammatory cytokine secretion by monocytes, with less than 2-fold potency drop compared to fused_WT IL-10 (h_DR2339_AA).

### Example 38. Monocyte IL10 Activity Assay: of CB0003 (N21E)

Human PBMCs were isolated from Leukopak obtained from a non-smoking human donor (Donor #072) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the h_DR2339_AA or h_CB0003_AA as described Table 26 below at concentrations ranging from 0.02 pM to 1000 nM for 48 hours at 37 °C. The description of h_DR2339_AA (SEQ ID NO:208) is provided in Example 29. The test article h_CB0003_AA was expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequence described in Table 26.

TABLE 26
h_CB0003_AA

| SEQ | | | |
|---|---|---|---|
| ID NO | Name | Description | AA Sequence |
| 222 | h_CB0003_AA | N21E/N21E | SPGQGTQSENSCTHFPGNLPEMLRDL |
| | | hIL10; GGS- | RDAFSRVKTFFQMKDQLDNLLLKES |
| | | His8 (SEQ ID | LLEDFKGYLGCQALSEMIQFYLEEV |
| | | NO: 23) | MPQAENQDPDIKAHVNSLGENLKTL |
| | | | RLRLRRCHRFLPCENKSKAVEQVKN |
| | | | AFNKLQEKGIYKAMSEFDIFINYIEAY |
| | | | MTMKIRNSPGQGTQSENSCTHFPGNL |
| | | | PEMLRDLRDAFSRVKTFFQMKDQLD |
| | | | NLLLKESLLEDFKGYLGCQALSEMIQ |
| | | | FYLEEVMPQAENQDPDIKAHVNSLG |
| | | | ENLKTLRLRLRRCHRFLPCENKSKAV |
| | | | EQVKNAFNKLQEKGIYKAMSEFDIFI |
| | | | NYIEAYMTMKIRNGGSHHHHHHHH |
| | | | (SEQ ID NO: 222) |

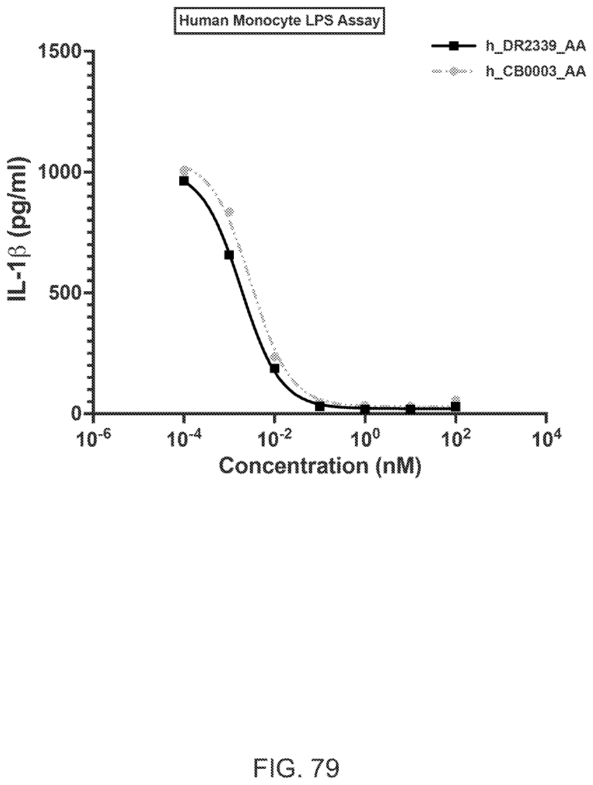

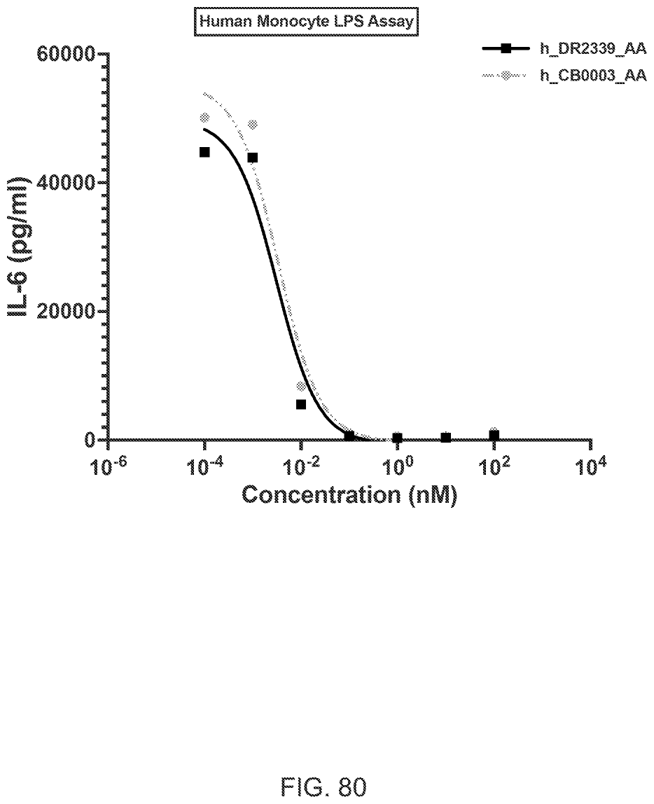

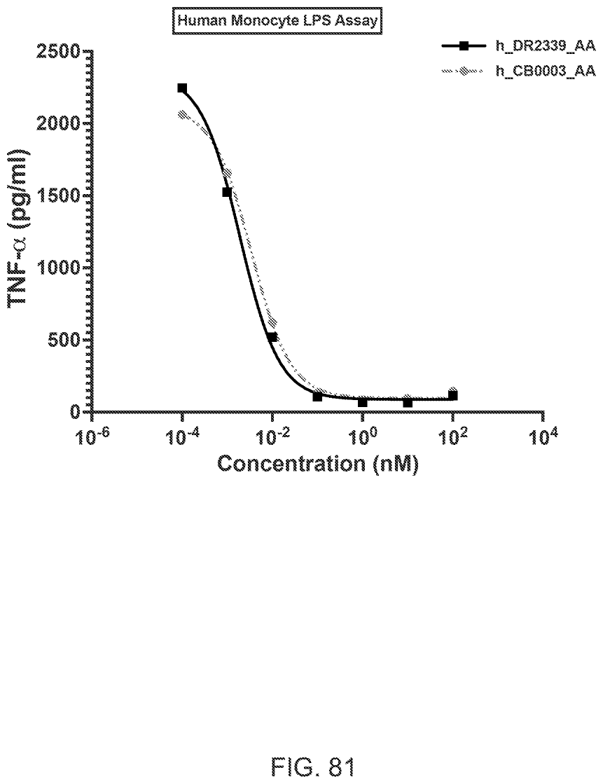

Post-treatment, cells were centrifuged at 400 g for 5 min and supernatants were collected. Supernatants were assessed for pro-inflammatory cytokines IL1b (FIG. 79), IL6 (FIG. 80) and TNFα (FIG. 81) using an MSD kit (Meso Scale Discovery, Catalog #K151A9H-4) in substantial accordance with the instructions provided by the manufacturer. The data was graphed using Prism software and is provided in FIGS. 79, 80 and 81 of the attached drawings. As illustrated by the data presented in provided in FIGS. 79, 80 and 81, the single point mutation N21E introduced into the fused format (h_CB0003_AA) inhibits LPS-induced proinflammatory cytokine secretion by monocytes, with less than 2-fold potency drop compared to fused h_WT IL-10 (h_DR2339_AA).

### Example 40. Monocyte IL10 Activity Assay of CB0001 (N21K)

Human PBMCs were isolated from Leukopak obtained from a non-smoking human donor (Donor #072) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the h_DR2339_AA or h_CB0001_AA as described Table 27 below at concentrations ranging from 0.02 pM to 1000 nM for 48 hours at 37 °C. The description of h_DR2339_AA (SEQ ID NO:208) is provided in Example 29. The test article h_CB0001_AA was expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequence described in Table 27.

TABLE 27
h_CB0001_AA

| SEQ | | | |
|---|---|---|---|
| ID NO | Name | Description | AA Sequence |
| 223 | h_CB0001_AA | N21K/N21K | SPGQGTQSENSCTHFPGNLPKMLRDL |
| | | hIL10; GGS- | RDAFSRVKTFFQMKDQLDNLLLKES |
| | | His8 (SEQ ID | LLEDFKGYLGCQALSEMIQFYLEEV |
| | | NO: 23) | MPQAENQDPDIKAHVNSLGENLKTL |
| | | | RLRLRRCHRFLPCENKSKAVEQVKN |
| | | | AFNKLQEKGIYKAMSEFDIFINYIEAY |
| | | | MTMKIRNSPGQGTQSENSCTHFPGNL |
| | | | PKMLRDLRDAFSRVKTFFQMKDQLD |
| | | | NLLLKESLLEDFKGYLGCQALSEMIQ |
| | | | FYLEEVMPQAENQDPDIKAHVNSLG |
| | | | ENLKTLRLRLRRCHRFLPCENKSKAV |
| | | | EQVKNAFNKLQEKGIYKAMSEFDIFI |
| | | | NYIEAYMTMKIRNGGSHHHHHHHH |
| | | | (SEQ ID NO: 223) |

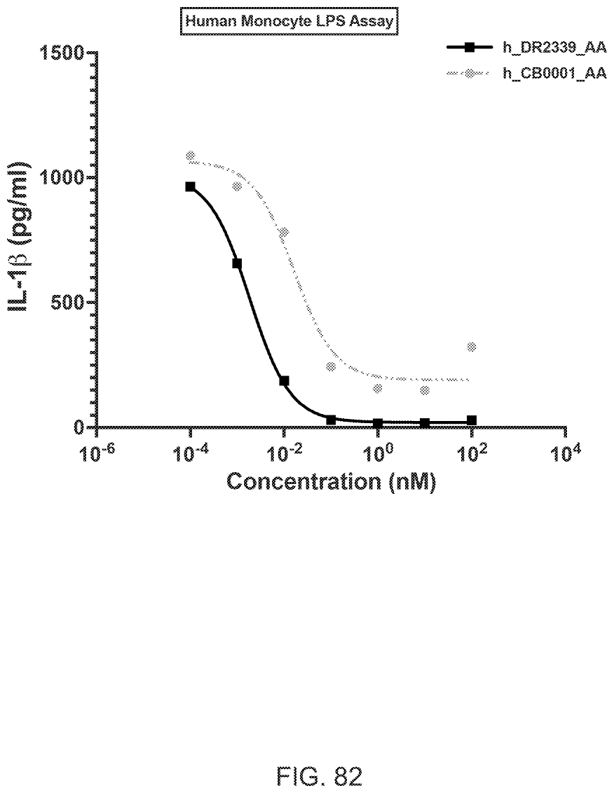

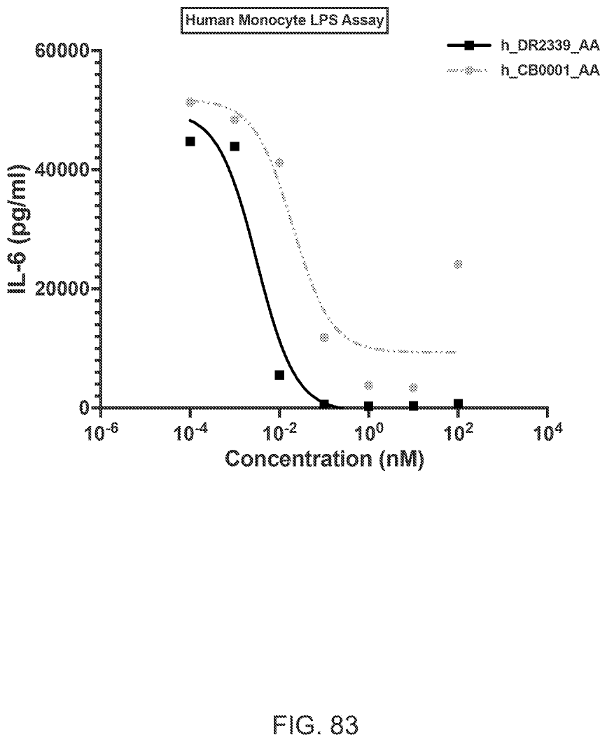

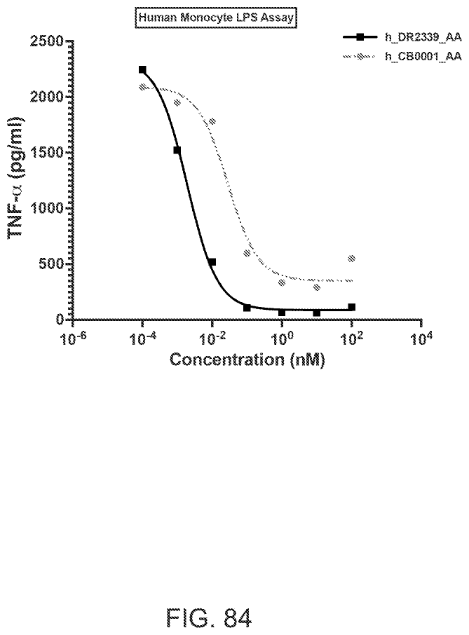

Post-treatment, cells were centrifuged at 400 g for 5 min and supernatants were collected. Supernatants were assessed for pro-inflammatory cytokines IL1b (FIG. 82), IL6 (FIG. 83) and TNFα (FIG. 84) using an MSD kit (Meso Scale Discovery, Catalog #K151A9H-4) in substantial accordance with the instructions provided by the manufacturer. The data was graphed using Prism software and is provided in FIGS. 82, 83 and 84 of the attached drawings. As illustrated by the data presented in provided in FIGS. 82, 83 and 84, the single point mutation N21K introduced into the fused format (h_CB0001_AA) inhibits LPS-induced proinflammatory cytokine secretion by monocytes, with approximately 10-fold lower potency compared to fused_WT IL-10 (h_DR2339_AA).

### Example 41. Monocyte IL10 Activity Assay of AG020 (D25K)

Human PBMCs were isolated from Leukopak obtained from a non-smoking human donor (Donor #072) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the h_DR2339_AA or h_AG020_AA as described Table 28 below at concentrations ranging from 0.02 pM to 1000 nM for 48 hours at 37 °C. The description of h_DR2339_AA (SEQ ID NO:208) is provided in Example 29. The test article h_AG020_AA was expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequence described in Table 28.

TABLE 28
h_AG020_AA

| SEQ | | | |
|---|---|---|---|
| ID NO | Name | Description | AA Sequence |
| 224 | h_AG020_AA | D25K/D25K | SPGQGTQSENSCTHFPGNLPNMLRKL |
| | | hIL10; GGS- | RDAFSRVKTFFQMKDQLDNLLLKES |
| | | His8 (SEQ ID | LLEDFKGYLGCQALSEMIQFYLEEV |
| | | NO: 23) | MPQAENQDPDIKAHVNSLGENLKTL |
| | | | RLRLRRCHRFLPCENKSKAVEQVKN |
| | | | AFNKLQEKGIYKAMSEFDIFINYIEAY |
| | | | MTMKIRNSPGQGTQSENSCTHFPGNL |
| | | | PNMLRKLRDAFSRVKTFFQMKDQLD |
| | | | NLLLKESLLEDFKGYLGCQALSEMIQ |
| | | | FYLEEVMPQAENQDPDIKAHVNSLG |
| | | | ENLKTLRLRLRRCHRFLPCENKSKAV |
| | | | EQVKNAFNKLQEKGIYKAMSEFDIFI |
| | | | NYIEAYMTMKIRNGGSHHHHHHHH |
| | | | (SEQ ID NO: 224) |

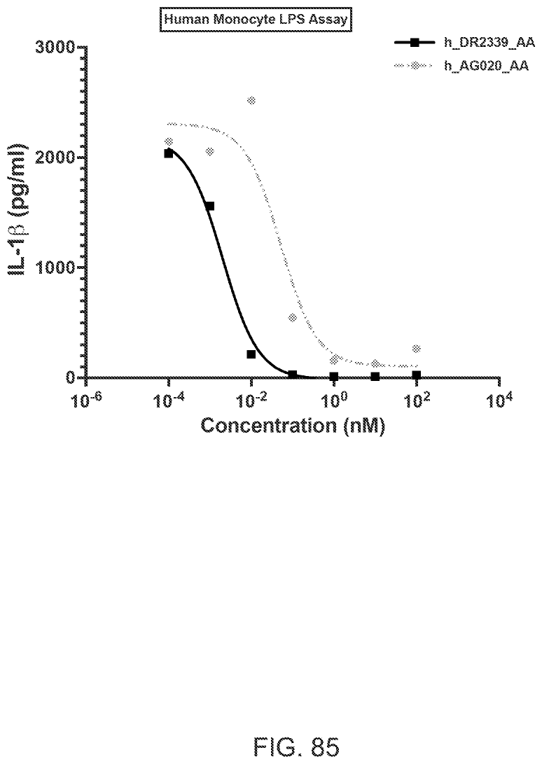

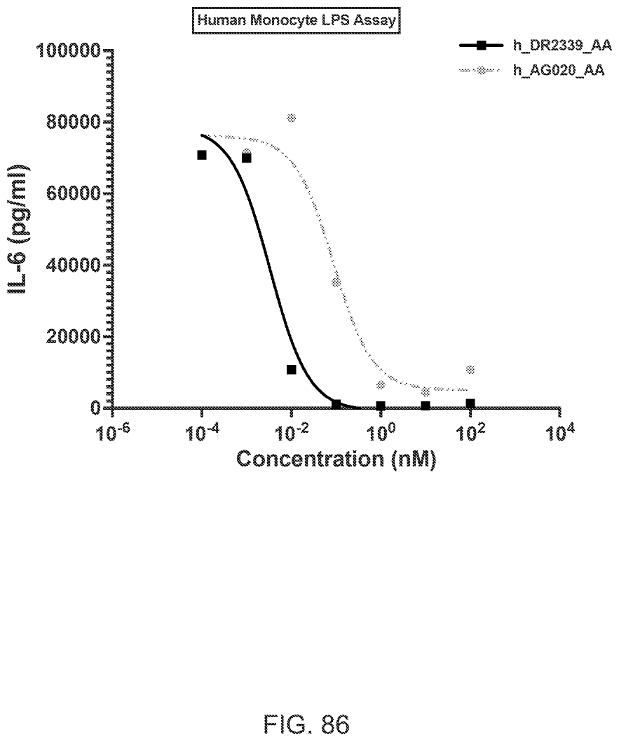

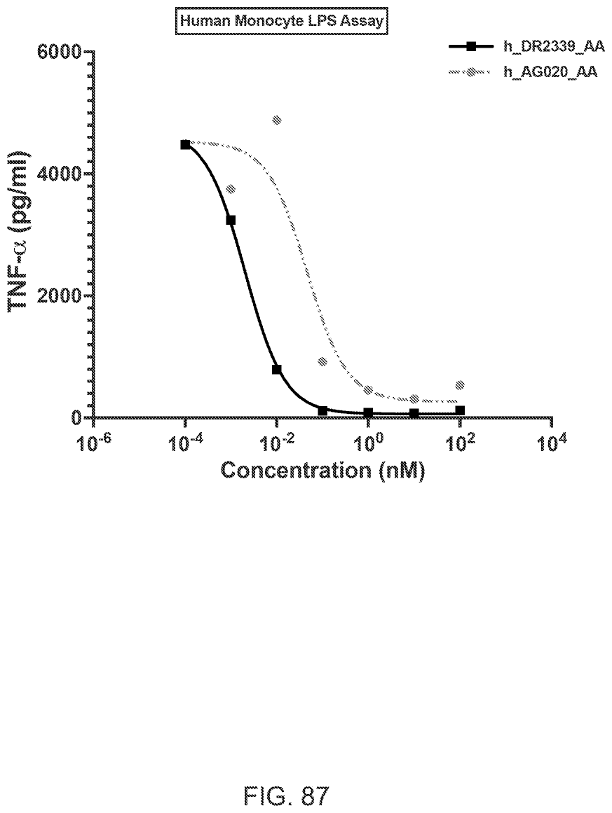

Post-treatment, cells were centrifuged at 400 g for 5 min and supernatants were collected. Supernatants were assessed for pro-inflammatory cytokines IL1b (FIG. 85), IL6 (FIG. 86) and TNFα (FIG. 87) using an MSD kit (Meso Scale Discovery, Catalog #K151A9H-4) in substantial accordance with the instructions provided by the manufacturer. The data was graphed using Prism software and is provided in FIGS. 85, 86, and 87 of the attached drawings. As illustrated by the data presented in provided in FIGS. 85, 86, and 87, the single point mutation D25K introduced into the fused format (h_AG020_AA) inhibits LPS-induced proinflammatory cytokine secretion by monocytes, with approximately 25-fold lower potency compared to fused_WT IL-10 (h_DR2339_AA).

### Example 42. Monocyte IL10 Activity Assay of CB0004 (M22D)

Human PBMCs were isolated from Leukopak obtained from a non-smoking human donor (Donor #072) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the h_DR2339_AA or h_CB0004_AA as described Table 29 below at concentrations ranging from 0.02 pM to 1000 nM for 48 hours at 37 °C. The description of h_DR2339_AA (SEQ ID NO:208) is provided in Example 29. The test article h_CB0004_AA was expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequence described in Table 29.

TABLE 29
h_CB0004_AA

| SEQ | | | |
|---|---|---|---|
| ID NO | Name | Description | AA Sequence |
| 225 | h_CB0004_AA | M22D/M22D | SPGQGTQSENSCTHFPGNLPDDLRDL |
| | | hIL10; GGS- | RDAFSRVKTFFQMKDQLDNLLLKES |
| | | His8 (SEQ ID | LLEDFKGYLGCQALSEMIQFYLEEV |
| | | NO: 23) | MPQAENQDPDIKAHVNSLGENLKTL |
| | | | RLRLRRCHRFLPCENKSKAVEQVKN |
| | | | AFNKLQEKGIYKAMSEFDIFINYIEAY |
| | | | MTMKIRNSPGQGTQSENSCTHFPGNL |
| | | | PDDLRDLRDAFSRVKTFFQMKDQLD |
| | | | NLLLKESLLEDFKGYLGCQALSEMIQ |
| | | | FYLEEVMPQAENQDPDIKAHVNSLG |
| | | | ENLKTLRLRLRRCHRFLPCENKSKAV |
| | | | EQVKNAFNKLQEKGIYKAMSEFDIFI |
| | | | NYIEAYMTMKIRNGGSHHHHHHHH |
| | | | (SEQ ID NO: 225) |

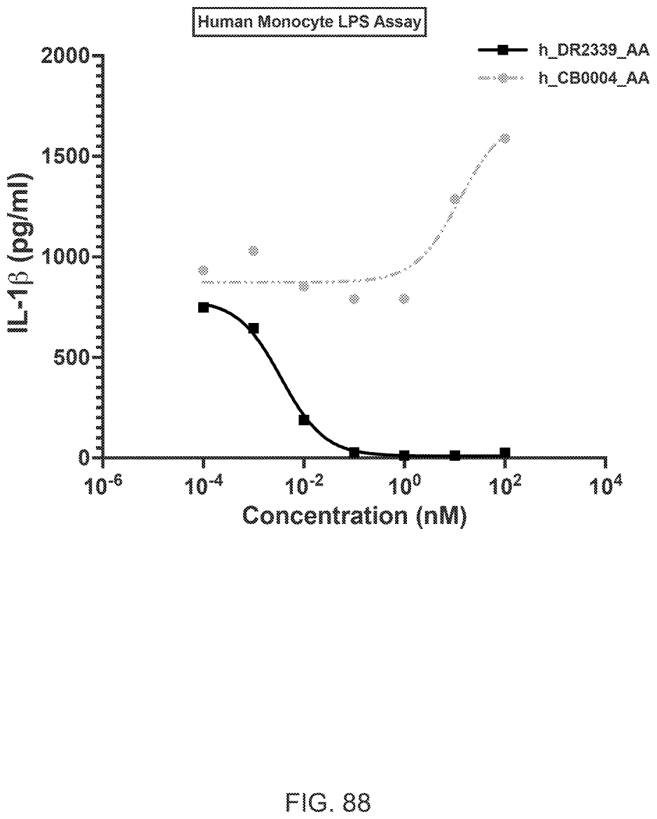

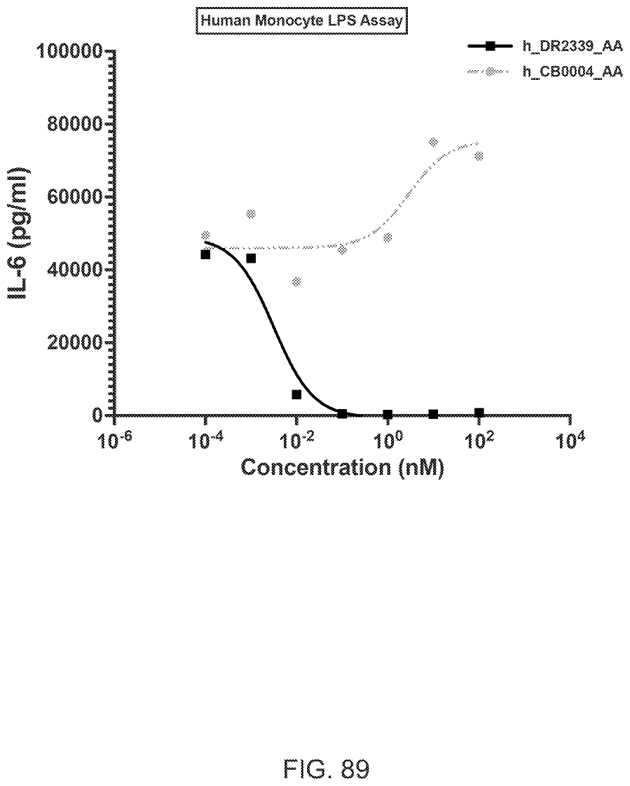

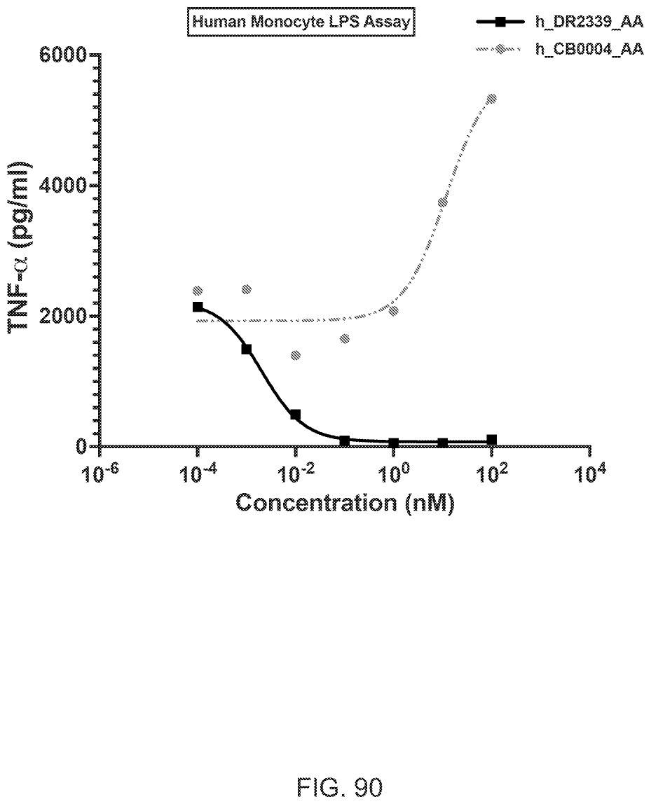

Post-treatment, cells were centrifuged at 400 g for 5 min and supernatants were collected. Supernatants were assessed for pro-inflammatory cytokines IL1b (FIG. 88), IL6 (FIG. 89) and TNFa (FIG. 90) using an MSD kit (Meso Scale Discovery, Catalog #K151A9H-4) in substantial accordance with the instructions provided by the manufacturer. The data was graphed using Prism software and is provided in FIGS. 88, 89 and 90 of the attached drawings. As illustrated by the data presented in provided in FIGS. 88, 89 and 90, the single point mutation M22D introduced into the fused format (h_CB0004_AA) does not inhibit LPS-induced proinflammatory cytokine secretion by monocytes.

### Example 43. Monocyte IL10 Activity Assay of CB0008 (M22W)

Human PBMCs were isolated from Leukopak obtained from a non-smoking human donor (Donor #072) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the h_DR2339_AA or h_CB0008_AA as described Table 30 below at concentrations ranging from 0.02 pM to 1000 nM for 48 hours at 37 °C. The description of h_DR2339_AA (SEQ ID NO:208) is provided in Example 29. The test article h_CB0008_AA was expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequence described in Table 30.

TABLE 30
h_CB0008_AA

| SEQ | | | |
|---|---|---|---|
| ID NO | Name | Description | AA Sequence |
| 226 | h_CB0008_AA | M22W/M22W | SPGQGTQSENSCTHFPGNLPDWLRDL |
| | | hIL10; GGS- | RDAFSRVKTFFQMKDQLDNLLLKES |
| | | His8 (SEQ ID | LLEDFKGYLGCQALSEMIQFYLEEV |
| | | NO: 23) | MPQAENQDPDIKAHVNSLGENLKTL |
| | | | RLRLRRCHRFLPCENKSKAVEQVKN |
| | | | AFNKLQEKGIYKAMSEFDIFINYIEAY |
| | | | MTMKIRNSPGQGTQSENSCTHFPGNL |
| | | | PDWLRDLRDAFSRVKTFFQMKDQLD |
| | | | NLLLKESLLEDFKGYLGCQALSEMIQ |
| | | | FYLEEVMPQAENQDPDIKAHVNSLG |
| | | | ENLKTLRLRLRRCHRFLPCENKSKAV |
| | | | EQVKNAFNKLQEKGIYKAMSEFDIFI |
| | | | NYIEAYMTMKIRNGGSHHHHHHHH |
| | | | (SEQ ID NO: 226) |

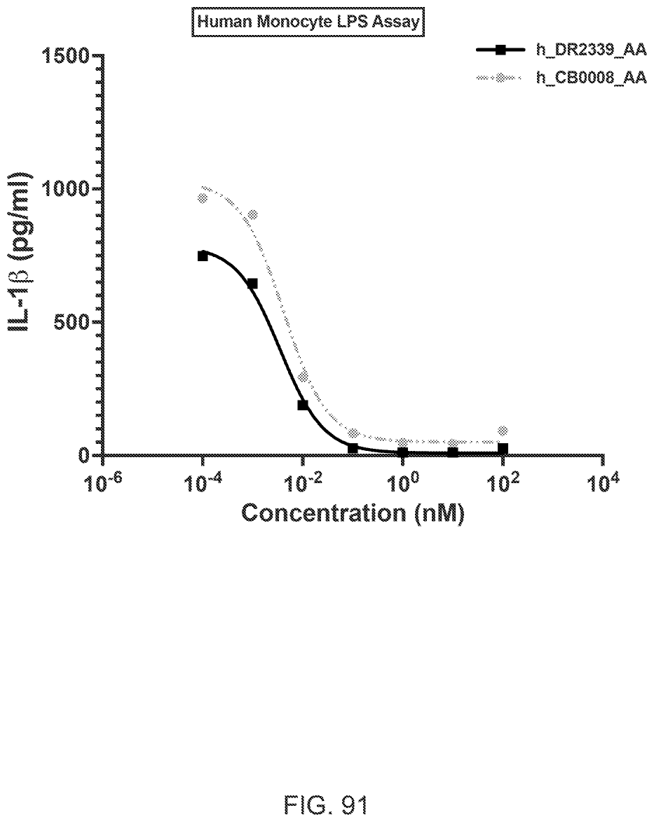

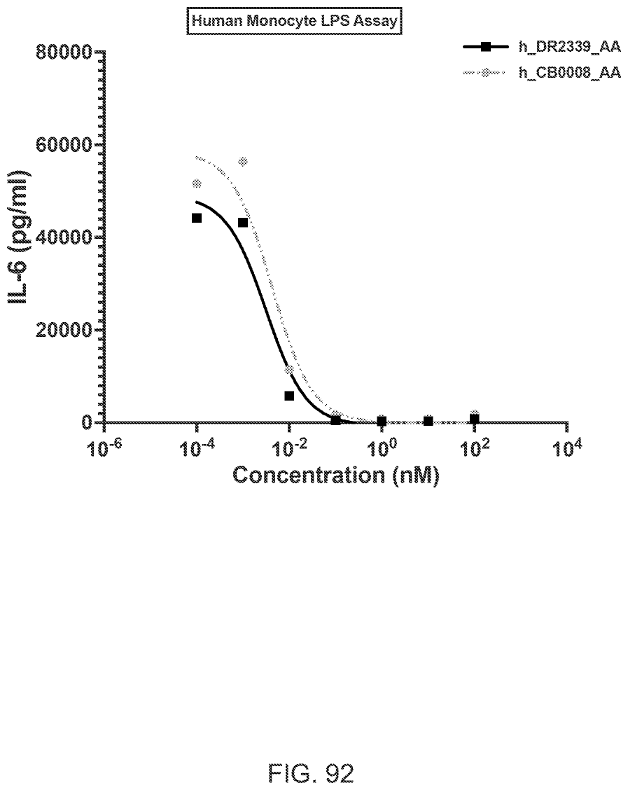

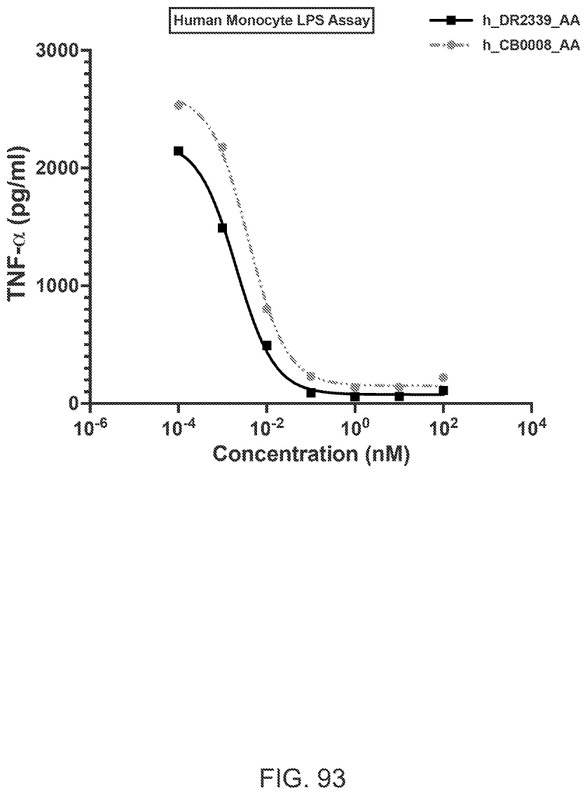

Post-treatment, cells were centrifuged at 400 g for 5 min and supernatants were collected. Supernatants were assessed for pro-inflammatory cytokines IL1b (FIG. 91), IL6 (FIG. 92) and TNFα (FIG. 93) using an MSD kit (Meso Scale Discovery, Catalog #K151A9H-4) in substantial accordance with the instructions provided by the manufacturer. The data was graphed using Prism software and is provided in FIGS. 91, 92 and 93 of the attached drawings. As illustrated by the data presented in provided in FIGS. 91, 92 and 93, the single point mutation M22W introduced into the fused format (h_CB0008_AA) inhibits LPS-induced proinflammatory cytokine secretion by monocytes, with less than 2-fold potency drop compared to fused_WT IL-10 (h_DR2339_AA).

### Example 44. Monocyte IL10 Activity Assay of CB0012 (T100C)

Human PBMCs were isolated from Leukopak obtained from a non-smoking human donor (Donor #072) by Ficoll® gradient purification. Human monocytes were purified from the PBMCs by positive selection using human CD14 Microbeads (Miltenyi Biotech, Catalog #130-050-201) in substantial accordance with the instructions provided by the manufacturer. Monocytes were contacted with 1 ng/ml of LPS (Millipore Sigma, Catalog #L2630) in the presence of the h_DR2339_AA or h_CB0012_AA as described Table 31 below at concentrations ranging from 0.02 pM to 1000 nM for 48 hours at 37 °C. The description of h_DR2339_AA (SEQ ID NO:208) is provided in Example 29. The test article h_CB0012_AA was expressed by transient transfection in Expi293 cells as a precursor using the MHSSALLCCLVLLTGVRA (SEQ ID NO:98) signal peptide which was removed by the mammalian host cell and purified using metal affinity chromatography to produce the sequence described in Table 31.

TABLE 31
h_CB0012_AA

| SEQ | | | |
|---|---|---|---|
| ID NO | Name | Description | AA Sequence |
| 227 | h_CB0012_AA | T100C/T100C | SPGQGTQSENSCTHFPGNLPNMLRDL |
| | | hIL10; GGS- | RDAFSRVKTFFQMKDQLDNLLLKES |
| | | His8 (SEQ ID | LLEDFKGYLGCQALSEMIQFYLEEV |
| | | NO: 23) | MPQAENQDPDIKAHVNSLGENLKCL |
| | | | RLRLRRCHRFLPCENKSKAVEQVKN |
| | | | AFNKLQEKGIYKAMSEFDIFINYIEAY |
| | | | MTMKIRNSPGQGTQSENSCTHFPGNL |
| | | | PNMLRDLRDAFSRVKTFFQMKDQLD |
| | | | NLLLKESLLEDFKGYLGCQALSEMIQ |
| | | | FYLEEVMPQAENQDPDIKAHVNSLG |
| | | | ENLKCLRLRLRRCHRFLPCENKSKAV |
| | | | EQVKNAFNKLQEKGIYKAMSEFDIFI |
| | | | NYIEAYMTMKIRNGGSHHHHHHHH |
| | | | (SEQ ID NO: 227) |

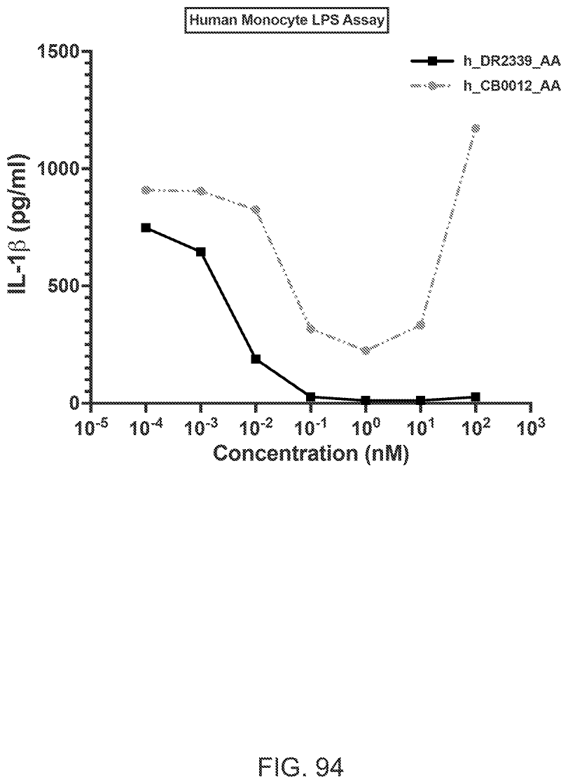

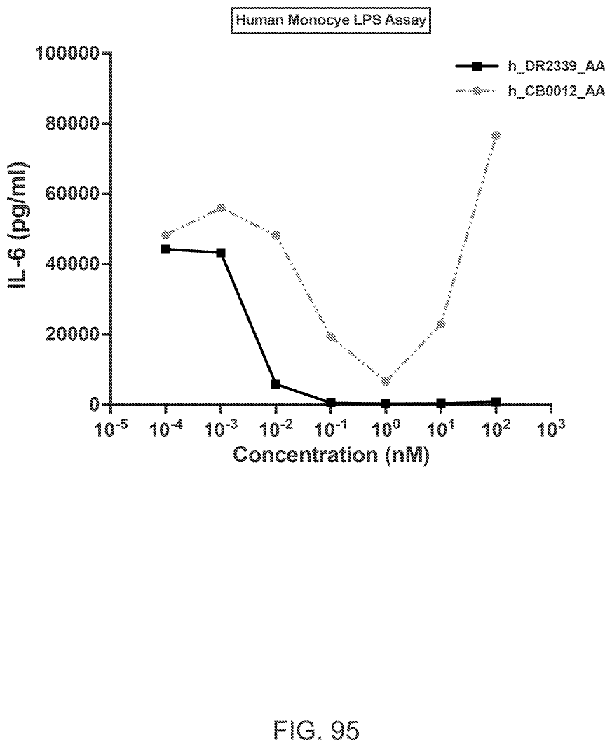

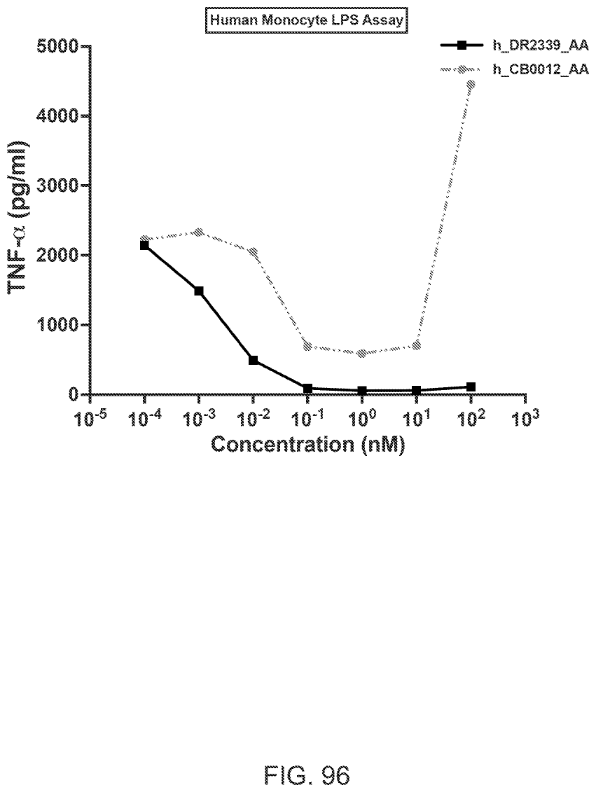

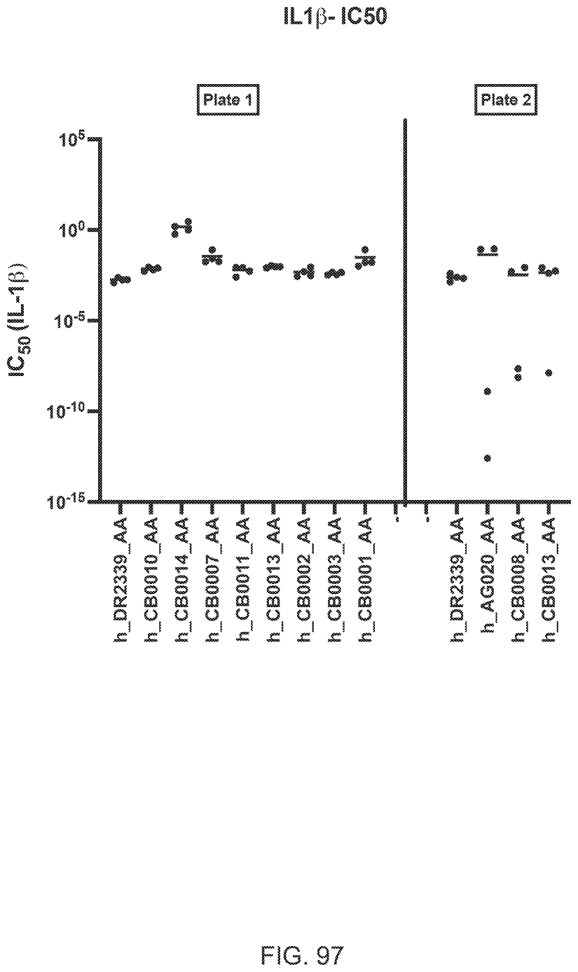

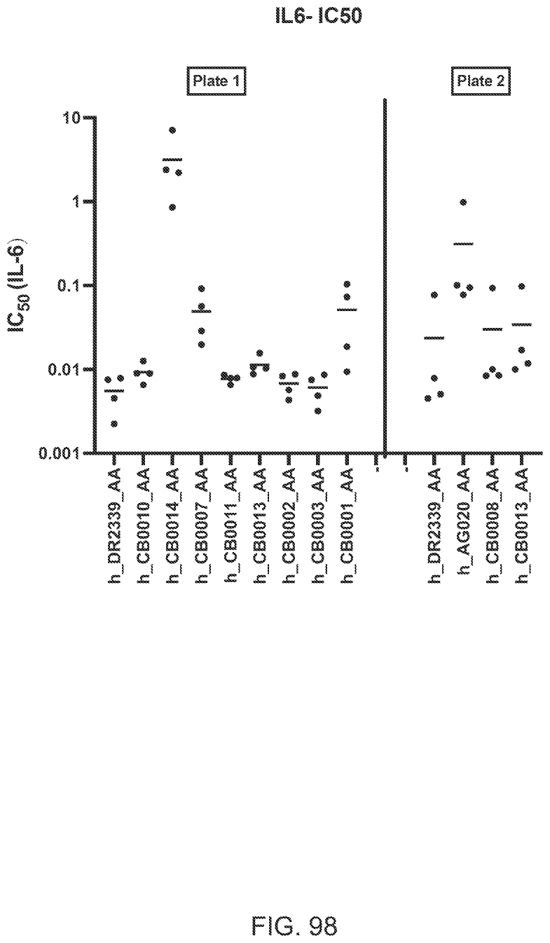

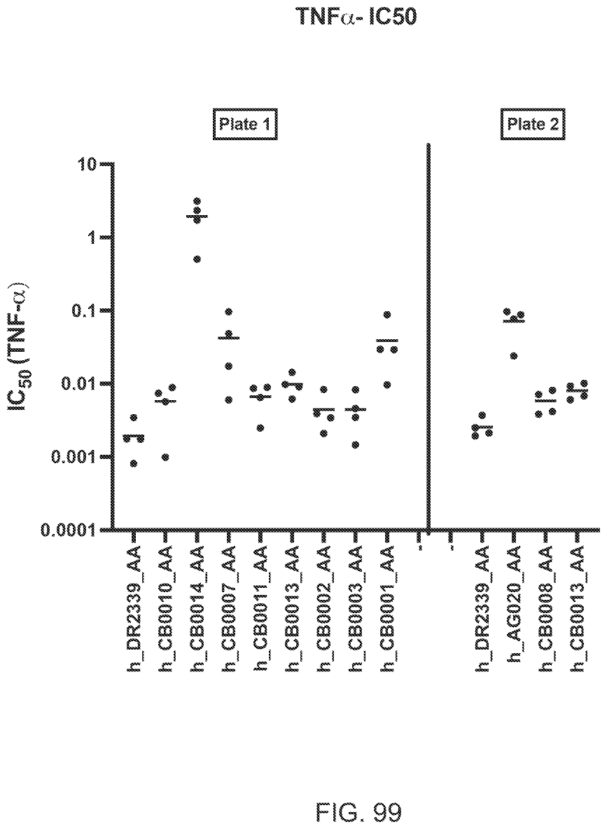

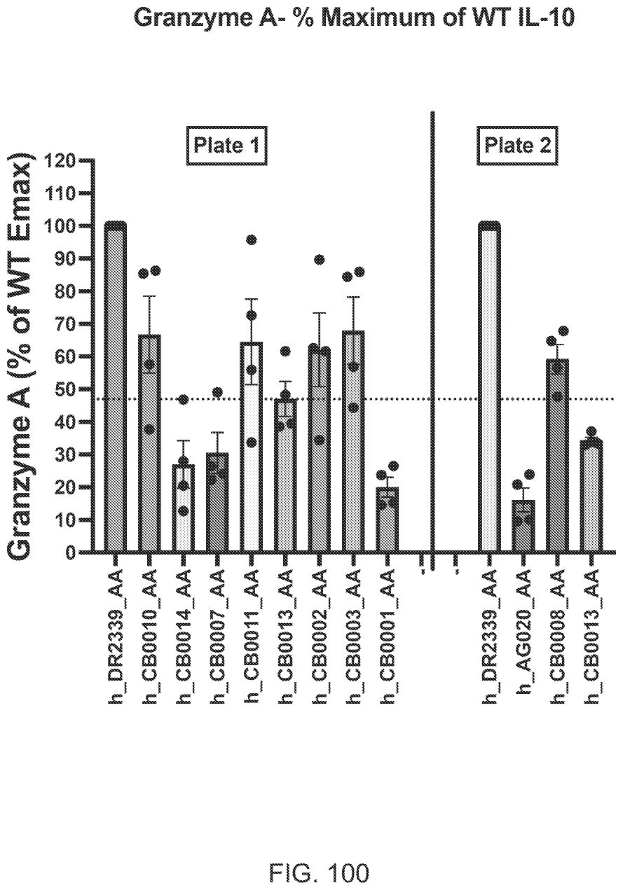

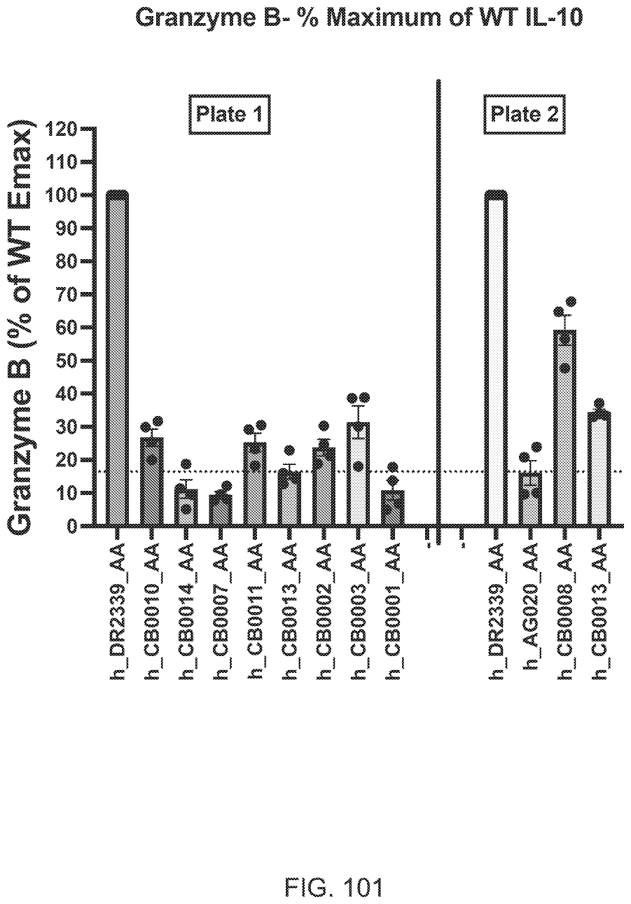

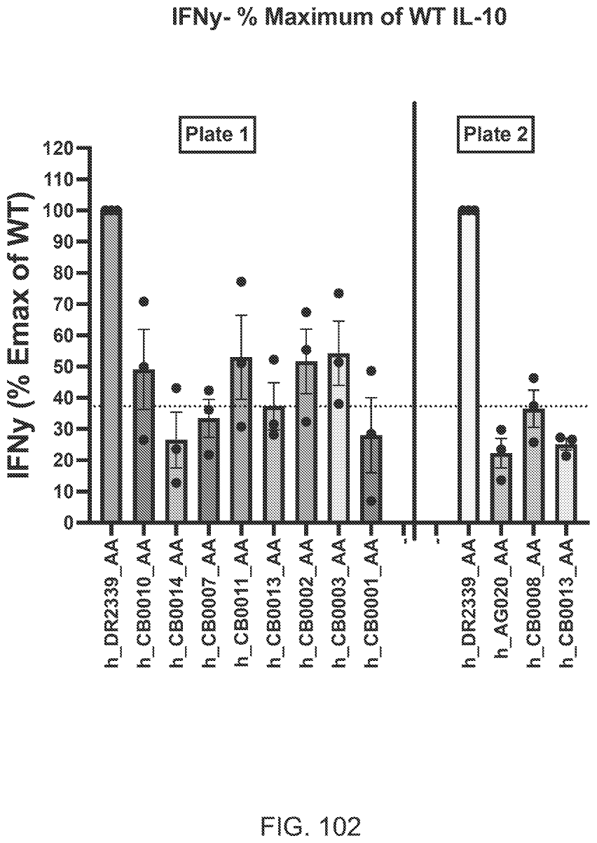

Post-treatment, cells were centrifuged at 400 g for 5 min and supernatants were collected. Supernatants were assessed for pro-inflammatory cytokines IL1b (FIG. 94), IL6 (FIG. 95) and TNFα (FIG. 96) using an MSD kit (Meso Scale Discovery, Catalog #K151A9H-4) in substantial accordance with the instructions provided by the manufacturer. The data was graphed using Prism software and is provided in FIGS. 94, 95 and 96 of the attached drawings. As illustrated by the data presented in provided in FIGS. 94, 95 and 96, the single point mutation T100C introduced into the fused format (h_CB0012_AA) does not completely inhibit LPS-induced proinflammatory cytokine secretion by monocytes and has a blocking effect above 1 nM concentration.

### Example 45. Monocyte IL10 Activity Assay

Human PBMCs were isolated from Leukopaks obtained from four non-smoking human donors (Donor #072, Donor #890, Donor #836 and Donor #838) by Ficoll gradient purification. Human Monocytes were purified from the PBMCs by positive selection using Human CD14 Microbeads (Miltenyi Biotech-130-050-201) following manufacturer's protocol. Monocytes were treated with 1 ng/ml of LPS (Millipore Sigma: L2630) in the presence of varying concentrations of the IL10 proteins (0.1 pM-100 nM) for 48 hours at 37 °C. Post-treatment, cells were spun down at 400 g for 5 min and supernatants were collected. Supernatants were assessed for pro-inflammatory cytokines using the MSD kit (Meso Scale Discovery: K151A9H-4) following manufacturer's protocol. The data was graphed using Prism software. IC50 values of the various molecules in inhibiting LPS-induced secretion of proinflammatory cytokines from 4 donors are plotted above.

### Example 46. CD8 T Cell Blasts IL10 Activity Assay

Human PBMCs were isolated from LRS chambers obtained from 4 non-smoking human donors by Ficoll gradient purification (Donor #072, Donor #890, Donor #836 and Donor #838). Human CD8 T cells were purified from the PBMCs by negative selection using a kit (Miltenyi Biotech-130-096-495) following manufacturer's protocol. Isolated CD8 T cells were blasted using the Human T cell activation and expansion kit (Miltenyi Biotech-130-091-441) in the presence of 100 pM Human IL-2 for 72 hours at 37 °C. The blasted CD8 T cells were washed and treated with varying concentrations of the IL10 proteins (0.1 pM-100 nM) for 72 hours at 37 °C. Post-treatment, cells were spun down at 400 g for 5 min and supernatants were collected. Supernatants were assessed for cytokines (IFNγ, Granzyme A and Granzyme B) using the MSD kit (Meso Scale Discovery: K151AEL-4) following manufacturer's protocol. The data was graphed using Prism software. The secretion of various cytokines is represented as values normalized to the percentage maximum secretion induced by fused_WT IL-10 (h_DR2339_AA) (where maximum secretion by fused_WT IL-10 (h_DR2339_AA) is set to 100%). No IFNγ secretion was observed with Donor 072 even in control samples and hence IFNγ data is plotted for an n=3 donors.

As shown by the data presented, the fused hIL10 polypeptides of the present disclosure are myeloid biased in function showing inhibition of (in some instances complete inhibition of) LPS induced secretion of proinflammatory cytokines from monocytes while being less potent at inducing cytokine release from activated CD8 T cell blasts as compared to the fused wild type hIL10 molecule.

### Example 47. CD8 T Cell Blast IL10 Activity Assay

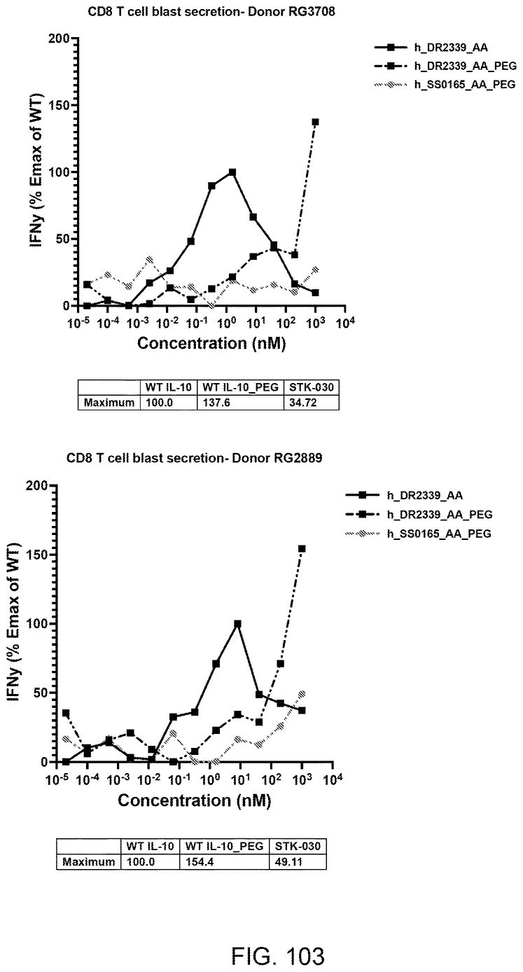

Human PBMCs were isolated from LRS chambers obtained from 2 non-smoking human donors by Ficoll gradient purification (Donor #RD2889, Donor #RG3708). Human CD8 T cells were purified from the PBMCs by negative selection using a kit (Miltenyi Biotech, Catalog #130-096-495) in substantial accordance with manufacturer's protocol. Isolated CD8 T cells were blasted using the Human T cell activation and expansion kit (Miltenyi Biotech, Catalog #130-091-441) in the presence of 100 pM human IL-2 for 72 hours at 37 °C. The blasted CD8 T cells were washed and treated with varying concentrations of the test articles h_DR2339_AA, h_DR2339_AA_PEG (40 kD N-terminal branched, 2×20 kD), and h_SS0165_AA_PEG (40 kD N-terminal branched, 2×20 kD) at concentrations ranging from 0.02 pM to 1000 nM for 72 hours at 37 °C. The test articles were prepared in substantial accordance with the foregoing Examples. Post-treatment, cells were centrifuged at 400 g for 5 min and supernatants were collected. Supernatants were assessed for cytokines (IFN□) using the MSD kit (Meso Scale Discovery: Catalog #K151AEL-4) following manufacturer's protocol. The data was graphed using Prism software and the data presented in FIG. 103 of the attached drawings. As illustrated, the secretion of cytokines is represented as values normalized to the percentage maximum secretion induced by the fused h_WT IL-10 (h_DR2339_AA) where maximum secretion by fused h_WT IL-10 (h_DR2339_AA) is set to 100%. Emax (Emax here refers to maximum observed response) of secreted IFNγ achieved with fused molecule with the T100L mutation (h_SS0165_AA) is <50% of Emax obtained with native, non-PEGylated WT IL-10 (h_DR2339_AA) tested at concentrations up to 1 μM.

### Example 48. CD8 T Cell Blasts IL10 Activity Assay

Human PBMCs were isolated from LRS chambers obtained from a non-smoking human donors by Ficoll gradient purification (Donor #522). Human CD8 T cells were purified from the PBMCs by negative selection using a kit (Miltenyi Biotech, Catalog #130-096-495) following manufacturer's protocol. Isolated CD8 T cells were blasted using the human T cell activation and expansion kit (Miltenyi Biotech, Catalog #130-091-441) in the presence of 100 pM human IL-2 for 72 hours at 37 °C. The following IL10 test articles, prepared in substantial accordance with the foregoing Examples, were evaluated in this experiment: (a) h_DR2339_AA; (b) h_SS0053_AA; (c) h_SS0260_AA (also referred to as h_CB0007_AA herein, SEQ ID NO:216); (d)_SS0258_AA (also referred to as h_CB0001_AA herein, SEQ ID NO:223); and (e) h_SS0259_AA (also referred to as h_CB0014_AA herein, SEQ ID NO:215).

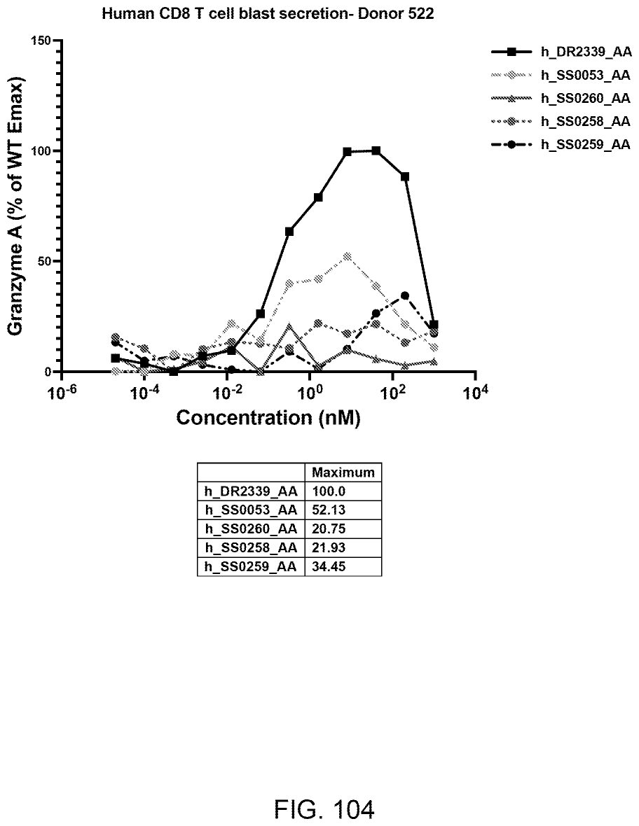

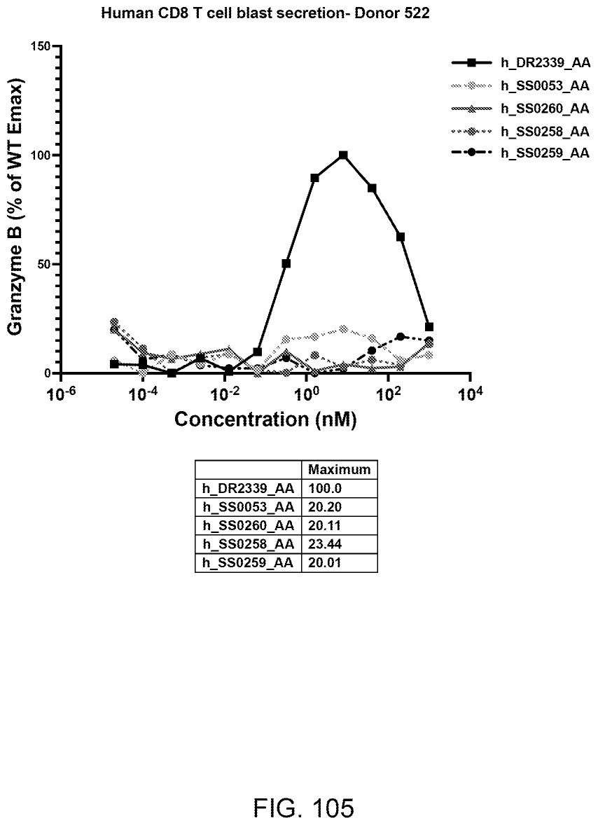

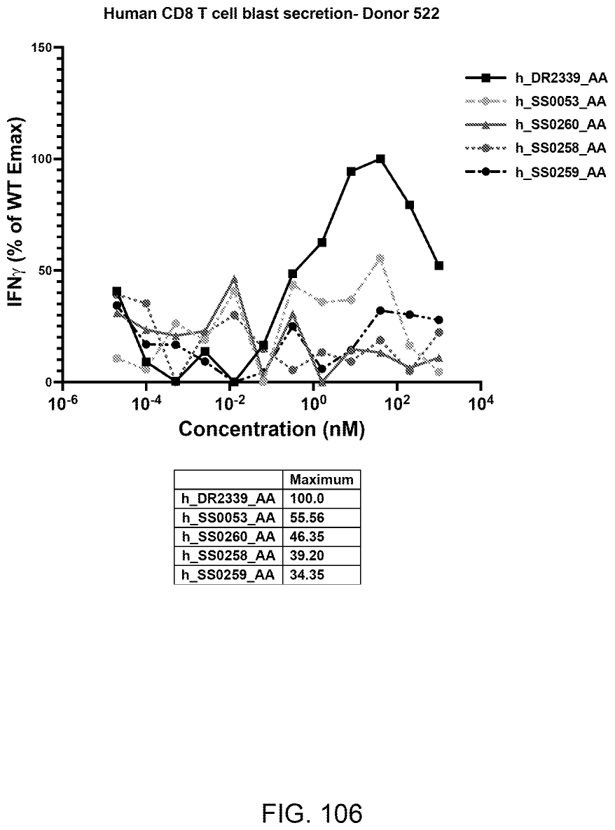

The blasted CD8 T cells were washed and treated with varying concentrations of the IL10 proteins ranging from 0.02 pM to 1000 nM for 72 hours at 37 °C. Post-treatment, cells were spun down at 400 g for 5 min and supernatants were collected. Supernatants were assessed for cytokines (IFNγ, Granzyme A and Granzyme B) using the MSD kit (Meso Scale Discovery: K151AEL-4) following manufacturer's protocol. The data was graphed using Prism software and presented in FIGS. 104, 105, and 106 of the attached drawings. The secretion of cytokines is represented as values normalized to the percentage maximum secretion induced by fused h_WT IL-10 (h_DR2339_AA) (where maximum secretion by fused h_WT IL-10 (h_DR2339_AA) is set to 100%). Eₘₐₓ (Eₘₐₓ here refers to maximum observed response) of secreted cytokines achieved by the various mutants in the fused format was less than the Eₘₐₓ of secretion observed by WT IL-10 (h_DR2339_AA) tested at concentrations up to 1 M.

### EXEMPLARY EMBODIMENTS

The disclosure also includes the following embodiments:
Embodiment 1. A fused human IL10 (hIL10) polypeptide comprising a polypeptide of the formula:

(hIL10A)-Lₙ-(hIL10B),
wherein:
   (a) hIL10A and hIL10B are each human IL10 (hIL10) sequences independently selected from the group consisting of a wild type hIL10 (SEQ ID NO:4) and hIL10 muteins, wherein the hIL10 muteins each independently comprises one or more amino acid substitutions at positions corresponding to residues T100, H14, N18, N21, M22, D25, R32, 593, and E96 of SEQ ID NO:4, optionally the hIL10A has an amino-terminal deletion of 1, 2, 3, 4, 5, 6, 7, 8, 9, or 10 amino acid residues relative to SEQ ID NO:4, and/or the hIL10B has an amino-terminal deletion of 1, 2, 3, 4, 5, 6, 7, 8, 9, or 10 amino acid residues relative to SEQ ID NO:4;
   (b) L is an amino acid linker of between 1 and 30 amino acids in length; and
   (c) n=0 (absent) or 1 (present).
Embodiment 2. The fused hIL10 polypeptide of embodiment 1, wherein:
   (a) the amino acid substitution at position T100 is selected from the group consisting of T100L, T100D, T100V, T100E, T100A, T100R, T100N, T100Q, T100E, T100I, T100K, T100M, and T100S;
   (b) the amino acid substitution at position H14 is selected from the group consisting of H14C, H14F, H14P, H14W, H14G, H14A, H14D, H14E, H14I, H14K, H14L, H14M, H14N, H14Q, H14R, H14S, H14T, H14Y, and H14V;
   (c) the amino acid substitution at position N18 is selected from the group consisting of N18Y, N18F, N18A, N18D, N18E, N18L, N18V, N18S, N18T, N18I, N18V, N18M, N18R, N18K, and N18H;
   (d) the amino acid substitution at position N21 is selected from the group consisting of N21A, N21R, N21Q, N21H, N21K, N21S, N21V, N21I, N21L, N21M, N21T N21C, N21D, and N21E;
   (e) the amino acid substitution at position M22 is selected from the group consisting of M22A, M22V, M22I, M22L, M22N, M22D, M22S, M22T, M22W, and M22Q;
   (f) the amino acid substitution at position R24 is selected from the group consisting of R24E, R24D, R24N, R24Q, R24A, R24S, and R24T;
   (g) the amino acid substitution at position D25 is selected from the group consisting of D25A, D25N, D25H, D25I, D25K, D25L, D25P, D25Q, and D25V;
   (h) the amino acid substitution at position D28 is selected from the group consisting of D28A, D28E, D28L, D28V, D28S, D28T, D28I, D28V, D28M, D28H, D28K, and D28R;
   (i) the amino acid substitution at position R32 is selected from the group consisting of R32A, R32D, R32E, R32L, R32V, R32S, R32T, R32I, R32V, R32M, R32N, R32Q, R32G, R32C, R32P, R32F, R32Y, and R32H;
   (j) the amino acid substitution at position E74 is selected from the group consisting of E74A, E74D, E74L, E74V, E74S, E74T, E741, E74V, E74M, E74H, E74K, and E74R;
   (k) the amino acid substitution at position H90 is selected from the group consisting of H90A, H90D, H90E, H90I, H90K, H90L, H90M, H90N, H90Q, H90R, H90S, H90T, H90Y, and H90V;
   (l) the amino acid substitution at position N92 is selected from the group consisting of N92D, N92Q, N92E, N92H, N92K, N92S, N92V, N92I, N92L, N92M, N92T, and N92A;
   (m) the amino acid substitution at position S93 is selected from the group consisting of S93E, S93A, S93R, S93N, S93D, S93Q, S93E, S93I, S93L, S93K, S93M, S93G, and S93V;
   (n) the amino acid substitution at position E96 is selected from the group consisting of E96C, E96F, E96Y, E96W, E96A, E96N, E96D, E96Q, E96H, E96K, and E96S; and
   (o) the amino acid substitution at position R104 is selected from the group consisting of R104A, R104W, R104Y, R104F, R104H, R104D, R104E, R104N, R104Q, R104S, R104T, R104I, R104L, R104V, and R104M.
Embodiment 3. The fused hIL10 polypeptide of embodiment 1 or 2, wherein at least one of the hIL10A and the hIL10B is an hIL10 mutein.
Embodiment 4. The fused hIL10 polypeptide of any one of embodiments 1 to 3, wherein the hIL10A and/or the hIL10B comprises the amino acid substitution T100L.
Embodiment 5. The fused hIL10 polypeptide of any one of embodiments 1 to 4, wherein the hIL10A and/or hIL10B comprises the amino acid substitution M22A or M22S.
Embodiment 6. The fused hIL10 polypeptide of any one of embodiments 1 to 5, wherein the hIL10A and/or hIL10B comprises the amino acid substitution D25K.
Embodiment 7. The fused hIL10 polypeptide of any one of embodiments 1 to 6, wherein the hIL10 mutein comprises a sequence having at least 95% identity to the sequence of SEQ ID NO:4.
Embodiment 8. The fused hIL10 polypeptide of any one of embodiments 1 to 7, wherein the hIL10A and hIL10B are the same.
Embodiment 9. The fused hIL10 polypeptide of any one of embodiments 1 to 7, wherein the hIL10A and hIL10B are different.
Embodiment 10. The fused hIL10 polypeptide of any one of embodiments 1 to 9, wherein n=1.
Embodiment 11. The fused hIL10 polypeptide of embodiment 10, wherein L is a GS linker.
Embodiment 12. The fused hIL10 polypeptide of embodiment 11, wherein the GS linker comprises a sequence of GGGSGSGSGSG (SEQ ID NO:19).
Embodiment 13. The fused hIL10 polypeptide of any one of embodiments 1 to 9, wherein n=0.
Embodiment 14. The fused hIL10 polypeptide of any one of embodiments 1 to 13, wherein the hIL10A and the hIL10B are each independently selected from a wild type hIL10 (SEQ ID NO:4) or an IL10 mutein comprising an amino acid substitution selected from the group consisting of M22A, M22S, D25K, and T100L.
Embodiment 15. The fused hIL10 polypeptide of embodiment 14, wherein the hIL10A and the hIL10B are the same.
Embodiment 16. The fused hIL10 polypeptide of embodiment 15, wherein the hIL10A and the hIL10B are both hIL10 muteins comprising the amino acid substitution T100L.
Embodiment 17. The fused hIL10 polypeptide of embodiment 16, wherein n is 0.
Embodiment 18. The fused hIL10 polypeptide of embodiment 17, wherein the fused hIL10 polypeptide comprises a sequence having at least 90% identity to the sequence of SEQ ID NO:44.
Embodiment 19. The fused hIL10 polypeptide of embodiment 18, wherein the fused hIL10 polypeptide comprises the sequence of SEQ ID NO:44.
Embodiment 20. The fused hIL10 polypeptide of any one of embodiments 1 to 19, wherein the fused hIL10 polypeptide is PEGylated.
Embodiment 21. The fused hIL10 polypeptide of any one of embodiments 1 to 20, wherein the fused hIL10 polypeptide is acylated.
Embodiment 22. The fused hIL10 polypeptide of any one of embodiments 1 to 21, wherein the fused hIL10 polypeptide is covalently linked to an Fc polypeptide.
Embodiment 23. The fused hIL10 polypeptide of embodiment 22, wherein the Fc polypeptide is an Fc domain from hIgG1, hIgG2, hIgG3, or hIgG4, or a variant thereof.
Embodiment 24. The fused hIL10 polypeptide of embodiment 22 or 23, wherein the Fc polypeptide comprises a sequence that is modified from a wild type Fc polypeptide sequence to reduce effector function.
Embodiment 25. The fused hIL10 polypeptide of any one of embodiments 1-24 wherein the fused hIL10 polypeptide exhibits a greater fraction of an activity of wt hIL10 in activated human monocytes than the fraction of the activity of wt hIL10 in activated human CD8 T cells.
Embodiment 26. The fused hIL10 polypeptide of embodiment 25, wherein the activity of wild-type hIL10 is induction of intracellular STAT3 signaling.
Embodiment 27. The fused hIL10 polypeptide of any one of embodiments 1-24, wherein the fused hIL10 polypeptide exhibits an Emax of at least 30%, optionally at least 40%, optionally at least 50% of level of activity of wild-type hIL10 in activated human monocytes wherein the activity of wild-type hIL10 in activated human monocytes is selected from the group consisting of inhibition of IL1P secretion and inhibition of TNFα secretion.
Embodiment 28. The fused hIL10 polypeptide of any one of embodiments 1-24, wherein the fused hIL10 polypeptide exhibits an Emax less than 30%, optionally less than 20%, optionally less than 10% of level of activity of wild-type hIL10 in activated human CD8 T cells wherein the activity of wild-type hIL10 in activated human CD8 T cells is selected from the group consisting of IFNg secretion, granzyme A secretion and granzyme B secretion.
Embodiment 29. A nucleic acid sequence encoding a fused hIL10 polypeptide of any one of embodiments 1-28.
Embodiment 30. The nucleic acid sequence of embodiment 29, wherein the nucleic acid sequence is an mRNA.
Embodiment 31. The nucleic acid sequence of embodiment 29, wherein the nucleic acid sequence is a DNA.
Embodiment 32. A vector comprising the nucleic acid sequence of any one of embodiments 29 to 31 operably linked with an expression control sequence.
Embodiment 33. The vector of embodiment 32, wherein the vector is a viral vector.
Embodiment 34. A cell transformed with the vector of embodiment 33.
Embodiment 35. The cell of embodiment 34, wherein the cell is a mammalian cell.
Embodiment 36. A pharmaceutical formulation comprising as an active ingredient: (a) the fused hIL10 polypeptide of any one of embodiments 1 to 28; (b) a nucleic acid sequence of embodiment 29; (c) a vector of embodiment 32, or (d) a cell of embodiment 34, and one or more pharmaceutically acceptable solvents, carriers, stabilizers, preservatives, or diluents.
Embodiment 37. A method of preventing or treating a mammalian subject suffering from a disease, disorder, or condition, the method comprising administering to said subject: (a) the fused hIL10 polypeptide of any one of embodiments 1 to 28; (b) a nucleic acid sequence of embodiment 29; (c) a vector of embodiment 32, or (d) a cell of embodiment 34, or (e) a pharmaceutical formulation of embodiment 36.
Embodiment 38. The method of embodiment 37, wherein the disease, disorder, or condition is an autoimmune disease, disorder, or condition.
Embodiment 39. The method of embodiment 38, wherein the autoimmune disease, disorder, or condition is an inflammatory bowel disease (IBD).
Embodiment 40. The method of embodiment 39, wherein the inflammatory bowel disease is Crohn's disease or ulcerative colitis.
Embodiment 41. The method of embodiment 38, wherein the disease, disorder, or condition is a cancer associated with chronic inflammation.

What is claimed is:

1. A fused human IL10 (hIL10) polypeptide comprising a polypeptide of the formula:
(hIL10A)-Lₙ-(hIL10B),
wherein:
(a) hIL10A and hIL10B are each human IL10 (hIL10) sequences independently selected from the group consisting of a wild-type hIL10 (SEQ ID NO:4) and hIL10 muteins, wherein the hIL10 muteins each independently comprises one or more amino acid substitutions at positions corresponding to residues T100, H14, N18, N21, M22, R24, D25, D28, R32, E74, H90, N92, S93, E96, and R104 of SEQ ID NO: 4, optionally the hIL10A has an amino-terminal deletion of 1, 2, 3, 4, 5, 6, 7, 8, 9, or 10 amino acid residues relative to SEQ ID NO:4, and/or the hIL10B has an amino-terminal deletion of 1, 2, 3, 4, 5, 6, 7, 8, 9, or 10 amino acid residues relative to SEQ ID NO:4;
(b) L is an amino acid linker of from 1 to 30 amino acids in length; and
(c) n=0 (absent) or 1 (present).

2. The fused hIL10 polypeptide of claim 1, wherein:
(a) the amino acid substitution at position T100 is selected from the group consisting of T100L, T100D, T100V, T100E, T100A, T100R, T100N, T100Q, T100E, T100I, T100K, T100M, and T100S;
(b) the amino acid substitution at position H14 is selected from the group consisting of H14C, H14F, H14P, H14W, H14G, H14A, H14D, H14E, H14I, H14K, H14L, H14M, H14N, H14Q, H14R, H14S, H14T, H14Y, and H14V:
(c) the amino acid substitution at position N18 is selected from the group consisting of N18Y, N18F, N18A, N18D, N18E, N18L, N18V, N18S, N18T, N18I, N18V, N18M, N18R, N18K, and N18H;
(d) the amino acid substitution at position N21 is selected from the group consisting of N21A, N21R, N21Q, N21H, N21K, N21S, N21V, N21I, N21L, N21M, N21T N21C, N21D, and N21E;
(e) the amino acid substitution at position M22 is selected from the group consisting of M22A, M22V, M22I, M22L, M22N, M22D, M22S, M22T, M22W, and M22Q;
(f) the amino acid substitution at position R24 is selected from the group consisting of R24E, R24D, R24N, R24Q, R24A, R24S, and R24T;
(g) the amino acid substitution at position D25 is selected from the group consisting of D25A, D25N, D25H, D25I, D25K, D25L, D25P, D25Q, and D25V;
(h) the amino acid substitution at position D28 is selected from the group consisting of D28A, D28E, D28L, D28V, D28S, D28T, D28I, D28V, D28M, D28H, D28K, and D28R;
(i) the amino acid substitution at position R32 is selected from the group consisting of R32A, R32D, R32E, R32L, R32V, R32S, R32T, R32I, R32V, R32M, R32N, R32Q, R32G, R32C, R32P, R32F, R32Y, and R32H;
(j) the amino acid substitution at position E74 is selected from the group consisting of E74A, E74D, E74L, E74V, E74S, E74T, E741, E74V, E74M, E74H, E74K, and E74R;
(k) the amino acid substitution at position H90 is selected from the group consisting of H90A, H90D, H90E, H90I, H90K, H90L, H90M, H90N, H90Q, H90R, H90S, H90T, H90Y, and H90V;
(l) the amino acid substitution at position N92 is selected from the group consisting of N92D, N92Q, N92E, N92H, N92K, N92S, N92V, N92I, N92L, N92M, N92T, and N92A;
(m) the amino acid substitution at position S93 is selected from the group consisting of S93E, S93A, S93R, S93N, S93D, S93Q, S93E, S93I, S93L, S93K, S93M, S93G, and S93V;
(n) the amino acid substitution at position E96 is selected from the group consisting of E96C, E96F, E96Y, E96W, E96A, E96N, E96D, E96Q, E96H, E96K, and E96S; and
(o) the amino acid substitution at position R104 is selected from the group consisting of R104A, R104W, R104Y, R104F, R104H, R104D, R104E, R104N, R104Q, R104S, R104T, R104I, R104L, R104V, and R104M.

3. The fused hIL10 polypeptide of claim 1 or 2, wherein at least one of the hIL10A and the hIL10B is an hIL10 mutein.

4. The fused hIL10 polypeptide of any one of claims 1-3, wherein hIL10A and the hIL10B are both hIL10 muteins.

5. The fused hIL10 polypeptide of any one of claims 1-4, wherein hIL10A and the hIL10B are the same.

6. The fused hIL10 polypeptide of any one of claims 1-4, wherein hIL10A and the hIL10B are different.

7. The fused hIL10 polypeptide of any one of claims 1-6, wherein n=0 (absent).

8. The fused hIL10 polypeptide of any one of claims 1-6, wherein n=1 (present) and L is a polypeptide having 1, 2, 3, 4, 5, 6, 7, 8, 9, 10, 11, 12, 13, 14 15, 16, 17, 18, 19, 20, 21, 22, 23, 24, 25, 26, 27, 28, 29, or 30 amino acids.

9. The fused hIL10 polypeptide of claim 8, wherein L is a GS linker.

10. The fused hIL10 polypeptide of claim 9, wherein the GS linker comprises a sequence of GGGSGSGSGSG (SEQ ID NO:19).

11. The fused hIL10 polypeptide of any one of claims 1 to 10, wherein the hIL10A and the hIL10B are each independently selected from a wild-type hIL10 (SEQ ID NO:4) or an IL10 mutein comprising an amino acid substitution selected from the group consisting of N21D, N21E, N21K, M22A, M22S, M22T, M22D, M22W, R24E, D25K, E96K, E96Q, T100E, T100C and T100L.

12. The fused hIL10 polypeptide of any one of claims 1 to 11, wherein the hIL10A and/or the hIL10B comprises the amino acid substitution T100L.

13. The fused hIL10 polypeptide of any one of claims 1 to 11, wherein the hIL10A and/or hIL10B comprises the amino acid substitution M22A or M22S.

14. The fused hIL10 polypeptide of any one of claims 1 to 11, wherein the hIL10A and/or hIL10B comprises the amino acid substitution E96Q.

15. The fused hIL10 polypeptide of any one of claims 1 to 11, wherein the hIL10A and/or hIL10B comprises the amino acid substitution R24E.

16. The fused hIL10 polypeptide of any one of claims 1 to 11, wherein the hIL10A and/or hIL10B comprises the amino acid substitution D25K.

17. The fused hIL10 polypeptide of any one of claims 1 to 11, wherein the hIL10A and/or hIL10B comprises the amino acid substitution N21K.

18. The fused hIL10 polypeptide of any one of claims 1 to 11, wherein hIL10A and/or hIL10B is each a polypeptide having at least 90% sequence identity to a polypeptide selected from the group consisting of SEQ ID NOS: 6, 7, 8, 9, 10, 11, 12, 13, 14, 15, 16, 17, 18, 148, 149, 150, 151, 152, 153, 154, 155, 156, 191, 192, 193, 194, 195, 196, 197, 198, 199, 200, 201, 202, 203, 204, and 205, optionally wherein one or both of hIL10A and hIL10B has an N-terminal deletion of 1, 2, 3, 4, 5, 6, 7, 8, 9, or 10 amino acid residues relative to the sequence of SEQ ID NO:4.

19. The fused hIL10 polypeptide of claim 18, wherein the hIL10A and hIL10B are the same.

20. The fused hIL10 polypeptide claim 18, wherein the hIL10A and hIL10B are different.

21. The fused hIL10 polypeptide of claim 19 or 20, wherein the hIL10A and the hIL10B each comprises an amino acid substitution selected from the group consisting of N21D, N21E, N21K, M22A, M22S, M22T, M22D, M22W, R24E, D25K, E96K, E96Q, T100E, T100C and T100L.

22. The fused hIL10 polypeptide of claim 19 or 20, wherein hIL10A is a polypeptide is selected from the group consisting of SEQ ID NOS:191, 192, 193, 194, 195, 196, 197, 198, 199, 200, 201, 202, 203, 204, and 205 and hIL10B is a polypeptide is selected from the group consisting of SEQ ID NOS: 6, 7, 8, 9, 10, 11, 12, 13, 14, 15, 16, 17, 18, 148, 149, 150, 151, 152, 153, 154, 155, and 156.

23. The fused hIL10 polypeptide of claim 21, wherein the hIL10A and the hIL10B are both hIL10 muteins comprising the amino acid substitution T100L.

24. The fused hIL10 polypeptide of claim 23, wherein the hIL10A and the hIL10B are both hIL10 muteins comprising the amino acid substitution T100L, hIL10A is a polypeptide having 100% sequence identity to SEQ ID NO: 204 and hIL10 B is a polypeptide having 100% sequence identity to SEQ ID NO: 15.

25. The fused hIL10 polypeptide of claim 21, wherein the fused hIL10 polypeptide is a polypeptide comprising an amino acid sequence having having at least 95%, alternatively at least 96%, alternatively at least 97%, alternatively at least 98%, alternatively at least 99%, alternatively 100% sequence identity to a polypeptide selected from the group consisting of SEQ ID NOS:24, 25, 26, 27, 28, 29, 30, 31, 32, 33, 34, 35, 36, 37, 38, 39, 40, 41, 42, 43, 44, 45, 46, 47, 48, 49, 50, 119, 157, 158, 159, 160, 161, 162, 163, 164, 165, 166, 167, 168, 169, 170, 171, 172, 173, 174, 175, 176, 177, 178, 179, 180, 181, 182, 183, 184, 185 and 186.

26. The fused hIL10 polypeptide of claim 21, wherein the fused hIL10 polypeptide comprises an amino acid sequence having 100% sequence identity to a polypeptide selected from the group consisting of SEQ ID NOS: 44, 123, 124, 157, 158, 159, 160, 161, 162, 163, 164, 165, 166, 167, 168, 169, 170, 171, 172, 173, 174, 175, 176, 177, 178, 179, 180, 181, 182, 183, 184, 185 and 186.

27. The fused hIL10 polypeptide of claim 23, wherein the fused hIL10 polypeptide comprises an amino acid sequence having at least 90% sequence identity to a polypeptide selected from the group consisting of SEQ ID NO: 44, SEQ ID NO: 123, SEQ ID NO: 124, SEQ ID NO: 170 and SEQ ID NO: 185.

28. The fused hIL10 polypeptide of claim 27, wherein the fused hIL10 polypeptide comprises a polypeptide having 100% identity to the amino acid sequence of SEQ ID NO: 44.

29. The fused hIL10 polypeptide of claim 27, wherein the fused hIL10 polypeptide comprises a polypeptide having 100% identity to the amino acid sequence of SEQ ID NO: 185.

30. The fused hIL10 polypeptide of any one of claims 1 to 29, wherein the fused hIL10 polypeptide modified to extend half-life in vivo.

31. The fused hIL10 polypeptide of claim 30, wherein the modification to extend half-life in vivo is selected from the group consisting of PEGylation, acylation, albumination or conjugation to an Fc polypeptide.

32. The fused hIL10 polypeptide of claim 31, wherein the modification to extend half-life in vivo is acylation.

33. The fused hIL10 polypeptide of claim 31, wherein the modification to extend half-life in vivo is conjugation to an Fc polypeptide.

34. The fused hIL10 polypeptide of claim 33, wherein the Fc polypeptide is an Fc domain from hIgG1, hIgG2, hIgG3, or hIgG4, or a variant thereof.

35. The fused hIL10 polypeptide of claim 33 or 34, wherein the Fe polypeptide comprises a sequence that is modified from a wild-type Fc polypeptide sequence to reduce effector function.

36. The fused hIL10 polypeptide of any one of claims 33-35, wherein the Fc polypeptide comprises one or more amino acid substitutions or deletions to promote heterodimerization.

37. The fused hIL10 polypeptide of claim 33, wherein the fused hIL10 polypeptide is a heterodimeric Fc molecule comprising a first polypeptide of the formula #1:
IL10FP-L1ₐ-UH1-Fc1 [1]
and a second polypeptide of the formula #2:
UH2-Fc2 [2]
wherein:
hIL10FP is a fused hIL10 polypeptide of the present disclosure (e.g., a fused hIL10 polypeptide of formula (hIL10A)-Lₙ-(hIL10B));
L1 is a linker and a is independently selected from 0 (absent) or 1 (present);
UH1 and UH2 are each an upper hinge domain of human immunoglobulin independently selected from the group consisting of the IgG1, IgG2, IgG3 and IgG4 upper hinge domains, optionally comprising the amino acid substitution C220S (EU numbering);
Fc1 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc2, and
Fc2 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc1,
and optionally wherein the polypeptide of formula [1] and the polypeptide of formula [2] are linked by at least one interchain disulfide bond.

38. The fused hIL10 polypeptide of claim 31, wherein the modification to extend half-life in vivo is PEGylation.

39. The fused hIL10 polypeptide of claim 38, wherein the PEG is a linear or branched polyethylene glycol molecule having a molecular weight of from about 2,000 to about 80.000 daltons, alternatively about 2,000 to about 70.000 daltons, alternatively about 5,000 to about 50,000 daltons, alternatively about 10,000 to about 50,000 daltons, alternatively about 20,000 to about 50,000 daltons, alternatively about 30,000 to about 50,000 daltons.

40. The fused hIL10 polypeptide of claim 39, wherein the PEG is linear.

41. The fused hIL10 polypeptide of claim 39, wherein the PEG is branched.

42. The fused hIL10 polypeptide of claim 39, wherein the PEG is a 40 kD branched PEG molecule comprising two 20 kD arms.

43. The fused hIL10 polypeptide of claim 39, wherein the PEG is covalently attached to the N-terminus of the polypeptide, optionally via a linker.

44. The fused hIL10 polypeptide of claim 39, wherein the PEG is a 40 kD branched PEG molecule comprising two 20 kD arms of the formula: 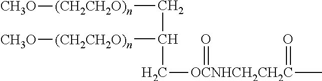
covalently bonded to the N-terminus of hIL10A, optionally via a linker.

45. The fused hIL10 polypeptide of claim 44, wherein the PEG covalently bonded to the N-terminus of hIL10A via an aldehyde linker.

46. The fused hIL10 polypeptide of any one of claims 1-45, wherein the fused hIL10 polypeptide exhibits a greater fraction of an activity of wild-type hIL10 in activated human monocytes than the fraction of the activity of wild-type hIL10 in activated human CD8 T cells.

47. The fused hIL10 polypeptide of claim 46, wherein the activity of wild-type hIL10 is induction of intracellular STAT3 signaling.

48. The fused hIL10 polypeptide of any one of claims 1-45, wherein the fused hIL10 polypeptide exhibits an Emax of at least 30%, optionally at least 40%, optionally at least 50% of level of activity of wild-type hIL10 in activated human monocytes wherein the activity of wild-type hIL10 in activated human monocytes is selected from the group consisting of inhibition of IL1b secretion and inhibition of TNFα secretion.

49. The fused hIL10 polypeptide of any one of claims 1-45, wherein the fused hIL10 polypeptide exhibits an Emax less than 30%, optionally less than 20%, optionally less than 10% of level of activity of wild-type hIL10 in activated human CD8 T cells wherein the activity of wild-type hIL10 in activated human CD8 T cells is selected from the group consisting of IFNγ secretion, granzyme A secretion and granzyme B secretion.

50. A nucleic acid sequence encoding a fused hIL10 polypeptide of any one of claims 1-45.

51. The nucleic acid sequence of claim 50, wherein the nucleic acid sequence is an mRNA.

52. The nucleic acid sequence of claim 50, wherein the nucleic acid sequence is a DNA.

53. A vector comprising the nucleic acid sequence of any one of claims 50 to 52 operably linked with an expression control sequence.

54. The vector of claim 53, wherein the vector is a viral vector.

55. A cell transformed with the vector of claim 53 or 54.

56. The cell of claim 55, wherein the cell is a mammalian cell.

57. A pharmaceutical formulation comprising as an active ingredient: (a) the fused hIL10 polypeptide of any one of claim 1 to 45; (b) a nucleic acid sequence of any one of claims 50-52; (c) a vector of claim 53 or 54, or (d) a cell of claim 55 or 56, and one or more pharmaceutically acceptable solvents, carriers, stabilizers, preservatives, or diluents.

58. A method of preventing or treating a mammalian subject suffering from a disease, disorder, or condition, the method comprising administering to said subject: (a) the fused hIL10 polypeptide of any one of claim 1 to 45; (b) a nucleic acid sequence of any one of claims 50-52; (c) a vector of claim 53 or 54, or (d) a cell of claim 55 or 56, or (e) a pharmaceutical formulation of claim 57.

59. The method of claim 58, wherein the disease, disorder, or condition is an autoimmune disease, disorder, or condition.

60. The method of claim 59, wherein the autoimmune disease, disorder, or condition is an inflammatory bowel disease (IBD).

61. The method of claim 60, wherein the IBD is Crohn's disease or ulcerative colitis.

62. The method of claim 58, wherein the disease, disorder, or condition is cancer.

63. The method of claim 62, wherein the cancer is a cancer arising from chronic inflammation.

64. The method of claim 58, wherein the disease, disorder, or condition is macrophage activation syndrome.

65. The method of claim 58, wherein the disease, disorder, or condition is interferon gamma induced anemia.

66. A method of making a fused human IL10 (hIL10) polypeptide of the formula (hIL10A)-Ln-(hIL10B), the method comprising the steps of transfecting a host cell with a vector of claim 53 or 54, culturing said cell in a culture medium under conditions to permit expression of the nucleic acid sequence encoding the fused hIL10 polypeptide, and isolating the fused hIL10 polypeptide from the culture medium, optionally further comprising the step of purification of the fused hIL10 polypeptide.

67. The method of claim 66, wherein the host cell is a procaryotic cell.

68. The method of claim 67, wherein the procaryotic cell is an E. coli cell.

69. The method of any one of claims 66-68, wherein the hIL10A is a polypeptide selected from the group consisting of SEQ ID NOS: 191, 192, 193, 194, 195, 196, 197, 198, 199, 200, 201, 202, 203, 204, and 205, and the hIL10B is a polypeptide selected from the group consisting of SEQ ID NOS: 6, 7, 8, 9, 10, 11, 12, 13, 14, 15, 16, 17, 18, 148, 149, 150, 151, 152, 153, 154, 155, and 156.

70. The method of claim 69, wherein the hIL10A is a polypeptide selected from the group consisting of SEQ ID NOS: 172, 173, 174, 175, 176, 177, 178, 179, 180, 181, 182, 183 184, 185, and 186.


SEQUENCE LISTING (227 sequences)

SEQ ID NO: 1 - protein, 557 aa, Homo sapiens
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 2 - protein, 306 aa, Homo sapiens
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 3 - protein, 330 aa, Mus sp.
MIPPPEKVRMNSVNFKNILQWEVPAFPKTNLTFTAQYESYRSFQDHCKRTASTQCDFSHLSKYGDYTVRVRAELADEHSEWVNVTFCPVEDTIIGPPEMQIESLAESLHLRFSAPQIENEPETWTLKNIYDSWAYRVQYWKNGTNEKFQVVSPYDSEVLRNLEPWTTYCIQVQGFLLDQNRTGEWSEPICERTGNDEITPSWIVAIILIVSVLVVFLFLLGCFVVLWLIYKKTKHTFRSGTSLPQHLKEFLGHPHHSTFLLFSFPPPEEAEVFDKLSIISEESEGSKQSPEDNCASEPPSDPGPRELESKDEAPSPPHDDPKLLTSTSEV

SEQ ID NO: 4 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 5 - skipped

SEQ ID NO: 6 - protein, 160 aa, synthetic construct
SPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 7 - protein, 150 aa, synthetic construct
SCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 8 - protein, 160 aa, synthetic construct
SPGQGTQSENSCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 9 - protein, 150 aa, synthetic construct
SCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 10 - protein, 160 aa, synthetic construct
SPGQGTQSENSCTHFPGNLPNALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 11 - protein, 150 aa, synthetic construct
SCTHFPGNLPNALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 12 - protein, 160 aa, synthetic construct
SPGQGTQSENSCTHFPGNLPNSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 13 - protein, 150 aa, synthetic construct
SCTHFPGNLPNSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 14 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRN

SEQ ID NO: 15 - protein, 150 aa, synthetic construct
SCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRN

SEQ ID NO: 16 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGQNLKTLRLRLRRCHRFLPCENKSKAVEQVKNAFNKLQEKGIYKAMSEFDIFINYIEAYMTMKIRN

SEQ ID NO: 17 - protein, 150 aa, synthetic construct
SCTHFPGNLPNMLRDLRDAFSRVKTFFQMKDQLDNLLLKESLLEDFKGYLGCQALSEMIQFYLEEVMPQAENQDPDIKAHVNSLGQNLKTLRLRLRRCHRFLPCENKSKAVEQVKNAFNKLQEKGIYKAMSEFDIFINYIEAYMTMKIRN

SEQ ID NO: 18 - protein, 153 aa, synthetic construct
SENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 19 - protein, 11 aa, synthetic construct
GGGSGSGSGSG

SEQ ID NO: 20 - protein, 11 aa, synthetic construct
NQMFDQKYDDP

SEQ ID NO: 21 - protein, 10 aa, Homo sapiens
SPGQGTQSEN

SEQ ID NO: 22 - protein, 7 aa, Homo sapiens
SPGQGTQ

SEQ ID NO: 23 - protein, 11 aa, synthetic construct
GGSHHHHHHHH

SEQ ID NO: 24 - protein, 321 aa, synthetic construct
SPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGSGSGSGSGSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 25 - protein, 321 aa, synthetic construct
SPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNTSPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 26 - protein, 321 aa, synthetic construct
SPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNNQMFDQKYDDPSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 27 - protein, 321 aa, synthetic construct
SPGQGTQSENSCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGSGSGSGSGSCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 28 - protein, 321 aa, synthetic construct
SPGQGTQSENSCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNTSPGQGTQSENSCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 29 - protein, 321 aa, synthetic construct
SPGQGTQSENSCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNNQMFDQKYDDPSCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 30 - protein, 321 aa, synthetic construct
SPGQGTQSENSCTHFPGNLPNALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGSGSGSGSGSCTHFPGNLPNALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 31 - protein, 321 aa, synthetic construct
SPGQGTQSENSCTHFPGNLPNALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNTSPGQGTQSENSCTHFPGNLPNALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 32 - protein, 321 aa, synthetic construct
SPGQGTQSENSCTHFPGNLPNALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNNQMFDQKYDDPSCTHFPGNLPNALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 33 - protein, 321 aa, synthetic construct
SPGQGTQSENSCTHFPGNLPNSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGSGSGSGSGSCTHFPGNLPNSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 34 - protein, 321 aa, synthetic construct
SPGQGTQSENSCTHFPGNLPNSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNTSPGQGTQSENSCTHFPGNLPNSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 35 - protein, 321 aa, synthetic construct
SPGQGTQSENSCTHFPGNLPNSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNNQMFDQKYDDPSCTHFPGNLPNSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 36 - protein, 321 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRNGGGSGSGSGSGSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRN

SEQ ID NO: 37 - protein, 321 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRN

SEQ ID NO: 38 - protein, 321 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRNNQMFDQKYDDPSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRN

SEQ ID NO: 39 - protein, 321 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGQNLKTLRLRLRRCHRFLPCENKSKAVEQVKNAFNKLQEKGIYKAMSEFDIFINYIEAYMTMKIRNGGGSGSGSGSGSCTHFPGNLPNMLRDLRDAFSRVKTFFQMKDQLDNLLLKESLLEDFKGYLGCQALSEMIQFYLEEVMPQAENQDPDIKAHVNSLGQNLKTLRLRLRRCHRFLPCENKSKAVEQVKNAFNKLQEKGIYKAMSEFDIFINYIEAYMTMKIRN

SEQ ID NO: 40 - protein, 321 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGQNLKTLRLRLRRCHRFLPCENKSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGQNLKTLRLRLRRCHRFLPCENKSKAVEQVKNAFNKLQEKGIYKAMSEFDIFINYIEAYMTMKIRN

SEQ ID NO: 41 - protein, 321 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGQNLKTLRLRLRRCHRFLPCENKSKAVEQVKNAFNKLQEKGIYKAMSEFDIFINYIEAYMTMKIRNNQMFDQKYDDPSCTHFPGNLPNMLRDLRDAFSRVKTFFQMKDQLDNLLLKESLLEDFKGYLGCQALSEMIQFYLEEVMPQAENQDPDIKAHVNSLGQNLKTLRLRLRRCHRFLPCENKSKAVEQVKNAFNKLQEKGIYKAMSEFDIFINYIEAYMTMKIRN

SEQ ID NO: 42 - protein, 321 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 43 - protein, 320 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 44 - protein, 320 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRN

SEQ ID NO: 45 - protein, 321 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSGSGSGSGSGSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 46 - protein, 318 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSGSGSGSSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 47 - protein, 321 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 48 - protein, 318 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 49 - protein, 320 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNTKPEPMDQYPSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 50 - protein, 321 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNNQMFDQKYDDPSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 51 - DNA, 963 bp, synthetic construct
  source 1..963: mol_type = other DNA
agccccggccaaggcacacagagcgagaactcatgtacccattttcccgggaacctgcccaacatgctgcggaagctgagagacgccttcagcagagtgaagactttctttcagatgaaggatcagctggacaaccttttactgaaggagagtctgctagaagacttcaagggatacctgggctgtcaagccttgtcggagatgattcaattctacctggaagaagtgatgccccaagctgagaaccaagaccccgatatcaaggcccatgtgaacagcctgggcgagaatctcaaaaccctgcgcttaaggctcagaagatgccacagatttctgccatgcgaaaacaagagcaaggcagtggaacaagtgaaaaacgccttcaacaagctgcaagagaagggcatctacaaagccatgagcgagttcgacattttcatcaactatattgaggcctacatgacgatgaagattcgaaatggcggagggtcgggctccggctctggcagcggctcctgcacccatttccccggcaacctgcccaatatgctgagaaagctgcgtgacgccttctctagagtaaaaaccttctttcagatgaaggatcagctggacaacttgttactgaaggagagcctgctcgaggacttcaaaggctacctgggctgccaagccctgagcgagatgattcagttttaccttgaagaggtgatgccccaagccgagaaccaagaccccgacatcaaagcccacgtcaactcgctgggcgaaaacctgaagactcttcggttgagacttcgaagatgtcatcggttcttgccctgcgagaataagagcaaggcggtggagcaagtgaagaacgcattcaataagctgcaagagaaaggtatctacaaggctatgagcgaatttgacatctttattaattacatcgaggcctatatgaccatgaaaatcagaaat

SEQ ID NO: 52 - DNA, 963 bp, synthetic construct
  source 1..963: mol_type = other DNA
agtcccggacaaggcacacagagcgagaacagctgcacgcatttccccggcaacctgcccaacatgctgcgtaaactcagagatgctttcagcagagtgaaaacgttcttccaaatgaaagaccaactcgataacctgttgcttaaggagtcactgctggaagatttcaaaggctatcttggctgtcaagccctgagcgaaatgatacagttctacctggaagaggtgatgcctcaagccgagaaccaagacccggatattaaggcccatgtaaattccctgggcgagaacctgaaaaccctcagactaagactgcgacgatgtcatagattcctaccctgtgaaaacaaatctaaggccgtggagcaagtgaagaatgcttttaacaaactgcaagagaagggcatttacaaggccatgagcgaatttgacatcttcatcaattacatcgaagcctacatgaccatgaaaattagaaacacgtcacccggccaaggcacacagtctgaaaacagctgcacccactttcccggcaaccttcccaacatgttgcgaaagctgagagacgcctttagccgcgtgaaaaccttcttccaaatgaaagatcagctggacaatctgctgctcaaggaaagcctgctcgaggacttcaagggctacctcggctgccaagccctcagcgagatgattcagttttaccttgaagaagtcatgcctcaagccgaaaaccaagaccccgatataaaagcccatgtgaacagcttgggcgagaacttaaagaccctgagattaagattacggcggtgccaccgattcttgccgtgcgagaacaagtcaaaggcggtcgagcaagtgaagaatgcattcaataaattacaagagaagggcatctataaggctatgagtgagttcgatattttcatcaactatatcgaggcttacatgacaatgaaaatcagaaac

SEQ ID NO: 53 - DNA, 963 bp, synthetic construct
  source 1..963: mol_type = other DNA
tcacccggccaaggcacacagagcgagaactcctgcacccacttccccgggaacttgcccaacatgctgcgcaagctgagagatgccttcagcagagtgaagactttctttcagatgaaggatcagcttgacaaccttctcctgaaggagagtctgctagaagacttcaagggatacctgggctgtcaagccttgtcggagatgattcagttttatctggaagaggtgatgccccaagctgagaatcaagaccccgacatcaaagcccatgtgaacagcctgggcgagaatctcaaaaccctgcgccttcgattgagaagatgccacagatttctgccatgcgaaaacaagagcaaggcagtggaacaagtgaaaaatgccttcaacaagctgcaagagaagggcatctacaaagccatgagcgagttcgacattttcatcaactacatagaggcttacatgaccatgaagatcagaaacaaccaaatgttcgatcagaagtacgacgaccctagctgcactcacttcccgggcaatctgccaaatatgttacgtaaactgcgggacgccttcagccgagttaaaactttctttcagatgaaggatcagctggacaacctgctgttaaaggagagcctgctggaggacttcaagggttatctgggctgccaagccctgagcgagatgatccaattttatctggaagaggtcatgccccaagccgaaaatcaagaccccgatattaaggcccatgtcaattctctcggggagaacctaaagaccttgcgccttcggttaagaagatgtcaccgctttctcccctgtgagaacaagtctaaggccgttgaacaagtgaagaacgccttcaataagctccaagagaaaggcatttataaggccatgtcggagttcgatatattcatcaattacatcgaggcgtatatgacaatgaagattaggaac

SEQ ID NO: 54 - DNA, 964 bp, synthetic construct
  source 1..964: mol_type = other DNA
agccccggccaaggcacacagagcgagaactcatgtacccattttcccgggaacctgcccaagatgctgcgggacctgagagacgccttcagcagagtgaagactttctttcagatgaaggatcagctggacaaccttttactgaaggagagtctgctagaagacttcaagggatacctgggctgtcaagccttgtcggagatgattcaattctacctggaagaagtgatgccccaagctgagaaccaagaccccgatatcaaggcccatgtgaacagcctgggcgagaatctcaaaaccctgcgcttaaggctcagaagatgccacagatttctgccatgcgaaaacaagagcaaggcagtggaacaagtgaaaaacgccttcaacaagctgcaagagaagggcatctacaaagccatgagcgagttcgacattttcatcaactatattgaggcctacatgacgatgaagattcgaaatggcggagggtcgggctccggctctggcagcggctcctgcacccatttccccggcaacctgcccaaaatgctgagagacctgcgtgacgccttctctagagtaaaaaccttctttcagatgaaggatcagctggacaacttgttactgaaggagagcctgctcgaggacttcaaaggctacctgggctgccaagccctgagcgagatgattcagttttaccttgaagaggtgatgccccaagccgagaaccaagaccccgacatcaaagcccacgtcaactcgctgggcgaaaacctgaagactcttcggttgagacttcgaagatgtcatcggttcttgccctgcgagaataagagcaaggcggtggagcaagtgaagaacgcattcaataagctgcaagagaaaggtatctacaaggctatgagcgaatttgacatctttattaattacatcgaggcctatatgaccatgaaaatcagaaatg

SEQ ID NO: 55 - DNA, 963 bp, synthetic construct
  source 1..963: mol_type = other DNA
agtcccggacaaggcacacagagcgagaacagctgcacgcatttccccggcaacctgcccaagatgctgcgtgatctcagagatgctttcagcagagtgaaaacgttcttccaaatgaaagaccaactcgataacctgttgcttaaggagtcactgctggaagatttcaaaggctatcttggctgtcaagccctgagcgaaatgatacagttctacctggaagaggtgatgcctcaagccgagaaccaagacccggatattaaggcccatgtaaattccctgggcgagaacctgaaaaccctcagactaagactgcgacgatgtcatagattcctaccctgtgaaaacaaatctaaggccgtggagcaagtgaagaatgcttttaacaaactgcaagagaagggcatttacaaggccatgagcgaatttgacatcttcatcaattacatcgaagcctacatgaccatgaaaattagaaacacgtcacccggccaaggcacacagtctgaaaacagctgcacccactttcccggcaaccttcccaagatgttgcgagacctgagagacgcctttagccgcgtgaaaaccttcttccaaatgaaagatcagctggacaatctgctgctcaaggaaagcctgctcgaggacttcaagggctacctcggctgccaagccctcagcgagatgattcagttttaccttgaagaagtcatgcctcaagccgaaaaccaagaccccgatataaaagcccatgtgaacagcttgggcgagaacttaaagaccctgagattaagattacggcggtgccaccgattcttgccgtgcgagaacaagtcaaaggcggtcgagcaagtgaagaatgcattcaataaattacaagagaagggcatctataaggctatgagtgagttcgatattttcatcaactatatcgaggcttacatgacaatgaaaatcagaaac

SEQ ID NO: 56 - DNA, 963 bp, synthetic construct
  source 1..963: mol_type = other DNA
tcacccggccaaggcacacagagcgagaactcctgcacccacttccccgggaacttgcccaagatgctgcgcgacctgagagatgccttcagcagagtgaagactttctttcagatgaaggatcagcttgacaaccttctcctgaaggagagtctgctagaagacttcaagggatacctgggctgtcaagccttgtcggagatgattcagttttatctggaagaggtgatgccccaagctgagaatcaagaccccgacatcaaagcccatgtgaacagcctgggcgagaatctcaaaaccctgcgccttcgattgagaagatgccacagatttctgccatgcgaaaacaagagcaaggcagtggaacaagtgaaaaatgccttcaacaagctgcaagagaagggcatctacaaagccatgagcgagttcgacattttcatcaactacatagaggcttacatgaccatgaagatcagaaacaaccaaatgttcgatcagaagtacgacgaccctagctgcactcacttcccgggcaatctgccaaaaatgttacgtgatctgcgggacgccttcagccgagttaaaactttctttcagatgaaggatcagctggacaacctgctgttaaaggagagcctgctggaggacttcaagggttatctgggctgccaagccctgagcgagatgatccaattttatctggaagaggtcatgccccaagccgaaaatcaagaccccgatattaaggcccatgtcaattctctcggggagaacctaaagaccttgcgccttcggttaagaagatgtcaccgctttctcccctgtgagaacaagtctaaggccgttgaacaagtgaagaacgccttcaataagctccaagagaaaggcatttataaggccatgtcggagttcgatatattcatcaattacatcgaggcgtatatgacaatgaagattaggaac

SEQ ID NO: 57 - DNA, 963 bp, synthetic construct
  source 1..963: mol_type = other DNA
agccccggccaaggcacacagagcgagaactcctgcacccacttccccgggaacctgcccaacgccctgcgggacctgagagacgccttcagcagagtgaagactttctttcagatgaaggatcagctggacaaccttcttctgaaggagagtctgctagaagacttcaaaggttacctgggctgtcaagccttgtcggagatgattcagttttatctggaagaggtcatgccccaagccgagaaccaagaccccgatatcaaagcccacgtaaatagcctgggagagaatctcaaaaccttacggcttcgactgagaagatgccacagatttctgccatgtgaaaacaaaagcaaagcagtggaacaagtgaaaaacgccttcaacaagctgcaagagaagggcatctataaagccatgagcgagttcgacattttcatcaattacatcgaggcctacatgaccatgaaaatcagaaacgggggcggctccggctctggcagcggcagcggtagttgcacccatttccccggcaacctgcccaatgcgctgagagacctaagagacgcctttagccgagttaagaccttctttcagatgaaggaccaactggacaacttactgctgaaggagagcctcctggaggactttaagggttatctgggttgccaagcccttagcgagatgatccaattctatctggaggaagtgatgccccaagcagagaatcaagaccccgacataaaggcccatgtcaacagcctgggggaaaacctgaaaacgctccgacttagattacggcgctgtcatagatttctgccctgcgaaaacaagtccaaggcggtggagcaagtgaagaatgcctttaacaagttacaagagaaaggcatctacaaagccatgtcggagttcgacatattcattaactacattgaagcatatatgaccatgaagatcagaaac

SEQ ID NO: 58 - DNA, 963 bp, synthetic construct
  source 1..963: mol_type = other DNA
agtcccggacaaggcacacagagcgagaacagctgcacgcatttccccggcaacctgcccaacgccctgcgtgatctcagagatgctttcagcagagtgaaaacgttcttccaaatgaaagaccaactcgataacctgttgcttaaggagtcactgctggaagatttcaaaggctatcttggctgtcaagccctgagcgaaatgatacagttctacctggaagaggtgatgcctcaagccgagaaccaagacccggatattaaggcccatgtaaattccctgggcgagaacctgaaaaccctcagactaagactgcgacgatgtcatagattcctaccctgtgaaaacaaatctaaggccgtggagcaagtgaagaatgcttttaacaaactgcaagagaagggcatttacaaggccatgagcgaatttgacatcttcatcaattacatcgaagcctacatgaccatgaaaattagaaacacgtcacccggccaaggcacacagtctgaaaacagctgcacccactttcccggcaaccttcccaacgccttgcgagacctgagagacgcctttagccgcgtgaaaaccttcttccaaatgaaagatcagctggacaatctgctgctcaaggaaagcctgctcgaggacttcaagggctacctcggctgccaagccctcagcgagatgattcagttttaccttgaagaagtcatgcctcaagccgaaaaccaagaccccgatataaaagcccatgtgaacagcttgggcgagaacttaaagaccctgagattaagattacggcggtgccaccgattcttgccgtgcgagaacaagtcaaaggcggtcgagcaagtgaagaatgcattcaataaattacaagagaagggcatctataaggctatgagtgagttcgatattttcatcaactatatcgaggcttacatgacaatgaaaatcagaaac

SEQ ID NO: 59 - DNA, 963 bp, synthetic construct
  source 1..963: mol_type = other DNA
tcacccggccaaggcacacagagcgagaactcctgcacccacttccccgggaacttgcccaacgccctgcgcgacctgagagatgccttcagcagagtgaagactttctttcagatgaaggatcagcttgacaaccttctcctgaaggagagtctgctagaagacttcaagggatacctgggctgtcaagccttgtcggagatgattcagttttatctggaagaggtgatgccccaagctgagaatcaagaccccgacatcaaagcccatgtgaacagcctgggcgagaatctcaaaaccctgcgccttcgattgagaagatgccacagatttctgccatgcgaaaacaagagcaaggcagtggaacaagtgaaaaatgccttcaacaagctgcaagagaagggcatctacaaagccatgagcgagttcgacattttcatcaactacatagaggcttacatgaccatgaagatcagaaacaaccaaatgttcgatcagaagtacgacgaccctagctgcactcacttcccgggcaatctgccaaatgccttacgtgatctgcgggacgccttcagccgagttaaaactttctttcagatgaaggatcagctggacaacctgctgttaaaggagagcctgctggaggacttcaagggttatctgggctgccaagccctgagcgagatgatccaattttatctggaagaggtcatgccccaagccgaaaatcaagaccccgatattaaggcccatgtcaattctctcggggagaacctaaagaccttgcgccttcggttaagaagatgtcaccgctttctcccctgtgagaacaagtctaaggccgttgaacaagtgaagaacgccttcaataagctccaagagaaaggcatttataaggccatgtcggagttcgatatattcatcaattacatcgaggcgtatatgacaatgaagattaggaac

SEQ ID NO: 60 - DNA, 963 bp, synthetic construct
  source 1..963: mol_type = other DNA
agccccggccaaggcacacagagcgagaactcctgcacccacttccccgggaacctgcccaacagcctgcgggacctgagagacgccttcagcagagtgaagactttctttcagatgaaggatcagctggacaaccttcttctgaaggagagtctgctagaagacttcaaaggctatctaggctgtcaagccttgtcggagatgattcagttttatctggaagaggttatgccccaagccgagaaccaagaccccgatatcaaagcgcatgtcaatagcctgggcgagaatctcaaaaccttacggcttcgactgagaagatgccacagatttctgccatgcgagaacaagagcaaggctgtggaacaagtgaaaaacgccttcaacaagctgcaagagaagggaatctacaaagcaatgagcgagttcgacattttcatcaattacatcgaggcctacatgaccatgaaaatcagaaacgggggcggctccggctctggcagcggcagcggttcctgcacccatttccccggcaacctgcccaattccctgagagacctaagagacgcctttagccgagttaagaccttctttcagatgaaggatcagcttgacaacttactgctgaaggagagcctcctggaggactttaagggctacctaggttgtcaagcactgagcgagatgatccaattctatctggaagaggtgatgccccaagctgaaaaccaagaccccgacataaaggcccatgtgaactcactgggggagaacctgaaaacgctccgacttagattacggcgctgtcatagatttctgccctgcgaaaacaagagcaaagccgtggagcaagtgaagaatgcctttaacaagttacaagagaaaggcatctacaaagccatgtcggagttcgacatattcattaactacattgaagcatatatgaccatgaagatcagaaac

SEQ ID NO: 61 - DNA, 963 bp, synthetic construct
  source 1..963: mol_type = other DNA
agtcccggacaaggcacacagagcgagaacagctgcacgcatttccccggcaacctgcccaacagcctgcgtgatctcagagatgctttcagcagagtgaaaacgttcttccaaatgaaagaccaactcgataacctgttgcttaaggagtcactgctggaagatttcaaaggctatcttggctgtcaagccctgagcgaaatgatacagttctacctggaagaggtgatgcctcaagccgagaaccaagacccggatattaaggcccatgtaaattccctgggcgagaacctgaaaaccctcagactaagactgcgacgatgtcatagattcctaccctgtgaaaacaaatctaaggccgtggagcaagtgaagaatgcttttaacaaactgcaagagaagggcatttacaaggccatgagcgaatttgacatcttcatcaattacatcgaagcctacatgaccatgaaaattagaaacacgtcacccggccaaggcacacagtctgaaaacagctgcacccactttcccggcaaccttcccaacagcttgcgagacctgagagacgcctttagccgcgtgaaaaccttcttccaaatgaaagatcagctggacaatctgctgctcaaggaaagcctgctcgaggacttcaagggctacctcggctgccaagccctcagcgagatgattcagttttaccttgaagaagtcatgcctcaagccgaaaaccaagaccccgatataaaagcccatgtgaacagcttgggcgagaacttaaagaccctgagattaagattacggcggtgccaccgattcttgccgtgcgagaacaagtcaaaggcggtcgagcaagtgaagaatgcattcaataaattacaagagaagggcatctataaggctatgagtgagttcgatattttcatcaactatatcgaggcttacatgacaatgaaaatcagaaac

SEQ ID NO: 62 - DNA, 963 bp, synthetic construct
  source 1..963: mol_type = other DNA
tcacccggccaaggcacacagagcgagaactcctgcacccacttccccgggaacttgcccaacagcctgcgcgacctgagagatgccttcagcagagtgaagactttctttcagatgaaggatcagcttgacaaccttctcctgaaggagagtctgctagaagacttcaagggatacctgggctgtcaagccttgtcggagatgattcagttttatctggaagaggtgatgccccaagctgagaatcaagaccccgacatcaaagcccatgtgaacagcctgggcgagaatctcaaaaccctgcgccttcgattgagaagatgccacagatttctgccatgcgaaaacaagagcaaggcagtggaacaagtgaaaaatgccttcaacaagctgcaagagaagggcatctacaaagccatgagcgagttcgacattttcatcaactacatagaggcttacatgaccatgaagatcagaaacaaccaaatgttcgatcagaagtacgacgaccctagctgcactcacttcccgggcaatctgccaaatagcttacgtgatctgcgggacgccttcagccgagttaaaactttctttcagatgaaggatcagctggacaacctgctgttaaaggagagcctgctggaggacttcaagggttatctgggctgccaagccctgagcgagatgatccaattttatctggaagaggtcatgccccaagccgaaaatcaagaccccgatattaaggcccatgtcaattctctcggggagaacctaaagaccttgcgccttcggttaagaagatgtcaccgctttctcccctgtgagaacaagtctaaggccgttgaacaagtgaagaacgccttcaataagctccaagagaaaggcatttataaggccatgtcggagttcgatatattcatcaattacatcgaggcgtatatgacaatgaagattaggaac

SEQ ID NO: 63 - DNA, 963 bp, synthetic construct
  source 1..963: mol_type = other DNA
agccccggccaaggcacacagagcgagaactcatgtacccattttcccgggaacctgcccaacatgctgcgggacctgagagacgccttcagcagagtgaagactttctttcagatgaaggatcagctggacaaccttttactgaaggagagtctgctagaagacttcaagggatacctgggctgtcaagccttgtcggagatgattcaattctacctggaagaagtgatgccccaagctgagaaccaagaccccgatatcaaggcccatgtgaacagcctgggcgagaatctcaaactgctgcgcttaaggctcagaagatgccacagatttctgccatgcgaaaacaagagcaaggcagtggaacaagtgaaaaacgccttcaacaagctgcaagagaagggcatctacaaagccatgagcgagttcgacattttcatcaactatattgaggcctacatgacgatgaagattcgaaatggcggagggtcgggctccggctctggcagcggctcctgcacccatttccccggcaacctgcccaatatgctgagagacctgcgtgacgccttctctagagtaaaaaccttctttcagatgaaggatcagctggacaacttgttactgaaggagagcctgctcgaggacttcaaaggctacctgggctgccaagccctgagcgagatgattcagttttaccttgaagaggtgatgccccaagccgagaaccaagaccccgacatcaaagcccacgtcaactcgctgggcgaaaacctgaagcttcttcggttgagacttcgaagatgtcatcggttcttgccctgcgagaataagagcaaggcggtggagcaagtgaagaacgcattcaataagctgcaagagaaaggtatctacaaggctatgagcgaatttgacatctttattaattacatcgaggcctatatgaccatgaaaatcagaaat

SEQ ID NO: 64 - DNA, 963 bp, synthetic construct
  source 1..963: mol_type = other DNA
agtcccggacaaggcacacagagcgagaacagctgcacgcatttccccggcaacctgcccaacatgctgcgtgatctcagagatgctttcagcagagtgaaaacgttcttccaaatgaaagaccaactcgataacctgttgcttaaggagtcactgctggaagatttcaaaggctatcttggctgtcaagccctgagcgaaatgatacagttctacctggaagaggtgatgcctcaagccgagaaccaagacccggatattaaggcccatgtaaattccctgggcgagaacctgaaactgctcagactaagactgcgacgatgtcatagattcctaccctgtgaaaacaaatctaaggccgtggagcaagtgaagaatgcttttaacaaactgcaagagaagggcatttacaaggccatgagcgaatttgacatcttcatcaattacatcgaagcctacatgaccatgaaaattagaaacacgtcacccggccaaggcacacagtctgaaaacagctgcacccactttcccggcaaccttcccaacatgttgcgagacctgagagacgcctttagccgcgtgaaaaccttcttccaaatgaaagatcagctggacaatctgctgctcaaggaaagcctgctcgaggacttcaagggctacctcggctgccaagccctcagcgagatgattcagttttaccttgaagaagtcatgcctcaagccgaaaaccaagaccccgatataaaagcccatgtgaacagcttgggcgagaacttaaagctgctgagattaagattacggcggtgccaccgattcttgccgtgcgagaacaagtcaaaggcggtcgagcaagtgaagaatgcattcaataaattacaagagaagggcatctataaggctatgagtgagttcgatattttcatcaactatatcgaggcttacatgacaatgaaaatcagaaac

SEQ ID NO: 65 - DNA, 963 bp, synthetic construct
  source 1..963: mol_type = other DNA
tcacccggccaaggcacacagagcgagaactcctgcacccacttccccgggaacttgcccaacatgctgcgcgacctgagagatgccttcagcagagtgaagactttctttcagatgaaggatcagcttgacaaccttctcctgaaggagagtctgctagaagacttcaagggatacctgggctgtcaagccttgtcggagatgattcagttttatctggaagaggtgatgccccaagctgagaatcaagaccccgacatcaaagcccatgtgaacagcctgggcgagaatctcaaactgctgcgccttcgattgagaagatgccacagatttctgccatgcgaaaacaagagcaaggcagtggaacaagtgaaaaatgccttcaacaagctgcaagagaagggcatctacaaagccatgagcgagttcgacattttcatcaactacatagaggcttacatgaccatgaagatcagaaacaaccaaatgttcgatcagaagtacgacgaccctagctgcactcacttcccgggcaatctgccaaatatgttacgtgatctgcgggacgccttcagccgagttaaaactttctttcagatgaaggatcagctggacaacctgctgttaaaggagagcctgctggaggacttcaagggttatctgggctgccaagccctgagcgagatgatccaattttatctggaagaggtcatgccccaagccgaaaatcaagaccccgatattaaggcccatgtcaattctctcggggagaacctaaagctgttgcgccttcggttaagaagatgtcaccgctttctcccctgtgagaacaagtctaaggccgttgaacaagtgaagaacgccttcaataagctccaagagaaaggcatttataaggccatgtcggagttcgatatattcatcaattacatcgaggcgtatatgacaatgaagattaggaac

SEQ ID NO: 66 - DNA, 963 bp, synthetic construct
  source 1..963: mol_type = other DNA
agccccggccaaggcacacagagcgagaactcatgtacccattttcccgggaacctgcccaacatgctgcgggacctgagagacgccttcagcagagtgaagactttctttcagatgaaggatcagctggacaaccttttactgaaggagagtctgctagaagacttcaagggatacctgggctgtcaagccttgtcggagatgattcaattctacctggaagaagtgatgccccaagctgagaaccaagaccccgatatcaaggcccatgtgaacagcctggggcagaatctcaaaaccctgcgcttaaggctcagaagatgccacagatttctgccatgcgaaaacaagagcaaggcagtggaacaagtgaaaaacgccttcaacaagctgcaagagaagggcatctacaaagccatgagcgagttcgacattttcatcaactatattgaggcctacatgacgatgaagattcgaaatggcggagggtcgggctccggctctggcagcggctcctgcacccatttccccggcaacctgcccaatatgctgagagacctgcgtgacgccttctctagagtaaaaaccttctttcagatgaaggatcagctggacaacttgttactgaaggagagcctgctcgaggacttcaaaggctacctgggctgccaagccctgagcgagatgattcagttttaccttgaagaggtgatgccccaagccgagaaccaagaccccgacatcaaagcccacgtcaactcgctgggccaaaacctgaagactcttcggttgagacttcgaagatgtcatcggttcttgccctgcgagaataagagcaaggcggtggagcaagtgaagaacgcattcaataagctgcaagagaaaggtatctacaaggctatgagcgaatttgacatctttattaattacatcgaggcctatatgaccatgaaaatcagaaat

SEQ ID NO: 67 - DNA, 963 bp, synthetic construct
  source 1..963: mol_type = other DNA
agtcccggacaaggcacacagagcgagaacagctgcacgcatttccccggcaacctgcccaacatgctgcgtgatctcagagatgctttcagcagagtgaaaacgttcttccaaatgaaagaccaactcgataacctgttgcttaaggagtcactgctggaagatttcaaaggctatcttggctgtcaagccctgagcgaaatgatacagttctacctggaagaggtgatgcctcaagccgagaaccaagacccggatattaaggcccatgtaaattccctggggcagaacctgaaaaccctcagactaagactgcgacgatgtcatagattcctaccctgtgaaaacaaatctaaggccgtggagcaagtgaagaatgcttttaacaaactgcaagagaagggcatttacaaggccatgagcgaatttgacatcttcatcaattacatcgaagcctacatgaccatgaaaattagaaacacgtcacccggccaaggcacacagtctgaaaacagctgcacccactttcccggcaaccttcccaacatgttgcgagacctgagagacgcctttagccgcgtgaaaaccttcttccaaatgaaagatcagctggacaatctgctgctcaaggaaagcctgctcgaggacttcaagggctacctcggctgccaagccctcagcgagatgattcagttttaccttgaagaagtcatgcctcaagccgaaaaccaagaccccgatataaaagcccatgtgaacagcttggggcagaacttaaagaccctgagattaagattacggcggtgccaccgattcttgccgtgcgagaacaagtcaaaggcggtcgagcaagtgaagaatgcattcaataaattacaagagaagggcatctataaggctatgagtgagttcgatattttcatcaactatatcgaggcttacatgacaatgaaaatcagaaac

SEQ ID NO: 68 - DNA, 963 bp, synthetic construct
  source 1..963: mol_type = other DNA
tcacccggccaaggcacacagagcgagaactcctgcacccacttccccgggaacttgcccaacatgctgcgcgacctgagagatgccttcagcagagtgaagactttctttcagatgaaggatcagcttgacaaccttctcctgaaggagagtctgctagaagacttcaagggatacctgggctgtcaagccttgtcggagatgattcagttttatctggaagaggtgatgccccaagctgagaatcaagaccccgacatcaaagcccatgtgaacagcctggggcagaatctcaaaaccctgcgccttcgattgagaagatgccacagatttctgccatgcgaaaacaagagcaaggcagtggaacaagtgaaaaatgccttcaacaagctgcaagagaagggcatctacaaagccatgagcgagttcgacattttcatcaactacatagaggcttacatgaccatgaagatcagaaacaaccaaatgttcgatcagaagtacgacgaccctagctgcactcacttcccgggcaatctgccaaatatgttacgtgatctgcgggacgccttcagccgagttaaaactttctttcagatgaaggatcagctggacaacctgctgttaaaggagagcctgctggaggacttcaagggttatctgggctgccaagccctgagcgagatgatccaattttatctggaagaggtcatgccccaagccgaaaatcaagaccccgatattaaggcccatgtcaattctctcgggcagaacctaaagaccttgcgccttcggttaagaagatgtcaccgctttctcccctgtgagaacaagtctaaggccgttgaacaagtgaagaacgccttcaataagctccaagagaaaggcatttataaggccatgtcggagttcgatatattcatcaattacatcgaggcgtatatgacaatgaagattaggaac

SEQ ID NO: 69 - DNA, 963 bp, synthetic construct
  source 1..963: mol_type = other DNA
tctccgggccaaggcacacagagcgagaacagctgcacgcacttccccggcaacctgcccaacatgctgcgggatctcagagatgctttcagcagagtgaaaacgttctttcagatgaaggaccaactcgacaacctgctgctgaaggagtcactgctggaagatttcaaaggctatcttggctgtcaagccctgagcgaaatgatacagttctacctggaagaggtgatgccacaagccgagaaccaagacccggatattaaggcccatgtaaattccctgggcgagaacctgaaactgctcagactgcgacttagaagatgtcaccgcttcctgccgtgcgagaacaagagcaaggcagtggagcaagtgaaaaacgcctttaacaaactgcaagagaaggggatctataaagccatgtccgagtttgacatttttattaactatatcgaggcctatatgaccatgaaaatcagaaacacaagccccggccaaggcactcagtctgagaactcgtgcacccattttcccggcaatctgcccaacatgctgagggacctgagagatgccttctcgagagtgaaaaccttctttcaaatgaaagatcagctggacaacttgctgctgaaagagagccttctggaggacttcaagggctatctcggctgccaagccctgagcgagatgattcagttttatctggaagaggtgatgccccaagccgaaaaccaagaccccgacatcaaggcccacgtcaacagcctgggtgagaacctgaagacccttcgactgagactgagacgatgtcatcggttcctgccctgtgagaacaaatcgaaggccgtagagcaagtgaagaatgcttttaataagctgcaagagaaaggaatatataaagctatgtcagagttcgacatctttatcaactacatcgaggcctacatgaccatgaagatccggaat

SEQ ID NO: 70 - DNA, 960 bp, synthetic construct
  source 1..960: mol_type = other DNA
agccccggccagggtacacagtcagagaatagttgcacacactttcctggcaatctgcctaatatgctacgcgatctcagggatgccttttctcgcgttaaaacttttttccaaatgaaggaccagcttgataacctgctactcaaagaaagccttttggaggattttaaggggtacctgggttgccaggccctgtcggaaatgattcagttttatttggaggaggtgatgccccaggcagaaaaccaagatcctgacatcaaggcacatgtaaattcccttggagagaacctcaaaacactccgtcttaggctccgacgctgccatcgattcctgccttgcgagaacaagtccaaggcagtggagcaagtcaagaacgcctttaataaattacaagagaagggcatctacaaagcgatgtccgagtttgacatcttcattaactacattgaagcttacatgaccatgaaaataaggaacagcccgggacagggcacccagagtgaaaactcttgtacgcacttccccgggaatctgcccaacatgctgagagatctgcgggacgccttctcaagagtgaagaccttcttccaaatgaaagaccagttggacaatctgctgctgaaggaaagcttattagaggatttcaaaggctatctaggatgtcaggctctgtcagaaatgatccagttctatctggaggaggtgatgccacaggccgagaatcaggacccagacattaaagcgcacgtcaattctctgggagaaaacctcaagactctccggttgcggctgagacggtgtcatagattcttgccatgtgaaaacaagtccaaagctgttgaacaggtgaaaaatgctttcaataagcttcaggaaaaggggatctacaaggccatgagtgagtttgacatatttatcaactacattgaggcatatatgaccatgaagatcaggaac

SEQ ID NO: 71 - DNA, 960 bp, synthetic construct
  source 1..960: mol_type = other DNA
agtcccggacaaggcacacagagcgagaacagctgcacgcatttccccggcaacctgcccaacatgctgcgtgatctcagagatgctttcagcagagtgaaaacgttcttccaaatgaaagaccaactcgataacctgttgcttaaggagtcactgctggaagatttcaaaggctatcttggctgtcaagccctgagcgaaatgatacagttctacctggaagaggtgatgcctcaagccgagaaccaagacccggatattaaggcccatgtaaattccctgggcgagaacctgaaactgctcagactaagactgcgacgatgtcatagattcctaccctgtgaaaacaaatctaaggccgtggagcaagtgaagaatgcttttaacaaactgcaagagaagggcatttacaaggccatgagcgaatttgacatcttcatcaattacatcgaagcctacatgaccatgaaaattagaaactcacccggccaaggcacacagtctgaaaacagctgcacccactttcccggcaaccttcccaacatgttgcgagacctgagagacgcctttagccgcgtgaaaaccttcttccaaatgaaagatcagctggacaatctgctgctcaaggaaagcctgctcgaggacttcaagggctacctcggctgccaagccctcagcgagatgattcagttttaccttgaagaagtcatgcctcaagccgaaaaccaagaccccgatataaaagcccatgtgaacagcttgggcgagaacttaaagctgctgagattaagattacggcggtgccaccgattcttgccgtgcgagaacaagtcaaaggcggtcgagcaagtgaagaatgcattcaataaattacaagagaagggcatctataaggctatgagtgagttcgatattttcatcaactatatcgaggcttacatgacaatgaaaatcagaaac

SEQ ID NO: 72 - DNA, 963 bp, synthetic construct
  source 1..963: mol_type = other DNA
agcccaggacagggaactcagagcgaaaactcttgtacacactttcctggaaatctgcccaacatgctgcgcgacctcagagatgccttcagccgcgtgaaaaccttcttccagatgaaggaccaactcgataacttgctgttaaaggaaagcctgttggaagatttcaagggctacctaggttgtcaggcattgtccgaaatgatacagttttacttggaggaagtcatgccccaagcagagaaccaagaccctgatatcaaagcccacgtgaatagtctcggagagaatttaaaaactctgcggctcagactccgacgatgtcataggtttctgccgtgcgagaacaagtcaaaggcagtggagcaggtaaagaacgcgttcaataagcttcaggaaaaagggatctacaaagctatgtctgagtttgacatctttataaattatatcgaggcttatatgacaatgaaaatcaggaatgggtccggttcgggctccggtagtgggtctggctcctgcacccatttccctggaaacctgccaaatatgctgcgggatctgcgtgacgcctttagcagagtgaagaccttcttccagatgaaagaccagctggacaacttgctactgaaggagtcacttctggaagactttaaagggtacctgggctgccaagccctgtccgagatgattcagttctacttagaagaagtgatgccccaggccgaaaatcaggacccagatattaaagctcatgttaacagtctaggcgagaatcttaaaacactccggctccggcttaggcgctgccacagatttcttccctgtgagaataagtctaaggcggttgagcaagtcaaaaacgctttcaataagctgcaggagaagggcatctataaggcaatgagcgagtttgatattttcattaactatatcgaagcctacatgacgatgaagattaggaac

SEQ ID NO: 73 - DNA, 954 bp, synthetic construct
  source 1..954: mol_type = other DNA
agccctggccaaggcacccagagcgaaaacagctgtacccatttcccgggcaatctgcccaatatgctccgcgatctaagagatgcattttcccgggtcaagacattctttcagatgaaggaccaactggataatttgcttctgaaggaatccctcctcgaagatttcaagggttacttggggtgccaggcactgtctgagatgatacagttctacctagaagaggttatgccacaagcggaaaatcaggacccagacattaaggcacacgtgaactctttaggtgagaacttgaagacactccggcttcggctccgtcgatgccatcgcttccttccatgtgagaacaagtccaaggctgttgagcaagtgaagaacgcttttaataaactacaggagaaaggcatctacaaggccatgtcagagtttgatattttcatcaattacatcgaagcctacatgacgatgaaaatcaggaatggaagcggttcgggatctgggagctcatgtactcatttccccggcaacctgcccaatatgctcagggacctcagagacgcctttagccgcgtgaaaaccttctttcagatgaaagaccagcttgacaacttgctgctgaaggaaagtttgttagaggatttcaaaggatacctggggtgtcaggccctgtccgaaatgatccaattttatctggaggaggtaatgcctcaggccgagaatcaggaccccgatattaaagcacacgtgaatagtctgggagaaaacttaaagactctgcgactgagactgaggaggtgccacagattcctgccttgcgaaaataaaagtaaagctgtcgagcaggtgaagaacgccttcaacaagcttcaggagaaggggatttataaagcgatgtctgagtttgacatatttattaactatatcgaagcttatatgacaatgaaaatccggaac

SEQ ID NO: 74 - DNA, 963 bp, synthetic construct
  source 1..963: mol_type = other DNA
agccccggccagggtacacagtcagagaatagttgcacacactttcctggcaatctgcctaatatgctacgcgatctcagggatgccttttctcgcgttaaaacttttttccaaatgaaggaccagcttgataacctgctactcaaagaaagccttttggaggattttaaggggtacctgggttgccaggccctgtcggaaatgattcagttttatttggaggaggtgatgccccaggcagaaaaccaagatcctgacatcaaggcacatgtaaattcccttggagagaacctcaaaacactccgtcttaggctccgacgctgccatcgattcctgccttgcgagaacaagtccaaggcagtggagcaagtcaagaacgcctttaataaattacaagagaagggcatctacaaagcgatgtccgagtttgacatcttcattaactacattgaagcttacatgaccatgaaaataaggaacactagcccgggacagggcacccagagtgaaaactcttgtacgcacttccccgggaatctgcccaacatgctgagagatctgcgggacgccttctcaagagtgaagaccttcttccaaatgaaagaccagttggacaatctgctgctgaaggaaagcttattagaggatttcaaaggctatctaggatgtcaggctctgtcagaaatgatccagttctatctggaggaggtgatgccacaggccgagaatcaggacccagacattaaagcgcacgtcaattctctgggagaaaacctcaagactctccggttgcggctgagacggtgtcatagattcttgccatgtgaaaacaagtccaaagctgttgaacaggtgaaaaatgctttcaataagcttcaggaaaaggggatctacaaggccatgagtgagtttgacatatttatcaactacattgaggcatatatgaccatgaagatcaggaac

SEQ ID NO: 75 - DNA, 954 bp, synthetic construct
  source 1..954: mol_type = other DNA
agccctgggcagggcacacaaagcgaaaactcttgtacccatttcccagggaatctccccaacatgctgcgagatctgagggatgctttcagtagggtgaagacctttttccaaatgaaggaccagctcgacaatctgttattaaaagagtcactgctggaggacttcaaaggctacttgggctgccaggctctgagtgaaatgatccagttctatttggaggaggttatgccccaagcagagaaccaggaccccgatatcaaggcgcacgtcaattccctaggcgagaatctcaagacgcttcggcttcgcctgcgccggtgccacaggtttctgccatgcgagaataagtcaaaggcggttgaacaggtgaagaacgccttcaacaaactccaggagaaaggaatctacaaggctatgtccgaatttgatatattcattaactatatcgaagcctacatgactatgaaaattagaaatggacaaggaacacagagcgagaactcttgtacccattttcctggtaatttacccaacatgctgagagacctgagggatgcattctccagagtgaagactttttttcagatgaaagaccagctcgataatctcctgctcaaagaaagtctactagaggatttcaagggctacctgggatgtcaggccctgagcgaaatgatccagttttatttggaagaggtgatgccgcaggctgagaatcaagaccctgacattaaggcccacgtcaactctctgggtgagaaccttaagacacttcggcttcgactgcggcgctgccatcgtttcttgccatgtgagaacaaatccaaagccgtagaacaggtgaagaatgcatttaacaaattgcaggagaaagggatatataaggcaatgtcggaatttgacatcttcatcaactacattgaagcctacatgaccatgaagattagaaat

SEQ ID NO: 76 - DNA, 960 bp, synthetic construct
  source 1..960: mol_type = other DNA
agcccaggacagggaacacagtccgaaaattcatgtacccacttcccggggaatctccccaatatgcttcgagacctgagggatgcgttctctagagttaagaccttcttccagatgaaggatcagttggataatctgctgctgaaggagagcctgctggaggattttaaagggtatttaggttgtcaagccctgtccgaaatgatccaattctaccttgaggaagtgatgcctcaggcagagaatcaagatcccgacatcaaggctcacgtgaactccctaggtgagaacctgaagaccttaaggttgagattgcgccggtgccataggttcctcccttgtgaaaataagagcaaagctgtcgaacaggtgaaaaacgcgtttaacaaactgcaggaaaaaggcatctacaaggcaatgtctgagttcgacatcttcataaactacatcgaggcttatatgactatgaaaattagaaacacaaaacccgagcccatggatcagtatccttcttgcacgcactttccgggcaacctcccaaacatgttacgcgatctgagggacgcatttagtcgcgtaaaaacctttttccagatgaaggaccagcttgacaatctcctgttgaaggagagtctgcttgaagattttaaaggatacctggggtgccaagccctctccgaaatgattcagttctatctggaagaggttatgccacaggccgagaatcaagacccagacattaaggctcatgtgaatagcctcggcgagaaccttaagacactgcgtctccggttgcgaagatgccatcggtttctaccttgtgaaaataaatcaaaggccgtggagcaggtcaagaacgccttcaacaagctacaggagaaaggcatatataaagcaatgtcggagtttgacatctttattaattacatcgaagcctacatgactatgaagattcggaac

SEQ ID NO: 77 - DNA, 963 bp, synthetic construct
  source 1..963: mol_type = other DNA
tctccaggccagggcacgcaatctgaaaatagctgcacacattttcctggcaacctccccaacatgcttagagacttaagggacgcatttagcagggttaagacattcttccagatgaaggatcagttggataatctcctcctgaaagagtccctactagaggactttaagggatacttaggttgtcaagccctgagcgaaatgatccagttctacctggaagaagtgatgccccaggccgagaaccaagatccagatattaaagcgcacgtcaactccctgggagaaaacctcaaaactctgcgcctgaggcttagacgctgtcatcgattcctgccgtgcgagaataaatcgaaggcggtggagcaagtgaaaaacgcttttaacaagttgcaggaaaaaggtatctacaaagctatgagcgagtttgacattttcataaactatatcgaggcttatatgaccatgaagatccggaacaaccaaatgtttgaccagaaatatgacgacccatcctgtacccatttccctgggaatctccctaacatgctcagagacctgcgggatgccttctcccgcgttaaaaccttttttcagatgaaggatcagttggataatctgcttctcaaggagtctcttcttgaagacttcaagggatacctggggtgccaggccctgtcagaaatgattcagttctatctagaggaggtgatgcctcaggctgagaatcaggaccccgatattaaagcccacgtcaacagtctgggggaaaatctgaagacactgcggttaaggttgcggcgatgtcacagattcttgccctgcgaaaataagtcaaaagcagtagagcaggtgaaaaacgcatttaataagctgcaggaaaagggcatttacaaggccatgagtgagttcgatatctttataaattacatcgaggcatatatgactatgaagatccgtaat

SEQ ID NO: 78 - protein, 324 aa, synthetic construct
QYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSSRGQYSREDNQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 79 - protein, 324 aa, synthetic construct
QYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKLLRMRLRRCHRFLPCENKSKAVEQVKSDFNKLQDQGVYKAMNEFDIFINCIEAYMMIKMKSSRGQYSREDNQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKLLRMRLRRCHRFLPCENKSKAVEQVKSDFNKLQDQGVYKAMNEFDIFINCIEAYMMIKMKS

SEQ ID NO: 80 - protein, 324 aa, synthetic construct
QYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKLLRMRLRRCHRFLPCENKSKAVEQVKSDFNKLQDQGVYKAMNEFDIFINCIEAYMMIKMKSSRGQYSREDNQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 81 - protein, 324 aa, synthetic construct
QYSREDNSCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSSRGQYSREDNQYSREDNSCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 82 - protein, 318 aa, synthetic construct
GNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSSRGQYSREDNQYSREDNSCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 83 - protein, 318 aa, synthetic construct
GNCTHFPVGQSHMLLELRTAFSQVKTFFQTKDQLDNILLTDSLMQDFKGYLGCQALSEMIQFYLVEVMPQAEKHGPEIKEHLNSLGEKLKLLRMRLRRCHRFLPCENKSKAVEQVKSDFNKLQDQGVYKAMNEFDIFINCIEAYMMIKMKSSRGQYSREDNQYSREDNSCTHFPVGQSHMLLELRTAFSQVKTFFQTKDQLDNILLTDSLMQDFKGYLGCQALSEMIQFYLVEVMPQAEKHGPEIKEHLNSLGEKLKLLRMRLRRCHRFLPCENKSKAVEQVKSDFNKLQDQGVYKAMNEFDIFINCIEAYMMIKMKS

SEQ ID NO: 84 - protein, 327 aa, synthetic construct
SRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSSRGQYSREDNQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 85 - protein, 327 aa, synthetic construct
SRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKLLRMRLRRCHRFLPCENKSKAVEQVKSDFNKLQDQGVYKAMNEFDIFINCIEAYMMIKMKSSRGQYSREDNQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKLLRMRLRRCHRFLPCENKSKAVEQVKSDFNKLQDQGVYKAMNEFDIFINCIEAYMMIKMKS

SEQ ID NO: 86 - protein, 317 aa, synthetic construct
EYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 87 - protein, 317 aa, synthetic construct
EYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKLLRMRLRRCHRFLPCENKSKAVEQVKSDFNKLQDQGVYKAMNEFDIFINCIEAYMMIKMKSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKLLRMRLRRCHRFLPCENKSKAVEQVKSDFNKLQDQGVYKAMNEFDIFINCIEAYMMIKMKS

SEQ ID NO: 88 - DNA, 974 bp, synthetic construct
  source 1..974: mol_type = other DNA
cagtacagcagagaagataataactgcacccacttccccgtagggcagagccatatgctgctggagctgagaacagcctttagccaagtcaagaccttcttccaaacgaaggatcagcttgacaatatactgctgaccgatagcctgatgcaagacttcaaaggttacctcggctgtcaagccctctccgagatgatccaattttacctggtggaggtgatgccgcaagccgaaaaacatggccccgagatcaaagaacatctgaacagtcttggtgaaaagctgaagaccctgcgaatgcggctgcgcagatgtcacagatttctaccgtgcgagaataagagcaaggccgtggagcaagtgaagagcgacttcaataagctgcaagaccaaggcgtttataaggcgatgaacgagttcgatatatttatcaactgcatagaagcctatatgatgatcaagatgaagagcagcagagggcagtactcgagagaggataatcagtacagccgagaagacaacaactgtacccacttccccgtgggtcaaagccacatgctgctagagctcagaacggccttcagccaagtgaagactttctttcaaaccaaggaccaactggacaacatcctgttaaccgactcactaatgcaagactttaaggggtacctgggttgccaagccctgagcgagatgattcagttctacctcgtggaggtcatgcctcaagccgaaaagcacggccccgagattaaagagcacttaaacagtctaggggagaaactgaagaccttgagaatgagattaagacggtgtcatagatttctcccctgtgagaacaaaagcaaagcggtggagcaagtgaagagcgattttaacaagctccaagatcaaggcgtatataaagcgatgaatgagttcgatatcttcattaactgcatcgaagcctatatgatgattaaaatgaaaagcgg

SEQ ID NO: 89 - DNA, 974 bp, synthetic construct
  source 1..974: mol_type = other DNA
cagtacagcagagaagataataactgcacccacttccccgtagggcagagccatatgctgctggagctgagaacagcctttagccaagtcaagaccttcttccaaacgaaggatcagcttgacaatatactgctgaccgatagcctgatgcaagacttcaaaggttacctcggctgtcaagccctctccgagatgatccaattttacctggtggaggtgatgccgcaagccgaaaaacatggccccgagatcaaagaacatctgaacagtcttggtgaaaagctgaagctgctgcgaatgcggctgcgcagatgtcacagatttctaccgtgcgagaataagagcaaggccgtggagcaagtgaagagcgacttcaataagctgcaagaccaaggcgtttataaggcgatgaacgagttcgatatatttatcaactgcatagaagcctatatgatgatcaagatgaagagcagcagagggcagtactcgagagaggataatcagtacagccgagaagacaacaactgtacccacttccccgtgggtcaaagccacatgctgctagagctcagaacggccttcagccaagtgaagactttctttcaaaccaaggaccaactggacaacatcctgttaaccgactcactaatgcaagactttaaggggtacctgggttgccaagccctgagcgagatgattcagttctacctcgtggaggtcatgcctcaagccgaaaagcacggccccgagattaaagagcacttaaacagtctaggggagaaactgaagctgttgagaatgagattaagacggtgtcatagatttctcccctgtgagaacaaaagcaaagcggtggagcaagtgaagagcgattttaacaagctccaagatcaaggcgtatataaagcgatgaatgagttcgatatcttcattaactgcatcgaagcctatatgatgattaaaatgaaaagcgg

SEQ ID NO: 90 - DNA, 974 bp, synthetic construct
  source 1..974: mol_type = other DNA
cagtacagcagagaagataataactgcacccacttccccgtagggcagtcacacatgctgctggagctgcggactgcctttagccaagtcaagaccttcttccaaacgaaggatcagcttgacaatatactgctgactgacagccttatgcaagacttcaaaggttacctcggctgtcaagccctctccgagatgatccaattttacctggtggaggtgatgccgcaagccgagaaacacggaccggagatcaaagaacatctgaacagtcttggtgaaaagctgaagctgctgagaatgcgcctgagaagatgccatagattcctgccctgcgagaacaagtccaaagcggttgaacaagtgaagagcgacttcaataagctgcaagaccaaggcgtgtataaggctatgaacgagttcgacatctttataaattgtatcgaggcgtacatgatgataaaaatgaaaagcagcagagggcagtactcgagagaggataatcaatacagcagagaagacaacaactgcactcactttcccgtgggtcagtcacatatgctcctggaactgagaaccgccttctcgcaagtgaagactttcttccaaaccaaggatcagctggacaacatcttgctcactgacagcctgatgcaagacttcaaaggctaccttggctgccaagccctgagcgaaatgattcagttctacctagttgaagtgatgcctcaagccgaaaagcacggccccgaaataaaggaacacctgaacagcttaggcgagaagctgaagacattgagaatgagattaagaagatgccacagattcttaccctgtgaaaataaaagcaaggcggtggagcaagtgaagagcgattttaacaagctccaagaccaaggcgtatataaagcgatgaacgagttcgacatcttcattaactgcatcgaagcatacatgatgattaagatgaagagcgg

SEQ ID NO: 91 - DNA, 972 bp, synthetic construct
  source 1..972: mol_type = other DNA
caatactcaagagaagataacagttgcacacatttccccgtagggcaaagtcacatgctgttggaattgcgcactgcattttctcaggtaaaaacctttttccaaaccaaagaccagcttgataacattcttctgaccgactcacttatgcaggacttcaaaggctaccttggctgccaagccctttctgaaatgatacaattctacttggtcgaggtcatgcctcaggcagaaaagcatggtccagaaatcaaagaacatttgaactccctgggagagaagcttaaaactcttaggatgcgccttcggagatgccatcgctttcttccatgcgagaataaaagcaaggccgtcgaacaagtaaagtccgactttaataagctgcaagaccaaggtgtctataaagcaatgaacgaatttgacatctttatcaattgtattgaggcctatatgatgatcaaaatgaaaagttcccgcgggcaatactcacgggaggacaatcaatattcccgagaagataattcttgtacccacttccccgtcggtcaaagccacatgcttcttgagctcagaactgcattctcccaagtcaagacttttttccaaacaaaagatcaacttgacaacatcctcttgactgactccttgatgcaagacttcaaaggttacctcggctgtcaagctctcagtgagatgattcaattctatttggtagaggttatgcctcaagccgagaagcatgggccagaaatcaaagagcaccttaactccctgggtgaaaaactcaagacccttcggatgaggcttcgcagatgtcaccggttcctcccatgcgagaataagtccaaggctgttgagcaagtgaaatcagacttcaacaaattgcaagaccaaggagtttacaaagcaatgaatgagttcgatatctttattaactgtatcgaagcatacatgatgattaaaatgaagagc

SEQ ID NO: 92 - DNA, 956 bp, synthetic construct
  source 1..956: mol_type = other DNA
ggcaactgtactcatttcccagtggggcaaagccatatgttgttggaattgcgcactgctttctctcaggtcaaaacattttttcagacaaaagaccaattggacaacattttattgacagatagtctgatgcaggattttaaggggtacttgggttgtcaagctctttctgaaatgatccagttttatctggttgaagttatgccacaggcggaaaagcacggaccggagatcaaggaacatttaaattcactgggcgaaaaacttaagactctgcgcatgcgcctgcgccgctgtcaccgttttctgccatgtgaaaataagtccaaggccgtagaacaggtgaaatctgacttcaacaaactgcaggaccagggggtgtacaaagcaatgaatgagtttgatatttttatcaattgtattgaagcctatatgatgatcaagatgaaatcatctcgcggtcaatacagccgcgaagacaaccagtattcacgtgaggacaattcgtgcacccacttcccggtgggccaatcgcatatgctgctggagttacgcacagccttctcgcaggtgaagactttttttcagactaaggatcagctggataatatcctgttgaccgattcattaatgcaagatttcaagggttacctgggttgccaggcgttatccgagatgattcaattctacttagtagaagtgatgccccaggctgaaaaacacggtcctgagattaaagagcacttgaacagtttgggagaaaagctgaagactcttcgtatgcgtctgcgccgctgccatcgctttttgccctgtgagaataaatccaaggccgtcgagcaggttaagtctgatttcaacaaattacaggatcaaggggtttacaaagcgatgaatgagtttgacatctttattaattgtatcgaggcttatatgatgattaagatgaagagtgg

SEQ ID NO: 93 - DNA, 954 bp, synthetic construct
  source 1..954: mol_type = other DNA
ggcaactgtactcatttcccagtggggcaaagccatatgttgttggaattgcgcactgctttctctcaggtcaaaacattttttcagacaaaagaccaattggacaacattttattgacagatagtctgatgcaggattttaaggggtacttgggttgtcaagctctttctgaaatgatccagttttatctggttgaagttatgccacaggcggaaaagcacggaccggagatcaaggaacatttaaattcactgggcgaaaaacttaagctactgcgcatgcgcctgcgccgctgtcaccgttttctgccatgtgaaaataagtccaaggccgtagaacaggtgaaatctgacttcaacaaactgcaggaccagggggtgtacaaagcaatgaatgagtttgatatttttatcaattgtattgaagcctatatgatgatcaagatgaaatcatctcgcggtcaatacagccgcgaagacaaccagtattcacgtgaggacaattcgtgcacccacttcccggtgggccaatcgcatatgctgctggagttacgcacagccttctcgcaggtgaagactttttttcagactaaggatcagctggataatatcctgttgaccgattcattaatgcaagatttcaagggttacctgggttgccaggcgttatccgagatgattcaattctacttagtagaagtgatgccccaggctgaaaaacacggtcctgagattaaagagcacttgaacagtttgggagaaaagctgaagctccttcgtatgcgtctgcgccgctgccatcgctttttgccctgtgagaataaatccaaggccgtcgagcaggttaagtctgatttcaacaaattacaggatcaaggggtttacaaagcgatgaatgagtttgacatctttattaattgtatcgaggcttatatgatgattaagatgaagagt

SEQ ID NO: 94 - DNA, 983 bp, synthetic construct
  source 1..983: mol_type = other DNA
agcagaggccagtacagcagagaagataataactgcacccacttccccgtagggcagagccatatgctgctggagctgagaacagcctttagccaagtcaagaccttcttccaaacgaaggatcagcttgacaatatactgctgaccgatagcctgatgcaagacttcaaaggttacctcggctgtcaagccctctccgagatgatccaattttacctggtggaggtgatgccgcaagccgaaaaacatggccccgagatcaaagaacatctgaacagtcttggtgaaaagctgaagaccctgcgaatgcggctgcgcagatgtcacagatttctaccgtgcgagaataagagcaaggccgtggagcaagtgaagagcgacttcaataagctgcaagaccaaggcgtttataaggcgatgaacgagttcgatatatttatcaactgcatagaagcctatatgatgatcaagatgaagagcagcagagggcagtactcgagagaggataatcagtacagccgagaagacaacaactgtacccacttccccgtgggtcaaagccacatgctgctagagctcagaacggccttcagccaagtgaagactttctttcaaaccaaggaccaactggacaacatcctgttaaccgactcactaatgcaagactttaaggggtacctgggttgccaagccctgagcgagatgattcagttctacctcgtggaggtcatgcctcaagccgaaaagcacggccccgagattaaagagcacttaaacagtctaggggagaaactgaagaccttgagaatgagattaagacggtgtcatagatttctcccctgtgagaacaaaagcaaagcggtggagcaagtgaagagcgattttaacaagctccaagatcaaggcgtatataaagcgatgaatgagttcgatatcttcattaactgcatcgaagcctatatgatgattaaaatgaaaagcgg

SEQ ID NO: 95 - DNA, 983 bp, synthetic construct
  source 1..983: mol_type = other DNA
agcagaggccagtacagcagagaagataataactgcacccacttccccgtagggcagagccatatgctgctggagctgagaacagcctttagccaagtcaagaccttcttccaaacgaaggatcagcttgacaatatactgctgaccgatagcctgatgcaagacttcaaaggttacctcggctgtcaagccctctccgagatgatccaattttacctggtggaggtgatgccgcaagccgaaaaacatggccccgagatcaaagaacatctgaacagtcttggtgaaaagctgaagctgctgcgaatgcggctgcgcagatgtcacagatttctaccgtgcgagaataagagcaaggccgtggagcaagtgaagagcgacttcaataagctgcaagaccaaggcgtttataaggcgatgaacgagttcgatatatttatcaactgcatagaagcctatatgatgatcaagatgaagagcagcagagggcagtactcgagagaggataatcagtacagccgagaagacaacaactgtacccacttccccgtgggtcaaagccacatgctgctagagctcagaacggccttcagccaagtgaagactttctttcaaaccaaggaccaactggacaacatcctgttaaccgactcactaatgcaagactttaaggggtacctgggttgccaagccctgagcgagatgattcagttctacctcgtggaggtcatgcctcaagccgaaaagcacggccccgagattaaagagcacttaaacagtctaggggagaaactgaagctgttgagaatgagattaagacggtgtcatagatttctcccctgtgagaacaaaagcaaagcggtggagcaagtgaagagcgattttaacaagctccaagatcaaggcgtatataaagcgatgaatgagttcgatatcttcattaactgcatcgaagcctatatgatgattaaaatgaaaagcgg

SEQ ID NO: 96 - DNA, 951 bp, synthetic construct
  source 1..951: mol_type = other DNA
gagtactccagagaagataataactgtacccactttcctgtgggccagagtcacatgctcctggagttgcgtaccgccttttcacaggtcaagacctttttccagactaaggaccagctggacaatatcctcctgaccgattccctgatgcaggactttaagggctatctgggctgccaagccctgtccgagatgatccagttctatctggtcgaagttatgcctcaagcagagaaacacggacctgagatcaaggaacatttgaacagcctcggggaaaagctgaagactcttcgtatgcgccttcgcagatgccatcggtttctcccgtgtgagaataagtccaaggcggtggagcaggtgaagagcgacttcaacaagttgcaggaccagggcgtttacaaagctatgaatgagttcgacatcttcatcaactgcatcgaggcttacatgatgattaagatgaaaagctctcgcggtcagtacagccgcgaggataataattgcacacatttccccgtcggccaatcacacatgcttctggagcttcggactgctttttcccaggtcaagactttctttcagacaaaggaccaactggacaacattctcttgaccgatagcctgatgcaggactttaaggggtatctgggttgtcaagcactgtccgagatgatccagttctacctcgtggaggtgatgcctcaagctgagaagcatggccccgagattaaggaacacctcaattcccttggcgagaagctgaagactctgcgcatgaggctgagaaggtgccaccgttttttgccttgtgagaacaagtctaaggccgtggaacaggtgaagagcgactttaacaagctccaggatcagggcgtatataaggccatgaacgagttcgatatttttataaattgcatcgaggcttacatgatgattaaaatgaagagc

SEQ ID NO: 97 - DNA, 952 bp, synthetic construct
  source 1..952: mol_type = other DNA
gagtattctcgggaggacaataattgcacccacttccctgtcggccagagccacatgcttctggagcttagaactgccttttcccaggttaagacattctttcaaaccaaggatcagctggacaacatcttgctgactgactctctgatgcaggacttcaaaggctatcttggttgccaggccctgtccgagatgatccagttctatctggtcgaagtgatgccacaggcagaaaagcatggccctgagatcaaggagcacctcaattccctgggcgaaaagctgaagctcctgcggatgcgtctgcgcagatgccatagattcctgccgtgtgagaacaagtctaaagccgtggagcaggtcaagagcgatttcaacaagctccaggaccagggggtttacaaggccatgaacgagttcgatatcttcattaactgcatcgaagcctacatgatgattaagatgaagtctagtagaggacagtacagcagagaggacaataactgcacacacttcccggtgggccagagtcacatgctgttggagctgcgcacggccttttcccaggtgaaaacttttttccagacgaaggaccagctcgataatatcctcttgaccgacagcctcatgcaggactttaaaggctacctgggctgtcaggctctctccgagatgatccagttctacctcgtcgaggtgatgccccaggcggagaagcacggtcctgagatcaaggagcacctgaactccctgggcgagaagctgaaactcttgcgtatgcgcctgcggcgttgtcacagatttctcccttgtgaaaacaagagcaaggctgttgaacaggtgaaatccgacttcaacaaacttcaagaccagggcgtctacaaggccatgaacgaatttgacatcttcataaattgcattgaagcctatatgatgattaagatgaagtcag

SEQ ID NO: 98 - protein, 18 aa, Homo sapiens
MHSSALLCCLVLLTGVRA

SEQ ID NO: 99 - protein, 18 aa, Mus sp.
MPGSALLCCLLLLTGMRI

SEQ ID NO: 100 - protein, 19 aa, Mus sp.
MGWSCIILFLVATATGVHS

SEQ ID NO: 101 - protein, 4 aa, synthetic construct
GSGG

SEQ ID NO: 102 - protein, 4 aa, synthetic construct
GGSG

SEQ ID NO: 103 - protein, 4 aa, synthetic construct
GGGS

SEQ ID NO: 104 - protein, 5 aa, synthetic construct
GGGGS

SEQ ID NO: 105 - protein, 6 aa, synthetic construct
GGGSGG

SEQ ID NO: 106 - protein, 6 aa, synthetic construct
GGGGSG

SEQ ID NO: 107 - protein, 8 aa, synthetic construct
GGGSGGGS

SEQ ID NO: 108 - protein, 9 aa, synthetic construct
GGGGSGGGS

SEQ ID NO: 109 - protein, 10 aa, synthetic construct
GGGGGSGGGS

SEQ ID NO: 110 - protein, 9 aa, synthetic construct
GGGSGGGGS

SEQ ID NO: 111 - protein, 10 aa, synthetic construct
GGGGSGGGGS

SEQ ID NO: 112 - protein, 5 aa, synthetic construct
GGGGA

SEQ ID NO: 113 - protein, 10 aa, synthetic construct
GGGGAGGGGS

SEQ ID NO: 114 - protein, 15 aa, synthetic construct
GGGGAGGGGSGGGGA

SEQ ID NO: 115 - protein, 8 aa, synthetic construct
GGGAGGGS

SEQ ID NO: 116 - protein, 8 aa, synthetic construct
GGGAGGGA

SEQ ID NO: 117 - protein, 8 aa, synthetic construct
GGGSGGGA

SEQ ID NO: 118 - protein, 9 aa, synthetic construct
GGGGAGGGS

SEQ ID NO: 119 - protein, 314 aa, synthetic construct
SENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 120 - DNA, 942 bp, synthetic construct
  source 1..942: mol_type = other DNA
tcagagaatagttgcacacactttcctggcaatctgcctaatatgctacgcgatctcagggatgccttttctcgcgttaaaacttttttccaaatgaaggaccagcttgataacctgctactcaaagaaagccttttggaggattttaaggggtacctgggttgccaggccctgtcggaaatgattcagttttatttggaggaggtgatgccccaggcagaaaaccaagatcctgacatcaaggcacatgtaaattcccttggagagaacctcaaaacactccgtcttaggctccgacgctgccatcgattcctgccttgcgagaacaagtccaaggcagtggagcaagtcaagaacgcctttaataaattacaagagaagggcatctacaaagcgatgtccgagtttgacatcttcattaactacattgaagcttacatgaccatgaaaataaggaacactagcccgggacagggcacccagagtgaaaactcttgtacgcacttccccgggaatctgcccaacatgctgagagatctgcgggacgccttctcaagagtgaagaccttcttccaaatgaaagaccagttggacaatctgctgctgaaggaaagcttattagaggatttcaaaggctatctaggatgtcaggctctgtcagaaatgatccagttctatctggaggaggtgatgccacaggccgagaatcaggacccagacattaaagcgcacgtcaattctctgggagaaaacctcaagactctccggttgcggctgagacggtgtcatagattcttgccatgtgaaaacaagtccaaagctgttgaacaggtgaaaaatgctttcaataagcttcaggaaaaggggatctacaaggccatgagtgagtttgacatatttatcaactacattgaggcatatatgaccatgaagatcaggaac

SEQ ID NO: 121 - protein, 5 aa, Homo sapiens
EPKSC

SEQ ID NO: 122 - protein, 5 aa, synthetic construct
EPKSS

SEQ ID NO: 123 - protein, 321 aa, synthetic construct
MSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRN

SEQ ID NO: 124 - protein, 319 aa, synthetic construct
PGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRN

SEQ ID NO: 125 - DNA, 54 bp, Homo sapiens
  source 1..54: mol_type = genomic DNA
atgcacagcagcgccctgctgtgctgcctggtgctgctgaccggcgtgagagcc

SEQ ID NO: 126 - DNA, 57 bp, Mus sp.
  source 1..57: mol_type = genomic DNA
atgggatggtcctgcatcatcctgtttctggtggctaccgccaccggtgtgcacagc

SEQ ID NO: 127 - protein, 10 aa, Homo sapiens
DKTHTCPPCP

SEQ ID NO: 128 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 800 aa, synthetic construct
  VARIANT 1..20: note = This region may encompass 1-20 residues
  VARIANT 21..40: note = This region may encompass 1-20 residues
  VARIANT 41..60: note = This region may encompass 1-20 residues
  VARIANT 61..80: note = This region may encompass 1-20 residues
  VARIANT 81..100: note = This region may encompass 1-20 residues
  VARIANT 101..120: note = This region may encompass 1-20 residues
  VARIANT 121..140: note = This region may encompass 1-20 residues
  VARIANT 141..160: note = This region may encompass 1-20 residues
  VARIANT 161..180: note = This region may encompass 1-20 residues
  VARIANT 181..200: note = This region may encompass 1-20 residues
  VARIANT 201..220: note = This region may encompass 1-20 residues
  VARIANT 221..240: note = This region may encompass 1-20 residues
  VARIANT 241..260: note = This region may encompass 1-20 residues
  VARIANT 261..280: note = This region may encompass 1-20 residues
  VARIANT 281..300: note = This region may encompass 1-20 residues
  VARIANT 301..320: note = This region may encompass 1-20 residues
  VARIANT 321..340: note = This region may encompass 1-20 residues
  VARIANT 341..360: note = This region may encompass 1-20 residues
  VARIANT 361..380: note = This region may encompass 1-20 residues
  VARIANT 381..400: note = This region may encompass 1-20 residues
  VARIANT 401..420: note = This region may encompass 1-20 residues
  VARIANT 421..440: note = This region may encompass 1-20 residues
  VARIANT 441..460: note = This region may encompass 1-20 residues
  VARIANT 461..480: note = This region may encompass 1-20 residues
  VARIANT 481..500: note = This region may encompass 1-20 residues
  VARIANT 501..520: note = This region may encompass 1-20 residues
  VARIANT 521..540: note = This region may encompass 1-20 residues
  VARIANT 541..560: note = This region may encompass 1-20 residues
  VARIANT 561..580: note = This region may encompass 1-20 residues
  VARIANT 581..600: note = This region may encompass 1-20 residues
  VARIANT 601..620: note = This region may encompass 1-20 residues
  VARIANT 621..640: note = This region may encompass 1-20 residues
  VARIANT 641..660: note = This region may encompass 1-20 residues
  VARIANT 661..680: note = This region may encompass 1-20 residues
  VARIANT 681..700: note = This region may encompass 1-20 residues
  VARIANT 701..720: note = This region may encompass 1-20 residues
  VARIANT 721..740: note = This region may encompass 1-20 residues
  VARIANT 741..760: note = This region may encompass 1-20 residues
  VARIANT 761..780: note = This region may encompass 1-20 residues
  VARIANT 781..800: note = This region may encompass 1-20 residues
  VARIANT 1..800: note = This sequence may encompass 1-20 GmSo repeating units, wherein m = 1 to 20, o = 1 to 20
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSS

SEQ ID NO: 130 - protein, 100 aa, synthetic construct
  VARIANT 1..100: note = This sequence may encompass 1-20 GSGGS repeating units
GSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGS

SEQ ID NO: 131 - protein, 1200 aa, synthetic construct
  VARIANT 1..20: note = This region may encompass 1-20 residues
  VARIANT 21..40: note = This region may encompass 1-20 residues
  VARIANT 41..60: note = This region may encompass 1-20 residues
  VARIANT 61..80: note = This region may encompass 1-20 residues
  VARIANT 81..100: note = This region may encompass 1-20 residues
  VARIANT 101..120: note = This region may encompass 1-20 residues
  VARIANT 121..140: note = This region may encompass 1-20 residues
  VARIANT 141..160: note = This region may encompass 1-20 residues
  VARIANT 161..180: note = This region may encompass 1-20 residues
  VARIANT 181..200: note = This region may encompass 1-20 residues
  VARIANT 201..220: note = This region may encompass 1-20 residues
  VARIANT 221..240: note = This region may encompass 1-20 residues
  VARIANT 241..260: note = This region may encompass 1-20 residues
  VARIANT 261..280: note = This region may encompass 1-20 residues
  VARIANT 281..300: note = This region may encompass 1-20 residues
  VARIANT 301..320: note = This region may encompass 1-20 residues
  VARIANT 321..340: note = This region may encompass 1-20 residues
  VARIANT 341..360: note = This region may encompass 1-20 residues
  VARIANT 361..380: note = This region may encompass 1-20 residues
  VARIANT 381..400: note = This region may encompass 1-20 residues
  VARIANT 401..420: note = This region may encompass 1-20 residues
  VARIANT 421..440: note = This region may encompass 1-20 residues
  VARIANT 441..460: note = This region may encompass 1-20 residues
  VARIANT 461..480: note = This region may encompass 1-20 residues
  VARIANT 481..500: note = This region may encompass 1-20 residues
  VARIANT 501..520: note = This region may encompass 1-20 residues
  VARIANT 521..540: note = This region may encompass 1-20 residues
  VARIANT 541..560: note = This region may encompass 1-20 residues
  VARIANT 561..580: note = This region may encompass 1-20 residues
  VARIANT 581..600: note = This region may encompass 1-20 residues
  VARIANT 601..620: note = This region may encompass 1-20 residues
  VARIANT 621..640: note = This region may encompass 1-20 residues
  VARIANT 641..660: note = This region may encompass 1-20 residues
  VARIANT 661..680: note = This region may encompass 1-20 residues
  VARIANT 681..700: note = This region may encompass 1-20 residues
  VARIANT 701..720: note = This region may encompass 1-20 residues
  VARIANT 721..740: note = This region may encompass 1-20 residues
  VARIANT 741..760: note = This region may encompass 1-20 residues
  VARIANT 761..780: note = This region may encompass 1-20 residues
  VARIANT 781..800: note = This region may encompass 1-20 residues
  VARIANT 801..820: note = This region may encompass 1-20 residues
  VARIANT 821..840: note = This region may encompass 1-20 residues
  VARIANT 841..860: note = This region may encompass 1-20 residues
  VARIANT 861..880: note = This region may encompass 1-20 residues
  VARIANT 881..900: note = This region may encompass 1-20 residues
  VARIANT 901..920: note = This region may encompass 1-20 residues
  VARIANT 921..940: note = This region may encompass 1-20 residues
  VARIANT 941..960: note = This region may encompass 1-20 residues
  VARIANT 961..980: note = This region may encompass 1-20 residues
  VARIANT 981..1000: note = This region may encompass 1-20 residues
  VARIANT 1001..1020: note = This region may encompass 1-20 residues
  VARIANT 1021..1040: note = This region may encompass 1-20 residues
  VARIANT 1041..1060: note = This region may encompass 1-20 residues
  VARIANT 1061..1080: note = This region may encompass 1-20 residues
  VARIANT 1081..1100: note = This region may encompass 1-20 residues
  VARIANT 1101..1120: note = This region may encompass 1-20 residues
  VARIANT 1121..1140: note = This region may encompass 1-20 residues
  VARIANT 1141..1160: note = This region may encompass 1-20 residues
  VARIANT 1161..1180: note = This region may encompass 1-20 residues
  VARIANT 1181..1200: note = This region may encompass 1-20 residues
  VARIANT 1..1200: note = This sequence may encompass 1-20 GmSoGm repeating units, wherein m = 1 to 20, o = 1 to 20
GGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGG

SEQ ID NO: 132 - protein, 2000 aa, synthetic construct
  VARIANT 1..20: note = This region may encompass 1-20 residues
  VARIANT 21..40: note = This region may encompass 1-20 residues
  VARIANT 41..60: note = This region may encompass 1-20 residues
  VARIANT 61..80: note = This region may encompass 1-20 residues
  VARIANT 81..100: note = This region may encompass 1-20 residues
  VARIANT 101..120: note = This region may encompass 1-20 residues
  VARIANT 121..140: note = This region may encompass 1-20 residues
  VARIANT 141..160: note = This region may encompass 1-20 residues
  VARIANT 161..180: note = This region may encompass 1-20 residues
  VARIANT 181..200: note = This region may encompass 1-20 residues
  VARIANT 201..220: note = This region may encompass 1-20 residues
  VARIANT 221..240: note = This region may encompass 1-20 residues
  VARIANT 241..260: note = This region may encompass 1-20 residues
  VARIANT 261..280: note = This region may encompass 1-20 residues
  VARIANT 281..300: note = This region may encompass 1-20 residues
  VARIANT 301..320: note = This region may encompass 1-20 residues
  VARIANT 321..340: note = This region may encompass 1-20 residues
  VARIANT 341..360: note = This region may encompass 1-20 residues
  VARIANT 361..380: note = This region may encompass 1-20 residues
  VARIANT 381..400: note = This region may encompass 1-20 residues
  VARIANT 401..420: note = This region may encompass 1-20 residues
  VARIANT 421..440: note = This region may encompass 1-20 residues
  VARIANT 441..460: note = This region may encompass 1-20 residues
  VARIANT 461..480: note = This region may encompass 1-20 residues
  VARIANT 481..500: note = This region may encompass 1-20 residues
  VARIANT 501..520: note = This region may encompass 1-20 residues
  VARIANT 521..540: note = This region may encompass 1-20 residues
  VARIANT 541..560: note = This region may encompass 1-20 residues
  VARIANT 561..580: note = This region may encompass 1-20 residues
  VARIANT 581..600: note = This region may encompass 1-20 residues
  VARIANT 601..620: note = This region may encompass 1-20 residues
  VARIANT 621..640: note = This region may encompass 1-20 residues
  VARIANT 641..660: note = This region may encompass 1-20 residues
  VARIANT 661..680: note = This region may encompass 1-20 residues
  VARIANT 681..700: note = This region may encompass 1-20 residues
  VARIANT 701..720: note = This region may encompass 1-20 residues
  VARIANT 721..740: note = This region may encompass 1-20 residues
  VARIANT 741..760: note = This region may encompass 1-20 residues
  VARIANT 761..780: note = This region may encompass 1-20 residues
  VARIANT 781..800: note = This region may encompass 1-20 residues
  VARIANT 801..820: note = This region may encompass 1-20 residues
  VARIANT 821..840: note = This region may encompass 1-20 residues
  VARIANT 841..860: note = This region may encompass 1-20 residues
  VARIANT 861..880: note = This region may encompass 1-20 residues
  VARIANT 881..900: note = This region may encompass 1-20 residues
  VARIANT 901..920: note = This region may encompass 1-20 residues
  VARIANT 921..940: note = This region may encompass 1-20 residues
  VARIANT 941..960: note = This region may encompass 1-20 residues
  VARIANT 961..980: note = This region may encompass 1-20 residues
  VARIANT 981..1000: note = This region may encompass 1-20 residues
  VARIANT 1001..1020: note = This region may encompass 1-20 residues
  VARIANT 1021..1040: note = This region may encompass 1-20 residues
  VARIANT 1041..1060: note = This region may encompass 1-20 residues
  VARIANT 1061..1080: note = This region may encompass 1-20 residues
  VARIANT 1081..1100: note = This region may encompass 1-20 residues
  VARIANT 1101..1120: note = This region may encompass 1-20 residues
  VARIANT 1121..1140: note = This region may encompass 1-20 residues
  VARIANT 1141..1160: note = This region may encompass 1-20 residues
  VARIANT 1161..1180: note = This region may encompass 1-20 residues
  VARIANT 1181..1200: note = This region may encompass 1-20 residues
  VARIANT 1201..1220: note = This region may encompass 1-20 residues
  VARIANT 1221..1240: note = This region may encompass 1-20 residues
  VARIANT 1241..1260: note = This region may encompass 1-20 residues
  VARIANT 1261..1280: note = This region may encompass 1-20 residues
  VARIANT 1281..1300: note = This region may encompass 1-20 residues
  VARIANT 1301..1320: note = This region may encompass 1-20 residues
  VARIANT 1321..1340: note = This region may encompass 1-20 residues
  VARIANT 1341..1360: note = This region may encompass 1-20 residues
  VARIANT 1361..1380: note = This region may encompass 1-20 residues
  VARIANT 1381..1400: note = This region may encompass 1-20 residues
  VARIANT 1401..1420: note = This region may encompass 1-20 residues
  VARIANT 1421..1440: note = This region may encompass 1-20 residues
  VARIANT 1441..1460: note = This region may encompass 1-20 residues
  VARIANT 1461..1480: note = This region may encompass 1-20 residues
  VARIANT 1481..1500: note = This region may encompass 1-20 residues
  VARIANT 1501..1520: note = This region may encompass 1-20 residues
  VARIANT 1521..1540: note = This region may encompass 1-20 residues
  VARIANT 1541..1560: note = This region may encompass 1-20 residues
  VARIANT 1561..1580: note = This region may encompass 1-20 residues
  VARIANT 1581..1600: note = This region may encompass 1-20 residues
  VARIANT 1601..1620: note = This region may encompass 1-20 residues
  VARIANT 1621..1640: note = This region may encompass 1-20 residues
  VARIANT 1641..1660: note = This region may encompass 1-20 residues
  VARIANT 1661..1680: note = This region may encompass 1-20 residues
  VARIANT 1681..1700: note = This region may encompass 1-20 residues
  VARIANT 1701..1720: note = This region may encompass 1-20 residues
  VARIANT 1721..1740: note = This region may encompass 1-20 residues
  VARIANT 1741..1760: note = This region may encompass 1-20 residues
  VARIANT 1761..1780: note = This region may encompass 1-20 residues
  VARIANT 1781..1800: note = This region may encompass 1-20 residues
  VARIANT 1801..1820: note = This region may encompass 1-20 residues
  VARIANT 1821..1840: note = This region may encompass 1-20 residues
  VARIANT 1841..1860: note = This region may encompass 1-20 residues
  VARIANT 1861..1880: note = This region may encompass 1-20 residues
  VARIANT 1881..1900: note = This region may encompass 1-20 residues
  VARIANT 1901..1920: note = This region may encompass 1-20 residues
  VARIANT 1921..1940: note = This region may encompass 1-20 residues
  VARIANT 1941..1960: note = This region may encompass 1-20 residues
  VARIANT 1961..1980: note = This region may encompass 1-20 residues
  VARIANT 1981..2000: note = This region may encompass 1-20 residues
  VARIANT 1..2000: note = This sequence may encompass 1-20 GmSoGmSoGm repeating units, wherein m = 1 to 20, o = 1 to 20
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGG

SEQ ID NO: 133 - protein, 480 aa, synthetic construct
  VARIANT 5..24: note = This region may encompass 1-20 residues
  VARIANT 29..48: note = This region may encompass 1-20 residues
  VARIANT 53..72: note = This region may encompass 1-20 residues
  VARIANT 77..96: note = This region may encompass 1-20 residues
  VARIANT 101..120: note = This region may encompass 1-20 residues
  VARIANT 125..144: note = This region may encompass 1-20 residues
  VARIANT 149..168: note = This region may encompass 1-20 residues
  VARIANT 173..192: note = This region may encompass 1-20 residues
  VARIANT 197..216: note = This region may encompass 1-20 residues
  VARIANT 221..240: note = This region may encompass 1-20 residues
  VARIANT 245..264: note = This region may encompass 1-20 residues
  VARIANT 269..288: note = This region may encompass 1-20 residues
  VARIANT 293..312: note = This region may encompass 1-20 residues
  VARIANT 317..336: note = This region may encompass 1-20 residues
  VARIANT 341..360: note = This region may encompass 1-20 residues
  VARIANT 365..384: note = This region may encompass 1-20 residues
  VARIANT 389..408: note = This region may encompass 1-20 residues
  VARIANT 413..432: note = This region may encompass 1-20 residues
  VARIANT 437..456: note = This region may encompass 1-20 residues
  VARIANT 461..480: note = This region may encompass 1-20 residues
  VARIANT 1..480: note = This sequence may encompass 1-20 GSGGSm repeating units, wherein m = 1 to 20
GSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSS

SEQ ID NO: 134 - protein, 480 aa, synthetic construct
  VARIANT 4..23: note = This region may encompass 1-20 residues
  VARIANT 28..47: note = This region may encompass 1-20 residues
  VARIANT 52..71: note = This region may encompass 1-20 residues
  VARIANT 76..95: note = This region may encompass 1-20 residues
  VARIANT 100..119: note = This region may encompass 1-20 residues
  VARIANT 124..143: note = This region may encompass 1-20 residues
  VARIANT 148..167: note = This region may encompass 1-20 residues
  VARIANT 172..191: note = This region may encompass 1-20 residues
  VARIANT 196..215: note = This region may encompass 1-20 residues
  VARIANT 220..239: note = This region may encompass 1-20 residues
  VARIANT 244..263: note = This region may encompass 1-20 residues
  VARIANT 268..287: note = This region may encompass 1-20 residues
  VARIANT 292..311: note = This region may encompass 1-20 residues
  VARIANT 316..335: note = This region may encompass 1-20 residues
  VARIANT 340..359: note = This region may encompass 1-20 residues
  VARIANT 364..383: note = This region may encompass 1-20 residues
  VARIANT 388..407: note = This region may encompass 1-20 residues
  VARIANT 412..431: note = This region may encompass 1-20 residues
  VARIANT 436..455: note = This region may encompass 1-20 residues
  VARIANT 460..479: note = This region may encompass 1-20 residues
  VARIANT 1..480: note = This sequence may encompass 1-20 GSGSmG repeating units, wherein m = 1 to 20
GSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSG

SEQ ID NO: 135 - protein, 460 aa, synthetic construct
  VARIANT 4..23: note = This region may encompass 1-20 residues
  VARIANT 27..46: note = This region may encompass 1-20 residues
  VARIANT 50..69: note = This region may encompass 1-20 residues
  VARIANT 73..92: note = This region may encompass 1-20 residues
  VARIANT 96..115: note = This region may encompass 1-20 residues
  VARIANT 119..138: note = This region may encompass 1-20 residues
  VARIANT 142..161: note = This region may encompass 1-20 residues
  VARIANT 165..184: note = This region may encompass 1-20 residues
  VARIANT 188..207: note = This region may encompass 1-20 residues
  VARIANT 211..230: note = This region may encompass 1-20 residues
  VARIANT 234..253: note = This region may encompass 1-20 residues
  VARIANT 257..276: note = This region may encompass 1-20 residues
  VARIANT 280..299: note = This region may encompass 1-20 residues
  VARIANT 303..322: note = This region may encompass 1-20 residues
  VARIANT 326..345: note = This region may encompass 1-20 residues
  VARIANT 349..368: note = This region may encompass 1-20 residues
  VARIANT 372..391: note = This region may encompass 1-20 residues
  VARIANT 395..414: note = This region may encompass 1-20 residues
  VARIANT 418..437: note = This region may encompass 1-20 residues
  VARIANT 441..460: note = This region may encompass 1-20 residues
  VARIANT 1..460: note = This sequence may encompass 1-20 GGGSm repeating units, wherein m = 1 to 20
GGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSS

SEQ ID NO: 136 - protein, 32 aa, synthetic construct
  VARIANT 5..8: note = This region may encompass 1-4 residues
  VARIANT 13..16: note = This region may encompass 1-4 residues
  VARIANT 21..24: note = This region may encompass 1-4 residues
  VARIANT 29..32: note = This region may encompass 1-4 residues
  VARIANT 1..32: note = This sequence may encompass 1-4 GGGGSm repeating units, wherein m = 1 to 4
GGGGSSSSGGGGSSSSGGGGSSSSGGGGSSSS

SEQ ID NO: 137 - protein, 28 aa, synthetic construct
  VARIANT 4..7: note = This region may encompass 1-4 residues
  VARIANT 11..14: note = This region may encompass 1-4 residues
  VARIANT 18..21: note = This region may encompass 1-4 residues
  VARIANT 25..28: note = This region may encompass 1-4 residues
  VARIANT 1..28: note = This sequence may encompass 1-4 GGGSm repeating units, wherein m = 1 to 4
GGGSSSSGGGSSSSGGGSSSSGGGSSSS

SEQ ID NO: 138 - protein, 28 aa, synthetic construct
  VARIANT 4..7: note = This region may encompass 1-4 residues
  VARIANT 11..14: note = This region may encompass 1-4 residues
  VARIANT 18..21: note = This region may encompass 1-4 residues
  VARIANT 25..28: note = This region may encompass 1-4 residues
  VARIANT 1..28: note = This sequence may encompass 1-4 GGGAm repeating units, wherein m = 1 to 4
GGGAAAAGGGAAAAGGGAAAAGGGAAAA

SEQ ID NO: 139 - protein, 32 aa, synthetic construct
  VARIANT 5..8: note = This region may encompass 1-4 residues
  VARIANT 13..16: note = This region may encompass 1-4 residues
  VARIANT 21..24: note = This region may encompass 1-4 residues
  VARIANT 29..32: note = This region may encompass 1-4 residues
  VARIANT 1..32: note = This sequence may encompass 1-4 GGGGAm repeating units, wherein m = 1 to 4
GGGGAAAAGGGGAAAAGGGGAAAAGGGGAAAA

SEQ ID NO: 140 - protein, 4 aa, synthetic construct
GGGA

SEQ ID NO: 141 - protein, 4 aa, Homo sapiens
SPGQ

SEQ ID NO: 142 - protein, 5 aa, Homo sapiens
SPGQG

SEQ ID NO: 143 - protein, 6 aa, Homo sapiens
SPGQGT

SEQ ID NO: 144 - protein, 8 aa, Homo sapiens
SPGQGTQS

SEQ ID NO: 145 - protein, 9 aa, Homo sapiens
SPGQGTQSE

SEQ ID NO: 146 - protein, 8 aa, synthetic construct
HHHHHHHH

SEQ ID NO: 147 - protein, 6 aa, synthetic construct
HHHHHH

SEQ ID NO: 148 - protein, 160 aa, synthetic construct
SPGQGTQSENSCTHFPGNLPDMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 149 - protein, 160 aa, synthetic construct
SPGQGTQSENSCTHFPGNLPEMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 150 - protein, 160 aa, synthetic construct
SPGQGTQSENSCTHFPGNLPDTLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 151 - protein, 160 aa, synthetic construct
SPGQGTQSENSCTHFPGNLPDDLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 152 - protein, 160 aa, synthetic construct
SPGQGTQSENSCTHFPGNLPDWLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 153 - protein, 160 aa, synthetic construct
SPGQGTQSENSCTHFPGNLPNMLEDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 154 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGKNLKTLRLRLRRCHRFLPCENKSKAVEQVKNAFNKLQEKGIYKAMSEFDIFINYIEAYMTMKIRN

SEQ ID NO: 155 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKELRLRLRRCHRFLPCENKSKAVEQVKNAFNKLQEKGIYKAMSEFDIFINYIEAYMTMKIRN

SEQ ID NO: 156 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKCLRLRLRRCHRFLPCENKSKAVEQVKNAFNKLQEKGIYKAMSEFDIFINYIEAYMTMKIRN

SEQ ID NO: 157 - protein, 320 aa, synthetic construct
SPGQGTQSENSCTHFPGNLPDMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 158 - protein, 320 aa, synthetic construct
SPGQGTQSENSCTHFPGNLPEMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPEMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 159 - protein, 320 aa, synthetic construct
SPGQGTQSENSCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 160 - protein, 320 aa, synthetic construct
SPGQGTQSENSCTHFPGNLPDALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 161 - protein, 320 aa, synthetic construct
SPGQGTQSENSCTHFPGNLPDSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 162 - protein, 320 aa, synthetic construct
SPGQGTQSENSCTHFPGNLPDTLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDTLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 163 - protein, 320 aa, synthetic construct
SPGQGTQSENSCTHFPGNLPDDLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDDLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 164 - protein, 320 aa, synthetic construct
SPGQGTQSENSCTHFPGNLPDWLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDWLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 165 - protein, 320 aa, synthetic construct
SPGQGTQSENSCTHFPGNLPNMLEDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPNMLEDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 166 - protein, 320 aa, synthetic construct
SPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 167 - protein, 320 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGKNLKTLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGKNLKTLRLRLRRCHRFLPCENKSKAVEQVKNAFNKLQEKGIYKAMSEFDIFINYIEAYMTMKIRN

SEQ ID NO: 168 - protein, 320 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGQNLKTLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGQNLKTLRLRLRRCHRFLPCENKSKAVEQVKNAFNKLQEKGIYKAMSEFDIFINYIEAYMTMKIRN

SEQ ID NO: 169 - protein, 320 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKELRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKELRLRLRRCHRFLPCENKSKAVEQVKNAFNKLQEKGIYKAMSEFDIFINYIEAYMTMKIRN

SEQ ID NO: 170 - protein, 320 aa, synthetic construct; identical to SEQ ID NO: 44

SEQ ID NO: 171 - protein, 320 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKCLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKCLRLRLRRCHRFLPCENKSKAVEQVKNAFNKLQEKGIYKAMSEFDIFINYIEAYMTMKIRN

SEQ ID NO: 172 - protein, 319 aa, synthetic construct
PGQGTQSENSCTHFPGNLPDMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 173 - protein, 319 aa, synthetic construct
PGQGTQSENSCTHFPGNLPEMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPEMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 174 - protein, 319 aa, synthetic construct
PGQGTQSENSCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 175 - protein, 319 aa, synthetic construct
PGQGTQSENSCTHFPGNLPDALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 176 - protein, 319 aa, synthetic construct
PGQGTQSENSCTHFPGNLPDSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 177 - protein, 319 aa, synthetic construct
PGQGTQSENSCTHFPGNLPDTLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDTLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 178 - protein, 319 aa, synthetic construct
PGQGTQSENSCTHFPGNLPDDLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDDLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 179 - protein, 319 aa, synthetic construct
PGQGTQSENSCTHFPGNLPDWLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDWLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 180 - protein, 319 aa, synthetic construct
PGQGTQSENSCTHFPGNLPNMLEDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPNMLEDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 181 - protein, 319 aa, synthetic construct
PGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 182 - protein, 319 aa, synthetic construct
PGQGTQSENSCTHFPGNLPNMLRDLRDAFSRVKTFFQMKDQLDNLLLKESLLEDFKGYLGCQALSEMIQFYLEEVMPQAENQDPDIKAHVNSLGKNLKTLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGKNLKTLRLRLRRCHRFLPCENKSKAVEQVKNAFNKLQEKGIYKAMSEFDIFINYIEAYMTMKIRN

SEQ ID NO: 183 - protein, 319 aa, synthetic construct
PGQGTQSENSCTHFPGNLPNMLRDLRDAFSRVKTFFQMKDQLDNLLLKESLLEDFKGYLGCQALSEMIQFYLEEVMPQAENQDPDIKAHVNSLGQNLKTLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGQNLKTLRLRLRRCHRFLPCENKSKAVEQVKNAFNKLQEKGIYKAMSEFDIFINYIEAYMTMKIRN

SEQ ID NO: 184 - protein, 319 aa, synthetic construct
PGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKELRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKELRLRLRRCHRFLPCENKSKAVEQVKNAFNKLQEKGIYKAMSEFDIFINYIEAYMTMKIRN

SEQ ID NO: 185 - protein, 319 aa, synthetic construct; identical to SEQ ID NO: 124

SEQ ID NO: 186 - protein, 319 aa, synthetic construct
PGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKCLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKCLRLRLRRCHRFLPCENKSKAVEQVKNAFNKLQEKGIYKAMSEFDIFINYIEAYMTMKIRN

SEQ ID NO: 187 - protein, 159 aa, Mus musculus
SRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMK

SEQ ID NO: 188 - protein, 168 aa, synthetic construct
AHHHHHHHHGSQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 189 - protein, 328 aa, synthetic construct
EYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSGGSHHHHHHHH

SEQ ID NO: 190 - protein, 328 aa, synthetic construct
EYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKLLRMRLRRCHRFLPCENKSKAVEQVKSDFNKLQDQGVYKAMNEFDIFINCIEAYMMIKMKSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKLLRMRLRRCHRFLPCENKSKAVEQVKSDFNKLQDQGVYKAMNEFDIFINCIEAYMMIKMKSGGSHHHHHHHH

SEQ ID NO: 191 - protein, 159 aa, synthetic construct
PGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 192 - protein, 159 aa, synthetic construct
PGQGTQSENSCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 193 - protein, 159 aa, synthetic construct
PGQGTQSENSCTHFPGNLPNALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 194 - protein, 159 aa, synthetic construct
PGQGTQSENSCTHFPGNLPNSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 195 - protein, 159 aa, synthetic construct
PGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRN

SEQ ID NO: 196 - protein, 159 aa, synthetic construct
PGQGTQSENSCTHFPGNLPNMLRDLRDAFSRVKTFFQMKDQLDNLLLKESLLEDFKGYLGCQALSEMIQFYLEEVMPQAENQDPDIKAHVNSLGQNLKTLRLRLRRCHRFLPCENKSKAVEQVKNAFNKLQEKGIYKAMSEFDIFINYIEAYMTMKIRN

SEQ ID NO: 197 - protein, 159 aa, synthetic construct
PGQGTQSENSCTHFPGNLPDMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 198 - protein, 159 aa, synthetic construct
PGQGTQSENSCTHFPGNLPEMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 199 - protein, 159 aa, synthetic construct
PGQGTQSENSCTHFPGNLPDTLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 200 - protein, 159 aa, synthetic construct
PGQGTQSENSCTHFPGNLPDDLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 201 - protein, 159 aa, synthetic construct
PGQGTQSENSCTHFPGNLPDWLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 202 - protein, 159 aa, synthetic construct
PGQGTQSENSCTHFPGNLPNMLEDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 203 - protein, 159 aa, synthetic construct
PGQGTQSENSCTHFPGNLPNMLRDLRDAFSRVKTFFQMKDQLDNLLLKESLLEDFKGYLGCQALSEMIQFYLEEVMPQAENQDPDIKAHVNSLGKNLKTLRLRLRRCHRFLPCENKSKAVEQVKNAFNKLQEKGIYKAMSEFDIFINYIEAYMTMKIRN

SEQ ID NO: 204 - protein, 159 aa, synthetic construct
PGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKELRLRLRRCHRFLPCENKSKAVEQVKNAFNKLQEKGIYKAMSEFDIFINYIEAYMTMKIRN

SEQ ID NO: 205 - protein, 159 aa, synthetic construct
PGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKCLRLRLRRCHRFLPCENKSKAVEQVKNAFNKLQEKGIYKAMSEFDIFINYIEAYMTMKIRN

SEQ ID NO: 206 - protein, 332 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRNGGSHHHHHHHH

SEQ ID NO: 207 - protein, 332 aa, synthetic construct
MSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRNGGSHHHHHHHH

SEQ ID NO: 208 - protein, 171 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGSHHHHHHHH

SEQ ID NO: 209 - protein, 331 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRNGGSHHHHHHHH

SEQ ID NO: 210 - protein, 331 aa, synthetic construct
SPGQGTQSENSCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGSHHHHHHHH

SEQ ID NO: 211 - protein, 331 aa, synthetic construct
SPGQGTQSENSCTHFPGNLPNMLEDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPNMLEDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGSHHHHHHHH

SEQ ID NO: 212 - protein, 331 aa, synthetic construct
SPGQGTQSENSCTHFPGNLPNALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPNALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGSHHHHHHHH

SEQ ID NO: 213 - protein, 331 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGKNLKTLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGKNLKTLRLRLRRCHRFLPCENKSKAVEQVKNAFNKLQEKGIYKAMSEFDIFINYIEAYMTMKIRNGGSHHHHHHHH

SEQ ID NO: 214 - protein, 331 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGQNLKTLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGQNLKTLRLRLRRCHRFLPCENKSKAVEQVKNAFNKLQEKGIYKAMSEFDIFINYIEAYMTMKIRNGGSHHHHHHHH

SEQ ID NO: 215 - protein, 331 aa, synthetic construct; identical to SEQ ID NO: 211

SEQ ID NO: 216 - protein, 331 aa, synthetic construct
SPGQGTQSENSCTHFPGNLPDALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGSHHHHHHHH

SEQ ID NO: 217 - protein, 331 aa, synthetic construct
SPGQGTQSENSCTHFPGNLPDSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGSHHHHHHHH

SEQ ID NO: 218 - protein, 331 aa, synthetic construct
SPGQGTQSENSCTHFPGNLPDTLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDTLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGSHHHHHHHH

SEQ ID NO: 219 - protein, 331 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKELRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKELRLRLRRCHRFLPCENKSKAVEQVKNAFNKLQEKGIYKAMSEFDIFINYIEAYMTMKIRNGGSHHHHHHHH

SEQ ID NO: 220 - protein, 331 aa, synthetic construct; identical to SEQ ID NO: 209

SEQ ID NO: 221 - protein, 331 aa, synthetic construct
SPGQGTQSENSCTHFPGNLPDMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGSHHHHHHHH

SEQ ID NO: 222 - protein, 331 aa, synthetic construct
SPGQGTQSENSCTHFPGNLPEMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPEMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGSHHHHHHHH

SEQ ID NO: 223 - protein, 331 aa, synthetic construct; identical to SEQ ID NO: 210

SEQ ID NO: 224 - protein, 331 aa, synthetic construct
SPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGSHHHHHHHH

SEQ ID NO: 225 - protein, 331 aa, synthetic construct
SPGQGTQSENSCTHFPGNLPDDLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDDLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGSHHHHHHHH

SEQ ID NO: 226 - protein, 331 aa, synthetic construct
SPGQGTQSENSCTHFPGNLPDWLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPDWLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGSHHHHHHHH

SEQ ID NO: 227 - protein, 331 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKCLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKCLRLRLRRCHRFLPCENKSKAVEQVKNAFNKLQEKGIYKAMSEFDIFINYIEAYMTMKIRNGGSHHHHHHHH